SEQUENCE LISTING (375 sequences)

SEQ ID NO: 1 - RNA, 21 nt, synthetic construct
  source 1..21: mol_type = other RNA
ccagggagactgctatttcat

SEQ ID NO: 2 - RNA, 21 nt, synthetic construct
  source 1..21: mol_type = other RNA
gtctcgttgaaatcctttgat

SEQ ID NO: 3 - RNA, 21 nt, synthetic construct
  source 1..21: mol_type = other RNA
ttgaaagaggagccctatttc

SEQ ID NO: 4 - RNA, 21 nt, synthetic construct
  source 1..21: mol_type = other RNA
tcacgaattgcccgaacatta

SEQ ID NO: 5 - RNA, 21 nt, synthetic construct
  source 1..21: mol_type = other RNA
cccattaggtgcccatattta

SEQ ID NO: 6 - RNA, 21 nt, synthetic construct
  source 1..21: mol_type = other RNA
cggttcacaacctcggtttag

SEQ ID NO: 7 - RNA, 21 nt, synthetic construct
  source 1..21: mol_type = other RNA
tcaccaaggagactgctattt

SEQ ID NO: 8 - RNA, 21 nt, synthetic construct
  source 1..21: mol_type = other RNA
caacgtaactttgctgaattt

SEQ ID NO: 9 - RNA, 21 nt, synthetic construct
  source 1..21: mol_type = other RNA
acctgaacaccaggctcttta

SEQ ID NO: 10 - RNA, 21 nt, synthetic construct
  source 1..21: mol_type = other RNA
tctcgcatcctcattggtaat

SEQ ID NO: 11 - RNA, 21 nt, synthetic construct
  source 1..21: mol_type = other RNA
tcgcatggcagcaagctaaat

SEQ ID NO: 12 - RNA, 21 nt, synthetic construct
  source 1..21: mol_type = other RNA
gccagtgaaagggtctaatat

SEQ ID NO: 13 - RNA, 21 nt, synthetic construct
  source 1..21: mol_type = other RNA
cctcctaagtagctgggatta

SEQ ID NO: 14 - RNA, 21 nt, synthetic construct
  source 1..21: mol_type = other RNA
cgcctgtaatcccagcacttt

SEQ ID NO: 15 - RNA, 21 nt, synthetic construct
  source 1..21: mol_type = other RNA
gcctcagtttcctcatctgta

SEQ ID NO: 16 - RNA, 21 nt, synthetic construct
  source 1..21: mol_type = other RNA
caatggcacaatcttggctca

SEQ ID NO: 17 - RNA, 21 nt, synthetic construct
  source 1..21: mol_type = other RNA
gcctgtaatcccagcacttta

SEQ ID NO: 18 - RNA, 21 nt, synthetic construct
  source 1..21: mol_type = other RNA
cccaaagtgctgggattacaa

SEQ ID NO: 19 - RNA, 21 nt, synthetic construct
  source 1..21: mol_type = other RNA
caagtagctgggactacagga

SEQ ID NO: 20 - RNA, 21 nt, synthetic construct
  source 1..21: mol_type = other RNA
cccaaagtgctgggattactt

SEQ ID NO: 21 - RNA, 21 nt, synthetic construct
  source 1..21: mol_type = other RNA
cagcagtggcagctgaaatat

SEQ ID NO: 22 - RNA, 21 nt, synthetic construct
  source 1..21: mol_type = other RNA
ccagtgaaagggtctaatata

SEQ ID NO: 23 - RNA, 21 nt, synthetic construct
  source 1..21: mol_type = other RNA
cagtgaaagggtctaatataa

SEQ ID NO: 24 - RNA, 21 nt, synthetic construct
  source 1..21: mol_type = other RNA
agtgaaagggtctaatataaa

SEQ ID NO: 25 - RNA, 21 nt, synthetic construct
  source 1..21: mol_type = other RNA
cctgaaagaggaaccttattt

SEQ ID NO: 26 - RNA, 21 nt, synthetic construct
  source 1..21: mol_type = other RNA
caacaggaaccttggattata

SEQ ID NO: 27 - RNA, 21 nt, synthetic construct
  source 1..21: mol_type = other RNA
acaggaaccttggattatatt

SEQ ID NO: 28 - RNA, 21 nt, synthetic construct
  source 1..21: mol_type = other RNA
caggaaccttggattatattt

SEQ ID NO: 29 - RNA, 21 nt, synthetic construct
  source 1..21: mol_type = other RNA
agctgtggctacttcaattta

SEQ ID NO: 30 - RNA, 21 nt, synthetic construct
  source 1..21: mol_type = other RNA
gctgtggctacttcaatttaa

SEQ ID NO: 31 - RNA, 21 nt, synthetic construct
  source 1..21: mol_type = other RNA
ctgtggctacttcaatttaaa

SEQ ID NO: 32 - RNA, 21 nt, synthetic construct
  source 1..21: mol_type = other RNA
tgtggctacttcaatttaaat

SEQ ID NO: 33 - RNA, 21 nt, synthetic construct
  source 1..21: mol_type = other RNA
gtggctacttcaatttaaatt

SEQ ID NO: 34 - RNA, 21 nt, synthetic construct
  source 1..21: mol_type = other RNA
attggacaacacttctataaa

SEQ ID NO: 35 - RNA, 21 nt, synthetic construct
  source 1..21: mol_type = other RNA
cacgggaaggagatgttaata

SEQ ID NO: 36 - RNA, 21 nt, synthetic construct
  source 1..21: mol_type = other RNA
acgggaaggagatgttaataa

SEQ ID NO: 37 - RNA, 21 nt, synthetic construct
  source 1..21: mol_type = other RNA
cgggaaggagatgttaataat

SEQ ID NO: 38 - RNA, 21 nt, synthetic construct
  source 1..21: mol_type = other RNA
gggaaggagatgttaataatt

SEQ ID NO: 39 - RNA, 21 nt, synthetic construct
  source 1..21: mol_type = other RNA
ggaaggagatgttaataatta

SEQ ID NO: 40 - RNA, 21 nt, synthetic construct
  source 1..21: mol_type = other RNA
tccctcattcttatgtaataa

SEQ ID NO: 41 - RNA, 21 nt, synthetic construct
  source 1..21: mol_type = other RNA
caagagagagtagggtttaaa

SEQ ID NO: 42 - RNA, 21 nt, synthetic construct
  source 1..21: mol_type = other RNA
cagtttcctcatctgtataat

SEQ ID NO: 43 - RNA, 21 nt, synthetic construct
  source 1..21: mol_type = other RNA
atttccagcacatagttaaat

SEQ ID NO: 44 - RNA, 21 nt, synthetic construct
  source 1..21: mol_type = other RNA
tttccagcacatagttaaata

SEQ ID NO: 45 - RNA, 21 nt, synthetic construct
  source 1..21: mol_type = other RNA
ttccagcacatagttaaatat

SEQ ID NO: 46 - RNA, 21 nt, synthetic construct
  source 1..21: mol_type = other RNA
tgaaggtgcactgagaataaa

SEQ ID NO: 47 - RNA, 21 nt, synthetic construct
  source 1..21: mol_type = other RNA
ttcccatgaatgcagttaata

SEQ ID NO: 48 - RNA, 21 nt, synthetic construct
  source 1..21: mol_type = other RNA
agggtcattacagagattaaa

SEQ ID NO: 49 - RNA, 21 nt, synthetic construct
  source 1..21: mol_type = other RNA
gggtcattacagagattaaat

SEQ ID NO: 50 - RNA, 21 nt, synthetic construct
  source 1..21: mol_type = other RNA
caggagggcgtggtgtttaaa

SEQ ID NO: 51 - RNA, 21 nt, synthetic construct
  source 1..21: mol_type = other RNA
tcccattaggtgcccatattt

SEQ ID NO: 52 - RNA, 21 nt, synthetic construct
  source 1..21: mol_type = other RNA
ccattaggtgcccatatttaa

SEQ ID NO: 53 - RNA, 21 nt, synthetic construct
  source 1..21: mol_type = other RNA
cattaggtgcccatatttaaa

SEQ ID NO: 54 - RNA, 21 nt, synthetic construct
  source 1..21: mol_type = other RNA
cgcatggcagcaagctaaata

SEQ ID NO: 55 - RNA, 21 nt, synthetic construct
  source 1..21: mol_type = other RNA
gcatggcagcaagctaaataa

SEQ ID NO: 56 - RNA, 21 nt, synthetic construct
  source 1..21: mol_type = other RNA
aggtgggcaagacccaataaa

SEQ ID NO: 57 - RNA, 21 nt, synthetic construct
  source 1..21: mol_type = other RNA
agaggagcggtgtggattaaa

SEQ ID NO: 58 - RNA, 21 nt, synthetic construct
  source 1..21: mol_type = other RNA
ctttgttactctgtgattaaa

SEQ ID NO: 59 - RNA, 21 nt, synthetic construct
  source 1..21: mol_type = other RNA
gttctccattctctcttatta

SEQ ID NO: 60 - RNA, 21 nt, synthetic construct
  source 1..21: mol_type = other RNA
ttctccattctctcttattaa

SEQ ID NO: 61 - RNA, 21 nt, synthetic construct
  source 1..21: mol_type = other RNA
cagtgaggtactagctattat

SEQ ID NO: 62 - RNA, 21 nt, synthetic construct
  source 1..21: mol_type = other RNA
agtgaggtactagctattatt

SEQ ID NO: 63 - RNA, 21 nt, synthetic construct
  source 1..21: mol_type = other RNA
gtgaggtactagctattatta

SEQ ID NO: 64 - RNA, 21 nt, synthetic construct
  source 1..21: mol_type = other RNA
tgaggtactagctattattat

SEQ ID NO: 65 - RNA, 21 nt, synthetic construct
  source 1..21: mol_type = other RNA
gaggtactagctattattatt

SEQ ID NO: 66 - RNA, 21 nt, synthetic construct
  source 1..21: mol_type = other RNA
tccagtctttgtttctataat

SEQ ID NO: 67 - RNA, 21 nt, synthetic construct
  source 1..21: mol_type = other RNA
acgccacgcatgtacattatt

SEQ ID NO: 68 - RNA, 21 nt, synthetic construct
  source 1..21: mol_type = other RNA
cgccacgcatgtacattatta

SEQ ID NO: 69 - RNA, 21 nt, synthetic construct
  source 1..21: mol_type = other RNA
gccacgcatgtacattattat

SEQ ID NO: 70 - RNA, 21 nt, synthetic construct
  source 1..21: mol_type = other RNA
ggcttgttcaagaagaatatt

SEQ ID NO: 71 - RNA, 21 nt, synthetic construct
  source 1..21: mol_type = other RNA
ttgtgtcactatcccatatat

SEQ ID NO: 72 - RNA, 21 nt, synthetic construct
  source 1..21: mol_type = other RNA
tgtgtcactatcccatatatt

SEQ ID NO: 73 - RNA, 21 nt, synthetic construct
  source 1..21: mol_type = other RNA
gtgtcactatcccatatatta

SEQ ID NO: 74 - RNA, 21 nt, synthetic construct
  source 1..21: mol_type = other RNA
tgtcactatcccatatattat

SEQ ID NO: 75 - RNA, 21 nt, synthetic construct
  source 1..21: mol_type = other RNA
gccaccacatccggctaattt

SEQ ID NO: 76 - RNA, 21 nt, synthetic construct
  source 1..21: mol_type = other RNA
tgcttggtgttaaagattaaa

SEQ ID NO: 77 - RNA, 21 nt, synthetic construct
  source 1..21: mol_type = other RNA
agtccaagggagagaaattat

SEQ ID NO: 78 - RNA, 21 nt, synthetic construct
  source 1..21: mol_type = other RNA
gtccaagggagagaaattatt

SEQ ID NO: 79 - RNA, 21 nt, synthetic construct
  source 1..21: mol_type = other RNA
tccaagggagagaaattatta

SEQ ID NO: 80 - RNA, 21 nt, synthetic construct
  source 1..21: mol_type = other RNA
ccaagggagagaaattattat

SEQ ID NO: 81 - RNA, 21 nt, synthetic construct
  source 1..21: mol_type = other RNA
caagggagagaaattattatt

SEQ ID NO: 82 - RNA, 21 nt, synthetic construct
  source 1..21: mol_type = other RNA
gagaagcaaaccaggtatata

SEQ ID NO: 83 - RNA, 21 nt, synthetic construct
  source 1..21: mol_type = other RNA
gatctgtcagaagccattaaa

SEQ ID NO: 84 - RNA, 21 nt, synthetic construct
  source 1..21: mol_type = other RNA
gactgggtcgggttgttattt

SEQ ID NO: 85 - RNA, 21 nt, synthetic construct
  source 1..21: mol_type = other RNA
tccagatgggagaagttattt

SEQ ID NO: 86 - RNA, 21 nt, synthetic construct
  source 1..21: mol_type = other RNA
ttattagagccactgaaattt

SEQ ID NO: 87 - RNA, 21 nt, synthetic construct
  source 1..21: mol_type = other RNA
tattagagccactgaaattta

SEQ ID NO: 88 - RNA, 21 nt, synthetic construct
  source 1..21: mol_type = other RNA
attagagccactgaaatttat

SEQ ID NO: 89 - RNA, 21 nt, synthetic construct
  source 1..21: mol_type = other RNA
gctcatcggtttatgtattat

SEQ ID NO: 90 - RNA, 21 nt, synthetic construct
  source 1..21: mol_type = other RNA
acctgtgatcgtgcctattaa

SEQ ID NO: 91 - RNA, 21 nt, synthetic construct
  source 1..21: mol_type = other RNA
tcgtgcctattaagcaattaa

SEQ ID NO: 92 - RNA, 21 nt, synthetic construct
  source 1..21: mol_type = other RNA
cggaacagtgcgaagattatt

SEQ ID NO: 93 - RNA, 21 nt, synthetic construct
  source 1..21: mol_type = other RNA
gactaaagggtgtcctattta

SEQ ID NO: 94 - RNA, 21 nt, synthetic construct
  source 1..21: mol_type = other RNA
actaaagggtgtcctatttat

SEQ ID NO: 95 - RNA, 21 nt, synthetic construct
  source 1..21: mol_type = other RNA
ctaaagggtgtcctatttata

SEQ ID NO: 96 - RNA, 21 nt, synthetic construct
  source 1..21: mol_type = other RNA
taaagggtgtcctatttataa

SEQ ID NO: 97 - RNA, 21 nt, synthetic construct
  source 1..21: mol_type = other RNA
cttcatactctgtgcttatta

SEQ ID NO: 98 - RNA, 21 nt, synthetic construct
  source 1..21: mol_type = other RNA
ggctcttagagtttctattaa

SEQ ID NO: 99 - RNA, 21 nt, synthetic construct
  source 1..21: mol_type = other RNA
agcagtggcagctgaaatatc

SEQ ID NO: 100 - RNA, 21 nt, synthetic construct
  source 1..21: mol_type = other RNA
gctgaaatatcctaaactaat

SEQ ID NO: 101 - RNA, 21 nt, synthetic construct
  source 1..21: mol_type = other RNA
gtatctgaggagctccataaa

SEQ ID NO: 102 - RNA, 21 nt, synthetic construct
  source 1..21: mol_type = other RNA
tatctgaggagctccataaag

SEQ ID NO: 103 - RNA, 21 nt, synthetic construct
  source 1..21: mol_type = other RNA
tggccagtgaaagggtctaat

SEQ ID NO: 104 - RNA, 21 nt, synthetic construct
  source 1..21: mol_type = other RNA
ggccagtgaaagggtctaata

SEQ ID NO: 105 - RNA, 21 nt, synthetic construct
  source 1..21: mol_type = other RNA
attgtgtatgtctgcagattt

SEQ ID NO: 106 - RNA, 21 nt, synthetic construct
  source 1..21: mol_type = other RNA
gacaagatgaagtggacatta

SEQ ID NO: 107 - RNA, 21 nt, synthetic construct
  source 1..21: mol_type = other RNA
acaagatgaagtggacattaa

SEQ ID NO: 108 - RNA, 21 nt, synthetic construct
  source 1..21: mol_type = other RNA
caagatgaagtggacattaag

SEQ ID NO: 109 - RNA, 21 nt, synthetic construct
  source 1..21: mol_type = other RNA
acctgaaagaggaaccttatt

SEQ ID NO: 110 - RNA, 21 nt, synthetic construct
  source 1..21: mol_type = other RNA
gcggtggcagtgtacagttat

SEQ ID NO: 111 - RNA, 21 nt, synthetic construct
  source 1..21: mol_type = other RNA
cggtggcagtgtacagttata

SEQ ID NO: 112 - RNA, 21 nt, synthetic construct
  source 1..21: mol_type = other RNA
ggtggcagtgtacagttatag

SEQ ID NO: 113 - RNA, 21 nt, synthetic construct
  source 1..21: mol_type = other RNA
cgaaggcagggatcctgatat

SEQ ID NO: 114 - RNA, 21 nt, synthetic construct
  source 1..21: mol_type = other RNA
gaaggcagggatcctgatatt

SEQ ID NO: 115 - RNA, 21 nt, synthetic construct
  source 1..21: mol_type = other RNA
aggcagggatcctgatatttg

SEQ ID NO: 116 - RNA, 21 nt, synthetic construct
  source 1..21: mol_type = other RNA
caccaaggagactgctatttc

SEQ ID NO: 117 - RNA, 21 nt, synthetic construct
  source 1..21: mol_type = other RNA
tcaacaggaaccttggattat

SEQ ID NO: 118 - RNA, 21 nt, synthetic construct
  source 1..21: mol_type = other RNA
atgatgatgtctctttgaaat

SEQ ID NO: 119 - RNA, 21 nt, synthetic construct
  source 1..21: mol_type = other RNA
tttgaaacaaggagaagaaat

SEQ ID NO: 120 - RNA, 21 nt, synthetic construct
  source 1..21: mol_type = other RNA
ttgaaacaaggagaagaaatt

SEQ ID NO: 121 - RNA, 21 nt, synthetic construct
  source 1..21: mol_type = other RNA
tgaaacaaggagaagaaattc

SEQ ID NO: 122 - RNA, 21 nt, synthetic construct
  source 1..21: mol_type = other RNA
acaaggagaagaaattcataa

SEQ ID NO: 123 - RNA, 21 nt, synthetic construct
  source 1..21: mol_type = other RNA
caaggagaagaaattcataat

SEQ ID NO: 124 - RNA, 21 nt, synthetic construct
  source 1..21: mol_type = other RNA
tgctgtgcttcatgaagttaa

SEQ ID NO: 125 - RNA, 21 nt, synthetic construct
  source 1..21: mol_type = other RNA
gctgtgcttcatgaagttaaa

SEQ ID NO: 126 - RNA, 21 nt, synthetic construct
  source 1..21: mol_type = other RNA
cgtggaacaaaggaatgaaat

SEQ ID NO: 127 - RNA, 21 nt, synthetic construct
  source 1..21: mol_type = other RNA
ccctgtgttatagtctgattt

SEQ ID NO: 128 - RNA, 21 nt, synthetic construct
  source 1..21: mol_type = other RNA
tatagtctgatttggtgttaa

SEQ ID NO: 129 - RNA, 21 nt, synthetic construct
  source 1..21: mol_type = other RNA
atagtctgatttggtgttaaa

SEQ ID NO: 130 - RNA, 21 nt, synthetic construct
  source 1..21: mol_type = other RNA
tagtctgatttggtgttaaac

SEQ ID NO: 131 - RNA, 21 nt, synthetic construct
  source 1..21: mol_type = other RNA
acccatagtgctgtccaatat

SEQ ID NO: 132 - RNA, 21 nt, synthetic construct
  source 1..21: mol_type = other RNA
cccatagtgctgtccaatatg

SEQ ID NO: 133 - RNA, 21 nt, synthetic construct
  source 1..21: mol_type = other RNA
ctagctgtggctacttcaatt

SEQ ID NO: 134 - RNA, 21 nt, synthetic construct
  source 1..21: mol_type = other RNA
tagctgtggctacttcaattt

SEQ ID NO: 135 - RNA, 21 nt, synthetic construct
  source 1..21: mol_type = other RNA
tggctacttcaatttaaattc

SEQ ID NO: 136 - RNA, 21 nt, synthetic construct
  source 1..21: mol_type = other RNA
gtggctagtgactgctgtatt

SEQ ID NO: 137 - RNA, 21 nt, synthetic construct
  source 1..21: mol_type = other RNA
tgtattggacggtacagatat

SEQ ID NO: 138 - RNA, 21 nt, synthetic construct
  source 1..21: mol_type = other RNA
cggtacagatatggaacattt

SEQ ID NO: 139 - RNA, 21 nt, synthetic construct
  source 1..21: mol_type = other RNA
ctattggacaacacttctata

SEQ ID NO: 140 - RNA, 21 nt, synthetic construct
  source 1..21: mol_type = other RNA
tattggacaacacttctataa

SEQ ID NO: 141 - RNA, 21 nt, synthetic construct
  source 1..21: mol_type = other RNA
cccaaaggcaaagaaactaaa

SEQ ID NO: 142 - RNA, 21 nt, synthetic construct
  source 1..21: mol_type = other RNA
tctaagacaaacctaagtaaa

SEQ ID NO: 143 - RNA, 21 nt, synthetic construct
  source 1..21: mol_type = other RNA
cacacgggaaggagatgttaa

SEQ ID NO: 144 - RNA, 21 nt, synthetic construct
  source 1..21: mol_type = other RNA
acacgggaaggagatgttaat

SEQ ID NO: 145 - RNA, 21 nt, synthetic construct
  source 1..21: mol_type = other RNA
ggatttcaccagcatagtata

SEQ ID NO: 146 - RNA, 21 nt, synthetic construct
  source 1..21: mol_type = other RNA
ctgtaagtccctcattcttat

SEQ ID NO: 147 - RNA, 21 nt, synthetic construct
  source 1..21: mol_type = other RNA
agtccctcattcttatgtaat

SEQ ID NO: 148 - RNA, 21 nt, synthetic construct
  source 1..21: mol_type = other RNA
gtccctcattcttatgtaata

SEQ ID NO: 149 - RNA, 21 nt, synthetic construct
  source 1..21: mol_type = other RNA
ccctcattcttatgtaataac

SEQ ID NO: 150 - RNA, 21 nt, synthetic construct
  source 1..21: mol_type = other RNA
acaagagagagtagggtttaa

SEQ ID NO: 151 - RNA, 21 nt, synthetic construct
  source 1..21: mol_type = other RNA
tcgtgtcctttggcatgttaa

SEQ ID NO: 152 - RNA, 21 nt, synthetic construct
  source 1..21: mol_type = other RNA
ctcagtttcctcatctgtata

SEQ ID NO: 153 - RNA, 21 nt, synthetic construct
  source 1..21: mol_type = other RNA
tcagtttcctcatctgtataa

SEQ ID NO: 154 - RNA, 21 nt, synthetic construct
  source 1..21: mol_type = other RNA
agtttcctcatctgtataatg

SEQ ID NO: 155 - RNA, 21 nt, synthetic construct
  source 1..21: mol_type = other RNA
ccagtcctaaggtgaacatta

SEQ ID NO: 156 - RNA, 21 nt, synthetic construct
  source 1..21: mol_type = other RNA
cagtcctaaggtgaacattaa

SEQ ID NO: 157 - RNA, 21 nt, synthetic construct
  source 1..21: mol_type = other RNA
agtcctaaggtgaacattaag

SEQ ID NO: 158 - RNA, 21 nt, synthetic construct
  source 1..21: mol_type = other RNA
agttacagacttagaacaatt

SEQ ID NO: 159 - RNA, 21 nt, synthetic construct
  source 1..21: mol_type = other RNA
gttacagacttagaacaattt

SEQ ID NO: 160 - RNA, 21 nt, synthetic construct
  source 1..21: mol_type = other RNA
ttacagacttagaacaatttc

SEQ ID NO: 161 - RNA, 21 nt, synthetic construct
  source 1..21: mol_type = other RNA
caatttccagcacatagttaa

SEQ ID NO: 162 - RNA, 21 nt, synthetic construct
  source 1..21: mol_type = other RNA
tccagcacatagttaaatatc

SEQ ID NO: 163 - RNA, 21 nt, synthetic construct
  source 1..21: mol_type = other RNA
agttaaatatccaggaaattc

SEQ ID NO: 164 - RNA, 21 nt, synthetic construct
  source 1..21: mol_type = other RNA
aggaaattctggtactgttat

SEQ ID NO: 165 - RNA, 21 nt, synthetic construct
  source 1..21: mol_type = other RNA
tttgtcttgtgatgccattaa

SEQ ID NO: 166 - RNA, 21 nt, synthetic construct
  source 1..21: mol_type = other RNA
ttgtcttgtgatgccattaac

SEQ ID NO: 167 - RNA, 21 nt, synthetic construct
  source 1..21: mol_type = other RNA
cccattggtgtcccaagaaat

SEQ ID NO: 168 - RNA, 21 nt, synthetic construct
  source 1..21: mol_type = other RNA
tcccaagaaatcgtgagaata

SEQ ID NO: 169 - RNA, 21 nt, synthetic construct
  source 1..21: mol_type = other RNA
attgaaggtgcactgagaata

SEQ ID NO: 170 - RNA, 21 nt, synthetic construct
  source 1..21: mol_type = other RNA
ttgaaggtgcactgagaataa

SEQ ID NO: 171 - RNA, 21 nt, synthetic construct
  source 1..21: mol_type = other RNA
gaaggtgcactgagaataaac

SEQ ID NO: 172 - RNA, 21 nt, synthetic construct
  source 1..21: mol_type = other RNA
attcccatgaatgcagttaat

SEQ ID NO: 173 - RNA, 21 nt, synthetic construct
  source 1..21: mol_type = other RNA
tcccatgaatgcagttaatag

SEQ ID NO: 174 - RNA, 21 nt, synthetic construct
  source 1..21: mol_type = other RNA
gcagttaatagccacagaaat

SEQ ID NO: 175 - RNA, 21 nt, synthetic construct
  source 1..21: mol_type = other RNA
agccacagaaatgtcacatta

SEQ ID NO: 176 - RNA, 21 nt, synthetic construct
  source 1..21: mol_type = other RNA
gccacagaaatgtcacattaa

SEQ ID NO: 177 - RNA, 21 nt, synthetic construct
  source 1..21: mol_type = other RNA
ccacagaaatgtcacattaag

SEQ ID NO: 178 - RNA, 21 nt, synthetic construct
  source 1..21: mol_type = other RNA
cagtgtctcacagggtcatta

SEQ ID NO: 179 - RNA, 21 nt, synthetic construct
  source 1..21: mol_type = other RNA
acagggtcattacagagatta

SEQ ID NO: 180 - RNA, 21 nt, synthetic construct
  source 1..21: mol_type = other RNA
cagggtcattacagagattaa

SEQ ID NO: 181 - RNA, 21 nt, synthetic construct
  source 1..21: mol_type = other RNA
ggtcattacagagattaaatg

SEQ ID NO: 182 - RNA, 21 nt, synthetic construct
  source 1..21: mol_type = other RNA
atcccattaggtgcccatatt

SEQ ID NO: 183 - RNA, 21 nt, synthetic construct
  source 1..21: mol_type = other RNA
tgcctcccaggttcaagtaat

SEQ ID NO: 184 - RNA, 21 nt, synthetic construct
  source 1..21: mol_type = other RNA
gcctcccaggttcaagtaatt

SEQ ID NO: 185 - RNA, 21 nt, synthetic construct
  source 1..21: mol_type = other RNA
cctcccaggttcaagtaattc

SEQ ID NO: 186 - RNA, 21 nt, synthetic construct
  source 1..21: mol_type = other RNA
catggcagcaagctaaataag

SEQ ID NO: 187 - RNA, 21 nt, synthetic construct
  source 1..21: mol_type = other RNA
caatatatgtgtgtgactatt

SEQ ID NO: 188 - RNA, 21 nt, synthetic construct
  source 1..21: mol_type = other RNA
gtgtgactattgaactctatt

SEQ ID NO: 189 - RNA, 21 nt, synthetic construct
  source 1..21: mol_type = other RNA
tcgtagactgcttgtactaat

SEQ ID NO: 190 - RNA, 21 nt, synthetic construct
  source 1..21: mol_type = other RNA
ctgcttgtactaatgtcattt

SEQ ID NO: 191 - RNA, 21 nt, synthetic construct
  source 1..21: mol_type = other RNA
ctaatgtcatttgcatcataa

SEQ ID NO: 192 - RNA, 21 nt, synthetic construct
  source 1..21: mol_type = other RNA
agataagaaaccgagagattt

SEQ ID NO: 193 - RNA, 21 nt, synthetic construct
  source 1..21: mol_type = other RNA
gtgcaacagggttggagaaat

SEQ ID NO: 194 - RNA, 21 nt, synthetic construct
  source 1..21: mol_type = other RNA
gttggagaaatcactacttta

SEQ ID NO: 195 - RNA, 21 nt, synthetic construct
  source 1..21: mol_type = other RNA
ttggagaaatcactactttaa

SEQ ID NO: 196 - RNA, 21 nt, synthetic construct
  source 1..21: mol_type = other RNA
tggagaaatcactactttaag

SEQ ID NO: 197 - RNA, 21 nt, synthetic construct
  source 1..21: mol_type = other RNA
ccgaaaggtgagatcacattt

SEQ ID NO: 198 - RNA, 21 nt, synthetic construct
  source 1..21: mol_type = other RNA
tgagatcacatttgctctaaa

SEQ ID NO: 199 - RNA, 21 nt, synthetic construct
  source 1..21: mol_type = other RNA
tcacatttgctctaaagaaat

SEQ ID NO: 200 - RNA, 21 nt, synthetic construct
  source 1..21: mol_type = other RNA
gtggtggtgtgtgcctgtaat

SEQ ID NO: 201 - RNA, 21 nt, synthetic construct
  source 1..21: mol_type = other RNA
ggagcgagagaagaaagaaat

SEQ ID NO: 202 - RNA, 21 nt, synthetic construct
  source 1..21: mol_type = other RNA
gaggtgggcaagacccaataa

SEQ ID NO: 203 - RNA, 21 nt, synthetic construct
  source 1..21: mol_type = other RNA
tggaagcacaggcccagaaat

SEQ ID NO: 204 - RNA, 21 nt, synthetic construct
  source 1..21: mol_type = other RNA
acagtaagcagcaaatcaata

SEQ ID NO: 205 - RNA, 21 nt, synthetic construct
  source 1..21: mol_type = other RNA
cagtaagcagcaaatcaataa

SEQ ID NO: 206 - RNA, 21 nt, synthetic construct
  source 1..21: mol_type = other RNA
agtaagcagcaaatcaataac

SEQ ID NO: 207 - RNA, 21 nt, synthetic construct
  source 1..21: mol_type = other RNA
ggaagtaacttggtgagatat

SEQ ID NO: 208 - RNA, 21 nt, synthetic construct
  source 1..21: mol_type = other RNA
gaagtaacttggtgagatatt

SEQ ID NO: 209 - RNA, 21 nt, synthetic construct
  source 1..21: mol_type = other RNA
tgagagagcaaacagcttaat

SEQ ID NO: 210 - RNA, 21 nt, synthetic construct
  source 1..21: mol_type = other RNA
gagagagcaaacagcttaatc

SEQ ID NO: 211 - RNA, 21 nt, synthetic construct
  source 1..21: mol_type = other RNA
gcctacagagttcactctaaa

SEQ ID NO: 212 - RNA, 21 nt, synthetic construct
  source 1..21: mol_type = other RNA
cacagagctttctagacataa

SEQ ID NO: 213 - RNA, 21 nt, synthetic construct
  source 1..21: mol_type = other RNA
acagagctttctagacataaa

SEQ ID NO: 214 - RNA, 21 nt, synthetic construct
  source 1..21: mol_type = other RNA
ggcatcattggtgacacaatt

SEQ ID NO: 215 - RNA, 21 nt, synthetic construct
  source 1..21: mol_type = other RNA
gcatcattggtgacacaattt

SEQ ID NO: 216 - RNA, 21 nt, synthetic construct
  source 1..21: mol_type = other RNA
gagtccataggacccgtttaa

SEQ ID NO: 217 - RNA, 21 nt, synthetic construct
  source 1..21: mol_type = other RNA
agtccataggacccgtttaac

SEQ ID NO: 218 - RNA, 21 nt, synthetic construct
  source 1..21: mol_type = other RNA
ttcgagggaaggaaggtattt

SEQ ID NO: 219 - RNA, 21 nt, synthetic construct
  source 1..21: mol_type = other RNA
tgctctagttagggctgattt

SEQ ID NO: 220 - RNA, 21 nt, synthetic construct
  source 1..21: mol_type = other RNA
ctgggcaaattgcatacattt

SEQ ID NO: 221 - RNA, 21 nt, synthetic construct
  source 1..21: mol_type = other RNA
gagaggagcggtgtggattaa

SEQ ID NO: 222 - RNA, 21 nt, synthetic construct
  source 1..21: mol_type = other RNA
cgaagtggtaatgccgtttat

SEQ ID NO: 223 - RNA, 21 nt, synthetic construct
  source 1..21: mol_type = other RNA
gaagtggtaatgccgtttatc

SEQ ID NO: 224 - RNA, 21 nt, synthetic construct
  source 1..21: mol_type = other RNA
gcctttgttactctgtgatta

SEQ ID NO: 225 - RNA, 21 nt, synthetic construct
  source 1..21: mol_type = other RNA
cctttgttactctgtgattaa

SEQ ID NO: 226 - RNA, 21 nt, synthetic construct
  source 1..21: mol_type = other RNA
ttgttactctgtgattaaatg

SEQ ID NO: 227 - RNA, 21 nt, synthetic construct
  source 1..21: mol_type = other RNA
ctctgtgattaaatggtattt

SEQ ID NO: 228 - RNA, 21 nt, synthetic construct
  source 1..21: mol_type = other RNA
taggtatcgcggtggtgaaat

SEQ ID NO: 229 - RNA, 21 nt, synthetic construct
  source 1..21: mol_type = other RNA
atcgcggtggtgaaatcaatt

SEQ ID NO: 230 - RNA, 21 nt, synthetic construct
  source 1..21: mol_type = other RNA
tcgcggtggtgaaatcaattt

SEQ ID NO: 231 - RNA, 21 nt, synthetic construct
  source 1..21: mol_type = other RNA
cgcggtggtgaaatcaatttg

SEQ ID NO: 232 - RNA, 21 nt, synthetic construct
  source 1..21: mol_type = other RNA
atcaatttgacaaccataaag

SEQ ID NO: 233 - RNA, 21 nt, synthetic construct
  source 1..21: mol_type = other RNA
tggttctccattctctcttat

SEQ ID NO: 234 - RNA, 21 nt, synthetic construct
  source 1..21: mol_type = other RNA
ggttctccattctctcttatt

SEQ ID NO: 235 - RNA, 21 nt, synthetic construct
  source 1..21: mol_type = other RNA
tctccattctctcttattaac

SEQ ID NO: 236 - RNA, 21 nt, synthetic construct
  source 1..21: mol_type = other RNA
gctcccaggacagtcctaatt

SEQ ID NO: 237 - RNA, 21 nt, synthetic construct
  source 1..21: mol_type = other RNA
ctcccaggacagtcctaattc

SEQ ID NO: 238 - RNA, 21 nt, synthetic construct
  source 1..21: mol_type = other RNA
ccgctgtcattcatcagattt

SEQ ID NO: 239 - RNA, 21 nt, synthetic construct
  source 1..21: mol_type = other RNA
ttcaaacctttgttgtcttat

SEQ ID NO: 240 - RNA, 21 nt, synthetic construct
  source 1..21: mol_type = other RNA
ggttgttgtgaaggttcaata

SEQ ID NO: 241 - RNA, 21 nt, synthetic construct
  source 1..21: mol_type = other RNA
gttgttgtgaaggttcaataa

SEQ ID NO: 242 - RNA, 21 nt, synthetic construct
  source 1..21: mol_type = other RNA
ttgttgtgaaggttcaataag

SEQ ID NO: 243 - RNA, 21 nt, synthetic construct
  source 1..21: mol_type = other RNA
tgtgaaggttcaataagttaa

SEQ ID NO: 244 - RNA, 21 nt, synthetic construct
  source 1..21: mol_type = other RNA
gtgaaggttcaataagttaat

SEQ ID NO: 245 - RNA, 21 nt, synthetic construct
  source 1..21: mol_type = other RNA
tgaaggttcaataagttaatg

SEQ ID NO: 246 - RNA, 21 nt, synthetic construct
  source 1..21: mol_type = other RNA
ctcccacagtacctgacatat

SEQ ID NO: 247 - RNA, 21 nt, synthetic construct
  source 1..21: mol_type = other RNA
tcccacagtacctgacatata

SEQ ID NO: 248 - RNA, 21 nt, synthetic construct
  source 1..21: mol_type = other RNA
cccacagtacctgacatatac

SEQ ID NO: 249 - RNA, 21 nt, synthetic construct
  source 1..21: mol_type = other RNA
tcagtgaggtactagctatta

SEQ ID NO: 250 - RNA, 21 nt, synthetic construct
  source 1..21: mol_type = other RNA
tgactgggccaaatcacttaa

SEQ ID NO: 251 - RNA, 21 nt, synthetic construct
  source 1..21: mol_type = other RNA
gctccagtctttgtttctata

SEQ ID NO: 252 - RNA, 21 nt, synthetic construct
  source 1..21: mol_type = other RNA
ctccagtctttgtttctataa

SEQ ID NO: 253 - RNA, 21 nt, synthetic construct
  source 1..21: mol_type = other RNA
ccagtctttgtttctataatg

SEQ ID NO: 254 - RNA, 21 nt, synthetic construct
  source 1..21: mol_type = other RNA
ccctacgatcagccatgttaa

SEQ ID NO: 255 - RNA, 21 nt, synthetic construct
  source 1..21: mol_type = other RNA
gccatgttaagtgattcatat

SEQ ID NO: 256 - RNA, 21 nt, synthetic construct
  source 1..21: mol_type = other RNA
catatcatgtctggtacttaa

SEQ ID NO: 257 - RNA, 21 nt, synthetic construct
  source 1..21: mol_type = other RNA
atatcatgtctggtacttaaa

SEQ ID NO: 258 - RNA, 21 nt, synthetic construct
  source 1..21: mol_type = other RNA
tcacgccacgcatgtacatta

SEQ ID NO: 259 - RNA, 21 nt, synthetic construct
  source 1..21: mol_type = other RNA
cacgccacgcatgtacattat

SEQ ID NO: 260 - RNA, 21 nt, synthetic construct
  source 1..21: mol_type = other RNA
ccacgcatgtacattattatg

SEQ ID NO: 261 - RNA, 21 nt, synthetic construct
  source 1..21: mol_type = other RNA
gtggcttgttcaagaagaata

SEQ ID NO: 262 - RNA, 21 nt, synthetic construct
  source 1..21: mol_type = other RNA
tggcttgttcaagaagaatat

SEQ ID NO: 263 - RNA, 21 nt, synthetic construct
  source 1..21: mol_type = other RNA
gcttgttcaagaagaatattg

SEQ ID NO: 264 - RNA, 58 nt, synthetic construct
  source 1..58: mol_type = other RNA
ccggccagtgaaagggtctaatatactcgagtatattagaccctttcactggtttttg

SEQ ID NO: 265 - RNA, 58 nt, synthetic construct
  source 1..58: mol_type = other RNA
ccggcctgaaagaggaaccttatttctcgagaaataaggttcctctttcaggtttttg

SEQ ID NO: 266 - RNA, 58 nt, synthetic construct
  source 1..58: mol_type = other RNA
ccggcaacaggaaccttggattatactcgagtataatccaaggttcctgttgtttttg

SEQ ID NO: 267 - RNA, 58 nt, synthetic construct
  source 1..58: mol_type = other RNA
ccggagctgtggctacttcaatttactcgagtaaattgaagtagccacagcttttttg

SEQ ID NO: 268 - RNA, 58 nt, synthetic construct
  source 1..58: mol_type = other RNA
ccggattggacaacacttctataaactcgagtttatagaagtgttgtccaattttttg

SEQ ID NO: 269 - RNA, 58 nt, synthetic construct
  source 1..58: mol_type = other RNA
ccggcacgggaaggagatgttaatactcgagtattaacatctccttcccgtgtttttg

SEQ ID NO: 270 - RNA, 58 nt, synthetic construct
  source 1..58: mol_type = other RNA
ccggtccctcattcttatgtaataactcgagttattacataagaatgagggatttttg

SEQ ID NO: 271 - RNA, 58 nt, synthetic construct
  source 1..58: mol_type = other RNA
ccggatttccagcacatagttaaatctcgagatttaactatgtgctggaaattttttg

SEQ ID NO: 272 - RNA, 58 nt, synthetic construct
  source 1..58: mol_type = other RNA
ccggtgaaggtgcactgagaataaactcgagtttattctcagtgcaccttcatttttg

SEQ ID NO: 273 - RNA, 58 nt, synthetic construct
  source 1..58: mol_type = other RNA
ccggttcccatgaatgcagttaatactcgagtattaactgcattcatgggaatttttg

SEQ ID NO: 274 - RNA, 21 nt, synthetic construct
  source 1..21: mol_type = other RNA
cgtcagtatcttgggcaagtt

SEQ ID NO: 275 - RNA, 21 nt, synthetic construct
  source 1..21: mol_type = other RNA
gccaaggaacaatccattgtt

SEQ ID NO: 276 - RNA, 58 nt, synthetic construct
  source 1..58: mol_type = other RNA
ccggcgtcagtatcttgggcaagttctcgagaacttgcccaagatactgacgtttttg

SEQ ID NO: 277 - RNA, 58 nt, synthetic construct
  source 1..58: mol_type = other RNA
ccgggccaaggaacaatccattgttctcgagaacaatggattgttccttggctttttg

SEQ ID NO: 278 - RNA, 21 nt, synthetic construct
  source 1..21: mol_type = other RNA
gccgtggacgagaaagaaata

SEQ ID NO: 279 - RNA, 21 nt, synthetic construct
  source 1..21: mol_type = other RNA
gctaatgttgacattgactta

SEQ ID NO: 280 - RNA, 57 nt, synthetic construct
  source 1..57: mol_type = other RNA
ccgggccgtggacgagaaagaaatactcgagtatttctttctcgtccacggcttttt

SEQ ID NO: 281 - RNA, 57 nt, synthetic construct
  source 1..57: mol_type = other RNA
ccgggctaatgttgacattgacttactcgagtaagtcaatgtcaacattagcttttt

SEQ ID NO: 282 - DNA, 20 bp, synthetic construct
  source 1..20: mol_type = other DNA
aatcccatcaccatcttcca

SEQ ID NO: 283 - DNA, 19 bp, synthetic construct
  source 1..19: mol_type = other DNA
tggctccacgacgtactca

SEQ ID NO: 284 - DNA, 20 bp, synthetic construct
  source 1..20: mol_type = other DNA
gtgtcggaggtgattccagt

SEQ ID NO: 285 - DNA, 20 bp, synthetic construct
  source 1..20: mol_type = other DNA
ctgcgcatctcatcatctgt

SEQ ID NO: 286 - DNA, 21 bp, synthetic construct
  source 1..21: mol_type = other DNA
tggaccttggaagagtgtgtt

SEQ ID NO: 287 - DNA, 23 bp, synthetic construct
  source 1..23: mol_type = other DNA
tgccaatttcaggttcttctgtg

SEQ ID NO: 288 - DNA, 20 bp, synthetic construct
  source 1..20: mol_type = other DNA
tcctgagtggcagaagaggt

SEQ ID NO: 289 - DNA, 20 bp, synthetic construct
  source 1..20: mol_type = other DNA
caggtactgtgggaggcatt

SEQ ID NO: 290 - DNA, 18 bp, synthetic construct
  source 1..18: mol_type = other DNA
tcgatcgcttcaccccct

SEQ ID NO: 291 - DNA, 25 bp, synthetic construct
  source 1..25: mol_type = other DNA
ctacttcatcaaatcttctttgcgt

SEQ ID NO: 292 - DNA, 20 bp, synthetic construct
  source 1..20: mol_type = other DNA
atgctcagcagcacaacata

SEQ ID NO: 293 - DNA, 20 bp, synthetic construct
  source 1..20: mol_type = other DNA
tccagttacccgacactgat

SEQ ID NO: 294 - DNA, 20 bp, synthetic construct
  source 1..20: mol_type = other DNA
tttcaacgccgactacgagg

SEQ ID NO: 295 - DNA, 20 bp, synthetic construct
  source 1..20: mol_type = other DNA
ctgcgcaaaagtcctgtgtg

SEQ ID NO: 296 - DNA, 20 bp, synthetic construct
  source 1..20: mol_type = other DNA
agctacctccctggttgtga

SEQ ID NO: 297 - DNA, 20 bp, synthetic construct
  source 1..20: mol_type = other DNA
gtggagggaggtctggttct

SEQ ID NO: 298 - DNA, 20 bp, synthetic construct
  source 1..20: mol_type = other DNA
ggaccctaacccagcatgag

SEQ ID NO: 299 - DNA, 22 bp, synthetic construct
  source 1..22: mol_type = other DNA
ggctgaagatgcagtgtcctta

SEQ ID NO: 300 - DNA, 21 bp, synthetic construct
  source 1..21: mol_type = other DNA
tctggccacagatgaggtaca

SEQ ID NO: 301 - DNA, 20 bp, synthetic construct
  source 1..20: mol_type = other DNA
caacattcccacactccagc

SEQ ID NO: 302 - DNA, 21 bp, synthetic construct
  source 1..21: mol_type = other DNA
ctggctgaattctgagggact

SEQ ID NO: 303 - DNA, 20 bp, synthetic construct
  source 1..20: mol_type = other DNA
ttctgtggcgcaaggagatt

SEQ ID NO: 304 - DNA, 20 bp, synthetic construct
  source 1..20: mol_type = other DNA
agatggccaagcccaagaaa

SEQ ID NO: 305 - DNA, 16 bp, synthetic construct
  source 1..16: mol_type = other DNA
ggtcacacggggctgc

SEQ ID NO: 306 - DNA, 21 bp, synthetic construct
  source 1..21: mol_type = other DNA
tttccaccaccaagtctctgc

SEQ ID NO: 307 - DNA, 21 bp, synthetic construct
  source 1..21: mol_type = other DNA
ccgaggcatgaaaggctagta

SEQ ID NO: 308 - DNA, 19 bp, synthetic construct
  source 1..19: mol_type = other DNA
tgcaaagcacaccacaagc

SEQ ID NO: 309 - DNA, 21 bp, synthetic construct
  source 1..21: mol_type = other DNA
gtgtgggaacgtgaactgaag

SEQ ID NO: 310 - DNA, 21 bp, synthetic construct
  source 1..21: mol_type = other DNA
gctcttcagcagtcatgtcct

SEQ ID NO: 311 - DNA, 20 bp, synthetic construct
  source 1..20: mol_type = other DNA
cccagacgagcctttgagaa

SEQ ID NO: 312 - DNA, 21 bp, synthetic construct
  source 1..21: mol_type = other DNA
agacagtaccagtcacctccc

SEQ ID NO: 313 - DNA, 20 bp, synthetic construct
  source 1..20: mol_type = other DNA
tcagcacttccccaggagag

SEQ ID NO: 314 - DNA, 19 bp, synthetic construct
  source 1..19: mol_type = other DNA
ccaaggcccatcagcttca

SEQ ID NO: 315 - DNA, 20 bp, synthetic construct
  source 1..20: mol_type = other DNA
gcagccacaaacttcatgct

SEQ ID NO: 316 - DNA, 21 bp, synthetic construct
  source 1..21: mol_type = other DNA
cgacacccctccactaaagat

SEQ ID NO: 317 - DNA, 21 bp, synthetic construct
  source 1..21: mol_type = other DNA
caaggggtgaagtcctcagtc

SEQ ID NO: 318 - DNA, 22 bp, synthetic construct
  source 1..22: mol_type = other DNA
tcagcttatggaacaagtggct

SEQ ID NO: 319 - DNA, 22 bp, synthetic construct
  source 1..22: mol_type = other DNA
ttctcaatggtgtcactcaggg

SEQ ID NO: 320 - DNA, 21 bp, synthetic construct
  source 1..21: mol_type = other DNA
cacacactctgtgaagtggga

SEQ ID NO: 321 - DNA, 21 bp, synthetic construct
  source 1..21: mol_type = other DNA
ggaatggagatcggagcactt

SEQ ID NO: 322 - DNA, 21 bp, synthetic construct
  source 1..21: mol_type = other DNA
tgtcttcaggaacactgtggg

SEQ ID NO: 323 - DNA, 20 bp, synthetic construct
  source 1..20: mol_type = other DNA
cccatgcttactggggaagt

SEQ ID NO: 324 - DNA, 20 bp, synthetic construct
  source 1..20: mol_type = other DNA
caatggcacaccctgggtaa

SEQ ID NO: 325 - DNA, 20 bp, synthetic construct
  source 1..20: mol_type = other DNA
cagaacaaagccagttccgc

SEQ ID NO: 326 - DNA, 20 bp, synthetic construct
  source 1..20: mol_type = other DNA
ttgggtggtcaaactgtccc

SEQ ID NO: 327 - DNA, 21 bp, synthetic construct
  source 1..21: mol_type = other DNA
tctgtgtggccatcaagactc

SEQ ID NO: 328 - DNA, 21 bp, synthetic construct
  source 1..21: mol_type = other DNA
aggtacagtccttgctccttc

SEQ ID NO: 329 - DNA, 21 bp, synthetic construct
  source 1..21: mol_type = other DNA
tttgggggctatagtgcctct

SEQ ID NO: 330 - DNA, 20 bp, synthetic construct
  source 1..20: mol_type = other DNA
ctgaggcagagcttgaacca

SEQ ID NO: 331 - DNA, 20 bp, synthetic construct
  source 1..20: mol_type = other DNA
tactgaagatggccacaggc

SEQ ID NO: 332 - DNA, 20 bp, synthetic construct
  source 1..20: mol_type = other DNA
tccagatgactaccacggct

SEQ ID NO: 333 - DNA, 22 bp, synthetic construct
  source 1..22: mol_type = other DNA
atgctggagttctctgtccttc

SEQ ID NO: 334 - DNA, 21 bp, synthetic construct
  source 1..21: mol_type = other DNA
atacaccactgaagccaggac

SEQ ID NO: 335 - DNA, 20 bp, synthetic construct
  source 1..20: mol_type = other DNA
agtggaggctgagtcacaat

SEQ ID NO: 336 - DNA, 20 bp, synthetic construct
  source 1..20: mol_type = other DNA
acatgtcccatcttggcagg

SEQ ID NO: 337 - DNA, 20 bp, synthetic construct
  source 1..20: mol_type = other DNA
ggtgctgagctctcaaacct

SEQ ID NO: 338 - DNA, 21 bp, synthetic construct
  source 1..21: mol_type = other DNA
aaagcacgatgtctggtttcc

SEQ ID NO: 339 - DNA, 20 bp, synthetic construct
  source 1..20: mol_type = other DNA
gtggagttcatgcgagtccc

SEQ ID NO: 340 - DNA, 20 bp, synthetic construct
  source 1..20: mol_type = other DNA
aggtgctggggagtccttta

SEQ ID NO: 341 - DNA, 20 bp, synthetic construct
  source 1..20: mol_type = other DNA
gtgtcctctgtgagaaggcg

SEQ ID NO: 342 - DNA, 20 bp, synthetic construct
  source 1..20: mol_type = other DNA
catctgagcaacgggaacac

SEQ ID NO: 343 - DNA, 21 bp, synthetic construct
  source 1..21: mol_type = other DNA
agctggggcaaagtcaacata

SEQ ID NO: 344 - DNA, 23 bp, synthetic construct
  source 1..23: mol_type = other DNA
agcttcttaactctacacacgca

SEQ ID NO: 345 - DNA, 24 bp, synthetic construct
  source 1..24: mol_type = other DNA
agccaggatcatggtgtaataaga

SEQ ID NO: 346 - DNA, 23 bp, synthetic construct
  source 1..23: mol_type = other DNA
tcctagaactttggacttcctgc

SEQ ID NO: 347 - DNA, 24 bp, synthetic construct
  source 1..24: mol_type = other DNA
cacaatgcagaagttagaaaggca

SEQ ID NO: 348 - DNA, 20 bp, synthetic construct
  source 1..20: mol_type = other DNA
tccagctctgcacctactga

SEQ ID NO: 349 - DNA, 20 bp, synthetic construct
  source 1..20: mol_type = other DNA
ggtccagattccgaggttgg

SEQ ID NO: 350 - DNA, 21 bp, synthetic construct
  source 1..21: mol_type = other DNA
aaacccagaaatcagccacct

SEQ ID NO: 351 - DNA, 20 bp, synthetic construct
  source 1..20: mol_type = other DNA
tccagcgtctgcctcattat

SEQ ID NO: 352 - DNA, 22 bp, synthetic construct
  source 1..22: mol_type = other DNA
ggtaaacttccccaacagtgtg

SEQ ID NO: 353 - DNA, 20 bp, synthetic construct
  source 1..20: mol_type = other DNA
tggcaccaccctgaaggaat

SEQ ID NO: 354 - DNA, 21 bp, synthetic construct
  source 1..21: mol_type = other DNA
gaggctatggaagtggtggac

SEQ ID NO: 355 - DNA, 20 bp, synthetic construct
  source 1..20: mol_type = other DNA
tgtcatagctgccactcccg

SEQ ID NO: 356 - DNA, 22 bp, synthetic construct
  source 1..22: mol_type = other DNA
actctgtcaggtccacattgag

SEQ ID NO: 357 - DNA, 21 bp, synthetic construct
  source 1..21: mol_type = other DNA
atcacagcttccttgcgcttt

SEQ ID NO: 358 - DNA, 21 bp, synthetic construct
  source 1..21: mol_type = other DNA
gtccagcactgggtcgatgat

SEQ ID NO: 359 - DNA, 22 bp, synthetic construct
  source 1..22: mol_type = other DNA
ctatgcccgtggtcattacacc

SEQ ID NO: 360 - DNA, 20 bp, synthetic construct
  source 1..20: mol_type = other DNA
gcttggcctgtggagtgatt

SEQ ID NO: 361 - DNA, 21 bp, synthetic construct
  source 1..21: mol_type = other DNA
ctcagaccacacagggaatgt

SEQ ID NO: 362 - DNA, 21 bp, synthetic construct
  source 1..21: mol_type = other DNA
gcaatgtgactgctggaaagg

SEQ ID NO: 363 - DNA, 23 bp, synthetic construct
  source 1..23: mol_type = other DNA
cgatcttcaatggacacattggc

SEQ ID NO: 364 - DNA, 20 bp, synthetic construct
  source 1..20: mol_type = other DNA
gcaggtgccaagcacatttt

SEQ ID NO: 365 - DNA, 20 bp, synthetic construct
  source 1..20: mol_type = other DNA
accccctttgggagaacatt

SEQ ID NO: 366 - DNA, 21 bp, synthetic construct
  source 1..21: mol_type = other DNA
gcgaacgttcttgtcttctgt

SEQ ID NO: 367 - DNA, 20 bp, synthetic construct
  source 1..20: mol_type = other DNA
gaagcgtgcagggttttagc

SEQ ID NO: 368 - DNA, 23 bp, synthetic construct
  source 1..23: mol_type = other DNA
ccactgaacacaaagacctttca

SEQ ID NO: 369 - DNA, 22 bp, synthetic construct
  source 1..22: mol_type = other DNA
gcagtcatggacagtctaaagc

SEQ ID NO: 370 - DNA, 22 bp, synthetic construct
  source 1..22: mol_type = other DNA
ttttctcaattagaggcccacc

SEQ ID NO: 371 - DNA, 25 bp, synthetic construct
  source 1..25: mol_type = other DNA
ctaacttcttctcagtgttgttctg

SEQ ID NO: 372 - DNA, 65 bp, synthetic construct
  source 1..65: mol_type = other DNA
gatccccccagggagactgctatttcatttcaagagatactttatcgtcagagggacctttttgc

SEQ ID NO: 373 - DNA, 65 bp, synthetic construct
  source 1..65: mol_type = other DNA
ggccgcaaaaaggtccctctgacgataaagtatctcttgaaatgaaatagcagtctccctggggg

SEQ ID NO: 374 - DNA, 20 bp, synthetic construct
  source 1..20: mol_type = other DNA
ttcaagcctattcgggtgtc

SEQ ID NO: 375 - DNA, 20 bp, synthetic construct
  source 1..20: mol_type = other DNA
cggggtgcatctaatcttgt

(19) United States
(12) Patent Application Publication
(10) Pub. No.: US 2026/0297583 A1
(43) Pub. Date: Oct. 1, 2026

(54) COMPOSITION AND METHOD FOR DEGENRATIVE MOTOR NEURON TREATMENT

(71) Applicant: Academia Sinica, Taipei (TW)

(72) Inventors: Jun-An CHEN, Taipei (TW); Ya-Ping YEN, Taipei (TW); Ting-Hsiang LUNG, Taipei (TW); Chuan-Che WU, Taipei (TW); Guan-Lin HUANG, Taipei (TW)

(73) Assignee: Academia Sinica, Taipei (TW)

(21) Appl. No.: 19/577,447

(22) Filed: Mar. 25, 2026

Related U.S. Application Data
(60) Provisional application No. 63/777,630, filed on Mar. 25, 2025.

(51) Int. Cl. C12N 15/113; A61K 31/205; A61K 31/42; A61K 31/519; A61K 31/52; A61K 31/714; A61P 25/28; C12N 15/85

(52) U.S. Cl. CPC C12N 15/113; A61K 31/205; A61K 31/42; A61K 31/519; A61K 31/52; A61K 31/714; A61P 25/28; C12N 15/85; C12N 2310/531; C12N 2750/14143

(57) ABSTRACT

The present invention provides a degenerative motor neuron treatment composition comprising one or more therapeutically effective amount of N⁶-methyladenosine (m6A) eraser inhibitor, a histone methylation regulator, or a methyl donor or a combination thereof. The present invention also provides a method of treatment of degenerative motor neuron condition or diseases related to, caused by or having symptoms of degenerative motor neuron.

### INCORPORATION BY REFERENCE OF A SEQUENCE LISTING XML

A Sequence Listing is provided herewith as a Sequence Listing XML, “AS_10NS_Degenerative_motor_neuron_treatment.xml” created on Mar. 11, 2026 and having a size of 332,136 bytes. The contents of the Sequence Listing XML are incorporated by reference herein in their entirety.

### TECHNICAL FIELD OF THE INVENTION

The present invention relates to composition and method for treatment of degenerative motor neuron conditions and diseases related to, caused by or having symptoms of degenerative motor neuron.

### BACKGROUND OF THE INVENTION

In recent years, the intricate world of RNA modifications, often referred to as the “epitranscriptome,” has emerged as a pivotal regulatory axis governing developmental processes ¹. Over 170 types of RNA modifications have been identified since the 1950s. High-throughput sequencing has revealed a diversity of messenger RNA (mRNA) modifications in various organisms ^{2,3}. Among these, m⁶A has received the most attention as it is the most abundant form of mRNA modification in mammals ⁴. Various studies have shown that m⁶A levels can be dynamic and reversible. m⁶A is deposited by the m⁶A methyltransferase complex (termed the “writer”), with METTL3 (methyltransferase-like 3) acting as the catalytically active methyltransferase and METTL14 playing an essential structural role in facilitating catalysis. The larger methyltransferase holo complex contains accessory units, including WTAP (Wilms tumor 1-associated protein), VIRMA (vir-like m⁶A methyltransferase associated), RBM15 (RNA-binding motif protein 15) and its paralog (RBM15B), ZC3H13, and HAKAI ⁵⁻¹¹. Conversely, m⁶A marks are subject to removal by m⁶A demethylases, aptly termed “erasers”. Notable players in this dynamic process include FTO (fat mass and obesity-associated protein) and ALKBH5 (alkB homolog 5) ¹²⁻¹⁵. This intricate interplay between writers and erasers crafts a finely tuned regulatory mechanism, orchestrating the reversible m⁶A modifications that play critical roles in shaping RNA function and developmental processes.

The m⁶A RNA modification acts on multiple molecular pathways, including in splicing, stability, nuclear export, localization, translational efficiency, and activation and decay of targeted mRNAs 5,7,16,17. Recent studies have shown that constitutive knockout of Mettl14—a key facilitator of the m6A methyltransferase complex—is embryonically lethal in mice, whereas conditional knockout (cKO) of Mettl14 in neural progenitor cells disrupts cortical development and leads to premature death in mice ^{18,19}. Remarkably, levels of m6A are relatively low in mouse brain tissue during embryogenesis, but drastically increase by adulthood ²⁰, suggesting that m6A RNA modification plays a unique role in the adult central nervous system. That latter finding has also raised the possibility that m6A might play an important role in adult RNA homeostasis, with imbalances potentially leading to the onset or progression of neurodegeneration. This hypothesis is supported by studies demonstrating a positive correlation between m6A modification and gene expression homeostasis across tissues, as well as tissue-type-specific aging-associated m6A dynamics in primates ²¹. In humans, functional impairment of m6A has also been shown to play a pivotal role in cancer ^{22,23}, cell fate transition and determination ^{24,25}, and disease ²⁶⁻²⁸. Although a concordant decrease in m6A RNA methylation of brain tissue (specifically, the cingulate gyrus) from an Alzheimer's disease (AD) mouse model and in human patient brain tissues has been reported ²⁹, whether m6A exerts a direct causative role in human neurodegeneration remains obscure.

To date, research efforts have focused almost exclusively on DNA sequencing (usually whole exome) to identify the genetic causes of neurodegenerative diseases. This is arguably the primary reason why the genetic and molecular bases for many neurodegenerative diseases remain unknown, as most neurodegenerative diseases are sporadic ^{30,31}. Accumulating evidence indicates that most aging-associated diseases, including amyotrophic lateral sclerosis (ALS), are linked to RNA metabolism, perhaps explaining why probing gene mutations by DNA sequencing fails to identify more ALS-causative genes ^{32,33}. Though dysregulated RNA processing has been identified in the majority of ALS patients, it remains unclear which aspects of RNA metabolism are critical and if they are directly causative of spinal motor neuron (MN) degeneration ^{31,34}. There are two major shortcomings of ALS-associated research efforts to date: (1) next-generation sequencing technologies are DNA-based and cannot directly sequence RNA or RNA isoforms with long reads and modifications, hindering analyses of RNA modifications from patient transcriptomes; and (2) only a small number of mouse models mimic to varying degrees the MN pathology of sporadic ALS (sALS), with most of them presenting relatively minor phenotypes when compared to familial ALS (fALS) models ³⁰. Accordingly, there is still no robust sALS animal model (>90% of ALS patients are sporadic) that fully recapitulates MN degeneration pathology. Although employing a gene mutation identified in ALS patients to generate an ALS murine model remains a robust methodology, it frequently only elicits some aspects of ALS pathology, occasionally resulting in excessively shortened lifespan 30. Therefore, to advance research in this field necessitates: (1) discovering common disease-causing mechanisms present in both familial and sporadic ALS patients; and (2) establishing an ALS animal model based on these mechanisms. Such efforts would aim to replicate the primary hallmarks of sporadic and familial ALS at molecular (e.g., TARDBP/TDP43 aggregation), cellular (e.g., motor neuron degeneration), and physiological levels (manifesting muscle weakness and shortened lifespan).

Recently, two studies emphasized the roles of m⁶A in ALS. Barmada's group reported that m⁶A hypermethylation modulates RNA binding by TDP43 and the disease pathogenesis of ALS and frontotemporal dementia (FTD) ³⁵, whereas Sun's group indicated that globally reduced m⁶A levels in C9ORF72-associated ALS and FTD dysregulate RNA metabolism and contribute to neurodegeneration 36. Although these two studies emphasize the importance of m⁶A homeostasis in ALS, their seemingly contradictory results necessitate further clarification. As both studies were performed primarily on cell models and postmortem spinal cord sections from patients, it is imperative to explore if manipulating m⁶A levels in animal models in vivo recapitulates ALS pathology.

Our experiments used two different sets of motor neuron Cre drivers to remove Mettl14, corroborating that m⁶A hypomethylation (hypo-m⁶A) elicits an ALS-like phenotype in vivo. Impairment of the m⁶A repertoire elicits dysregulation of many known ALS-related pathways, with a concomitant change in the chromatin landscape of spinal MNs. Additionally, iPSC-derived MNs from several familial and sporadic ALS patients exhibited hypo-m⁶A, and restoration of m⁶A homeostasis by means of a small molecule largely spared the MNs from degeneration in both familial and sporadic ALS contexts. Most importantly, intrathecal delivery of Fto-shRNA to knock down Fto, an m⁶A eraser enzyme, ameliorated the motor deficits of SOD1^{G93A} mice (an ALS mouse model) and extended their lifespan. Accordingly, we speculate that m⁶A hypomethylation contributes to ALS and that restoring the m⁶A reservoir can mitigate the symptoms of sporadic and familial ALS. Therefore, there is a need for a composition and method of treatment of ALS and other degenerative motor neuron conditions and diseases based on restoration of m⁶A reservoir in subjects suffering from sporadic and familial ALS as well as other degenerative motor neuron conditions and diseases.

### SUMMARY OF THE INVENTION

The present invention provides a degenerative motor neuron treatment composition comprising one or more therapeutically effective amount of N⁶-methyladenosine (m⁶A) eraser inhibitor, or a histone methylation regulator, or a methyl donor or a combination thereof capable of inhibiting or reversing motor neuron degeneration in a subject.

The present invention further provides a method of treatment of degenerative motor neuron (MN) of a subject comprising the step of administering a therapeutically effective amount of the composition of claim 1 to the subject.

The present invention also provides a MN degeneration model comprising a subject deficient in METTL3 expression, METTL14 expression, or a combination thereof.

### BRIEF DESCRIPTION OF THE DRAWINGS

FIG. 1 illustrates the expression of m⁶A writers and erasers in human ALS iPSC-derived motor neurons. (A and B) Heatmaps (A) and corresponding box plots (B, left) of the m⁶A ‘writer’ complex core components in the iPSC-derived motor neurons (iPSC~MNs) of randomly selected controls (n=336) and ALS patients (both familial and sporadic, n=855). Other m⁶A modification enzymes, such as METTL16 for ncRNAs or METTL5 for rRNAs remain unchanged in ALS iPSC~MNs (B, right). Data were analyzed using the transcriptomic database from Answer ALS. Box plots indicate the interquartile range (IQR) with the central line revealing the median value, and the vertical lines extending to the extreme values in the group. (C) Heatmaps of the m⁶A ‘writer’ complex core components in the post-mortem cortex samples of randomly selected healthy controls (n=10) and sporadic ALS patients (n=26) of the GSE122650 database. (D) Heatmaps of the m⁶A ‘writer’ complex core components in SOD1 and C9ORF72 iPSC~MNs and healthy controls (n=3, respectively). Data was derived from the GSE132972 and GSE173115 databases. (E) qPCR analysis of m⁶A writers (METTL3 and METTL14) and erasers (FTO and ALKBH5) in human ALS iPSC~MNs upon degeneration induced by CPA treatment (see Methods for details). Note the general trend of downregulation for the m⁶A writers and upregulation of erasers upon MN degeneration in the ALS-associated lines compared to their isogenic rescue controls. Data are presented as mean±S.D., n≥3, with significant P values from two-tailed t-tests. N.S., non-significant.

FIG. 2 shows that m⁶A RNA modification levels are downregulated in ALS. (A) Timeline of MN differentiation in isogenic control (Ctrl) and ALS (SOD1^{+/L144F}, C9ORF72^{exp~800 G4C2}, TDP43^{G298S}) iPSC lines. CPA (cyclopiazonic acid) was applied to accelerate MN degeneration (annotated as basal time point day 0). NF: neurotrophic factors. (B~D) MN degeneration index (D.I.) and m⁶A RNA methylation levels of Ctrl and ALS iPSC~MNs. m⁶A percentages of Ctrl/ALS types at indicated time points were normalized to those at day 0. ALS iPSC~MNs undergo dramatic degeneration from day 7 to day 21 post-CPA treatment, while a significant reduction in m⁶A level was already exhibited at day 4. The D.I. measures neurite fragmentation. It is calculated by dividing the total area covered by neurite fragments by the total neurite area, and values range from 0 (completely intact) to 1 (completely fragmented). (E~G) Quantification of the m⁶A ratio in mRNAs from day 4 post-CPA treatment of isogenic control (Ctrl) and ALS (SOD1^{+/L144F}, C90RF72^{exp~800 G4C2}, TDP43^{G298S}) iPSC lines. The mRNAs were extracted by poly(A) purification. The panels at right show representative images of an m⁶A dot blot and methylene blue staining (for loading controls). All data are presented as mean±S.D., n≥3 independent experiments, significant P values from two-tailed t-tests. N.S., non-significant.

FIG. 3 shows that loss of m⁶A methyltransferases METTL3 and METTL14 leads to neurodegeneration. (A) Timeline of STM2457 (20 μM)-mediated inhibition of m⁶A modification during MN differentiation of wild-type iPSC lines. STM2457, which inhibits METTL3-mediated m⁶A modification (METTL3 inhibitor), was applied for 4 days to accelerate MN degeneration. (B~E) ALS MN degeneration upon addition of a METTL3 inhibitor (STM2457) for one week. (B and C) Inhibition of METTL3-mediated m⁶A modification in human iPSC~MNs results in a sharp decline in m⁶A levels, as assayed by mRNA ELISA (B) and mRNA m⁶A dot blot (C). m⁶A methylation was normalized to the vehicle (DMSO), which served as a non-stressed control. Note, the dramatic neurite degeneration revealed by SMI32 in (D). Scale bar, 200 μm. (E) Quantification of the degeneration index (D.I.) at an indicated time point normalized to the vehicle control. (F) Timeline of LV-mediated degeneration of MNs. (G and H) Knockdown of METTL3 and METTL14 significantly increases the degeneration of iPSC-MNs. (G) Upper panel shows representative immunostainings of SMI32 from LV-infected iPSC-MNs. In the middle panel, a binarized image of neurons with cell bodies removed and neurite fragments. In the lower panel, an enlarged region of the binarized neurite image. Scale bar, 200 μm. (H) Quantification of the results from (G). The degeneration index (D.I.) measures neurite fragmentation. It is calculated by dividing the total area covered by neurite fragments by the total neurite area, and values range from 0 (completely intact) to 1 (completely fragmented). All data are presented as mean±S.D., n≥3 independent experiments, significant P values from two-tailed t-tests.

FIG. 4 illustrates the knockdown efficiency test for shMETTL3 and shMETTL14. (A) Knockdown of METTL3 and METTL14 in HEK293T cells by two individual shRNA clones leading to downregulation of METTL3 and METTL14, as determined by qPCR (A), with a sharp decline of m⁶A mRNA levels, as assayed by dot blot (B).

FIG. 5 illustrates the phenotypic characterization of Olig2-Cre; Mettl14^{floxed} and ChAT-Cre; Mettl14^{floxed} mutant mice. (A and B) Immunostainings of Mett114, Olig2, and ChAT in the spinal cord sections of P24 Olig2-Cre; Mettl14^{foxed} and littermate control mice (A), together with respective quantification (B). The ventral horn is framed by the dashed squares in the middle column and is zoomed out in the rightmost column. (C) Kaplan-Meier survival curves show that Olig2-Cre; Mettl14^{floxed} mice die postnatally (i.e., before P30) compared to littermate controls. (D and E) Specifications of neuronal progenitors (Irx3^{on}, Pax6^{on}/p0~p2, Olig2^{on}/pMN, and Nkx2.2^{on}/p3) and generic MNs (Isl1(2)^{on} or Mnx1^{on}) are not affected in Olig2-Cre; Mettl14^{floxed} spinal cords at E10.5. (F and G) Immunostaining for Foxp1, Mnx1, and Isl1 at E13.5 reveals comparable numbers of columnar MN subtypes between spinal cords of Ctrl and Olig2-Cre; Mettl14^{floxed} mice. (H and I) Expression of OPC (oligodendrocyte precursors) and astrocyte markers in E17.5 control and Olig2-Cre; Mettl14^{floxed} mice. The ventral horn is framed by the dashed squares in the middle column and is zoomed out in the rightmost column. Scale bars, 100 μm. (J) Roentgenograms of mice revealing obvious kyphosis in the ChAT-Cre; Mettl14^{floxed} mice. Purple dotted lines depict the hindlimb shape; green dotted lines depict the mouse spine. All data are presented as mean±S.D., n≥3 mice, with significant P values from two-tailed t-tests. N.S., non-significant.

FIG. 6 illustrates the phenotypic characterization of ChAT-Cre; Mettl14^{floxed} mutant mice. (A) Body weight of male and female ChAT-Cre; Mettl14^{floxed} and littermate control mice. (B) Kaplan-Meier survival curves reflect that both male and female ChAT-Cre; Mettl14^{floxed} mice die prematurely compared to littermate controls. (C and D) Immunostaining (C) and quantification (D) of lumbar ChAT^{on} MN numbers reveal a gradual reduction starting after P100 and a significant loss of ChAT-Cre; Mettl14^{floxed} MNs at P160 (n=3 mice; Scale bar, 100 μm). (E and F) Preferential loss of the cholinergic C-bouton nerve terminals of motor neurons in the ChAT-Cre; Mettl14^{floxed} mice from P70 (E), with respective quantification in (F). Scale bar, 200 μm. Data are presented as mean±S.D., n=3 mice, with significant P values from two-tailed t-tests. N.S., non-significant.

FIG. 7 illustrates the molecular characterization of ChAT-Cre; Mettl14^{floxed} mutant mice. Images illustrate microglial activation, as determined by immunostaining for Iba1 (A), and quantification (B) of lumbar Iba1^{on} numbers at the ventral region, revealing significant microglial activation in ChAT-Cre; Mettl14^{floxed} mice compared to littermate controls (n=5 mice; Scale bar, 100 μm). (C) Tdp43 (green) is localized in the nucleus of the MNs of control mice. In the ChAT-Cre; Mettl14^{floxed} mutant mice, numerous Tdp43 inclusions exist in the cytoplasm (arrow). High magnifications of the highlighted Tdp43 aggregates in MNs are shown in the rightmost panels. Respective quantification is presented in (D). (E and G) Representative z-stack confocal images of neuromuscular junctions (NMJs) in gastrocnemius (GA) muscles dissected from P150 ChAT-Cre; Mettl14^{floxed} and littermate control mice. Motor nerves were visualized using a combination of SV2/NF (2H3) (green) and post-synaptic AChRs with α-BTX (magenta). Arrowheads identify denervated synapses, abnormal axonal swellings (E), and smaller endplates (G). (F and H) Quantification of the denervation ratio of NMJs and endplate area from (E and G). Scale bar, 100 μm. Data are presented as mean±S.D., n≥5 mice, with significant P values from two-tailed t-tests.

FIG. 8 illustrates the profiling phenotypes of ChAT-Cre; Mettl14^{floxed} mice from postnatal to adult stages. Images illustrating immunostaining (A) and quantification (B) of lumbar Iba1^{on} numbers in the ventral region reveal no significant microglial activation in P30~P120 ChAT-Cre; Mettl14^{floxed} mice compared with littermate controls. (C) Tdp43 (green) is localized in the nucleus of the MNs in P10~P100 ChAT-Cre; Mettl14^{floxed} mice. Respective quantification is presented in (D). (E) RNA binding protein Fus is localized in the nucleus of the MNs of normal mice. In the ChAT-Cre; Mettl14^{floxed} mutant mice, numerous Fus inclusions exist in the cytoplasm. Respective quantification is presented in (F). All data are presented as mean±S.D., n=3 mice, with significant P values from two-tailed t-tests. N.S., non-significant.

FIG. 9 shows that m⁶A-deficient mice display motor deficits that recapitulate ALS. (A) Schematic illustration of the behavioral tests conducted to assess motor functions. Created with BioRender.com. (B and C) Locomotor coordination on an accelerating rotarod is displayed as the rotation speed at which mice fell off (B) and the latency to fall (C). There was a significant decrease in locomotor activity at P70 and thereafter for males and at P100 and thereafter for females. (D) Forelimb grip strengths for ChAT-Cre; Mettl14^{floxed} male and female mutant mice. (E) Travel pathways (red) of representative trajectory diagrams filmed for 10 and 60 minutes in the open field test arena (square perimeter) for the early-onset (P70) and disease progression (P160) stages of ChAT-Cre; Mettl14^{floxed} and littermate control mice. (F) Total distance traveled in the open field test. (G) Schematic illustration of the behavioral tests from the treadmill conducted to assess motor function. Created with BioRender.com. (H and I) Stride width (usually mediated by interneurons) is not compromised, whereas stride length (mediated by motor neurons) is drastically reduced in the ChAT-Cre; Mettl14^{floxed} mice (Speed=15 cm/sec). Data are presented as mean±S.D., n=3-12 mice, significant P values from two-tailed t-tests. N.S., non-significant.

FIG. 10 shows that ChAT-Cre; Mettl14^{floxed} mice display overt motor deficits but no obvious defect in interneuron-mediated coordination. (A) Limb coordination and gait analysis were assayed by treadmill walking. ChAT-Cre; Mettl14^{floxed} mice display comparable temporal parameters of hindlimb stance, swing, brake, and propulsion time to controls, but their hindlimb walking ability is seriously compromised. Speed=15 cm/see (n=3 mice). (B) ChAT-Cre; Mettl14^{floxed} mice display a gradual decrease of total distance traveled in the open field test. Data are presented as mean±S.D., n=6 mice, with significant P values from two-tailed t-tests. N.S., non-significant.

FIG. 11 shows that Nanopore direct RNA-seq identifies ALS risk genes as m⁶A modified. (A~C) Validation of motor neuron maturation in a conditioned medium. (A) Timeline of in vitro differentiation and maturation from ESC~MNs. (B) The heatmap from qPCR verification shows that several feature adult motor neuron genes (Slc5a7, Syn1, Fos, Col5a3, Dmp1, and Bag3) are upregulated over time. (C) Upper panel: Immunostaining of Syn1 in Day 7 and Day 12 ESC~MNs. SMI32 labels the motor neurons and neurites. Scale bar, 100 μm. Lower panel: Quantifications reveal a significant increase of neurite complexity in the Day 12 mature ESC~MNs. (D) Overview of the experimental workflow for conducting Nanopore Direct RNA sequencing on mature ESC~MNs. (E) Motif preference of m⁶A peaks identifies the DRACH consensus motif (D=A, T, or G, R=A or G, and H=A, T, or C). Metagene profile of enrichment of m⁶A-modified sites across the mRNA transcriptome. 5′UTR, 5′ untranslated region; CDS, coding sequence; 3′UTR, 3′ untranslated region. Replicates 1, 2, and 3 (R1, R2, and R3) represent the triplicate biological repeats. (F) The heatmap from Nanopore Direct RNA sequencing shows that several feature adult genes (ChAT, Spp1, Bag, Slc5a7, Fos, and Syn1) are more enriched in the Day 12 mature ESC~MNs compared to embryonic genes (Isl1, Olig2, and Mnx1). (G) KEGG pathway analysis of the m⁶A-modified MN epitranscriptome reveals distinct biological pathways related to neurodegenerative diseases. (H) Schematic for analyzing the m⁶A-modified MN epitranscriptome, showing that 42% of m⁶A-modified genes are ALS risk genes. (I) Cartoon illustration of experiments in J~M. (J and L) Stoichiometry of the m⁶A sites detected in Tardbp and Atp13a2. (K and M) Verification of the predicted m⁶A-modified sites in Tardbp and Atp13a2 by m⁶A-RIP qPCR of selected m⁶A mRNA-modified sites (high confidence sites) and non-m⁶A-bound RNAs (low confidence sites). Points represent individual biological experiments. All data are presented as mean±S.D., n=3, with significant P values from two-tailed t-tests. N.S., non-significant.

FIG. 12 illustrates the identification of m⁶A-modified genes contributing to motor neuron degeneration. (A) Overview of the experimental workflow for single nucleus multiomics. (B) Immunohistochemistry showing localization of SUN1-sfGFP-Myc in MNs that carry R26-CAG-LSL-Sun1 sfGFP-myc together with a Cre driver. Scale bar, 200 μm. (C) Uniform manifold approximation and projection (UMAP) representation of all nuclei that passed quality filtering. Dimensionality reduction and clustering were performed based on gene expression (RNA, left), chromatin accessibility (ATAC, middle), and weighted nearest neighbor (WNN) integration of RNA and ATAC data (right). Clusters are color-coded and annotated using label transfer prediction, referencing Blum et al., 2021. (D) Major cell type proportions are unaffected at P100~P120 in Sun1^{sfGFP}; ChAT-Cre; Mettl14^{floxed} mice, a stage before MN degeneration. (E) Schematic for cross-referencing DEGs, particularly those down-regulated in Sun1^{sfGFP}; ChAT-Cre; Mettl14^{floxed} MNs, and the m⁶A-modified MN epitranscriptome. The resulting data reveals distinct biological pathways (Gene Ontology, right) and KEGG (F) that might cause MN degeneration in the Sun1^{sfGFP}; ChAT-Cre; Mettl14^{floxed} mice. (G and H) Schematic for analyzing the dot-plot data (G), with the outcome (H) showing ALS disease risk genes displaying significant changes in expression in Sun1^{sfGFP}; ChAT-Cre; Mettl14^{floxed} mice in each cholinergic neuronal subtype.

FIG. 13 shows the sequencing output and mapping metrics for reads from three mouse ESC-derived MN biological samples. (A) Summary of the sequencing output and mapping metrics for reads from three mouse ESC-derived MN biological samples. (B) Verification of the predicted m⁶A-modified sites by m⁶A-RIP and IgG-RIP qPCR of selected m⁶A mRNA-modified sites.

FIG. 14 illustrates the analysis of the single-nucleus multiome of Sun1^{sfGFP}; ChAT-Cre; Mettl14^{floxed} mice. (A) Violin plots show the UMI and gene counts, percentage of reads mapped to mitochondrial genes, ATAC fragments, TSS enrichment, nucleosome signal, and blacklist fraction of single nuclei collected in our study. (B and C) Integration of all data, including control (Ctrl) and Sun1^{sfGFP}; ChAT-Cre; Mettl14^{floxed} (KO) samples, reveals no batch effects in our single-cell analysis. (D) Principal component analysis (PCA) of the cholinergic neuronal subtype from Ctrl and KO samples. PC1 and PC2 largely segregate samples based on their cell type identity, whereas PC3 distinguishes Ctrl and KO samples, suggesting that Sun1^{sfGFP}; ChAT-Cre; Mettl14^{floxed} introduce changes to the transcriptome and chromatin accessibility. (E) Violin plots showing expression patterns of known marker genes (rows) in each cell type (columns).

FIG. 15 illustrates the adult skeletal motor neuron (MN) subtype annotations. (A) UMAP plots showing skeletal MN subtype heterogeneity. (B) Clusters were annotated based on label transfer prediction from a published single-cell study (Blum et al., 2021). Violin plots show high prediction scores for each subtype, except for undetermined. (C) Violin plots showing expression patterns of known marker genes (rows) in each cluster (columns). (D) Comparison of MN subtype proportions in Ctrl and Sun1^{sfGFP}; ChAT-Cre; Mettl14^{floxed} samples. (E and F) UMAP of known subtype marker genes labels a subset of skeletal MNs. Chodl and Sv2a label fast and slow α MNs, and Kcnq5 distinguishes fast-fatigable a MNs.

FIG. 16 illustrates the identification of m⁶A-modified DEGs in Sun1^{sfGFP}; ChAT-Cre; Mettl14^{floxed} mice. (A and D) Gene ontology (GO) analysis of the down-regulated (A) and up-regulated (D) genes in the Sun1^{sfGFP}; ChAT-Cre; Mettl14^{floxed} mice that are predicted to be m⁶A-modified. (B, C, and E) Violin plots show differential expression of selected genes from each GO term. (F) The proportion of differentially expressed genes with open, closed, or neutral nearby ATAC peaks.

FIG. 17 illustrates the increase of repressive histone modification marks and closed chromatin regions in ChAT-Cre; Mettl14^{floxed} mice. (A) Schematic for cross-referencing of DEGs, particularly those up-regulated in Sun1^{sfGFP}; ChAT-Cre; Mettl14^{floxed} MNs, and the m⁶A-modified MN epitranscriptome, with the outcome revealing distinct biological pathways (Gene Ontology, right) that might increase repressive histone modification and the DNA damage response in the ChAT-Cre; Mettl14^{floxed} mice. (B~E) Representative images illustrating a dramatic increase in repressive H3K9me3 mark (B) and DNA damage yH2AX (D) signals. Quantifications of lumbar H3K9me3^{on} (C) and lumbar γH2AX (E) signal intensities in the ventral regions of ChAT-Cre; Mettl14^{floxed} mice compared to littermate controls (n≥4 mice; Scale bars, 50 μm). (F) The bar plot shows changes in the number of peaks and distribution of their annotated genomic locations in cholinergic neuronal subtypes derived from SunsfGFP; ChAT-Cre; Mettl14^{floxed} mice and control (Ctrl) snATACseq data.

FIG. 18 illustrates the increased repressive histone modification marks (H3K9me3) and DNA damage response (γH2AX) in ChAT-Cre; Mettl14^{floxed} mice. (A and B) Representative images show that all selected MNs display increased signals of H3K9me3 (A), γH2AX (B), and corresponding DAPI (C) in the ChAT-Cre; Mettl14^{floxed} mice. Scale bars, 5 μm. (D) Spearman's correlation of peak-gene changes in each cholinergic neuronal subtype.

FIG. 19 shows that an m⁶A eraser inhibitor efficiently rescues human ALS iPSC-derived MNs from premature death by restoring dysregulated genes caused by hypo-m⁶A. (A) Schematic illustration of the m⁶A biogenesis pathway and the applied FTO inhibitor (FB23-2) with their corresponding targeting pathways. (B) Representative images of FB23-2 rescuing the MN degeneration associated with ALS. Scale bar, 200 μm. (C and D) Quantifications of m⁶A mRNA methylation levels (C) and degeneration index values (D) at an indicated time point and compared to the CPA treatment. Note the significant rescue of the degeneration index upon applying FB23-2 to CPA-stressed C9ORF72^{exp}, SOD1^{+/L144F} TDP43^{G2985}, and sALS iPSC~MNs. Data are presented as mean±S.D., n=3, significant P values from two-way ANOVA. N.S., non-significant. (E) Heatmaps of normalized expression level between stress-treated (CPA) ALS-relevant lines with or without subsequent FB23-2 treatment, revealing restorations of many ALS-related m⁶A modifications (highlighted with rectangles) to control (vehicle) levels for the FB23-2-treated groups. A z-score normalization was performed on the normalized read counts across samples for each gene after stress-treatment (CPA) with or without subsequent FB23-2 treatment. Samples were normalized to the vehicle control to reveal the normalized expression level.

FIG. 20 show that an m⁶A eraser inhibitor efficiently rescues the histone modification and chromatin remodeling-related genes of human ALS iPSC-derived MNs by restoring dysregulated genes caused by hypo-m⁶A. (A~C) Gene ontology (GO) analysis of the up-regulated genes in ALS iPSC~MNs revealed chromatin remodeling, DNA damage, synapse organization, and other pathways in the ChAT-Cre; Mettl14^{floxed} mice. Heatmaps of normalized expression levels between stress-treated (CPA) ALS-relevant lines with or without subsequent FB23-2 treatment, revealing restoration to control levels (vehicle, highlighted with rectangles) of many histone-related (D) and chromatin remodeling-related (E) m⁶A modifications for the FB23-2-treated groups. A z-score normalization was performed on the normalized read counts across samples for each gene after stress treatment (CPA) with or without subsequent FB23-2 treatment. Samples have been normalized to the vehicle control to reveal the normalized expression level.

FIG. 21 shows that gene therapy of adult SOD1^{G93A} mice by overexpressing Fto-shRNA is sufficient to protect neuromuscular function and delay disease onset. (A and B) Overview of the experimental strategy. (C) Western blot reveals that Fto protein expression is reduced in lumbar spinal cords after scAAV9-shFto injection. (D and E) Kaplan-Meier survival curves revealing prolongation of the onset of weight decline in SODIG93A mice (from ~140 days to ~155 days), with lifespans extended by ~10% (from ~170 days to ~187 days), following scAAV9-shFto injection. (F~I) MN number is rescued (F, with quantification in H), and gliosis is reduced (G, with quantification in I) upon restoring m⁶A level after scAAV9-shFto injection of SOG1^{G93A}, SOG1^{G93A}; scAAV9-shFto, and Ctrl mice at P140. Scale bars, 100 μm. (J) Illustration of the recording setup for evoked CMAPs in the gastrocnemius anterior (GA) muscle after stimulation of the sciatic nerve (left), with neuromuscular function assayed according to evoked CMAPs in the GA muscle. The CMAP amplitude (averages of maximum peak-to-peak values) is already reduced in SOD1^{G93A} mice at P60 and gradually declines further over time, whereas scAAV9-shFto treatment significantly ameliorates neuromuscular function at P160 (mean±S.D., n=5~9 for each group; two-tailed t-tests) (right). (K and L) Motor coordination and muscle strength are enhanced by scAAV9-shFto injection, as assayed by rotarod test at P60~P160 (K) (mean±S.D., n=7~9 for each group; two-tailed t-tests), and by grip strength test (L) (mean±S.D., n=5~9 for each group; two-tailed t-tests). N.S., non-significant. Illustrations in J~L were created with BioRender.com.

FIG. 22 shows that shRNA-mediated Fto inhibition can restore m⁶A levels. (A) Western blots show that all selected Fto-shRNAs can efficiently knock down Fto, with corresponding m⁶A hypermethylation in C2C12 cells (B and C). (D) Successful scAAV9-mediated expression was confirmed by detecting GFP in the ChAT^{on} regions in the P100 lumbar spinal cord of a control mouse injected with scAAV9-EGFP. Higher magnification of the highlighted area is shown in the panel at right. Scale bars, 200 μm. All data are presented as mean±S.D., n≥3, with significant P values from two-tailed t-tests. N.S., non-significant.

FIG. 23 shows that an increase of H3K9me3 is in MNs of SOD1^{G93A} and is reduced after scAAV9-shFto treatment. (A) Representative images illustrating H3K9me3 marks (yellow arrowheads) in the ventral horn of different sets of Ctrl and SOD1^{G93A} mice, w/o scAAV9-shFto injections. (B) Quantifications of lumbar H3K9me3^{on} (n=3 mice; Scale bars, 20 μm). Data are presented as mean±S.D., n=3 mice, with significant P values from two-tailed t-tests. N.S., non-significant.

FIG. 24 illustrates the DNA damage marker γH2AX in MNs of SOD1^{G93A}; AAV-shFto mice. (A) Representative images illustrating γH2AX marks (yellow arrowheads) in the ventral horn of Ctrl and SOD1^{G93A} mice. (B) Quantification of the intensity of lumbar γH2AX^{on} signal (n=3 mice, quantified for all γH2AX^{on} ChAT^{on} double-positive cells; scale bars, 20 μm). Data are presented as mean±S.D., n=3 mice, with significant P values from two-tailed t-tests. N.S., non-significant.

FIG. 25 summarizes the phenotypes of ChAT-Cre; Mettl14^{floxed} mice and possible mechanism leading to MN degeneration. (A) Timeline of manifestations of the ALS-like phenotype in the ChAT-Cre; Mettl14^{floxed} mice. Created with BioRender.com. (B) A schematic model showing how global m⁶A hypomethylation in ALS might lead to MN degeneration and the possible intervention to restore the m⁶A reservoir, thereby ameliorating ALS symptoms.

FIG. 26 illustrates the effects of m⁶A-targeting small molecules in rescuing MN degeneration associated with ALS. (A) Treatment scheme and the biological role of the m⁶A-targeting small molecules (IOX1, FB23-2, SAM) in ALS iPSCs (B) Representative images of the selected small molecules rescuing the MN degeneration associated with ALS. Scale bar, 200 μm. (C) Quantification of FOXP1^{on}/ISL1^{on} numbers at indicated time points. Data are normalized to the CPA treatment group.

FIG. 27 illustrates the shRNA-FTO inhibition effect in HEK293T cells through the qPCR test. Nucleotide sequences and the targeting sites of shFTO a-j are listed in Table 1-2.

FIG. 28 illustrates the effects of m⁶A-targeting small molecules in rescuing MN degeneration associated with ALS. (A) Treatment scheme and the biological role of the histone methyltransferase (HMT) inhibitor, particularly for H3K9me3 (chaetocin and F5446) in SOD1^{+/L144F} ALS iPSCs~differentiated motor neurons (B) Representative images of the selected small molecules rescuing the MN degeneration associated with SOD1^{+/L144F} ALS iPSCs~differentiated motor neurons. Scale bar, 100 μm. (C) Quantification of degenerated index at indicated time points compared with CPA accelerated degenerative motor neurons. Data are normalized to the CPA treatment group.

FIG. 29 illustrates the effect of methyl donors in rescuing degeneration in ALS iPSC-derived MNs. (A) Treatment scheme of the methyl donors in ALS iPSC-derived MNs (B) Representative images of the selected methyl donors rescuing the MN degeneration associated with ALS iPSC-derived MNs. Scale bar, 100 μm (C) Quantification of degenerated index at indicated time points compared with CPA accelerated degenerative MNs. Dara are normalized to the CPA treatment group.

### DETAILED DESCRIPTION OF THE INVENTION

As used in this specification and in claims which follow, the singular forms “a”, “an” and “the” include plural referents unless the context clearly indicates otherwise. Thus, for example, reference to “an ingredient” includes mixtures of ingredients, reference to “an active pharmaceutical agent” includes more than one active pharmaceutical agent, and the like.

As used herein, the term “about” as a modifier to a quantity is intended to mean±20%, ±15%, ±10% or ±5% inclusive of the quantity being modified.

As used herein, the term “subject,” “individual” or “patient” is used interchangeably herein, which refers to a vertebrate, preferably a mammal, more preferably a human. Mammals include, but are not limited to, murines, simians, humans, farm animals, sport animals, and pets.

As used herein, the term “effective amount” or “a therapeutically effective amount” of a drug or pharmacologically active agent comprises administering an amount necessary to achieve a desired result. The exact amount required will vary from subject to subject, depending on the species, age, general condition of the subject, the severity of the disease, the particular active agent, its mode of administration, the desired outcome, and the like. In certain embodiments of the present invention, a “therapeutically effective amount” of a compound or pharmaceutical composition is that amount effective for inhibiting progression or reversing of any disease or condition disclosed herein in a subject or a biological sample (e.g., in cells). In certain embodiments, condition or disease progression is inhibited by about 10%, about 20%, about 30%, about 40%, about 50%, about 60%, about 70%, about 80%, about 90%, about 95%, or about 99% or any percentages or percentage ranges falling within these values. In certain embodiments, the compound inhibits condition or disease progression by at least about 25%, at least about 50%, at least about 75%, or at least about 90%. In certain embodiments of the present invention, a “therapeutically effective amount” refers to an amount of a composition sufficient to reversal of disease or condition. In certain embodiments, the condition or disease is reversed by about 1%, about 5%, about 10%, about 20%, about 30%, about 40%, about 50%, about 60%, about 70%, about 80%, about 90%, about 95%, about 99% or any percentages or percentage ranges falling within these values.

As used herein, “sequence identity” and “% identity,” refers to the value determined by comparing two optimally aligned sequences over a comparison window, wherein a portion of the sequence in the comparison window may comprise additions or deletions as compared to the reference sequence for optimal alignment of the two sequences.

The number of positions at which identical amino acid residues or nucleic acids occur in both sequences is determined, yielding the number of matched positions, which is divided by the total number of positions in the window of comparison and the result multiplied by 100 to yield the percentage of sequence identity. The comparison window is the entire length of the sequence being referred to unless indicated otherwise.

As used herein, the term “target” or “targeting” a polynucleotide or gene comprises regulating the expression level, biological function, or a combination thereof, of the polynucleotide or gene. In an embodiment, the regulation comprises direct regulation or indirect regulation. Indirect regulation comprises regulating expression level, biological function, or a combination thereof, of the polynucleotide or gene through regulation of a regulator of the polynucleotide or gene. The term “target” or “targeting” a sequence can mean targeting a sequence comprising part of the polynucleotide or gene being targeted.

The present invention provides a degenerative motor neuron treatment composition comprising a therapeutically effective amount of N⁶-methyladenosine (m⁶A) regulator, a histone methylation regulator, a methyl donor, or a combination thereof. In an embodiment, the degenerative motor neuron treatment composition is capable of treating degenerative motor neuron condition in a subject or diseases such as ALS related to, caused by or having the symptoms of degenerative motor neuron in a subject. In an embodiment, the m⁶A regulator of the present invention comprises a m⁶A eraser inhibitor, a m⁶A writer activator, or a combination thereof. In an embodiment, the m⁶A regulator of the present invention consists of m⁶A eraser inhibitor.

In an embodiment, the m⁶A eraser inhibitor comprises a small molecule compound, an oligonucleotide, or a combination thereof capable of targeting a m⁶A eraser comprising fat mass and obesity-associated protein (FTO), alkB homolog 5 (ALKBH5), or a combination thereof. In an embodiment, the m⁶A eraser inhibitor comprises FB23-2, MO-I-500, IOX1, FTO-IN-3, an oligonucleotide capable of targeting one or more sequences listed in Table 1, or a combination thereof. In an embodiment, the oligonucleotide capable of targeting one or more sequences listed in Table 1 comprises a shRNA where nucleotide sequence of the shRNA is at least about 80%, about 85%, about 90%, about 95%, or about 100% identical to a nucleotide sequence listed in Table 2. In an embodiment, the oligonucleotide capable of targeting one or more sequences listed in Table 1 is capable of down-regulating expression of genes associated with one or more sequences listed in Table 1.

TABLE 1
Target sequences (a clone ID indicates a TRCN ID or a start position
of NM_001080432.3)

| SEQ ID | | |
|---|---|---|
| NO. | clone ID | Target sequence |
| 1 | TRCN0000183897_mmuFTO | CCAGGGAGACTGCTATTTCAT |
| 2 | TRCN0000277193_mmuFTO | GTCTCGTTGAAATCCTTTGAT |
| 3 | TRCN0000277143_mmuFTO | TTGAAAGAGGAGCCCTATTTC |
| 4 | TRCN0000246247_hsaFTO | TCACGAATTGCCCGAACATTA |
| 5 | TRCN0000246248_hsaFTO | CCCATTAGGTGCCCATATTTA |
| 6 | TRCN0000246249_hsaFTO | CGGTTCACAACCTCGGTTTAG |
| 7 | TRCN0000246250_hsaFTO | TCACCAAGGAGACTGCTATTT |
| 8 | TRCN0000255402_hsaFTO | CAACGTAACTTTGCTGAATTT |
| 9 | TRCN0000255403_hsaFTO | ACCTGAACACCAGGCTCTTTA |
| 10 | TRCN0000255404_hsaFTO | TCTCGCATCCTCATTGGTAAT |
| 11 | TRCN0000255405_hsaFTO | TCGCATGGCAGCAAGCTAAAT |
| 12 | TRCN0000257473_hsaFTO | GCCAGTGAAAGGGTCTAATAT |
| 13 | TRCN0000165774_hsaSNX29P1 | CCTCCTAAGTAGCTGGGATTA |
| 14 | TRCN0000222574_hsaERAP2 | CGCCTGTAATCCCAGCACTTT |
| 15 | TRCN0000165205_hsaYIF1B | GCCTCAGTTTCCTCATCTGTA |
| 16 | TRCN0000165704_hsaLOC652276 | CAATGGCACAATCTTGGCTCA |
| 17 | TRCN0000078113_hsaLIAS | GCCTGTAATCCCAGCACTTTA |
| 18 | TRCN0000155836_hsaKLHL30 | CCCAAAGTGCTGGGATTACAA |
| 19 | TRCN0000264189_hsaLINC01098 | CAAGTAGCTGGGACTACAGGA |
| 20 | TRCN0000141025_hsaEID2B | CCCAAAGTGCTGGGATTACTT |
| 21 | 147 | CAGCAGTGGCAGCTGAAATAT |
| 22 | 384 (a) | CCAGTGAAAGGGTCTAATATA |
| 23 | 385 | CAGTGAAAGGGTCTAATATAA |
| 24 | 386 | AGTGAAAGGGTCTAATATAAA |
| 25 | 671 (b) | CCTGAAAGAGGAACCTTATTT |
| 26 | 1009 (c) | CAACAGGAACCTTGGATTATA |
| 27 | 1011 | ACAGGAACCTTGGATTATATT |
| 28 | 1012 | CAGGAACCTTGGATTATATTT |
| 29 | 1994 (d) | AGCTGTGGCTACTTCAATTTA |
| 30 | 1995 | GCTGTGGCTACTTCAATTTAA |
| 31 | 1996 | CTGTGGCTACTTCAATTTAAA |
| 32 | 1997 | TGTGGCTACTTCAATTTAAAT |
| 33 | 1998 | GTGGCTACTTCAATTTAAATT |
| 34 | 2156 (e) | ATTGGACAACACTTCTATAAA |
| 35 | 2334 (f) | CACGGGAAGGAGATGTTAATA |
| 36 | 2335 | ACGGGAAGGAGATGTTAATAA |
| 37 | 2336 | CGGGAAGGAGATGTTAATAAT |
| 38 | 2337 | GGGAAGGAGATGTTAATAATT |
| 39 | 2338 | GGAAGGAGATGTTAATAATTA |
| 40 | 2473 (g) | TCCCTCATTCTTATGTAATAA |
| 41 | 2559 | CAAGAGAGAGTAGGGTTTAAA |
| 42 | 2635 | CAGTTTCCTCATCTGTATAAT |
| 43 | 2727 (h) | ATTTCCAGCACATAGTTAAAT |
| 44 | 2728 | TTTCCAGCACATAGTTAAATA |
| 45 | 2729 | TTCCAGCACATAGTTAAATAT |
| 46 | 2997 (i) | TGAAGGTGCACTGAGAATAAA |
| 47 | 3063 (j) | TTCCCATGAATGCAGTTAATA |
| 48 | 3225 | AGGGTCATTACAGAGATTAAA |
| 49 | 3226 | GGGTCATTACAGAGATTAAAT |
| 50 | 3270 | CAGGAGGGCGTGGTGTTTAAA |
| 51 | 3367 | TCCCATTAGGTGCCCATATTT |
| 52 | 3369 | CCATTAGGTGCCCATATTTAA |
| 53 | 3370 | CATTAGGTGCCCATATTTAAA |
| 54 | 3793 | CGCATGGCAGCAAGCTAAATA |
| 55 | 3794 | GCATGGCAGCAAGCTAAATAA |
| 56 | 4609 | AGGTGGGCAAGACCCAATAAA |
| 57 | 6032 | AGAGGAGCGGTGTGGATTAAA |
| 58 | 6089 | CTTTGTTACTCTGTGATTAAA |
| 59 | 6433 | GTTCTCCATTCTCTCTTATTA |
| 60 | 6434 | TTCTCCATTCTCTCTTATTAA |
| 61 | 6924 | CAGTGAGGTACTAGCTATTAT |
| 62 | 6925 | AGTGAGGTACTAGCTATTATT |
| 63 | 6926 | GTGAGGTACTAGCTATTATTA |
| 64 | 6927 | TGAGGTACTAGCTATTATTAT |
| 65 | 6928 | GAGGTACTAGCTATTATTATT |
| 66 | 7015 | TCCAGTCTTTGTTTCTATAAT |
| 67 | 7129 | ACGCCACGCATGTACATTATT |
| 68 | 7130 | CGCCACGCATGTACATTATTA |
| 69 | 7131 | GCCACGCATGTACATTATTAT |
| 70 | 7212 | GGCTTGTTCAAGAAGAATATT |
| 71 | 7231 | TTGTGTCACTATCCCATATAT |
| 72 | 7232 | TGTGTCACTATCCCATATATT |
| 73 | 7233 | GTGTCACTATCCCATATATTA |
| 74 | 7234 | TGTCACTATCCCATATATTAT |
| 75 | 8141 | GCCACCACATCCGGCTAATTT |
| 76 | 8298 | TGCTTGGTGTTAAAGATTAAA |
| 77 | 8516 | AGTCCAAGGGAGAGAAATTAT |
| 78 | 8517 | GTCCAAGGGAGAGAAATTATT |
| 79 | 8518 | TCCAAGGGAGAGAAATTATTA |
| 80 | 8519 | CCAAGGGAGAGAAATTATTAT |
| 81 | 8520 | CAAGGGAGAGAAATTATTATT |
| 82 | 9130 | GAGAAGCAAACCAGGTATATA |
| 83 | 9326 | GATCTGTCAGAAGCCATTAAA |
| 84 | 9597 | GACTGGGTCGGGTTGTTATTT |
| 85 | 10225 | TCCAGATGGGAGAAGTTATTT |
| 86 | 10522 | TTATTAGAGCCACTGAAATTT |
| 87 | 10523 | TATTAGAGCCACTGAAATTTA |
| 88 | 10524 | ATTAGAGCCACTGAAATTTAT |
| 89 | 10712 | GCTCATCGGTTTATGTATTAT |
| 90 | 10874 | ACCTGTGATCGTGCCTATTAA |
| 91 | 10882 | TCGTGCCTATTAAGCAATTAA |
| 92 | 11290 | CGGAACAGTGCGAAGATTATT |
| 93 | 11410 | GACTAAAGGGTGTCCTATTTA |
| 94 | 11411 | ACTAAAGGGTGTCCTATTTAT |
| 95 | 11412 | CTAAAGGGTGTCCTATTTATA |
| 96 | 11413 | TAAAGGGTGTCCTATTTATAA |
| 97 | 11452 | CTTCATACTCTGTGCTTATTA |
| 98 | 11485 | GGCTCTTAGAGTTTCTATTAA |
| 99 | 148 | AGCAGTGGCAGCTGAAATATC |
| 100 | 158 | GCTGAAATATCCTAAACTAAT |
| 101 | 198 | GTATCTGAGGAGCTCCATAAA |
| 102 | 199 | TATCTGAGGAGCTCCATAAAG |
| 103 | 381 | TGGCCAGTGAAAGGGTCTAAT |
| 104 | 382 | GGCCAGTGAAAGGGTCTAATA |
| 105 | 536 | ATTGTGTATGTCTGCAGATTT |
| 106 | 589 | GACAAGATGAAGTGGACATTA |
| 107 | 590 | ACAAGATGAAGTGGACATTAA |
| 108 | 591 | CAAGATGAAGTGGACATTAAG |
| 109 | 670 | ACCTGAAAGAGGAACCTTATT |
| 110 | 750 | GCGGTGGCAGTGTACAGTTAT |
| 111 | 751 | CGGTGGCAGTGTACAGTTATA |
| 112 | 752 | GGTGGCAGTGTACAGTTATAG |
| 113 | 815 | CGAAGGCAGGGATCCTGATAT |
| 114 | 816 | GAAGGCAGGGATCCTGATATT |
| 115 | 818 | AGGCAGGGATCCTGATATTTG |
| 116 | 897 | CACCAAGGAGACTGCTATTTC |
| 117 | 1008 | TCAACAGGAACCTTGGATTAT |
| 118 | 1081 | ATGATGATGTCTCTTTGAAAT |
| 119 | 1118 | TTTGAAACAAGGAGAAGAAAT |
| 120 | 1119 | TTGAAACAAGGAGAAGAAATT |
| 121 | 1120 | TGAAACAAGGAGAAGAAATTC |
| 122 | 1124 | ACAAGGAGAAGAAATTCATAA |
| 123 | 1125 | CAAGGAGAAGAAATTCATAAT |
| 124 | 1274 | TGCTGTGCTTCATGAAGTTAA |
| 125 | 1275 | GCTGTGCTTCATGAAGTTAAA |
| 126 | 1310 | CGTGGAACAAAGGAATGAAAT |
| 127 | 1725 | CCCTGTGTTATAGTCTGATTT |
| 128 | 1733 | TATAGTCTGATTTGGTGTTAA |
| 129 | 1734 | ATAGTCTGATTTGGTGTTAAA |
| 130 | 1735 | TAGTCTGATTTGGTGTTAAAC |
| 131 | 1959 | ACCCATAGTGCTGTCCAATAT |
| 132 | 1960 | CCCATAGTGCTGTCCAATATG |
| 133 | 1992 | CTAGCTGTGGCTACTTCAATT |
| 134 | 1993 | TAGCTGTGGCTACTTCAATTT |
| 135 | 1999 | TGGCTACTTCAATTTAAATTC |
| 136 | 2091 | GTGGCTAGTGACTGCTGTATT |
| 137 | 2106 | TGTATTGGACGGTACAGATAT |
| 138 | 2115 | CGGTACAGATATGGAACATTT |
| 139 | 2154 | CTATTGGACAACACTTCTATA |
| 140 | 2155 | TATTGGACAACACTTCTATAA |
| 141 | 2225 | CCCAAAGGCAAAGAAACTAAA |
| 142 | 2284 | TCTAAGACAAACCTAAGTAAA |
| 143 | 2332 | CACACGGGAAGGAGATGTTAA |
| 144 | 2333 | ACACGGGAAGGAGATGTTAAT |
| 145 | 2437 | GGATTTCACCAGCATAGTATA |
| 146 | 2466 | CTGTAAGTCCCTCATTCTTAT |
| 147 | 2471 | AGTCCCTCATTCTTATGTAAT |
| 148 | 2472 | GTCCCTCATTCTTATGTAATA |
| 149 | 2474 | CCCTCATTCTTATGTAATAAC |
| 150 | 2558 | ACAAGAGAGAGTAGGGTTTAA |
| 151 | 2607 | TCGTGTCCTTTGGCATGTTAA |
| 152 | 2633 | CTCAGTTTCCTCATCTGTATA |
| 153 | 2634 | TCAGTTTCCTCATCTGTATAA |
| 154 | 2636 | AGTTTCCTCATCTGTATAATG |
| 155 | 2674 | CCAGTCCTAAGGTGAACATTA |
| 156 | 2675 | CAGTCCTAAGGTGAACATTAA |
| 157 | 2676 | AGTCCTAAGGTGAACATTAAG |
| 158 | 2709 | AGTTACAGACTTAGAACAATT |
| 159 | 2710 | GTTACAGACTTAGAACAATTT |
| 160 | 2711 | TTACAGACTTAGAACAATTTC |
| 161 | 2725 | CAATTTCCAGCACATAGTTAA |
| 162 | 2730 | TCCAGCACATAGTTAAATATC |
| 163 | 2740 | AGTTAAATATCCAGGAAATTC |
| 164 | 2752 | AGGAAATTCTGGTACTGTTAT |
| 165 | 2834 | TTTGTCTTGTGATGCCATTAA |
| 166 | 2835 | TTGTCTTGTGATGCCATTAAC |
| 167 | 2885 | CCCATTGGTGTCCCAAGAAAT |
| 168 | 2895 | TCCCAAGAAATCGTGAGAATA |
| 169 | 2995 | ATTGAAGGTGCACTGAGAATA |
| 170 | 2996 | TTGAAGGTGCACTGAGAATAA |
| 171 | 2998 | GAAGGTGCACTGAGAATAAAC |
| 172 | 3062 | ATTCCCATGAATGCAGTTAAT |
| 173 | 3064 | TCCCATGAATGCAGTTAATAG |
| 174 | 3074 | GCAGTTAATAGCCACAGAAAT |
| 175 | 3083 | AGCCACAGAAATGTCACATTA |
| 176 | 3084 | GCCACAGAAATGTCACATTAA |
| 177 | 3085 | CCACAGAAATGTCACATTAAG |
| 178 | 3214 | CAGTGTCTCACAGGGTCATTA |
| 179 | 3223 | ACAGGGTCATTACAGAGATTA |
| 180 | 3224 | CAGGGTCATTACAGAGATTAA |
| 181 | 3227 | GGTCATTACAGAGATTAAATG |
| 182 | 3366 | ATCCCATTAGGTGCCCATATT |
| 183 | 3525 | TGCCTCCCAGGTTCAAGTAAT |
| 184 | 3526 | GCCTCCCAGGTTCAAGTAATT |
| 185 | 3527 | CCTCCCAGGTTCAAGTAATTC |
| 186 | 3795 | CATGGCAGCAAGCTAAATAAG |
| 187 | 3985 | CAATATATGTGTGTGACTATT |
| 188 | 3995 | GTGTGACTATTGAACTCTATT |
| 189 | 4015 | TCGTAGACTGCTTGTACTAAT |
| 190 | 4022 | CTGCTTGTACTAATGTCATTT |
| 191 | 4031 | CTAATGTCATTTGCATCATAA |
| 192 | 4086 | AGATAAGAAACCGAGAGATTT |
| 193 | 4207 | GTGCAACAGGGTTGGAGAAAT |
| 194 | 4217 | GTTGGAGAAATCACTACTTTA |
| 195 | 4218 | TTGGAGAAATCACTACTTTAA |
| 196 | 4219 | TGGAGAAATCACTACTTTAAG |
| 197 | 4245 | CCGAAAGGTGAGATCACATTT |
| 198 | 4253 | TGAGATCACATTTGCTCTAAA |
| 199 | 4258 | TCACATTTGCTCTAAAGAAAT |
| 200 | 4398 | GTGGTGGTGTGTGCCTGTAAT |
| 201 | 4518 | GGAGCGAGAGAAGAAAGAAAT |
| 202 | 4608 | GAGGTGGGCAAGACCCAATAA |
| 203 | 4721 | TGGAAGCACAGGCCCAGAAAT |
| 204 | 5054 | ACAGTAAGCAGCAAATCAATA |
| 205 | 5055 | CAGTAAGCAGCAAATCAATAA |
| 206 | 5056 | AGTAAGCAGCAAATCAATAAC |
| 207 | 5399 | GGAAGTAACTTGGTGAGATAT |
| 208 | 5400 | GAAGTAACTTGGTGAGATATT |
| 209 | 5465 | TGAGAGAGCAAACAGCTTAAT |
| 210 | 5466 | GAGAGAGCAAACAGCTTAATC |
| 211 | 5568 | GCCTACAGAGTTCACTCTAAA |
| 212 | 5653 | CACAGAGCTTTCTAGACATAA |
| 213 | 5654 | ACAGAGCTTTCTAGACATAAA |
| 214 | 5688 | GGCATCATTGGTGACACAATT |
| 215 | 5689 | GCATCATTGGTGACACAATTT |
| 216 | 5765 | GAGTCCATAGGACCCGTTTAA |
| 217 | 5766 | AGTCCATAGGACCCGTTTAAC |
| 218 | 5821 | TTCGAGGGAAGGAAGGTATTT |
| 219 | 5870 | TGCTCTAGTTAGGGCTGATTT |
| 220 | 5896 | CTGGGCAAATTGCATACATTT |
| 221 | 6031 | GAGAGGAGCGGTGTGGATTAA |
| 222 | 6059 | CGAAGTGGTAATGCCGTTTAT |
| 223 | 6060 | GAAGTGGTAATGCCGTTTATC |
| 224 | 6087 | GCCTTTGTTACTCTGTGATTA |
| 225 | 6088 | CCTTTGTTACTCTGTGATTAA |
| 226 | 6091 | TTGTTACTCTGTGATTAAATG |
| 227 | 6097 | CTCTGTGATTAAATGGTATTT |
| 228 | 6142 | TAGGTATCGCGGTGGTGAAAT |
| 229 | 6147 | ATCGCGGTGGTGAAATCAATT |
| 230 | 6148 | TCGCGGTGGTGAAATCAATTT |
| 231 | 6149 | CGCGGTGGTGAAATCAATTTG |
| 232 | 6161 | ATCAATTTGACAACCATAAAG |
| 233 | 6431 | TGGTTCTCCATTCTCTCTTAT |
| 234 | 6432 | GGTTCTCCATTCTCTCTTATT |
| 235 | 6435 | TCTCCATTCTCTCTTATTAAC |
| 236 | 6565 | GCTCCCAGGACAGTCCTAATT |
| 237 | 6566 | CTCCCAGGACAGTCCTAATTC |
| 238 | 6690 | CCGCTGTCATTCATCAGATTT |
| 239 | 6796 | TTCAAACCTTTGTTGTCTTAT |
| 240 | 6851 | GGTTGTTGTGAAGGTTCAATA |
| 241 | 6852 | GTTGTTGTGAAGGTTCAATAA |
| 242 | 6853 | TTGTTGTGAAGGTTCAATAAG |
| 243 | 6857 | TGTGAAGGTTCAATAAGTTAA |
| 244 | 6858 | GTGAAGGTTCAATAAGTTAAT |
| 245 | 6859 | TGAAGGTTCAATAAGTTAATG |
| 246 | 6894 | CTCCCACAGTACCTGACATAT |
| 247 | 6895 | TCCCACAGTACCTGACATATA |
| 248 | 6896 | CCCACAGTACCTGACATATAC |
| 249 | 6923 | TCAGTGAGGTACTAGCTATTA |
| 250 | 6983 | TGACTGGGCCAAATCACTTAA |
| 251 | 7013 | GCTCCAGTCTTTGTTTCTATA |
| 252 | 7014 | CTCCAGTCTTTGTTTCTATAA |
| 253 | 7016 | CCAGTCTTTGTTTCTATAATG |
| 254 | 7076 | CCCTACGATCAGCCATGTTAA |
| 255 | 7087 | GCCATGTTAAGTGATTCATAT |
| 256 | 7103 | CATATCATGTCTGGTACTTAA |
| 257 | 7104 | ATATCATGTCTGGTACTTAAA |
| 258 | 7127 | TCACGCCACGCATGTACATTA |
| 259 | 7128 | CACGCCACGCATGTACATTAT |
| 260 | 7132 | CCACGCATGTACATTATTATG |
| 261 | 7210 | GTGGCTTGTTCAAGAAGAATA |
| 262 | 7211 | TGGCTTGTTCAAGAAGAATAT |
| 263 | 7213 | GCTTGTTCAAGAAGAATATTG |

TABLE 2
shRNA sequences

| SEQ ID NO. | clone ID | shRNA sequence |
|---|---|---|
| 264 | shRNA_384 (a) | CCGGCCAGTGAAAGGGTCTAATATACTCGAG |
| | | TATATTAGACCCTTTCACTGGTTTTTG |
| 265 | shRNA_671 (b) | CCGGCCTGAAAGAGGAACCTTATTTCTCGAG |
| | | AAATAAGGTTCCTCTTTCAGGTTTTTG |
| 266 | shRNA_1009 (c) | CCGGCAACAGGAACCTTGGATTATACTCGAG |
| | | TATAATCCAAGGTTCCTGTTGTTTTTG |
| 267 | shRNA_1994 (d) | CCGGAGCTGTGGCTACTTCAATTTACTCGAGT |
| | | AAATTGAAGTAGCCACAGCTTTTTTG |
| 268 | shRNA_2156 (e) | CCGGATTGGACAACACTTCTATAAACTCGAG |
| | | TTTATAGAAGTGTTGTCCAATTTTTTG |
| 269 | shRNA_2334 (f) | CCGGCACGGGAAGGAGATGTTAATACTCGAG |
| | | TATTAACATCTCCTTCCCGTGTTTTTG |
| 270 | shRNA_2473 (g) | CCGGTCCCTCATTCTTATGTAATAACTCGAGT |
| | | TATTACATAAGAATGAGGGATTTTTG |
| 271 | shRNA_2727 (h) | CCGGATTTCCAGCACATAGTTAAATCTCGAG |
| | | ATTTAACTATGTGCTGGAAATTTTTTG |
| 272 | shRNA_2997 (i) | CCGGTGAAGGTGCACTGAGAATAAACTCGAG |
| | | TTTATTCTCAGTGCACCTTCATTTTTG |
| 273 | shRNA_3063 (j) | CCGGTTCCCATGAATGCAGTTAATACTCGAG |
| | | TATTAACTGCATTCATGGGAATTTTTG |

In an embodiment, the m⁶A writer activator comprises a small molecule compound, an oligonucleotide, or a combination thereof capable of targeting a m⁶A writer comprising methyltransferase like 3 (METTL3), METTL5, METTL14, METTL16, or a combination thereof.

In an embodiment, the methyl donor comprises choline, betaine chloride, folic acid (vitamin B9), vitamin B6, vitamin B12, S-adenosyl-L-methionine such as but not limited to S-adenosyl-L-methionine tosylate, or a combination thereof.

In an embodiment, the histone methylation regulator comprises a histone H3 lysine 9 trimethylation (H3K9me3) inhibitor. In an embodiment, the histone methylation regulator comprises chaetocin, F5446, GSK2879522, UNC0638, or a combination thereof.

In an embodiment, the degenerative motor neuron treatment composition of the present invention comprises a therapeutically effective amount of m⁶A regulator, methyl donor, or a combination thereof wherein molar ratio of the m⁶A regulator to the methyl donor ranges from about 200000:1 to about 1:10 such as about 200000:1, about 150000:1, about 100000:1, about 80000:1, about 60000:1, about 40000:1, about 20000:1, about 10000:1, about 8000:1, about 6000:1, about 4000:1, about 2000:1, about 1500:1, about 1000:1, about 800:1, about 600:1, about 400:1, about 200:1, about 150:1, about 100:1, about 50:1, about 25:1, about 10:1, about 5:1, about 2:1, about 1:1, about 1:2, about 1:4, about 1:6, about 1:8, about 1:10, or any ratio or ratio ranges falling within these ratio values.

In an embodiment, the degenerative motor neuron treatment composition of the present invention comprises a therapeutically effective amount of m⁶A regulator, methyl donor, or a combination thereof wherein molar ratio of the m⁶A regulator to the methyl donor ranges from about 20000:1 to about 100:1 such as about 20000:1, about 18000:1, about 16000:1, about 14000:1, about 12000:1, about 10000:1, about 8000:1, about 6000:1, about 4000:1, about 2000:1, about 1000:1, about 900:1, about 800:1, about 700:1, about 600:1, about 500:1, about 450:1, about 400:1, about 350:1, about 300:1, about 250:1, about 200:1, about 175:1, about 150:1, about 125:1, about 100:1, or any ratio or ratio ranges falling within these ratio values.

In an embodiment, the degenerative motor neuron treatment composition of the present invention comprises a therapeutically effective amount of m⁶A regulator, histone methylation regulator, or a combination thereof wherein molar ratio of the m⁶A regulator to the histone methylation regulator ranges from about 1000000:1 to about 1:1000000 such as about 1000000:1, about 500000:1, about 100000:1, about 50000:1, about 10000:1, about 5000:1, about 1000:1, about 500:1, about 100:1, about 50:1, about 10:1, about 5:1, about 1:1, about 1:5, about 1:10, about 1:50, about 1:100, about 1:500, about 1:1000, about 1:5000, about 1:10000, about 1:50000, about 1:100000, about 1:500000, about 1:1000000, or any ratio or ratio ranges falling within these ratio values.

In an embodiment, the degenerative motor neuron treatment composition of the present invention comprises a therapeutically effective amount of m⁶A regulator, histone methylation regulator, or a combination thereof wherein molar ratio of the m⁶A regulator to the histone methylation regulator ranges from about 1000:1 to about 1:1 such as about 1000:1, about 800:1, about 600:1, about 400:1, about 200:1, about 100:1, about 90:1, about 80:1, about 70:1, about 60:1, about 50:1, about 45:1, about 40:1, about 35:1, about 30:1, about 25:1, about 20:1, about 15:1, about 10:1, about 8:1, about 6:1, about 5:1, about 4:1, about 3:1, about 2:1, about 1:1, or any ratio or ratio ranges falling within these ratio values.

In an embodiment, the degenerative motor neuron treatment composition of the present invention comprises a therapeutically effective amount of histone methylation regulator, methyl donor, or a combination thereof wherein molar ratio of the histone methylation regulator and the methyl donor ranges from about 1000000:1 to about 1:1000000 such as about 1000000:1, about 500000:1, about 100000:1, about 50000:1, about 10000:1, about 5000:1, about 1000:1, about 500:1, about 100:1, about 50:1, about 10:1, about 5:1, about 1:1, about 1:5, about 1:10, about 1:50, about 1:100, about 1:500, about 1:1000, about 1:5000, about 1:10000, about 1:50000, about 1:100000, about 1:500000, about 1:1000000, or any ratio or ratio ranges falling within these ratio values.

In an embodiment, the degenerative motor neuron treatment composition of the present invention comprises a therapeutically effective amount of histone methylation regulator, methyl donor, or a combination thereof wherein molar ratio of the histone methylation regulator to the methyl donor ranges from about 1000:1 to about 1:1 such as about 1000:1, about 800:1, about 600:1, about 400:1, about 200:1, about 100:1, about 90:1, about 80:1, about 70:1, about 60:1, about 50:1, about 45:1, about 40:1, about 35:1, about 30:1, about 25:1, about 20:1, about 15:1, about 10:1, about 8:1, about 6:1, about 5:1, about 4:1, about 3:1, about 2:1, about 1:1, or any ratio or ratio ranges falling within these ratio values.

In an embodiment, the degenerative motor neuron treatment composition of the present invention comprising a therapeutically effective amount of IOX1 and S-adenosyl-L-methionine tosylate wherein molar ratio of the IOX1 to the S-adenosyl-L-methionine tosylate ranges from about 200000:1 to about 100:1 such as such as about 200000:1, about 150000:1, about 100000:1, about 80000:1, about 60000:1, about 40000:1, about 20000:1, about 10000:1, about 8000:1, about 6000:1, about 4000:1, about 2000:1, about 1000:1, about 900:1, about 800:1, about 700:1, about 600:1, about 500:1, about 400:1, about 300:1, about 200:1, about 100:1, or any ratio or ratio ranges falling within these ratio values.

In an embodiment, the degenerative motor neuron treatment composition of the present invention comprising a therapeutically effective amount of IOX1 and S-adenosyl-L-methionine tosylate wherein molar ratio of the IOX1 to the S-adenosyl-L-methionine tosylate ranges from about 50000:1 to about 1000:1 such as about 50000:1, about 45000:1, about 40000:1, about 35000:1, about 30000:1, about 25000:1, about 20000:1, about 18000:1, about 16000:1, about 14000:1, about 12000:1, about 10000:1, about 8000:1, about 6000:1, about 5000:1, about 4000:1, about 3000:1, about 2500:1, about 2000:1, about 1500:1, about 1000:1, or any ratio or ratio ranges falling within these ratio values.

In an embodiment, the degenerative motor neuron treatment composition of the present invention comprises a therapeutically effective amount of FB23-2 and S-adenosyl-L-methionine tosylate wherein molar ratio of the FB23-2 to the S-adenosyl-L-methionine tosylate ranges from about 10000:1 to about 100:1 such as such as about 10000:1, about 8000:1, about 6000:1, about 4000:1, about 2000:1, about 1500:1, about 1000:1, about 800:1, about 600:1, about 500:1, about 400:1, about 300:1, about 200:1, about 100:1, or any ratio or ratio ranges falling within these ratio values.

In an embodiment, the degenerative motor neuron treatment composition comprising a therapeutically effective amount of FTO-IN-3 and S-adenosyl-L-methionine tosylate wherein molar ratio of the FTO-IN-3 to the S-adenosyl-L-methionine tosylate ranges from about 100000:1 to about 1:10 such as about 100000:1, about 75000:1, about 50000:1, about 25000:1, about 10000:1, about 7500:1, about 5000:1, about 2500:1, about 1000:1, about 750:1, about 500:1, about 250:1, about 100:1, about 75:1, about 50:1, about 25:1, about 10:1, about 8:1, about 6:1, about 4:1, about 2:1, about 1:1, about 1:2, about 1:4, about 1:6, about 1:10, or any ratio or ratio ranges falling within these ratio values.

In an embodiment, the degenerative motor neuron treatment composition of the present invention comprises a therapeutically effective amount of FTO-IN-3 and S-adenosyl-L-methionine tosylate wherein molar ratio of the FTO-IN-3 to the S-adenosyl-L-methionine tosylate ranges from about 10000:1 to about 100:1 such as about 10000:1, about 9000:1, about 8000:1, about 7000:1, about 6000:1, about 5000:1, about 4000:1, about 3000:1, about 2000:1, about 1800:1, about 1600:1, about 1400:1, about 1200:1, about 1000:1, about 900:1, about 800:1, about 700:1, about 600:1, about 500:1, about 450:1, about 400:1, about 350:1, about 300:1, about 250:1, about 200:1, about 150:1, about 100:1, or any ratio or ratio ranges falling within these ratio values.

In an embodiment, the degenerative motor neuron treatment composition of the present invention comprises a therapeutically effective amount of IOX1 and FB23-2 wherein molar ratio of the IOX1 to the FB23-2 ranges from about 500000:1 to about 2:1 such as about 500000:1, about 250000:1, about 100000:1, about 75000:1, about 50000:1, about 25000:1, about 10000:1, about 7500:1, about 5000:1, about 2500:1, about 1000:1, about 800:1, about 600:1, about 400:1, about 200:1, about 100:1, about 80:1, about 60:1, about 40:1, about 20:1, about 15:1, about 10:1, about 8:1, about 6:1, about 4:1, about 2:1, or any ratio or ratio ranges falling within these ratio values.

In an embodiment, the degenerative motor neuron treatment composition of the present invention comprises a therapeutically effective amount of IOX1 and FB23-2 wherein molar ratio of the IOX1 to the FB23-2 ranges from about 50000:1 to about 1000:1 such as about such as about 50000:1, about 45000:1, about 40000:1, about 35000:1, about 30000:1, about 25000:1, about 20000:1, about 18000:1, about 16000:1, about 14000:1, about 12000:1, about 10000:1, about 8000:1, about 6000:1, about 5000:1, about 4000:1, about 3000:1, about 2500:1, about 2000:1, about 1500:1, about 1000:1, or any ratio or ratio ranges falling within these ratio values.

In an embodiment, the degenerative motor neuron treatment composition of the present invention comprises a therapeutically effective amount of IOX1 and FTO-IN-3 wherein molar ratio of the IOX1 to the FTO-IN-3 ranges from about 200000:1 to about 100:1 such as about 200000:1, about 150000:1, about 100000:1, about 80000:1, about 60000:1, about 40000:1, about 20000:1, about 10000:1, about 8000:1, about 6000:1, about 4000:1, about 2000:1, about 1000:1, about 900:1, about 800:1, about 700:1, about 600:1, about 500:1, about 400:1, about 300:1, about 200:1, about 100:1, or any ratio or ratio ranges falling within these ratio values.

In an embodiment, the degenerative motor neuron treatment composition of the present invention comprises IOX1 and FTO-IN-3 wherein molar ratio of the IOX1 to the FTO-IN-3 ranges from about 50000:1 to about 1000:1 such as about 50000:1, about 45000:1, about 40000:1, about 35000:1, about 30000:1, about 25000:1, about 20000:1, about 18000:1, about 16000:1, about 14000:1, about 12000:1, about 10000:1, about 8000:1, about 6000:1, about 5000:1, about 4000:1, about 3000:1, about 2500:1, about 2000:1, about 1500:1, about 1000:1, or any ratio or ratio ranges falling within these ratio values.

In an embodiment, the degenerative motor neuron treatment composition of the present invention comprises FB23-2 and FTO-IN-3 wherein molar ratio of the FB23-2 to the FTO-IN-3 ranges from about 10000:1 to about 100:1 such as such as about 10000:1, about 8000:1, about 6000:1, about 4000:1, about 2000:1, about 1500:1, about 1000:1, about 800:1, about 600:1, about 500:1, about 400:1, about 300:1, about 200:1, about 100:1, or any ratio or ratio ranges falling within these ratio values.

In an embodiment, the degenerative motor neuron treatment composition of the present invention comprises FB23-2, IOX1 and S-adenosyl-L-methionine tosylate wherein molar ratio of the FB23-2 to the IOX1 and to the S-adenosyl-L-methionine tosylate is about 1000:20000:1.

In an embodiment, the degenerative motor neuron treatment composition of the present invention comprises FB23-2 and betaine chloride wherein molar ratio of the FB23-2 to the betaine chloride ranges from about 50:1 to about 1:5 such as about 50:1, about 45:1, about 40:1, about 35:1, about 30:1, about 25:1, about 20:1, about 15:1, about 10:1, about 5:1, about 4:1, about 3:1, about 2:1, about 1:1, about 1:2, about 1:3, about 1:4, about 1:5, or any ratio or ratio ranges falling within these ratio values.

In an embodiment, the degenerative motor neuron treatment composition of the present invention comprises FB23-2 and folic acid (vitamin B9) wherein molar ratio of the FB23-2 to the folic acid (vitamin B9) ranges from about 100:1 to about 1:5 such as about 100:1, about 90:1, about 80:1, about 70:1, about 60:1, about 50:1, about 45:1, about 40:1, about 35:1, about 30:1, about 25:1, about 20:1, about 15:1, about 10:1, about 5:1, about 4:1, about 3:1, about 2:1, about 1:1, about 1:2, about 1:3, about 1:4, about 1:5, or any ratio or ratio ranges falling within these ratio values.

In an embodiment, the degenerative motor neuron treatment composition of the present invention comprises FB23-2, vitamin B12, and S-adenosyl-L-methionine tosylate wherein molar ratio of the FB23-2 to the vitamin B12 and to the S-adenosyl-L-methionine tosylate is about 10:3:1.

In an embodiment, the degenerative motor neuron treatment composition of the present invention comprises FB23-2, betaine chloride, and folic acid (vitamin B9) wherein molar ratio of the FB23-2 to the betaine chloride and to the folic acid (vitamin B9) is about 10:3:1.

In an embodiment, the degenerative motor neuron treatment composition of the present invention comprises vitamin B12 and S-adenosyl-L-methionine tosylate wherein molar ratio of the vitamin B12 to the S-adenosyl-L-methionine tosylate ranges from about 50:1 to about 1:5 such as about 50:1, about 45:1, about 40:1, about 35:1, about 30:1, about 25:1, about 20:1, about 15:1, about 10:1, about 5:1, about 4:1, about 3:1, about 2:1, about 1:1, about 1:2, about 1:3, about 1:4, about 1:5, or any ratio or ratio ranges falling within these ratio values.

In an embodiment, the degenerative motor neuron treatment composition of the present invention comprises m⁶A regulator and chaetocin wherein molar ratio of the m⁶A regulator to the chaetocin ranges from about 2000:1 to about 1:5000 such as about 2000:1, about 1500:1, about 1000:1, about 500:1, about 100:1, about 10:1, about 1:1, about 1:10, about 1:100, about 1:500, about 1:1000, about 1:1500, about 1:2000, about 1:2500, about 1:3000, about 1:3500, about 1:4000, about 1:4500, about 1:5000, or any ratio or ratio ranges falling within these ratio values.

In an embodiment, the degenerative motor neuron treatment composition of the present invention comprises a m⁶A regulator and F5446 wherein molar ratio of the m⁶A regulator to the F5446 ranges from about 5000:1 to about 1000:1 such as about 5000:1, about 4500:1, about 4000:1, about 3500:1, about 3000:1, about 2500:1, about 2000:1, about 1500:1, about 1000:1, or any ratio or ratio ranges falling within these ratio values.

In an embodiment, the degenerative motor neuron treatment composition of the present invention further comprises a nanoparticle, wherein the nanoparticle is capable of serving as a delivery vehicle of at least one m⁶A regulator, at least one histone methylation regulator, or at least one methyl donor. In an embodiment, the nanoparticle is capable of serving as the delivery vehicle of at least an oligonucleotide capable of targeting a m⁶A regulator of the present invention or downregulating expression of m⁶A eraser. In an embodiment, the nanoparticle comprises a virus such as but not limited to adeno-associated virus (AAV) or any variant or isoform thereof. In an embodiment, the AAV comprises AAV1, AAV2, AAV3, AAV4, AAV5, AAV6, AAV7, AAV8, AAV9, AAV10, scAAV8, scAAV9, scAAV10, scAAV11, AAVrh8, AAVrh10, or AAV Hu68.

The present invention also provides a method of treatment of motor neuron (MN) degeneration of a subject comprising the step of administering a therapeutically effective amount of any embodiment of the degenerative motor neuron treatment composition of the present invention to the subject. In an embodiment, the MN in the subject to be targeted by the composition of the present invention, or the target MN, comprises a lower MN comprising a somatic MN, a branchial MN or a general visceral MN, an upper MN, or a combination thereof. In an embodiment, the target MN originates from brain cerebral cortex, brain stem, spinal cord, or a combination thereof of the subject. In an embodiment, the target MN comprises a MN from a subject diagnosed with a motor neuron disease (MND). In an embodiment, the MND comprises amyotrophic lateral sclerosis (ALS) comprising familial ALS, sporadic ALS, or a combination thereof, progressive bulbar palsy, primary lateral sclerosis, progressive muscular atrophy, spinal muscular atrophy, Kennedy's disease, or post-polio syndrome. In an embodiment, ALS comprises C9orf72-associated ALS, SOD1-associated ALS, TDP43-associated ALS, FUS-associated ALS, ALS associated with abnormalities derived from one or more genes comprising CCNF, NEK1, VCP, SQSTM1, OPTN, UBQLN2, PFN1, TUBA4A, MATR3, CHCHD10, TBK1, KIF5A, NEFH, NEFH, SETX, DCTN1, VAPB, CHMP2B, SPG11, HNRNPA2B1, HNRNPA1, CFAP410, ANXA11, ERLIN1, GLT8D1, DNAJC7, HTT, or SPTLC1, or a combination thereof.

In an embodiment, the target MN comprises a MN of the subject wherein the MN comprises a deficiency in a m⁶A writer. In an embodiment, the target MN comprises a MN comprising a deficiency in a m⁶A methyltransferase. In an embodiment, the target MN comprises a MN comprising a deficiency in METTL3, METTL5, METTL 14, METTL16, VIRMA, HAKAI, ZC3H13, RBM15/15B, WTAP, or a combination thereof. In an embodiment, the target MN comprises an overexpression in a m⁶A eraser. In an embodiment, the target MN comprises an overexpression in FTO, ALKBH5, or a combination thereof. In an embodiment, the target MN comprises m⁶A hypomethylation.

In an embodiment, the administration of a therapeutically effective amount of any embodiment of the degenerative motor neuron treatment composition of the present invention to a target MN of a subject restores the m⁶A level in the target MN of the subject to at least about 50%, about 55%, about 60%, about 65%, about 70%, about 75%, about 80%, about 85%, about 90%, about 95%, or about 100% of the m⁶A level in a MN of the subject when healthy. In an embodiment, the administration of a therapeutically effective amount of any embodiment of the degenerative motor neuron treatment composition of the present invention to a target MN of a subject restores the m⁶A level in the target MN of the subject to at least about 50%, about 55%, about 60%, about 65%, about 70%, about 75%, about 80%, about 85%, about 90%, about 95%, or about 100% of the m⁶A level in a MN of the subject when not suffering from a MND. In an embodiment, the administration of a therapeutically effective amount of any embodiment of the degenerative motor neuron treatment composition of the present invention to a target MN of a subject restores the m⁶A level in the target MN of the subject to at least about 50%, about 55%, about 60%, about 65%, about 70%, about 75%, about 80%, about 85%, about 90%, about 95%, or about 100% of the m⁶A level of an average subject. In an embodiment, the administration of a therapeutically effective amount of any embodiment of the degenerative motor neuron treatment composition of the present invention to a target MN of a subject restores the m⁶A level in the target MN of a subject to at least about 50%, about 55%, about 60%, about 65%, about 70%, about 75%, about 80%, about 85%, about 90%, about 95%, or about 100% of the m⁶A level of an average healthy human being having the same biometrics of the subject such as age, sex, height, weight, etc. or a combination thereof.

In an embodiment, the administration of a therapeutically effective amount of any embodiment of the degenerative motor neuron treatment composition of the present invention to a target MN of a subject increases the m⁶A level in the target MN of the subject.

In an embodiment, the administration of a therapeutically effective amount of any embodiment of the degenerative motor neuron treatment composition of the present invention to a target MN of a subject increases the m⁶A level in the target MN of the subject by at least about 5%, about 10%, about 15%, about 20%, about 25%, about 30%, about 40%, about 50%, about 60%, about 70%, about 80%, about 90%, about 100%, about 150%, about 200%, about 250%, about 300%, about 350%, about 400%, about 450%, or about 500%.

In an embodiment, the m⁶A modification sites targeted or affected by the administration of a therapeutically effective amount of any embodiment of the degenerative motor neuron treatment composition of the present invention to a subject are located at genetic sequences associated with one or more genes of List 1. In an embodiment, the m⁶A modification sites targeted or affected by the administration of a therapeutically effective amount of any embodiment of the degenerative motor neuron treatment composition of the present invention to a subject is located at genetic sequences associated with one or more genes of List 2.

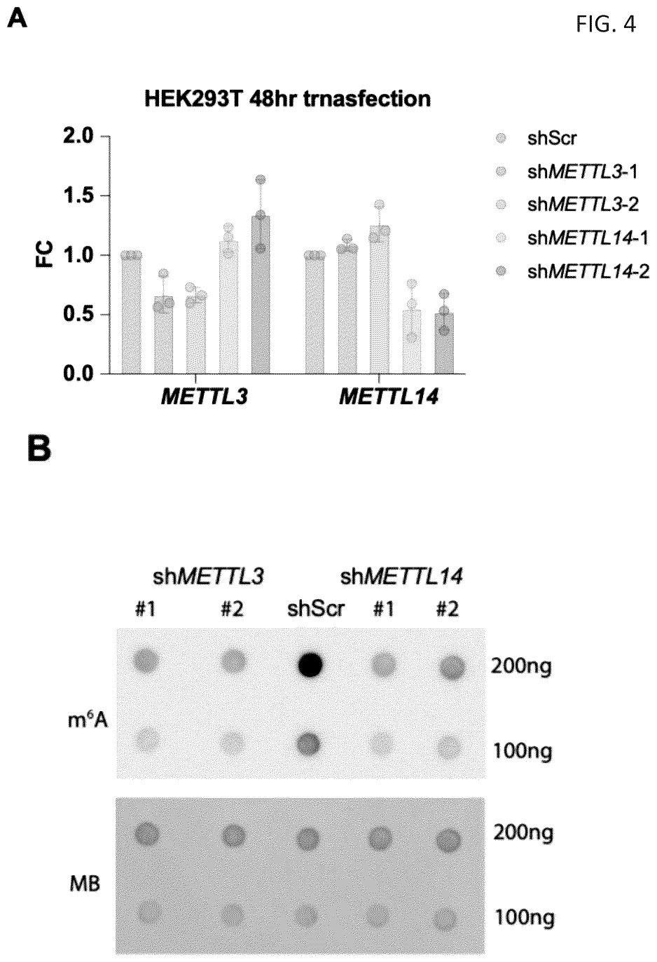

List 1. Gnai3, Cdc45, Narf, Cav2, Klf6, Scmh1, Wnt9a, Xpo6, Brat1, Gna12, Ccnd2, Gpr107, Nalcn, Drp2, Mid2, Scpep1, Mnt, Hddc2, Tpd5211, Pemt, Cdh1, Bcl6b, Arvcf, Comt, Dbt, Dazap2, Rnf17, Trappc10, Ccm2, Tbrg4, Fap, Lck, Galnt1, Mkrn2, Raf1, Septin1, Acvrl1, Grasp, Acvr1b, Tom112, Itga5, Wdr77, Dynlt1c, Cld, Gm2a, Clcn4, Sema4f, Hk2, Sez6, Lox13, Kat2b, Dnmt31, Aire, Icosl, Sult5a1, Chmpla, Rpa1, Serpinf1, Polr3d, Tcf7, Ddx3x, Slc5a5, Car4, Txnrd3, Zfp512b, Dnajc5, Tpd5212, Fmr1, Bcl11a, Dlg3, Gnb11, Mmp11, Smarcb1, Hiplr, Nsun5, Top1mt, Hoxa4, Oxall, Ilf2, Chtop, Mapk7, Epn2, B9d1, Sec24b, N4bp3, Rmnd5b, Nhp2, Zfp276, Clq14, Luzp1, Poldip2, Ift20, Col6a1, Pcbp3, Araf, Cfp, Uxt, Mcm3ap, Pont, Gmcl1, Oas1c, Calm1, Agpat3, Sema6b, Uhrf1, Dpp9, Lsr, Hpn, Ckb, Sp1, Myg1, Rarg, Pfdn5, Rrp15, Rnd2, Fndc5, Acp5, Fbxo9, Vps50, Hars, Zmat2, Glmp, Snd1, Col18a1, Slc19a1, Mrp110, Tcf25, Tubb6, Irx2, Foxm1, Itfg2, Tulp3, Gtf2 h4, Tubb5, El12, Jup, Fkbp10, Nom1, Ergic1, Tcea3, Csn3, Ifrd1, Brpf1, Hoxc6, Gstt1, Ddt, Ddx18, Akt1, Tcirg1, Naglu, Coasy, Smo, Chordc1, Rtcb, Pwp1, Capns1, Lrp3, Folr1, Clpb, Zdhhc4, Rac1, Nup214, Aif11, Ltbp1, Ugp2, Trmt1, Nacc1, Nfix, Slcla5, Fam50a, Grik3, Gria3, Sipa112, Pdzd4, Idh3g, Pnck, Ssr4, Fam98a, Trappc6a, Supt6, Sdf2, Ccne1, Hsf2 bp, Bbc3, Acp2, Rapsn, Celf2, Ddb2, Sf3a1, Stat6, Smg9, Paxip1, Rmnd5a, Mov10, Ppmlj, Rhoc, Tead3, Ppard, Def6, Peg3, Zim1, Metrn, Lmf1, Ciao3, Angpt14, Dbf4, Daxx, Cd320, Ipo4, Tm9sf1, Irf9, Gmpr2, Mdp1, Dhrs1, Ncan, Tmem161a, Armc6, Borcs8, Slc25a42, Snx9, Ranbp3, Nr2f6, Usel, Ocell, Dyrklb, Braf, Ndufb2, Hltf, Prpf6, Rgs19, Rgs20, Snrpd1, Tchp, Tiam1, Puf60, Golga2, Uck1, Axl, Tgfb1, Ccdc97, Shh, Pdcd21, Gtf2f1, Denndlc, Med6, Prkd1, Prkra, Fkbp7, Plekha3, Ykt6, Baz1b, Pex6, Meal, Kdelr1, Ggct, Jag2, Btbd6, Nudt14, Flii, Top3a, Atg4d, Qtrt1, Hdgf12, Chafla, Adprh, Mcm2, Tpra1, Nab1, Adgre5, Il17ra, Lamb1, Arrdc2, Ppplr17, Timm44, Ap2a2, Pnkp, Med25, Bcam, Clptm1, Relb, Tomm40, Apoe, Hbp1, Prkar2b, Cdkn1b, Klf4, Ap1 m1, Rab8a, Hmgn2, Fam32a, Elf3, Stard3n1, Stk11, Efna2, Atp5d, Ppp5c, Cdk12, Lipe, Cnot11, Slc2a3, Foxj2, Dgcr2, Irf3, Bc12112, Sh3g11, Yju2, Ranbp2, Abcf3, Mrp14, Keap1, Ap1m2, Btbd2, Csnk1g2, Abhd17a, Mob3a, Cacnb3, Fkbp11, Ddx23, Pld3, Grik5, Rabac1, Prkcsh, Elav13, Nosip, Rps11, Supt5, Timm50, Med29, Ncstn, Pex19, Phyhip, Prodh, Ess2, Slc25al, Ddr1, Ier3, As3mt, Homer3, Crtc1, Rps6ka1, Taf61, Insig2, Kpna6, Manla, Itpkc, Coq8b, Kif20a, Gcdh, Calr, Nfat5, Nob1, Bax, Tfam, Zfp81, Efnb3, Ssbp2, Fancl, Brinp2, Gstm5, Gstm7, Cavin1, Map3k11, Akt2, Dnaja3, Hmox2, Cdip1, Cwc15, Dnmt1, Ppan, Angpt12, Trmt10a, Etv1, Psap, Ikbkg, Pax2, Atn1, Phb2, Ptpn6, Eno2, Emg1, Lpcat3, Clcn3, Utp20, Spic, Cul3, Large1, Ddx56, Tmed4, Bid, Ralb, Ppplcc, Dnajb11, Tbccd1, Clec11a, Zfp324, Gab2, Corolc, Tax1bp1, Pkn2, Sgce, Chn2, Wwox, Slbp, Aqp1, Arid3b, Polr2e, Myo9b, Hdac9, Rab11a, Eif2b2, Dlst, Pgf, Ulk2, Dgkq, Chmp2b, Plod3, Mapk13, Srpk1, Lbr, Slc25a18, Thop1, Apba3, Pias4, Map2k1, Sgta, Tmem242, Dtx2, Hnrnpa2b1, Ccdc130, Mri1, Sgsh, Sh2b2, Pex5, Cd44, Slcla2, Wdr1, Ccn4, Ndrg1, Man2b1, Klf5, Wdr83, Prdx2, Polr2a, Senp3, Plekha8, Spc25, Ripk4, Ufd1, Letm1, Cadm3, Slcla6, Slcla3, Crbn, Msh6, Fbxo11, Tb12, Bud23, Nid1, Mcm5, Mprip, Cic, Pafah1b3, Prkaca, Ddx39, Dnajb1, Usp40, Evx1, Kbtbd4, Celf1, Por, Igf1r, Trim28, Ube2m, Ctr9, Eif4g2, Pcytla, Zfp592, Snx6, Mthfd2, Kit, Tomm401, Pan2, Cs, Ampd3, Ctcf, Pard6a, Tfap4, Ranbp1, Rsl1d1, Ergic3, Uqcc1, Ncoa2, Nr2c2, Smpd4, Kctd20, Ephb3, Rcn1, Trap1, Naa60, Ankrd13d, Dhx34, Napa, Crkl, Clint1, Cdkall, Rhox6, Htra1, Zbtb17, Fam131c, Fblim1, Ccdc159, Mob1b, Atp6v1b2, Eps1511, Aamp, Tmbim1, Arpc2, Kmt2b, Tmem147, Susd2, Crip2, Crip1, Pgrmc1, Gcat, Elovl1, Pfdn2, Rnf114, C330007P06Rik, Srm, Epha2, Rbm14, Actn3, Zdhhc24, Bbs1, Pdk1, Ptbp1, Mvd, Stimate, Sfmbt1, Slc4a3, Cdt1, Runx1t1, Hfe, Abhd1, Slc5a6, Aplp1, Qrich1, P4htm, Pola1, Pknox1, Gmnn, Zfp184, Cdk4, B4galnt1, Mettl1, Tspan31, Krt23, Cnp, Sulf2, Stambp, Hap1, P3 h4, Ctnnb1, Vars, Abhd16a, Neu1, Ddah2, Clic1, Lsm2, Pole, Atpla2, Stxla, Zfp775, Scube2, Mrp149, Dennd2c, Mark3, Gatad1, M6pr, Fance, Tinf2, Dus31, Gtpbp3, Tgfbr1, Homer1, Arpp19, Bcl211, Khsrp, Ccdc124, 0610009B22Rik, Zfp655, Ankrd26, Hnrnpa0, Prrg2, Hnrnph1, Id3, Aridla, Crlf1, Ctsd, Hmgcl11, Fzd3, Midlip1, Ap2s1, Fgfrl1, Emc10, Clstn3, Fnbp4, Scrn3, Tle1, Ubc, Prpf31, Sertad1, Carhsp1, Elk3, Nfib, Ubqln4, Rbfox1, Rps18, Ncaph2, Hipk1, Gprc5b, Rala, Vps72, Gabpa, Bmp7, Dynll1, Pdcl, Ap1b1, Bcl2113, Lnpk, Rarres2, Syn2, Elk1, Tnpo1, Rnmt, 6330409D20Rik, Cdk9, Tor2a, Fpgs, Cbx5, GOs2, Pla2g12b, Bcr, Tfcp2, Ubp1, Cilk1, Sdhb, Snap47, Kdsr, Vps4b, Taz, Aldh3a2, Tmem115, Hyal2, Slc3a2, Nxf1, Stx5a, Raver1, 2610507B11Rik, AI597479, Myt1, Gm266, Dazl, Pigyl, Rbm25, Psen2, Acaalb, Plcd1, Fads1, Lmbr1, Cars, Kdelr3, Apip, Pdhx, Vac14, Akt1s1, Adssl1, Brf1, Odc1, Thg11, Pabpc4, Zfp296, Sugp1, Fsd1, Fuz, Sptbn4, Pgam1, Gltp, Bnip2, Scarf2, Brms11, Med15, Dhdds, Crybg2, Ubxn11, Tjap1, Nanog, Tmem167, Mplkip, Kif11, Phox2b, Ttl15, Zfp715, Adgrl1, Amotl1, Etv5, Gfod2, Enkd1, Atp6v0d1, Ptprs, Slc41a1, Iglon5, Tmem175, Tango2, Aldhla2, Ndufs2, Snx31, Atraid, Cad, Sh3bp51, Pten, Timm23, Trnt1, Ehmt2, Elof1, Med16, St3gal1, Tmem259, Clip3, My12, Dedd, Nit1, Tvp23b, Dnajc7, Pigl, Glis2, Bicc1, Map3k4, Piezo1, Rnf166, Srp19, Wdfy2, Rbsn, Mrps25, Dguok, Csf1, Strip1, Kifla, Slc25a11, Cmc2, Chfr, Hoxa2, Tex261, Nol3, Fhod1, Plekhg4, 4931428F04Rik, Tppp3, Msh3, E2f4, Surf4, Dnajb9, Naf1, Yes1, Ppp1cb, Gorasp2, Tsen15, Efr3a, Acsf3, Galns, Gata2, Rabl6, Fbxw5, Slc25a13, Ube2i, Tsr3, Lrrk1, Aldhla3, Sirt2, Hnrnpl, Nolc1, Casd1, Mtmr1, Hmgb3, Map2, Abcal, Lage3, Ubl4a, Gadd45b, Endog, Clpx, Trabd, Dennd6b, Atf6b, Notch4, Rnf5, Fam163a, Mocs2, Lpl, Ttbk1, Srf, Gata3, Lama5, Mrp132, Arnt2, Cers2, Capza2, Tab2, Cnih1, Med28, Nfe212, Prpsap1, Lta4h, Bola1, Castor2, Wnt11, Adss, Fli1, Camk1g, Utp25, H2-M3, Nelfcd, Ctsz, Prelid3b, Sertad4, Ube2a, Slc25a5, Kcnq2, Eefla2, Pls3, Mpped2, Nkap, E2f3, Cr1l, Plgrkt, Dyrk3, Mapkapk2, Lamp2, H3f3b, Nup50, Sulf1, Srsf6, Plcg1, Kctd5, Etaal, Sdc4, Cyth1, Rnd3, Nt5c3b, Coa3, Med24, Exosc10, Vps53, Dnttip1, Acot8, Vtn, Traf4, Stac2, Cacnb4, Zfp207, Timp2, Zc3h18, Top2b, Rarb, Igfbp4, Cdc6, Tnfaip1, Cd40, Slc35c2, Zfp334, Elmo2, Ttpal, Serinc3, Thal, Pgs1, Birc5, Pigt, Wfdc2, Dbndd2, Slc12a5, Ghdc, Pltp, Slc12a7, Ctsa, Zswim1, Slc12a4, Crk, Cox7c, Mlx, Retreg3, Jph2, Dhx58, Rab5a, Nkiras2, Ppp2r5c, Ift52, Myb12, Eya2, Gdap111, Ptgis, Ddx27, Cyth3, Rac3, Rrp7a, Med131, Mafk, Med1, Stard3, Ro60, Stk4, Zcchc10, Tomm34, Anxa6, Zkscan6, Shroom1, Kif3a, Septin8, Mapt, Gid4, Myolb, Dhx40, Clqbp, 6330403K07Rik, Adora2b, Ncor1, Cenpv, Ctdnep1, Phf23, G3bp1, Sparc, Mief2, Tbx3, Dync1 h1, Lsp1, Avpil, Pank3, Wwc1, Mrp158, Mrpl45, Irf1, Slc22a5, P4ha2, Arrb1, Natd1, Map2k3, Ywhah, Sart3, Nars2, Dnah1, Plod1, Rab3d, Mfsd3, Slc25a22, Atp6ap1, Dnase111, Scrn1, BC005537, Isyna1, Mdh2, Rnf145, Atp6vle1, Chtf18, Mapls, Tmem129, Nop9, Psmc3ip, Grb7, Gdpd2, D8Ertd738e, Fkbp8, Ddx39b, Gipc1, Arhgef25, Cdc37, Cops6, Ap4 m1, Gyg, Slc6a8, Arid3a, Ubxn6, Mydgf, Akt3, Lyst, Slc35e1, Polr2i, Fbxo5, Hdac2, Frk, Rwdd1, Hey2, Hint3, Trmt11, Pcmt1, Mtres1, Sec63, Snx3, Cep5711, Cd164, Smpd2, Mical1, Zbtb24, Wasf1, Gtf3c6, Slc16a10, Rev31, Fyn, Tube1, Lama4, Prep, Fam184a, Asfla, Qrs11, Rtn4ip1, Smpdl3a, Reep3, Serinc1, Hsf2, Nts, Dcbld1, Ccdc59, Sim1, P4ha1, Septin10, Zwint, Ube2d1, Slc17a8, Cdk1, Atp2b1, Arid5b, Uhrf1bp11, Dusp6, Tmpo, Psen1, Ikbip, Hbs11, Ccn2, Cdk17, Ntn4, Ndufa12, Tmcc3, Cep83, Socs2, Nudt4, Nuak1, Tcp1112, Timp3, Hsp90b1, Ascl1, Washc3, Dram1, Sirt1, Herc4, Hnrnph3, Rufy2, Ccar1, Ddx21, Srgn, Vps26a, Supv311, Hkdc1, Fam241b, Aifm2, Macroh2a2, Tysnd1, Sarla, Eif4ebp2, Sgpl1, Pcbd1, Unc5b, Slc29a3, Vsir, Lrig3, Anapc16, Ddit4, Dnajb12, Micul, Cand1, Tbk1, Pno1, Srgap1, Egfr, Usp15, Vps54, Tbc1d15, Rab21, 2310011J03Rik, Peli1, Apc2, Slcla4, Rabla, Pwwp3a, Meis1, Uqcr11, Tcf3, Frs2, Yeats4, Cobl, Rab36, Snrpd3, Nav3, Ddc, Cpm, Mdm2, Csrp2, Mknk2, Cabin1, Ap3d1, Sf3a2, Wif1, Timm13, Vps13d, Llph, Helb, Prmt2, Dip2a, Hmg20b, Fzr1, Ncln, Col6a2, Txnrd1, Ppmlm, D10Wsu102e, Wdr82, Pofut2, Sumo3, Hint1, Fbxw11, Stk10, Pfkl, Pus10, Rhbdf1, Pex13, Cfap410, Mpg, Ahsa2, Nprl3, Xpo1, Cpeb4, Stc2, Asb3, Cdc34, Tpgs1, Chac2, Madcam1, Erlec1, Shc2, Sptbn1, Mdh1, Ccng1, Nudcd2, Polrmt, Hmmr, Acs16, Cyfip2, Mgat1, Ppp2ca, Flt4, Hnrnpab, Phykpl, Hspa4, Cnot6, Gfpt2, Zfp354a, Mapk9, Canx, Rack1, Rasgeflc, Rufy1, Rnf130, Rad50, Mrnip, Clk4, Ube2b, Cdkn2aipnl, Med7, Tnip1, Upp1, Slu7, Hus1, Pttg1, Zfp607a, Tns3, Igfbp3, Pes1, Adcyl, Guk1, Eif4enif1, Trim11, Ogdh, Rtn4, Mtif2, Rps27a, Cfap36, Ppp4r3b, Pnpt1, Pold2, Zkscan17, Aebp1, Polm, Pgam2, Dbnl, Mrps24, Dynl12, Xbp1, Supt4a, 2810021J22Rik, Rnf187, Cnot8, Sap301, Galnt10, Mfap3, Fam114a2, Prpsap2, Acaca, Shmt1, Llgl1, Drg2, Srebf1, Bzw2, Elac2, Twistnb, Atxn711, Sypl, Pdia6, Nampt, Rock2, Matn3, Fam49a, Snx13, Sdc1, Lpin1, Pum2, Nrcam, Trib2, Hs1bp3, Smc6, Amz2, Rdh14, Klh129, Trappc12, Rnaseh1, Ubxn2a, Rsad2, Rnf144a, Id2, Mboat2, Ncoa1, Dus41, Rrm2, Bcap29, Cenpo, Klf11, Adcy3, Dnajc27, Efr3b, Cbll1, Pomc, Dnmt3a, Kif3c, Sh3y11, Sntg2, Pxdn, Tlk2, Ftsj3, Psmd12, Slc9a3r1, Jpt1, Unk, Trim47, Mrp138, Fbf1, Acox1, Ten1, Srp68, Exoc7, Ube20, Txndc17, Rhbdf2, Pimreg, Mxra7, Mfsd11, Sec1411, Blmh, Timm22, Nxn, Spag9, Luc713, Lrrc59, Slc35b1, Cacnb1, Fbx120, Nr1d1, Aurkb, Myh10, Ntn1, Stx8, Adprm, Top2a, Acly, Kat2a, Stat5b, Dcakd, Nmt1, Scfd1, Coch, Ap4s1, Dtd2, Ston2, Gtf2a1, Selll, Dnaaf2, Nemf, L2hgdh, Trappc6b, Pnn, Spata7, Npas3, Polr2h, Prorp, Psma6, Nfkbia, Mbip, Sptlc2, Vipas39, Snw1, Nova1, Mthfd1, Ppp2r5e, Rab15, Atl1, Sav1, Tmx1, Timm9, Dhrs7, Ppmla, Six6, Mnat1, Prkch, Hifla, Snapc1, Vtilb, Arg2, Zfp3611, Smoc1, Pcnx, Pacs2, Mta1, Wdr37, Gtpbp4, Zmynd11, Cdca71, Efcab11, Tdp1, Psmc1, Nrde2, Pitrm1, Pfkp, Net1, Tshz3, Dcaf4, Coq6, Entpd5, Ylpm1, Mlh3, Tmed10, Fos, Tgfb3, Angel1, Evl, Yy1, Wars, Meg3, Hsp90aa1, Fdft1, Tecpr2, Cinp, Traf3, Cdc42bpb, Exoc314, Tnfaip2, Eif5, Ppp1r13b, Xrcc3, Klc1, Atp5mpl, Kif26a, Hecw1, Ggps1, Gng4, Gli3, Trim27, Zkscan3, Carmill, Mrs2, Exoc2, Gcnt2, Elov12, Nedd9, Hivep1, Serpinb9b, Ripk1, Pxdc1, Prpf4b, Eci2, Rpp40, Ssr1, Riok1, Snrnp48, Slc35b3, Aopep, Fancc, Ptch1, Ercc612, Sfxn1, Habp4, Ctsl, Prx12c, Cdk20, Mxd3, Txndc15, Zfp729a, Ptdss1, Mterf3, Fastkd3, Cetn3, Hnrnpk, Conh, Rasa1, 2210016F16Rik, Golm1, Agtpbp1, Dapk1, Nkd2, Trip13, Cep72, Pdcd6, Sdha, Ccdc127, Erap1, Cast, Rhobtb3, Srd5a1, Nsun2, Slf1, Med10, Mrp136, Lpcat1, Clptm1l, Vcan, Xrcc4, Zcchc9, Slc30a5, Rad17, Marveld2, Gtf2 h2, Serf1, Smn1, Ptcd2, Ankra2, Arhgef28, Hexb, Gfm2, Polk, Col4a3 bp, Hmgcr, Poc5, Iqgap2, Crhbp, Aggf1, Otp, Ap3b1, Scamp1, Jmy, Kif2a, Ercc8, Depdc1b, Rab3c, Plk2, Thbs4, Serinc5, Mtx3, Zfyve16, Erbin, Nln, Trappc13, Trim23, Ppwd1, Cwc27, Sreklip1, Parp8, Emb, Mrps30, Fgf10, Slc4a7, Ptprg, 4930452B06Rik, Pdhb, Kctd6, Map3k1, Il6st, Ddx4, Plpp1, Gpx8, BC067074, Ndufs4, Fst, Kat6b, Samd8, Vdac2, Nkiras1, Comtd1, Ube2e1, Dlg5, Nglyl, Oxsm, Glud1, Bmprla, Nid2, Nudt13, Ecd, Dnajc9, Anxa7, Ppp3cb, Zswim8, Camk2g, Vcl, Ap3 ml, Txndc16, Eroll, Psmc6, Bmp4, Tmem254b, Ppif, Arf4, Hacl1, Eaf1, Mett16, Capn7, Bap1, Phf7, Dph3, Oxnad1, Nisch, Glt8d1, Spcs1, Kpna3, Spryd7, Rnaseh2b, Pspc1, Ctsb, Gata4, Zmym2, Prkcd, Eeflakmt1, Xpo4, Tdh, Tkt, Pinx1, Lats2, Dcpla, Sap18, Ska3, Mrp157, Zdhhc20, Fgf9, Ints9, Ext13, Cab391, Cdadc1, Atp8a2, Amer2, Spata13, Mipep, Wnt5a, Esd, Zc3h13, Slc25a30, Gpalpp1, Nufip1, Tsc22d1, Enox1, Dnajc15, Epstil, Akap11, Tdrd3, Naa16, Diaph3, Mtrf1, Wbp4, Sugt1, Elp3, Scara5, Esco2, Ccdc25, Ephx2, Trim35, Stmn4, Dpys12, Ppp2r2a, Ebf2, Nefm, Nefl, Entpd4b, Bora, Tnfrsf10b, Rhobtb2, Bin3, Pdlim2, Sorbs3, Ppp3cc, Slc39a14, Fam160b2, Bmp1, Dmtn, Xpo7, Fgf17, Dok2, Gfra2, Rb1, Rcbtb2, Med4, Sucla2, Uch13, Rbm26, Rnf219, Ednrb, Fbx13, Cln5, Tgds, Gpr180, Dnajc3, Mbnl2, Nipbl, Osmr, Dab2, Ttc33, Lrp10, Fbxo4, Drosha, 6030458C11Rik, Pdzd2, Golph3, Zfr, Npr3, Ripk3, Zscan26, Cmbl, Ankrd33b, Amacr, Skor1, Rai14, Brix1, Rad1, Nadk2, Mtdh, Laptm4b, Sdc2, Trio, Ank, Marchf11, Retreg1, Myo10, Rnf19a, Pabpc1, Ywhaz, Grh12, Atp6v1c1, Fzd6, Slc25a32, Lrp12, Utp23, Rad21, Cdh10, Matn2, Pop1, Stk3, Khdrbs3, Eif3e, Ebag9, Myc, Washc5, Sqle, Mtss1, Fbxo32, Wdyhv1, Atad2, Zhx1, Tbcld31, Derl1, Has2, Mtbp, Mrpl13, Col14al, Asap1, Fam49b, Rangap1, Twf1, Nell2, Slc38a2, Srebf2, Slc38a4, Rpap3, Rapgef3, Hdac7, Col2a1, Hoxc5, Gtsf1, Pdelb, Ppplrla, Txndc11, Litaf, Nubp1, 1810013L24Rik, Il1rap, Nudt1611, Mgrn1, Srl, Fgf12, Zfp251, Hes1, Zfp263, Atp13a3, Glyrl, Tmem44, Lsg1, Rogdi, 4930451G09Rik, Eef2kmt, Ercc4, Gpt, Apod, Cyc1, Sharpin, Maf1, Hgh1, Fbx16, Slc52a2, Gpaal, Oplah, Grina, Plec, Scrib, Tsta3, Pycrl, Naprt, Ly6h, Ly6e, Lynx1, Ptk2, Mapk12, Lmf2, Chkb, Mapk8ip2, Arsa, Rabl2, Kif21a, Yaf2, Zcrb1, Prkdc, Ube2v2, Fopnl, Ndel, Mpv171, Pdxdc1, Ntan1, Rrn3, Parn, B3gnt5, Hira, Mrp140, Gbel, Dgcr8, Trmt2a, Riox2, Cldnd1, Klh122, Tomm70a, Slc7a4, Thap7, Lztr1, Snap29, Sdf211, Dlg1, Senp5, Ypell, Ncbp2, Top3b, Pak2, Wdr53, Dnmll, Yars2, Tfrc, Itgb5, Abcc5, Gtf2e1, Ap2 ml, Clcn2, Slc49a4, Ehhadh, Senp2, Tmem41a, D16Ertd472e, Cxadr, Usp25, Rfc4, Eif4a2, St6gal1, Pr12a1, Mrp139, Atp5j, App, Adamts1, Adamts5, Ets2, Dyrkla, Vps26c, Arl13b, Pros1, Psmg1, Brwd1, Pigp, Ripply3, Chaf1b, Doplb, Cbr3, Setd4, Rcan1, Runx1, Itsn1, Donson, Son, Gart, Tmem50b, Ifngr2, Ifnar1, Il10rb, Ifnar2, Synj1, Paxbp1, Sodl, Scaf4, Kans12, Adcy6, Enah, Lmbr11, Prpf40b, Fmnl3, Nckap51, Tmbim6, Faim2, Racgap1, Asic1, Smarcd1, Cox14, Cers5, Lima1, Larp4, Dip2b, Atf1, Slc11a2, Slc4a8, Scn8a, Nr4a1, Krt7, Csad, Soat2, Amhr2, Map3k12, Tarbp2, Rttn, Cdknla, Nus1, Cep89, Mospd1, Akirin1, Cxcl13, Gtf2ird1, Noct, Abcc1, Fhl1, Rfc2, Denr, Prmt5, Nagpa, Get1, Ivnslabp, Rpp14, Slc38al, P3 h3, Serping 1, Serinc2, Scg5, Kcnk5, Creld2, Twf2, Ube2j2, Marchf5, Ache, Pocla, Trip6, Agap3, Tmem176a, Nfatc4, Esx1, Pisd, Tpil, Rab12, Celsr3, Pcbp4, Cdca3, Ogfod2, Mrps23, Rhebl1, Rsph3b, Rps6ka2, Slc25a27, Tnfrsf21, Cenpq, Tbc1d5, Cdc51, Aars2, Mrp114, Sgo1, Slc29a1, Nfkbie, Vegfa, Gtpbp2, Clic5, Enpp4, Rrp36, Ptk7, Cnpy3, Taf8, Gucala, Bysl, Foxp4, Nfya, Brd4, Stk38, Ppil1, Cpne5, Pi16, Mtch1, Pim1, Ccdc167, Cmtr1, Glo1, Slc37a1, Wdr4, Ndufv3, Cbs, Sik1, Arhgap28, Epb4113, Akap8, Myl12a, Wiz, Lpin2, Emilin2, Smchd1, Cyp4f13, Ndc80, Lbh, Galnt14, Ehd3, Dpy30, Spast, Slc30a6, Prkd3, Yipf4, Birc6, Crim1, Vit, Ttc27, Eif2ak2, Cebpz, Ndufaf7, Pja2, Qpct, Man2a1, Cyp1b1, Vapa, Hnrnpll, Ralbp1, Srsf7, Twsg1, Washc1, Washc2, Nrxn1, Cacnalh, Lrpprc, Prepl, Srbd1, Rhoq, Mcfd2, Msh2, Spsb3, Mapk8ip3, Pot1b, Sox8, Tmem8, Axin1, Fam234a, Luc71, Dusp1, Bnip1, Phf1, Plin3, Kdm4b, Rfx2, Uqcc2, Mllt1, Snrpc, Taf11, Anks1, Zfp523, Fkbp5, Nudt12, Cul2, Bambi, Mtpap, Map3k8, Svil, Zeb1, Sos1, Map4k3, Tmem178, Thumpd2, Pkdcc, Cox7a21, Thada, Adcyap1, Polr2d, Adad2, Mapre2, Thoc1, Esco1, Adamts10, Kifc5b, Cdh2, Tapbp, Pfdn6, Vps52, Ring1, Slc39a7, Brd2, Tap2, Etf1, Nelfe, Bag6, Prrc2a, Wdr33, Atp6v1g2, Riok3, Npc1, Zfp521, Impact, Ttc39c, Atat1, Gnl1, Mrps18b, Dele1, Hdac3, Trim26, Gabbr1, Yipf5, Tcerg1, Dpys13, Mbd2, Smad4, Sec11c, Grp, Pmaip1, Gnal, Impa2, Afg312, Prelid3a, Spire1, Psmg2, Ptpn2, Cep192, Ldlrad4, Setbp1, Galr1, Me2, Mapk4, Cxxc1, Mbd1, Smad2, Sall3, Atp9b, Rbfa, Gm16286, Csnkla1, Il17b, Pcyox11, Grpel2, Napg, Txnl1, Fech, Nedd41, Prrc1, Tcof1, Tmx3, Cdx1, Pdgfrb, Hmgxb3, Gnaq, Psat1, Cndp2, Timm21, Cyb5a, Incenp, Fth1, Fads2, Tmem138, Tmem216, Ms4a4d, Mrpl16, Osbp, Fam111a, Gna14, Rfk, Carnmt1, Ccdc86, Prpf19, Tmem132a, Ddb1, Fen1, Zfand5, Cemip2, Rtn3, At13, Naa40, Otub1, Ehd1, Atg2a, Ppp2r5b, Fas, Ak3, Gpha2, Snx15, Jak2, Sac3d1, Cdca5, Zfpl1, Vps51, Tm7sf2, Ankrd1, Syvn1, 1133, Tnks2, Frmd8, Rad9a, Dpf2, Rps6 kb2, Ighmbp2, Pola2, Coro1b, Dmrt1, Eiflad, Chka, Tmem134, Pitpnm1, Sf3b2, Pacs1, Cdk2ap2, Grk2, Klc2, Acy3, Gm49405, Cnih2, Cbwd1, Aldh3b1, Rcel, Pcx, Minpp1, Apba1, Cptla, Mrp111, Mus81, Ppp6r3, Efemp2, Fibp, Lrp5, Smarca2, Vldlr, Rnaseh2c, Kat5, Rela, Slc1a1, Ehbp111, Fam89b, Ltbp3, Scyll, Capn1, Arl2, Men1, Map4k2, Sf1, Rps6ka4, Prdx5, Esrra, Gpr137, Bad, Plcb3, Vegfb, Dnajc4, Rcor2, Mark2, Spindoc, Zdhhc6, Hhex, Cyp26a1, Eif3a, Noc31, Hells, Aldh18a1, Tctn3, Conj, Slit1, Smndc1, Mxi1, Xpnpep1, Trim8, Sfxn2, Maoa, Efhc2, Fundc1, Taf5, Pcgf6, Nr0b1, Slk, Sfr1, Gsto1, 3110040N11Rik, Btbd1, Hdgfl3, Bhlhe22, P4 hb, Arhgdia, Ints4, Alyref, Pcyt2, Sirt7, Tollip, Pycr1, Aspscr1, Cenpx, Lrrc45, Mob2, Maged1, Fasn, Arhgap19, Dus11, Gps1, Zdhhc16, Rfng, Slc16a3, Csnkld, Ogfod3, Ubtd1, Wdr45b, Fn3k, R3hcc11, Lox14, Hps1, Got1, Cutc, Cpn1, Chuk, Cwf1911, Bloc1s2-ps, Scd2, Ndufb8, Mrp143, Actrla, Hexa, Arih1, Adpgk, Parp6, Tbl1x, Ribc1, Hsd17b10, Huwel, Tsr2, Fgd1, Gn131, Maged2, Apex2, Pfkfb1, Tro, Abhd6, Flnb, Polr3a, Sat1, Acot9, Ptprj, Banp, Jph3, Itgb8, Sp4, Padi3, Kdm5c, Sbds, Rabgef1, Pa2g4, Esyt1, Smarcc2, Chmp6, Baiap2, Rnf41, Nabp2, Tepsin, Cnpy2, Faap100, Atp5b, Prim1, Nab2, Shmt2, R³hdm2, Gli1, Ddit3, Mbd6, Ttc4, Pip4k2c, Hdhd2, Pias2, St8sia5, Rnf165, Atp5a1, Atp23, Usp33, Clnsla, Paip1, Ric8a, Psmd13, Ifitm1, Ptdss2, Irf7, Hras, Tmem80, Pidd1, Pnpla2, Cd151, Chid1, Tmem192, Apool, Chm, Crcp, Asl, Gusb, Sumf2, Clybl, Farp1, Dock9, Tnrc6c, Tmc6, Tk1, Cant1, Rbfox3, Cbx2, Cbx8, Gaa, Eif4a3, Nptx1, Rptor, Tma16, Dach2, Bach1, Cct8, Usp16, Phf6, Ccdc51, Atrip, Shisa5, Pfkfb4, Arhgef9, Rps6ka6, Marchf8, Wdr73, Nmb, Sec11a, Pigq, Rab40c, Mcrip2, Rhot2, Jmjd8, Wdr24, Gng13, Prps2, Sdc3, Hadha, Tyms, Plk4, Mfsd8, Larp1b, Itih5, Atp5c1, Taf3, St8sia2, Slco3a1, Pgm1, Slc25a10, Hgs, Rassf3, Itgb1, Nrp1, Pard3, Sec61a2, Zfp282, Pdia4, Iscu, Fam20c, Prkar1b, Pdgfa, Dnaaf5, Get4, Xiap, Stag2, Higd2a, Thoc3, Smad7, Myo5b, Casp12, Gria4, Kbtbd3, Aasdhppt, Alkbh8, Sox17, Lypla1, Rblcc1, Sgk3, Cops5, Stau2, Rdh10, Terf1, Tfap2b, Paqr8, Tmem14a, Tram1, Lactb2, Ube2w, Tmem70, Pikfyve, Mett121a, Creb1, Klf7, Adam23, Eef1b2, Ndufs1, Nrp2, Maip1, Mob4, Coq10b, Sf3b1, Slc39a10, Slc40a1, Wdr75, Ikzf2, Lancl1, Acadl, Kansl11, Rpe, Raph1, Carf, Wdr12, Nop58, Sumo1, Stradb, Trak2, Casp8, Cflar, Clk1, Ppil3, Nif311, Orc2, Sgo2a, Col5a2, Col3a1, Poglut2, Ercc5, Tex30, Map4k4, Eif5b, Stk17b, Asnsd1, Osgepl1, Pms1, Gls, Stat1, Nabp1, Tmeff2, Zfp142, Rhbdd1, Col9a1, Mff, Fam135a, Smap1, Ogfrl1, Agfg1, Nhej1, Sphkap, Rnf25, Bcs11, Cnot9, Ctdsp1, Pnkd, Igfbp5, Xrcc5, Atic, Fn1, Bard1, Zfand2b, Abcb6, Ankzf1, Stk16, Tuba4a, Dnajb2, Ptprn, Speg, Des, Dnpep, Obsl1, Stk11ip, Trip12, Sp100, Itm2c, 2810459M11Rik, Psmd1, Ncl, Epha4, Pde6d, Cops7b, Nppc, Farsb, Ecel1, Mrp144, Eif4e2, Ngef, Ndufa10, Rnpepl1, Capn10, Mterf4, Pask, Ppp1r7, Stk25, Bok, Thap4, Atg4b, Dtymk, Ing5, Inpp5d, Atg1611, Lrrfip1, Scly, Asb1, Hdac4, Relch, Gin1, Ccdc93, Slc35f5, Lypd1, Tmem163, Cont2, Ubxn4, Mcm6, Cdc73, Tsn, Nifk, Ptpn4, Dbi, 3110009E18Rik, Steap3, Nr5a2, Ddx59, Csrp1, Srgap2, Ar18a, Eif2d, Ube2t, Nucks1, Rbbp5, Lrrn2, Cyb5r1, Adipor1, Myog, Lhx4, Xpr1, Cdc42bpa, Ahctf1, Tfb2m, Kif26b, Parp1, Acbd3, Sdccag8, Capn2, Trp53bp2, Srp9, Nvl, Pycr2, Opn3, Rgs7, Ifi211, Dusp23, Cfap45, Copa, Dcaf8, Vang12, Mpz11, Smyd2, Ptpn14, Cenpf, Kctd3, Esrrg, Eprs, Iars2, Mark1, Nek2, Lpgat1, Ppp2r5a, Pacc1, Atf3, Batf3, Tatdn3, Angel2, Traf5, Plxna2, Usf1, Nmt2, Acbd7, Suv39 h2, Cfap126, Meig1, Fam107b, Frmd4a, Dusp12, Sephs1, Atf6, Uhmk1, Mcm10, Uap1, Ddr2, Hsd17b7, Ccdc3, Nuf2, Aldh9a1, Mgst3, Prdx6, Trdmt1, Cubn, Rsul, Nek6, Psmb7, Ppp6c, Golga1, Arpc51, Orc4, Kif5c, Mmadhc, Mindy3, Pfkfb3, Abi2, Pkn3, Zbtb43, Slc2a8, Lrsam1, Fam129b, Stxbp1, Dpm2, St6galnac6, Eng, Gtf3c5, Ak1, Slc25a25, Ptges2, Ralgds, Dnm1, Nr4a2, Gpd2, Olfm1, Acvr1, Upp2, Fibcd1, Abl1, Fubp3, Torlb, Torla, BC005624, Crat, Dolpp1, Ntmt1, Sh3glb2, Hspa5, Gapvd1, Psmd5, Zeb2, Phf19, Rab14, Gsn, Stom, Dab2ip, Mrrf, Grb14, Rbm18, Gca, Ifih1, Psmd14, Strbp, Notch1, Nmi, Arl6ip6, Anapc2, Ssna1, Rbms1, Marchf7, Baz2b, Wdsub1, Pkp4, Galnt3, Nup35, Nckap1, Dnajc10, Itprid2, Itga4, Ube2e3, Dyncli2, Cwc22, Cybrd1, Hat1, Stk39, Cers6, Ssrp1, Lrp2, Slc43a3, Slc43al, Smtnl1, Ube216, Clp1, Med19, Tfpi, Fastkd1, Itgav, Phospho2, Zc3h15, Mtx2, Hoxd8, Atf2, Sp3, Itga6, Arl14ep, Slc12a6, Emc4, Katnbl1, Lpcat4, Ccdc34, Lin7c, Traf6, Dnajc24, Pax6, Depdc7, Cstf3, Hipk3, Fbxo3, Caprin1, Nat10, Pamr1, Trim44, Api5, Ttc17, Hsd17b12, Ext2, Fbn1, Cops2, Meis2, Cd82, Tspan18, Chst1, Pex16, Mapk8ip1, Creb311, Mdk, Harbil, Atg13, Arhgap1, Arfgap2, Pacsin3, Mtch2, Rad51, Knstrn, Mrps5, Bub1, Nop56, Cpxml, Lpin3, Rrbp1, Fam110a, Rbck1, Mapre1, Fam210b, Aurka, Cstf1, Pkia, Stmn2, Rtf2, Tpd52, Rael, Rbm38, Zbp1, Rab22a, Zdbf2, Stx16, Gnas, Phactr3, Impa1, Fabp5, Snx16, Chmp4c, Ptpn1, Atp9a, Sall4, Lrrcc1, Zfp64, E2f5, Car13, Car3, Dok5, Car2, Ntsr1, Mrgbp, Col9a3, Gid8, Arfgap1, Chrna4, Helz2, Zgpat, Zbtb46, Oprl1, Pcmtd2, Raly, Ahcy, Itch, Mtfr1, Map1lc3a, Ggt7, Acss2, Gss, Procr, Mmp24, Eif6, Nfs1, Rbm39, Epb4111, Aar2, Tbl1xr1, Ndrg3, Dsn1, Samhd1, Rbl1, Rpn2, Ghrh, Src, Ttil, Rprd1b, Ralgapb, Fam83d, Dhx35, Skil, Pik3ca, Zfp639, Mfn1, Gnb4, Ttc14, Ncoa3, Fxr1, Nceh1, Ect2, Lrrc34, Sec62, Dcun1d1, Mccc1, Acad9, Spata5, Ufm1, Postn, Supt20, Mbnl1, Dhx36, Gfm1, Mfsd1, Schip1, Ift80, Kpna4, Ppm11, Nmd3, Bche, Dclk1, Nbea, Tm4sf1, Wwtrl, Pfn2, Tsc22d2, Etfdh, Eif2a, 4930579G24Rik, Mme, Slc33al, Gmps, Kcnab1, Ssr3, Ccnl1, Ptx3, Serpini1, Pdcd10, Dclre1b, Olfm13, Syt6, Nras, Sike1, Prpf38b, Stxbp3, Gpsm2, Clcc1, Ampd2, Gstm4, Ahcyl1, Kcnc4, Dram2, Ddx20, Ints3, Gasklb, Tmem144, Slc35a3, Sass6, Vcam1, Ext12, Colllal, Neurog2, Larp7, Prss12, Rnpc3, Hadh, Trim2, Sfrp2, Casp6, Plrg1, Pla2g12a, Gucylb1, Ppa2, Ctso, Ints12, Gstcd, Pdgfc, Pitx2, Enpep, Dkk2, Papss1, Fubp1, Dnajb4, Ptgfr, Pmf1, Sh3d19, Gatb, Fbxw7, Mett114, Bcar3, Gclm, Abcd3, F3, Rwdd3, Ptbp2, Rap1gds1, Tspan5, Eif4e, Mttp, Dapp1, Ppp3ca, Nfkb1, Manba, Wls, Depdcla, Cth, Zranb2, Adgrl2, Rpf1, Ctbs, Bc110, Ccn1, Cryz, Asph, Trp53inp1, Ccne2, Gem, Fam92a, Pip4p2, Calb1, Decr1, Nbn, Mmp16, Cpne3, Tmem68, Rps20, Nsmaf, Coq3, Pnisr, Sdcbp, Tstd3, Conc, Odf21, Ndufaf4, Lmo4, Gbp3, Gbp2, Pdlim5, Rngtt, Ube2j1, Rragd, Casp8ap2, Map3k7, Epha7, Slc35al, Smim8, C9orf72, Rnf20, Smc2, Polrle, Exosc3, Ncbp1, Trmo, Anp32b, Nans, Coro2a, Nr4a3, Erp44, Invs, Tex10, Tmeff1, Brinp1, Fmn2, Zfp618, Slc44a5, Tnc, Txn1, Pappa, Astn2, Ptgr1, Ugcg, Ptbp3, Hsd12, Snx30, Wdr31, Alad, Pole3, Kdm4c, Dmac1, Ptprd, Mpdz, Zdhhc21, Dnaja1, Aptx, Slc44a1, B4galt1, Fktn, Chmp5, Tmem38b, Nfx1, Rad23b, Elp1, Epb4114b, Enho, Stom12, Tesk1, Tln1, Glipr2, Snapc3, Psip1, Plin2, Txndc12, Pum1, Pdpn, Lrrc38, Pramef12, Tnfrsf1b, Echdc2, Czib, Lrrc42, Col9a2, Exo5, Dyrk2, Hivep3, Ppcs, Ccdc30, Ybx1, Tfap2c, P3 h1, Svbp, Slc2a1, Rragc, Mycbp, Macf1, Ppie, Trit1, Mycl, Mfsd2a, Cap1, Ppt1, Ephb2, Eloa, Pithd1, Gale, Fucal, Pnrc2, Srsf10, Kif2c, Lrrc41, Aldh4a1, Mrto4, Pqlc2, Capzb, Cda, Pink1, Ddost, Hp1bp3, Eif4g3, Hspg2, Alpl, Zcchc17, Tinagl1, Hcrtr1, Pef1, Adgrb2, Hpca, Ptp4a2, Azin2, Khdrbs1, Ak2, Rnf19b, Ccdc28b, Phc2, Tmem234, Zfp362, Hdac1, Nipal3, Srrm1, Yars, Sfpq, Tmem50a, Maco1, AU040320, Ncdn, Psmb2, Zfp593, Ago3, Sh3bgrl3, Trappc3, Gpn2, Map7d1, Gpatch3, Slc9a1, Mrps15, Meaf6, Gnl2, Rspo1, Cdca8, Epha10, Fam76a, Stx12, Ppp1r8, Rpa2, Smpdl3b, Eya3, Yrdc, Mtf1, Sesn2, Rcc1, Traulap, Gmeb1, Epb41, Utp11, Ptpru, Mecr, Srsf4, Casp9, Fbxo42, Necap2, Padi2, Xrcc2, Prkag2, Abcf2, Nub1, Tmub1, Fastk, Ube4b, Pgd, Slc4a2, Cdk5, Abcb8, Dffa, Casz1, Nos3, H6pd, Slc25a33, Klh17, Ctnnbip1, Lzic, Mtor, Fam126a, Rint1, Fbxo44, Mad212, Kmt2e, Draxin, Agtrap, Mthfr, Mfn2, Miip, Lrrc47, Atad3a, Rer1, Ski, Sdf4, 5730480H06Rik, Adgra3, Trmt44, Rgs12, Lrpap1, Add1, Rnf4, Nelfa, Nkx1-1, Man2b2, Ppp2r2c, Crmp1, Evc, Nsg1, Rab28, Dnajb6, Fos12, Rbks, Slc4alap, Eif2b4, Snx17, Ppm1g, Nrbp1, Slc30a3, Cgref1, Emilin1, Agb15, Mapre3, Dpys15, Slc35f6, Anapc4, Cenpa, Lias, Commd8, Slc30a9, Pdgfra, Pigg, Mtf2, Dipkla, Spp1, Sparc11, Nudt9, Tpst2, Tfip11, Srrd, Asphd2, Nos1, Rfc5, Wsb2, Shroom3, Tctn2, Gtf2 h3, Eif2b1, Ddx55, Rilpl1, Cdk2ap1, Rilp12, Snrnp35, Arl6ip4, G3bp2, Pitpnm2, Kntc1, Slc15a4, Rsrc2, Zcchc8, Stx2, Ran, Nipsnap2, Diablo, Vps33a, Mmp17, Bcl7a, Sfswap, Wdr66, Cct6a, Rhof, Mapkapk5, Acad10, Brap, Fam168a, Vps29, Fam216a, Gpn3, Arpc3, Anapc7, Atp2a2, P2rx7, Ift81, Camkk2, Anapc5, Rnf34, Kdm2b, Ncor2, Dhx37, Aacs, Pgam5, Ankle2, Golga3, Ddx51, Ep400, Pus1, Gpc2, Ulk1, Prkab1, Cit, Rab35, Chek2, Sirt4, Pxn, Triap1, Gatc, Srsf9, Cabp1, Acads, Ints1, Spp13, Psmg3, Mad111, Mrm2, 2210016L21Rik, Snx8, Oas12, Mmab, Radil, Ube3b, Wipi2, Actb, Fscn1, Zfp12, Ung, Usp30, Rbm19, Plbd2, Ddx54, Rita1, Rasal1, Dtx1, Oas1b, Rph3a, Aimp2, Eif2ak1, Rpl6, Erp29, Arpcla, Arpc1b, Ptcd1, Cpsf4, Zkscan14, Rnf6, Cdk8, Polrld, Colla2, Auts2, Limk1, Eln, Bcl7b, Cul1, Ezh2, Rbm28, Lrwd1, Cux1, Ephb4, Actl6b, Gnb2, Gigyf1, Pop7, Pcolce, Gm20605, Agfg2, Tsc22d4, Mepce, Zkscan1, Irf5, Hibadh, Adcyap1r1, Nt5c3, Fkbp9, Tmem209, Av19, Herc6, Abcg2, Tmem176b, Igf2bp3, Tra2a, Npy, Gsdme, Osbp13, Nfe213, Trim24, Hoxa1, Ccdc184, Mad211, Mrps33, Smarcad1, Mkrn1, Gfpt1, Nfu1, Anxa4, Pcyox1, Add2, Dusp11, Cct7, Pradc1, Zfp638, Fbx114, Prickle2, Adamts9, Lrig1, 1700003E16Rik, Kbtbd8, Wdr54, Ino80b, Wbp1, Mogs, Mrp153, Mlap, Pole4, Tacr1, Mrp119, Aplf, Rab43, Cnbp, Copg1, Tmf1, Hmces, Uba3, Frmd4b, Foxp1, Chl1, Ruvbl1, Sec61a1, Abtb1, Plxna1, Aldh111, Slc41a3, Nup210, Wnt7a, Xpc, Tmem43, Grip2, Sumf1, Itpr1, Bhlhe40, Edem1, Ar18b, Ret, Gdf3, Ptms, Tmccl, Bms1, Zfp248, Gabarapl1, Rad52, Adipor2, Wnt5b, Erc1, Ccdc77, Magohb, Ybx3, Etv6, Lrp6, Dusp16, Ddx47, Fam234b, Emp1, Grin2b, Atf7ip, Wbp11, Arhgdib, Ptpro, Strap, Plekha5, Aebp2, Golt1b, Kcnj8, Rad18, Srgap3, Thumpd3, Bcat1, Mtmr14, Ogg1, Camk1, Etnk1, C2cd5, Il17rc, St8sia1, Creld1, Sec13, Atp2b2, Slc6a11, Slc6al, Atg7, Tamm41, Cand2, Mbd4, Ift122, Pianp, Ing4, Mrp151, Ltbr, Tnfrsfla, D6Wsu163e, Prmt8, Tspan11, Tspan9, Fkbp4, Slc8a2, Zfp606, Zik1, Mark4, Ercc2, Rtn2, Ppmln, Vasp, Qpctl, Dmpk, Dmwd, Nova2, Pdcd5, Uri1, Rp128, U2af2, Zfp273, Psd3, Zfp719, Faap24, Rhpn2, Slc7a10, Kctd15, Asb7, Snrpal, Pcsk6, Tars12, Tjp1, Fam189a1, Apba2, Mtmr10, Furin, Hddc3, Unc45a, Iqgap1, Sema4b, Idh2, Nr2f2, Synm, Ttc23, Lrrc28, Mef2a, Ctsc, Nox4, Rasgrp4, Fam98c, Psmd8, Nfkbib, Lrfn1, Pak4, Psmc4, Zfp626, Mfge8, Aen, Det1, Mrps11, Mrp146, Tmem126b, Eed, Zfand6, Sh3g13, Ddias, Rab30, Anapc15, Gm45837, Arl6ip1, Smg1, Nucb2, Ipo5, 1110004F10Rik, Calca, Pde3b, Qprt, Pagrla, Mvp, Cdipt, Sez612, Kctd13, Stard10, Ino80e, Fchsd2, Ppp4c, Plekhb1, Rab6a, Mrpl48, Corola, Sgf29, Cln3, Lipt2, Pold3, Rabep2, Pgm211, Syt3, Eif3c, Myh14, Spns1, Rps3, Dgat2, Il4ra, Psmal, Kdm8, Thap12, Copb1, Zkscan2, Far1, Acer3, Myo7a, Arhgap17, Disp1, Parva, Pak1, Rbbp6, BC017158, Slc5a2, Tgfbli1, Rnf141, Adm, Fus, Prss8, Kat8, Bckdk, Stx1b, Fbx119, Bcl7c, Phkg2, Rnf40, Prr14, 9130019022Rik, Bcat2, Nomo1, Lamtor1, Rgs10, Tiall, Bag3, Fgfr2, Ate1, Tacc2, Acadsb, Plk1, Dctn5, Ndufab1, Ubfd1, Ears2, Gga2, Mett19, Cdr2, Mrp117, Polr3e, Arfip2, Uqcrc2, Rrp8, Ilk, Tmem159, Trim30a, Lyrm1, Rexo5, Eri2, Chst15, Oat, Thumpd1, Lhpp, Fam53b, Abraxas2, Trim21, Zranb1, Ctbp2, Rrm1, Knop1, Vps351, Stim1, Pgap2, Mki67, Atp6ap2, Usp9x, Cask, Swap70, Wee1, Tmem9b, Akip1, Denn2b, Tub, Ndufb11, Cdk16, Usp11, Dock11, Cul4b, Elf4, Rab33a, Enox2, Igsf1, Stk26, Gpc4, Rbmx, Fgf13, Mcf2, Ebp, Rpgr, 3830417A13Rik, Mtcp1, Brcc3, Rab39b, Msn, Heph, Ophn1, Stard8, Efnb1, Pbdc1, Magee1, Magt1, Itm2a, 2610002M06Rik, Cstf2, Gla, Acs14, Pak3, Dcx, Lrch2, Phka2, Slc7a3, Map3k15, Rps6ka3, Zmym3, Nono, Taf1, Chic1, Flna, Abcb7, Gpm6b, Zfp185, Rbbp7, Syap1, Ms13, Ctps2, Zfp275, Zrsr2, Haus7, Bgn, Abcd1, Vegfd, Piga, Asb9, Hcfc1, Renbp, Naa10, Irak1, Mecp2, Plxna3, Fam3a, G6pdx, Dkc1, Nxf7, Morf412, Zcchc18, Psmd10, Tsc22d3, Prps1, Morc4, Rnf128, Coprs, Eif4ebp1, Vegfc, Aga, Saraf, Gpat4, Sfrp1, Tm2d2, Slit2, Tenm3, Dctd, Wwc2, Rwdd4a, Mak16, Msmo1, Klh12, Galnt7, Scrg1, Hpgd, Ednra, Tmem184c, Sin3b, Slc25a4, Cbr4, Sh3rf1, N4bp1, Cbln1, Heatr3, Adcy7, Brd7, Nkd1, Sall1, Rb12, Aktip, Eif2ak3, Gins3, Setd6, Got2, Cdh11, Smad1, Lsm6, Slc10a7, Tnpo2, Vps35, Orc6, Gpt2, Dnaja2, Itfg1, Tecr, Zfp330, Gab1, Smarca5, Txnl4b, Pmfbp1, Zfp821, Ist1, Ap1g1, Phlpp2, Irx3, Irx5, Mmp2, Gnaol, St3gal2, Amfr, Cog4, Nudt21, Bbs2, Cenpn, Mt2, Mt1, Herpud1, Fam192a, Arl2 bp, Cx3cl1, Coq9, Adgrg1, Katnb1, Mmp15, Tmem38a, B3gnt3, Jak3, Slc27al, Mapllc3b, Emc8, Babam1, Gins2, Zdhhc7, Usp10, Cotl1, Klh136, Taflc, Mast3, Pik3r2, Mbtps1, Rab3a, Mphosph6, Ntpcr, Mau2, Pbx4, Lpar2, Ints10, Dctn1, Cdh5, Cmtm3, Nael, Rrad, Cbfb, Tradd, D230025D16Rik, Hsd11b2, Psmb10, Dus2, Pla2g15, Slc7a6, Smpd3, Zfp90, Vps4a, Cog8, Nip7, Mtmr2, Terf2, Cep57, Cyb5b, Mrella, Ankrd49, Panx1, 4931406C07Rik, Taf1d, Kars, Adat1, Gabarap12, Tmem231, Chst5, Tmem170, Cfdp1, Wdr59, Aars, Cdh15, Bmper, Afg311, Acad8, Ccsap, Acta1, Urb2, Cog2, Agt, Arv1, 2310022B05Rik, 2810004N23Rik, Gnpat, Sprtn, Egln1, Vps26b, Jam3, St14, Aplp2, Birc3, Dcun1d5, Sesn3, Cul5, Kcnj5, St3gal4, Dcps, Rpusd4, Acat1, Rdx, Zpr1, Pus3, Olfm2, Tyk2, Pde4a, Ilf3, Tmed1, Scg3, Yipf2, Lysmd2, Carm1, Tmod2, Smarca4, Gnb5, Ldlr, Kank2, Dock6, Polr2m, Rab27a, Sltm, Nedd4, Rnf111, Ccnb2, Myole, Mns1, Fam81a, Tcf12, Anxa2, Cgnl1, Ice2, Rp9, Itga11, Fem1b, Cln6, Anp32a, Glce, Phip, Kif23, Lca5, Elov14, Bckdhb, Idh3a, Tle3, Acsbg1, Imp3, Thsd4, Ptpn9, Ireb2, Pkm, Man2c1, 1700017B05Rik, Psma4, Chrna3, Fam219b, Mpi, Ube2q2, Ulk3, Fbxo22, Csk, Tmem266, Clk3, Is12, Rcn2, Tspan3, Stra6, Tmem30a, Hmg20a, Col12a1, Stoml1, Loxl1, Nptn, Neol, Mto1, Gsta4, Elov15, Gclc, Lrrc1, Tmed3, Rasgrf1, Tpm1, Zic1, Plscr1, Lactb, Plod2, Aph1b, Usp3, Ciao2a, Ppib, Csnk1g1, Trip4, Rbpms2, Spg21, Parp16, Dpp8, Igdcc3, Dis31, Snapc5, Rpl4, Zwilch, Smad3, 2300009A05Rik, Pias1, U2surp, Atr, Tfdp2, Atp1b3, Rasa2, Ube2cbp, Tbx18, Syncrip, Crtap, Cmtm6, Dync1li1, Cmtm7, Stt3b, Tgfbr2, Eomes, Slc25a36, Clstn2, Copb2, Mrps22, Pik3cb, Faim, Armc8, Dbr1, Mras, Cdc25a, Nme6, Map4, Dhx30, Smarcc1, Cspg5, Scap, Nradd, Pthlr, Tdgf1, Lrrfip2, Mlh1, Dclk3, Trib1, Stac, Pdcd6ip, Fbx12, Myd88, Wdr48, Gorasp1, Csrnp1, Rpsa, Slc25a38, Pccb, Amot12, Cep63, Ephb1, Ryk, Slco2al, Rab6b, Srprb, Trf, Topbp1, Uba5, Nphp3, Mrp13, Nudt16, Astel, Atp2c1, Manf, Nckipsd, Ip6k2, Prkar2a, Slc25a20, Usp4, Nt5m, Srek1, Em14, Flcn, Atxn21, Chsyl, Gpr19, Prorsd1, Cd59a, Malt1, Oas2, Lmo2, Exd2, Syde1, Mdfi, Mansc1, Abtb2, Mier3, Snx33, Ablim3, Inppl1, Ccdc88a, Tpcn1, D430042009Rik, Hey1, Gpbp1, Slc8b1, Ilvbl, Gtf3c1, Troap, Alas1, Pdxk, Lamal, Cdv3, Slc10a3, Arap1, Fanca, Ank2, Pwp2, 2410131K14Rik, Prr51, Zswim6, Abcc4, Rnft2, Fbxw8, Smap2, Cyb5r4, Arhgef17, Acs13, Rims3, Rangrf, Nfyc, Fbxo21, Slc16al, Cspg4, Lrig2, Hspa13, Nup93, Rasgrp2, Ap4b1, Pebp1, Inha, Fam207a, Chpf, Foxj3, Nlrp4f, Mycbp2, Asic4, Ogfod1, Trim33, Nfatc1, Gmppa, Afap111, Eif31, Npat, Lmo7, Resp18, Pfkm, Gas7, Senp1, Nol12, Cdc14b, Lss, Slc7a6os, 3830406C13Rik, Slc35d2, Atg9a, Ybey, Podx12, Cnppd1, Dis3, Mgll, Lpcat2, Ttc28, Eefsec, Wnt6, Scaf11, Arid2, Ttl14, Stk36, Ptprm, Rpgrip11, Noc41, Lpp, Dpyd, Fbx18, Galnt9, Gstt2, Ctdp1, Dnm2, Chst2, Map2k4, Rtp4, Pus71, Prrg3, Usp37, Fntb, Frrs1, Arhgap44, Clasp2, Agl, Nudt15, Gucd1, Antxr1, Mcee, Terf2ip, Gtpbp6, Armcx2, Specc11, Lpar6, Adamts15, Zbtb1, Armcx1, Crlf2, Fndc3a, Cdc14a, Casp4, Gtf2a2, Znrf1, Dph5, Myo6, Tmem35a, Igf2bp2, Ndn, Lgi3, Rfwd3, Pcgf3, Hacd3, Piwil2, Vps8, Ddx19b, Zfp7, Ucp2, Arhgap39, Fcsk, Smyd5, Lrrc24, Ccar2, Foxn3, Vav3, Lrrc14, Sf3b3, Spr, Fndc3c1, Fkbpl, Gadd45gip1, Rbm4b, Mtss2, Tmem1311, Nrxn2, Exoc6b, Asb13, Atp6vlh, Tasor2, Alg3, Tcea1, Ppp1r16a, Tpbpa, Mrp115, Engase, Cdk10, Lgals3 bp, Pxk, Map9, Ccp110, Zdhhc15, Usp36, Gucylal, Chmp2a, Gdel, Rbm34, Vhl, Ndufb7, Ppp3r1, Tnks1bp1, Zfp446, Ttc37, Kcnk1, Slc66a2, Scaper, Cpsf1, Fancd2, Cct2, Rpap1, Fgd5, Galnt17, Farp2, Zdhhc5, Fst15, Ctnnd1, Meak7, Golim4, Kctd7, Tpst1, Srsf2, Pomt2, Exoc3, Ogt, Scx, Zfc3 h1, Cond3, Irf2bpl, Zbed5, Hsdl1, Chmp7, Lsm3, R3hcc1, Chchd4, Lox12, Mrps17, Atm, Gpc1, Foxj1, Usp54, Slc25a37, Senp6, Agpat1, Flvcr2, Exosc4, Ints14, Setd5, Jdp2, Nek9, Tmem132d, Kif4, Iqsec1, Trim59, Tmem132c, Kctd9, Plcg2, Fam151b, Ina, Ube2f, Gtf2 h5, Smc4, Cpne2, Tulp4, Cmip, Fbxo15, Pja1, Neurl1b, Gcsh, Zfp707, Zxdc, Cops8, Tmem30b, Gulo, Eda2r, Scara3, Diaph2, Uaca, Gbx2, Poglut3, Edil3, Pcnx4, Hmgxb4, Zfp395, Ice1, Fbxo16, Zfp418, Rsrc1, Hdx, Washc4, Ccpg1, Tent4a, Slc41a2, Myo5a, 2700049A03Rik, Mon2, Chst11, Ppmlh, Mtrr, Tiparp, Ankrd12, Lrrn1, Cacnala, Xpot, Pbx2, Dbn1, Nlrp4c, Neurod1, Gns, Ppp1r21, Mrps10, Adgrb1, Dgkh, Nostrin, Nagk, Sirt6, Mast4, Map4k5, Vax2, Gna11, G6pc3, Ccdc122, Unc13a, Sos2, Colgalt1, Grip1, Tet3, Larp6, Mfsd12, Fam214a, Ankrd27, Myl12b, Mrp134, Cactin, Cog3, Pip5klc, Pygol, Tjp3, Rtkn, Mrp154, Cog7, Atcay, Vat1, Eef2, Foxn2, Dpp4, Rundc1, Zbtb7a, Bazla, Map2k2, Kri1, Dhx57, Zc4 h2, Eef2k, Xkr6, Mtmr9, Becn1, Fam167a, Egln3, Gcfc2, Arhgap5, Nubpl, Elmod2, Ints6, Zc3h12c, Tanc1, Plekhh3, Heatr5a, Fam124a, Tubg1, Arl6ip5, Chrnb4, Epn1, Sppl2b, Lsm7, Spcs2, Ccdc106, Pdk3, Trim13, Scai, Oaz1, Eogt, Pcyt1b, Hectd1, Tut7, Pkig, Tpbg, Arx, Plekhj1, Nat14, G2e3, Klh135, Mid1, Gnptab, Gdpd5, Sec31a, Fbxo33, Uvrag, Pdzrn3, Parpbp, Rmi1, Gm49322, 1810055G02Rik, Shq1, Denndla, Klf16, Oser1, Emsy, Crb2, Tmem98, Rabgap1, Haus8, Foxa1, Fignl1, Rcbtb1, Slc25a21, Mbd3, Fthl17a, Tgfbi, Tstd2, Reep6, Tdrd7, Prdm4, Eif1, Capn5, Igfbp11, Mpv1712, Aldh1b1, Pcdh17, Ankrd11, Dcaf10, Chrna5, Fam174c, Trmt10b, Frmpd1, Ric8b, Midn, Rsf1, Grhpr, Cbarp, Aamdc, Zcchc7, Zswim4, Sbno2, Tnfsf9, Melk, Isg15, Rnf38, Arhgap45, Alg8, Usp35, Prkx, Wdr18, Selenoo, Bbs10, Tmem161b, Fbxo45, Dym, Dync1li2, Krt20, Cd9912, Ggta1, R3hdm4, Cep19, Zdhhc17, Twist1, Mlc1, Tk2, Pim3, Polr3g, Plppr3, Lysmd3, Ids, Palm, Syt1, Zhx3, Cox8a, Rnf126, Dennd5a, Cd276, Bbs9, H2-Q4, Cog5, Aldh5a1, Ibtk, Fbxw2, Tdp2, Apex1, Tmem591, Ints61, Fnip1, Mett125, Prtg, Zfp57, Fmnl2, Ptpn23, Zranb3, Ar15a, Vezt, Rab3gap1, Mbnl3, Lmo1, Metap2, Rpp251, Rfxank, Hoxc9, Mgat5, Prickle1, Pphln1, Sostdc1, Gatad2a, H1f2, Sapcd1, Ankmy2, Gxylt1, Arhgap36, Ndufa13, Rif1, Sh3bp4, Znrdlas, Pdzrn4, Skal, Kctd1, Ube2r2, Gmip, Rbm43, Pnpla8, Fst14, Lrrk2, 9530068E07Rik, Macrod1, Lrrn3, Slc2a13, BC031181, Lzts1, Skpla, Srp72, Kidins220, Igsf10, Ubac1, Csgalnact1, Serbp1, Abt1, Gadd45a, Ppp1r11, Gng12, Arsi, Pcdh8, Gpi1, Tbcc, Siah2, Thap11, Lum, Wtip, Megf11, Btg1, Eea1, Rnf13, Commd2, Greb1, Ppfibp2, Sbf1, Cdc42ep3, Slc38a7, Rnf113a1, Cnot1, Iqce, Lgi4, Ppp6r2, Ndrg4, Ttyh3, Upf3b, Fxyd7, Spg20, Chst12, Plxnb2, Eepd1, Eipr1, Rfxap, Mgat4b, Atp13a2, Alg5, Nudt1, Tbcld9b, Dennd3, Cenpt, Tmtc3, Cc2dla, Ago2, Zcchc12, Cab39, Cyld, Micall2, Zscan12, Rbm42, Oxsr1, Cox6b1, Dnajc12, Wdr44, Decr2, Anln, Tent4b, Rps271, Klhl13, Cneplr1, Sun1, Jmjd4, Topors, Ssx2ip, Lpar3, Plch1, Psenen, Siahla, Lin37, Mrp141, Hspb6, Wnt4, Mrp155, Dchs1, Syde2, Smad6, 2410004B18Rik, Dna2, Phkb, Acaa2, Arhgap33, Arhgef26, Gtdc1, Zfp157, Neto2, Unc93b1, Trim67, Zfp280c, Ttc7, Stox1, Stag3, Aifm1, 4921524J17Rik, Kdm1a, Elac1, Map11, Kifbp, Lrfn3, Bcorl1, Spryd3, Cnpy4, Tmem177, Anapc10, Taf6, Tfb1m, Zdhhc9, Pml, Trim3, Otud4, Nxt1, Asap3, Zfp39, Tns2, Zfp113, Hk1, Frem2, Zscan21, Wdr62, Mmaa, Foxa2, Zfp146, Tspan15, Apbb1, Inhbb, Smpd1, Cavin3, Sh3glb1, Rbmxl1, Rnf139, Trmt12, Slc35b2, Rab11fip3, Dcaf15, Socs5, Zcwpw1, Setd7, Fam91a1, Prmt9, Arhgap10, Ddx1, Ndufc1, Mgarp, Ppp1r14a, Mycn, Nodal, Elf2, Rbm17, Atg101, Islr, Spry1, Syn1, Spred3, Clic4, Zfp692, Mex3c, Aagab, Kin, Rsrp1, 4932438A13Rik, Gemin5, Tmem97, Galnt6, Stag1, Ldlrap1, Ttc13, Man1c1, Tacc3, Jade3, Tap1, Bbs7, Capn15, Larp1, Hand1, Map4k1, Fam53a, Slc9a7, Taf2, Chst7, Paqr7, Nudt22, Letmd1, Uvssa, Sf3b6, Ccn3, Pafah2, Kdm6a, Ctbp1, Spon2, Atp11b, Icam1, Ranbp10, Endod1, Depdc5, Slc30a1, Cep85, Tulp1, Azin1, Ints7, Klf10, Tedc1, Dtl, Thoc2, Asx12, Cib2, Bcdin3d, Rapgef6, Plekhg2, Zdhhc18, Vash2, Mcrs1, Gch1, Kdf1, Atoh8, Wdtcl, Kcnk2, Cldn11, Cdkn3, Slc25a43, Zbtb42, Zfp60, Prkci, Vps13b, Ldah, Rfx7, Inf2, Esyt3, Ahdc1, Ddhd1, Cisd1, Fermt2, Gnpnat1, Mrps26, Eefla1, Phyhipl, Xkr8, Ptger2, Actr5, Slc16a9, Slc32al, Dzip11, Apopt1, Vopp1, Fam76b, D630003M21Rik, Ctnna1, Tgm2, Tspan14, Ppmlk, Rtkn2, Cpe, Aim2, Ackr1, Jmjdlc, Dusp8, Pcdh18, H2az1, Rcor1, Bri3 bp, 1700018B24Rik, Ndufv1, Ddx60, Scarb1, Wdr20, Rflna, Zc3h7a, Ninj1, Snn, Ccdc92, Sh3rf3, Rmi2, Rara, Dhx38, Igsf9, Parp11, Acd, Hpf1, Ccdc138, Wipf2, Rapgef11, Mindy4, Atp6v0a2, Dennd4c, Tigar, Igsf8, Socs1, Gcc2, Haus6, Dexi, Kmt2c, Nod1, Cdkn2aip, Cntln, Galnt11, Fkbp14, Tmem237, Sbno1, Trappc11, Phf20, Cdon, Fam210a, Mphosph9, Ccdc50, Tmem181a, Notch3, Prdm1, Gstp2, Spon1, Atg5, P3 h2, Ttc39b, Fam126b, Chpf2, Btbd10, Hand2, Fbxo8, Pgap3, Mfsd14b, Bend3, F11r, Pdss2, Cep250, Sobp, Usp38, Ncapd2, Hoxa5, Gdf5, Slc22a23, Iffo1, Fau, Nop2, Ostm1, Snx10, Afg11, Spats21, Edem2, Kcnh2, 1700066M21Rik, Trpc4ap, Sesn1, Txlng, Zfp384, Plc11, Arl5c, Lasp1, Ncoa6, Pcp411, Sbf2, Gmds, Trp53inp2, Pigu, Rras, Mpp6, Gpr162, Txnip, Pmepa1, Foxf2, Scaf1, Foxq1, Cdc16, FIG. 4, Hdhd3, Poli, Usp5, Spsb2, Abhd17c, Uqcrfs1, Olfml2b, Chmp4b, Cdk19, Polr21, Prr3, Mesd, Gdf15, Rpf2, Tbc1d17, Cls1, Armc10, C1rl, Rgs4, Zfp280d, Atf5, Inip, Cul7, Fndc4, Ef11, Rad9b, Susd4, Sct, Pptc7, Akap12, Tctn1, Cep851, Slc35f1, Ripor1, Samd10, Dock10, Phrf1, Mcl1, Rassf7, Med30, Tm6sf1, Nepn, Degs1, Lrrc56, Pold1, Ramac, Rnh1, Herc1, Lpar1, Arfrp1, Scube3, Paklip1, Rtel1, Josd2, Mapkap1, Taf51, Dsel, Gmeb2, Pbx3, Bud31, Pakap, Mboat1, Wdr26, Mvb12b, Angpt16, Abcf1, Alpk3, Ptpn3, Lmx1b, Vill, Ephx1, Smurf1, Stap2, Lefty1, Zscan2, Sde2, Trmt112, Ctnnal1, Hace1, Abitram, Tmem214, Ralgps1, Vars2, Phb, Ythdf1, Itpkb, Baiap211, Garn13, Zcchc2, Zfhx3, Mrps34, Shfl, Man2a2, Ctul, Prl3b1, Zfp653, Rpl12, Plc12, Dido1, Rccd1, Tcf15, Sccpdh, Prc1, Edc3, Slco4al, Ube213, Rabggtb, Blocls5, Cables2, Txndc5, Ube3c, Bmp6, Usp6n1, Pigk, Foxred1, Osbp12, Ak5, Anpep, Atpsckmt, Psmd7, Zzz3, Mtg2, Zfp503, Ss1811, Rreb1, En2, Marchf6, Nexn, Lsm14b, Nrn1, Taf4, Cdc123, Zc3hcl, Whrn, Sart1, Shd, Fam102a, Akna, Cmc1, Naif1, Dap, Polg, AW209491, Fanci, 1110008P14Rik, Adk, Abhd2, Ciz1, Daglb, Metrnl, Rp1391, Gpatch2, Arid4b, Ppp1r10, Tbcd, Tbce, Sec24d, Isg20, Tgfb2, B3galnt2, E130309D02Rik, Lyplal1, Pomt1, Vstm2b, Prrc2b, Npep11, Megf9, Foxk2, Azi2, Fndc3b, Cybc1, Cdk5rap2, Hexdc, Ndst2, Minar1, Rftn1, Rab3gap2, Igfbp2, Tex19.1, Tex19.2, Smarcall, Exosc2, Fut11, Picalm, Sec24c, Marchf4, Hlx, Dusp10, Cdh6, Mreg, Neil3, Prss23, Heatr5b, Alg1, Prpf18, Morc3, Ppl, Mtmr12, Slc9a8, Prrx2, Tnrc18, Nt5dc1, Nrtn, Asb6, Tspyl4, Dse, Nup133, Uhrf1bp1, Ptpa, Cyp7b1, Zup1, Mmd2, Stau1, Ubald1, Myo9a, Fam149b, Rcan2, Tmem246, Stub1, Prex1, Ap5z1, Hnrnpu, Ccdc151, Zfp189, Coro7, Mrpl12, Vasn, Rxrb, Spout1, Oxldl, Zmynd8, Capsl, Tbcld13, Zer1, Msantd3, Ncoa7, Usp53, Nploc4, Trp53rka, Fnbp11, Prkrip1, Slx4, Alg2, Bahcc1, Exo1, Dnttip2, Dnajc28, Dnajc11, Ypel5, Galnt12, Cep131, Zfp597, Zfand1, Cplane1, Ncoa5, Zc3h10, Tbc1d2, My16b, Wdr70, Olig2, Arhgap29, Zfp335, Pcif1, Endov, Rere, Slc44a3, Neur12, Slc26a11, Cited2, Spsb1, Coq10a, Rhbdd2, Urb1, Pik3cd, Clstn1, Hip1, Ccdc40, Zfp292, Tbc1d16, Ankrd13c, Cbx4, Timeless, Gnaz, Slc7a5, Fkbp6, Ythdf2, Stat2, Negr1, Ift88, Rbms2, Orc3, Baz2a, Flywch1, Zmym5, Pnrc1, Ndp, Maob, Hs2st1, Kcns1, Ikzf5, Alkbh3, 2310057M21Rik, Ankrd6, Scamp2, Nemp1, Pclaf, Zfp704, Trappc5, Lrp1, Gbp7, Nxph4, Klhdc4, Plekhal, Bach2, Accs, Pacsin1, Ndufa412, BC052040, Hey1, Ddx58, Cchcr1, Zfp770, Slc25a24, Fam102b, Mars1, Skiv21, Ufl1, Bcor, Trim41, Pex2, Aqr, Map3k10, Fbx14, Dtx3, Large2, Usp45, Arglu1, Os9, Mybbpla, Blvrb, Rabggta, Dgkz, Dxo, Xaf1, Ltbp4, Lrfn2, Marchf9, Abcg5, Ambra1, Pvr, Tsfm, Zfp609, Abhd11, Necab1, Ckap5, Otud6b, Mett127, Plppr2, Apoc1, Ofd1, 1110051M20Rik, Ranbp17, Bace2, Kazn, Apobec1, Tmem51, Pck2, Gemin8, Dok4, Ppip5k2, Rimklb, Tasor, Oaz2, Efhd2, Sh3bgr, Phc1, Madd, Col16a1, Dnajc16, Cyp2s1, Sh3pxd2b, Klc3, Il17rd, Virma, Scamp5, Hnrnpul1, Esrp1, Cep126, Ppp1r131, Ints8, Siah1b, Appl1, Spen, Snrnp25, Oard1, Cept1, Ttc3, Clqtnf4, Iqcc, Iqsec3, S100g, Em12, Tex264, Dennd6a, Hlcs, Snrpd2, Six5, Szrd1, Psme4, Plekhh2, Reps2, Dlk1, Erf, Bsdc1, Crocc, Ino80d, Begain, Wdr25, Tmem205, Kcnd3, Atpla3, S100pbp, Arhgef1, Rcc2, Rps19, Arhgef101, Igsf21, Sh3kbp1, Abhd4, Npnt, Trim62, Map7d2, Ghitm, Irgq, Fam117b, Ramp3, Wdr43, Wwp1, Pif1, Nacad, Fzd7, Grid1, Rwdd2b, Ostc, Elmo1, Ccdc8, Pde9a, Nbl1, Msantd4, H2az2, Smcla, Ripk2, Tmco4, Osgin2, Otud3, Zmiz2, Prkd2, Rpusd1, Pla2g2d, Trir, Yeats2, Clvs1, Arhgap11a, Elov16, Phf8, Ublcp1, Chd7, Vps41, Mul1, Wnk3, Zfp236, Car8, Uspl1, Dmx12, Tox, Ttc1, Cdk13, Gtf3c3, Inhba, Pcf11, Ankrd42, Rap1gap, Tmem29, Rg12, Ccdc9, Cldn5, Wapl, Pik3r1, Meis3, Hibch, Conb1, Utp4, Tardbp, Dcp1b, Ago1, Hspb8, Nop53, Camsap2, Mbp, Bicdl1, Ggact, Fam104a, Mrps27, Gcn1, Ddx24, Klf8, Pcca, Rragb, Rims1, Pyroxd1, Bivm, Amot, Zic5, Alg13, Pi4ka, Coro2b, Rnf10, Pde3a, Utp15, Tpp2, Ubac2, Ppp2r2d, Enc1, Cirl, Tram2, Phlda3, Fam169a, Oasl1, Fhdc1, Rhod, Mcm3, Ankrd13a, Ndufa7, Shisa4, Git2, Lman1, Gpr45, Fam222a, AW549877, Agrn, Mvk, Mfsd9, Pkp2, Dcaf17, Spidr, Mett18, Rapgef5, Zbed3, Tlk1, Acacb, Wdr41, Mat2b, Csgalnact2, Zfp386, Svop, Hnrnpf, Arsb, Zfp239, Kank3, Uggt2, Inpp5f, Ssh1, Rassf4, Ppig, Cox10, Klh123, Dzip1, Tent2, Armc5, Nfrkb, Cmklr1, Slc35f2, Chchd7, Kdm5b, 0610010F05Rik, Fbxo38, Zfand4, Ammecr1, Lyn, Tmc7, Grk3, Pm20d1, Cilp, Is11, Nxt2, Sestd1, Pelo, Ttc19, Ehbp1, Setdla, Hps4, Specc1, Ar115, Arel1, Gn13, Gjb5, Osbp16, Lgalsl, Snx18, Gjb3, Fam83g, Smim12, Dlgap3, Tsen2, Agps, Nfkbil1, Fbrs, Dhx29, Mfap4, Zfp532, Mindy2, Zmym4, Dctpp1, Zfp410, Tbc1d8b, Leo1, Clspn, Tbc1d10b, Cd2bp2, Usp22, Pwwp3b, Kcnj12, Sin3a, Adprhl2, Dhrs7b, Mier2, Ube2q1, Thsd7b, Cux2, Ipo11, Sh2b3, Fam199x, Kdm7a, Atxn2, Hirip3, Stk40, Oscp1, Zfyvel, Itpr3, Alkbh5, Arrdc4, Ypel3, Zc3h12a, Mapk6, Dnalil, Atpaf2, Naa25, Map3k9, Trafd1, Ttc9, Hectd4, Id1, Bex2, Tmem108, Maneal, Hebp1, Exog, Aqp11, Hecw2, Krbal, Mcts2, Snail, Alkbh6, Serpinb6b, Trp53rkb, Tom1, Aida, Foxo4, Egflam, Sbk1, Notum, Borcs5, Nhlrc3, Gng2, Nemp2, Zfp513, Tmem18, Fam89a, Zfp866, Tubalc, Mobla, Tmem186, Crppa, Ppp2r3a, Pyurf, Zmpste24, Upf2, Tmem64, Pigv, Tmem11, B3galnt1, D17H6S53E, Fbrsl1, Gprasp1, 2510009E07Rik, Usp48, Epop, Pgp, Tril, Rai2, Zc2hcla, Fbx17, Mdgal, Vps37d, 1700019D03Rik, Pld6, Tox3, Diras1, Dpyl913, Kcns3, Femla, Rpl7, Ptpn11, Rtn4r, Lysmd4, Tmem145, Mgat5b, Taf10, Zmym1, Slc36a4, S1pr2, Klh115, Thrap3, Orai3, 2900052L18Rik, Mgat2, Gls2, Cilp2, Mrp150, Irf2bp1, Cep68, Zrsr1, Rsbn1, Bmerb1, Phetal, Arf6, 1810030007Rik, Nkrf, Bclaf3, Foxo1, Gpr146, Dnajc21, Pced1b, Nrnll, Cnr1, Ubr3, Neurog3, Ackr3, Alkbh2, Phlpp1, Snhg11, Cxxc4, Tigd3, Sptssa, N6amt1, Zfp507, Shisa2, Fam160a2, Tnfaip811, Zfand3, 2510039018Rik, Bod1, Tram111, Tceal3, 9930012K11Rik, Acp1, Garem2, Smim7, Zbtb39, Swi5, Cnrip1, Csrnp2, Pard6b, Zbtb7c, Tmem201, Ccnjl, Kcnj10, Gemin7, 9930104L06Rik, Serpinbla, D1Ertd622e, Scml4, Hjurp, Zfp36, Setd2, Isca1, Zfp354c, Shb, Lsm11, Lemd2, Zfp444, Coa4, Uqcrq, 4930503L19Rik, Zfp367, Ubtd2, Pwwp2a, Fzd5, Gm4779, Prkce, Megf8, Lrfn4, Rpsa-ps2, Zc2hclc, E130308A19Rik, Magi1, Kmt5b, Saysd1, Rpl18a, Tubb2b, Bola3, AI467606, Sox3, Shroom2, Cirbp, Dpyl914, Vcpip1, Nudt18, Hs6st1, 1110012L19Rik, Med26, Zfp688, Zfp574, Morn2, Klhdc9, Zfp691, Cenph, Dcaf1211, Insig1, Preb, Lhfpl2, Proser2, Cenpe, Zfp423, Tmem88, Cxcr4, Epcam, Akrle1, 2410002F23Rik, Hnrnph2, Tmem60, Cox19, 2810002D19Rik, Trrap, Sys1, Pou3f3, Kcna5, Ddx28, Penk, St71, Frrs11, Glb1, Zfp553, Dbx2, Sh3tc2, Zfp629, Pid1, Plekha7, Cdc42ep2, Mfsd5, Spred2, Col27a1, Pqlc3, Gm21411, Mms221, Basp1, Whamm, Zfp3612, Zswim3, Ptprcap, Serpinb9, Lyrm2, Slitrk6, Tmem268, Mrps12, Cavin2, Wnk1, Slc25a51, Eif4g1, Onecut2, Polr2k, Stard5, Snx12, Otulin, Eid2, Mosmo, Cep295, Tmem229b, Olig1, Zfp84, Scaf8, Pnma2, Plaur, Scand1, Ankle1, Zfp62, Stxbp6, Hs3st2, Slc25a23, Rp137a, Zfp322a, Gjb2, Rpl27a, Asphd1, Rbp1, Kcnk6, Lrrc75a, Hnrnpal, Cmtr2, Camk2n1, 5930403N24Rik, Clqtnf2, Fam43a, Spin2c, Zfp319, Unc119b, 4930430F08Rik, Lyrm4, Prr12, Ticrr, Tmem215, Bdh1, Hrk, Olfml2a, Zfp316, Rbm12b1, Mtfr11, C87436, Chtf8, Csnk2a2, Hmga1, Foxc2, Gprc5a, Ccdc66, Mrps7, Fam83h, Rhoj, 8030474K03Rik, Epm2aip1, Riox1, Zfp787, Lrrc75b, Ddit41, Slc39a3, Brox, Ckap4, Vat11, Pramef8, Mbtps2, Irgm1, Zfp740, Mageb16, Nqo2, Zbtb21, Tshz1, Mars2, Spsb4, 1110032F04Rik, Zfp41, Spata2, Tmem164, Ngrn, Lrrc4b, Rnf31, Ticam1, Cd24a, Zfp654, Tet1, Khnyn, Ythdf3, Gm527, Mageh1, Taf9b, H2bc6, C2cd3, Ptgs1, Emc6, Sfn, Kcne4, Zfp286, Tdg-ps, Zfp768, B4gat1, Atmin, Tgif1, Flrt2, Rexo1, AI837181, Xxylt1, Arl4a, Dynlrb1, Zfp30, Rnf152, Adamts12, Cldn4, Tspyl1, Mis18bp1, Fbxo28, Tmem41b, Lxn, Tyw3, Mafa, Zfp235, A430005L14Rik, 2810006K23Rik, Cd3eap, Ms1312, Rpsa-ps10, Rtl3, Yipf6, Olfr1388, Ppp1r2, Ubiad1, Bola2, Utf1, Fancb, Lrrc49, Atg1612, Slc38a9, Akap10, Trim16, Cdca4, Bri3, Lonp2, Usp14, Tshz2, Ankrd16, Trappc9, Marcks11, Palm3, Gigyf2, Timm8a1, Arxes2, Ldlrad3, Pirt, Arf1, Tenm4, Gm4737, Taf13, Rbm15, Arid4a, Col6a3, Kmt2d, Mcmbp, Asb8, Cracr2b, Amigo2, Fbxo10, Rnf149, Crebrf, Dip2c, Rbm33, Syngr2, Sacs, Zfp738, Dleu7, Frmd6, Ankrd46, Pskh1, Ric3, Pou4f1, Arxes1, Pdp2, F2r, Osr1, Fam171b, Gli2, Mlf1, Timm29, Nup12, Cyp4f16, Ccdc57, Inka2, Mypop, Zbtb8b, Fitm2, Nrip1, Tyw5, Mmgt2, Sp8, Mlec, E130311K13Rik, Ctxn1, Lemd3, Rhno1, Mex3d, Ccdc6, Lurap11, Zfp454, Ggnbp1, Prss50, Foxo3, Rpl29, Dcun1d3, Cyb561d1, Slc25a47, Zfp710, Efna5, Fkrp, Zfp689, Cdca2, Tada3, Yjefn3, Clgalt1c1, Nrsn1, Elfn1, Atxn712, Ndnf, Tmem121, Mterf2, Armcx3, Acap2, Bmyc, Zadh2, Sephs2, Dcaf5, Agtrla, Fam110b, Fam183b, Pdp1, Tigd2, Gm7324, Kcnk3, Mblac1, Iba57, Prmt6, Ccdc71, Zfp2, Kmt5a, Retreg2, Ogfr, Chchd10, Cyp20a1, Aff4, Tmem67, Rps23, Tceall, Clip1, Fzd9, Polrla, Tmie, Lingo1, Ap5b1, Zfpm1, Tsku, Zbtb45, 2310022A10Rik, Zbtb5, Bdp1, Aftph, Zbtb14, Urgcp, Orail, Zfp668, Trex1, Zfp646, Rpl36al, Zfp672, Zfp280b, Fzd4, Bag5, Sertad2, Armcx4, Zbtb12, Arl4c, Calhm5, Rlf, 2810025M15Rik, Vcpkmt, Rasd1, Tmem17, 2610318N02Rik, Slc35c1, H2ax, Pgrmc2, Rpp38, Ccdc137, Plekhf2, Glt1d1, Lrrc25, Ppp1r3d, Pitpnb, Rap2c, Fh14, Tmx2, Grem2, Rspryl, Grrp1, Ubqln2, Slc9b1, Gm867, Lsm10, Eif5a2, Lgr4, Evalb, Snip1, Il17d, Pigm, Hic2, Heatr1, Tmem220, Dscam, Fzd2, Foxc1, Rictor, Nsun3, Amer1, Snx21, Septin6, Kif7, C77080, Armcx6, Tcf19, Pnma5, Fam118b, Fbx122, Fam171a1, B230217C12Rik, Fam241a, Lamtor4, Kcnb1, Maml1, Lrrc4c, Zfp61, Micos10, Ubald2, Vstm4, Gpatch11, Prkaal, Emilin3, Scg2, Plekho2, Arhgap42, Vamp8, Ptges, Gplbb, Tmem37, Ccdc126, Fam131a, Slc29a4, Cdh20, Zfp623, Zfp940, Pdik11, Tatdn1, Tvp23a, Tmem123, Map10, Amigo1, Gja1, Creg2, Gdpgp1, Selenon, Gatd1, Hs3st1, Zfp455, Olfml1, P4ha3, Fbll1, Lingo3, Mblac2, Fam71e1, Gimap9, Camk2n2, Nat2, Adnp, Rpusd3, Ercc61, Bzw1, Tceanc, Rnf7, Gen1, Msrb3, Swsap1, Islr2, Nhlh1, Jagn1, Trapla, Zgrf1, Gdf6, Pcmtd1, Usp42, Taf7, Pcdh19, Nup160, 2900026A02Rik, Rab11fip5, Plekhm3, Spryd4, Commd1, Six1, Plpp7, Zbtb22, Tacstd2, Ppplr37, Bbs12, Crebzf, Cbr1, Irf2bp2, Zfp786, Mafg, Fam181b, Rps19bp1, Usp29, Zfp579, Tceal8, Mical3, Rap2a, B3gnt2, Lrrc3, Coa6, Trim32, Pcbp1, Tmem198, Senp8, Wdcp, Rp131-ps13, Mett15, Rinl, Fam229b, Xrcc1, Tubgcp6, Nlgn2, Sox12, Rtl8c, Arf3, Sox6, Spats2, Smim11, Tagap1, Klf13, Zfp217, Pard3b, Mtcl1, D630039A03Rik, Fox06, Rbm12b2, Ppp4r2, Rps10, Plpp2, Acvr2a, Serpinb6c, Hbb-y, Gm14964, Opa3, Zeb2os, Zfp622, Taf9, Cdc42se2, Ltn1, Doc2a, Lrrc15, Ankrd44, Immt, Cemip, Tmem106c, Actn2, Nrros, Acot4, Rft1, Mogat2, Ezr, Rexo4, 2610001J05Rik, B4galt3, Tmco1, Zfp961, Cherp, Epha3, Robo2, Nup188, Pbx1, Magi3, Adarb2, Hook2, Serp2, Adam17, Isoc2b, Pcdh15, Asap2, Rnf103, Zmat1, Rap1b, Jun, Tln2, Tnrc6a, BC004004, Map1b, Suclg1, Repin1, Zfp212, Oasla, 1700030K09Rik, Atad2b, Sael, Junb, Reep1, Msl1, Rnaseh2a, Ctif, Fbxo31, Rnf157, C130021I20Rik, Gas1, Mrp135, Ube2q11, Uba2, Creb5, Krcc1, Sv2b, Brsk2, Jam2, Cfap300, 2700081015Rik, Lins1, Tmem248, Yap1, Socs3, Chmp3, Supt71, Mapk11, Spag16, Fes, Cdh22, Rpl18-ps2, Bcl3, Dand5, Ddx10, Rab4b, Pom121, AI854703, Gatd3a, Dis312, Ficd, Zfp952, Hunk, Mapk14, Thoc7, Ggcx, Kdm3a, Tnfaip6, Tcf4, Usp21, Kcnip1, 3110082117Rik, Smagp, Ier2, Eif3k, Zfand2a, Zfp472, Rpia, Dennd4a, Plxnb1, Gper1, Mast1, Tmem39b, Ptrh1, Chd8, Ubxn7, Grwd1, Camk2d, Nudcd3, Txlna, Fpgt, Sh2d4a, Ech1, Mat2a, Cyhr1, Shisa6, Cnn3, Adnp2, Tmem179, Tceal5, Ercc6, Skp2, Spock3, Gzmm, Tprkb, Fgfr3, Msi1, Lifr, Prr141, Eapp, Cpsf3, Rhob, Zfp747, Dnajc8, Spcs3, Slc30a7, A930005H10Rik, Atpif1, Tmem120b, Tle5, Vapb, Vsnl1, Lclat1, Dedd2, Parp4, Zfp867, Cggbp1, Kdm2a, Slc8a1, Zfp869, Tmem119, Adam10, Zscan22, Zfp771, Vmac, Phactrl, Msra, Zfp182, Fsd11, Set, Kctd18, Klhl18, Cadm4, Actn4, Elp6, Rnd1, Pcnx3, Dsp, Zfp667, Dmac21, Zkscan4, Kcnmb4, Zfp174, Migal, Agap1, Cntn1, Commd5, Rab39, Cgrrf1, Klf2, Fam72a, Sertad3, Zfp811, Ankrd17, Pld5, Wdr86, Kcmf1, Zfp101, Ntrk2, Tmem245, Ctsj, Mrfap1, Zfp93, Sec23ip, Tns1, Snupn, Pr12c5, Stam2, Fut9, Fbx06, Map6, Nap115, Maf, Sogal, Cpsf6, Zcchc24, Kxd1, Ct55, Zfp459, B4galnt4, Zfp580, Dach1, Klh125, Gpc3, Mett14, Cope, Klh126, Slain1, Ubl7, Rras2, Ghr, Gemin6, Eef1d, Abcd2, Tcf711, Mta3, Zfp1, Rnf181, Izumo4, Per2, Tmem150a, Zfp277, Irs1, Zkscan5, A430033K04Rik, Zfp709, Mia3, Otulinl, Eif3b, St3gal5, H2-T22, Fez2, Trim34a, Rdh9, Socs6, Cnot10, Col8a2, Snx32, Cfl1, Pgpep1, R3hdm1, Pardog, Cebpg, Kcnq3, Lrif1, Cep70, Zfp981, Usp39, Sertm1, Tyw1, Usp34, Lig1, Adap1, Slit3, Tgoln1, Cyct, Hoxaas2, Mok, 5730596B20Rik, Med121, Cd248, Chn1, Foxk1, Cebpb, Rab31, Ccdc18, Pign, Rlim, Tug1, Zfp658, Trnp1, Chd9, Them6, Retsat, Kdm5d, Gpr173, Ilrun, Elmod3, A930024E05Rik, Capg, Dnah9, Hmga2, Setd3, St8sia3, Pcbp2, Gulp1, H2bu2, Immp21, Sipa1, Cep162, Tatdn2, Jmjd6, Ide, Erollb, Zfp607b, Ebf1, Zfp180, Nat8f1, Cntrl, Npm1, Txnl4a, Chd6, Ado, Dph6, Ccdc189, Gsk3a, Scn3a, Slc44a2, Dmac2, Aak1, Mett115, Rbbp4, Gm6139, Bcl2, Cep170, Chst3, Uxs1, Birc2, Mrpl18, Zfp759, Nsd2, Las11, E2f6, Fam136a, Bloc1s2, Ecel, Pus7, Zfp317, Lgals8, Zbtb8os, Arl16, Gm6807, Prdm2, Brd9, Gapdh, Bloc1s3, Pkn1, Zfp746, Mex3b, A830018L16Rik, Prtn3, Gm6169, Ddx49, Bak1, Xlr3a, Abat, Zfp329, Camk2b, Fgf18, Zfp809, Mdn1, Palld, Zfp729b, Tpm3-rs7, Gstm1, Xlr3c, Sez61, Smco4, Zfp980, Zfp846, Arhgap35, Usf2, Cryzl1, Idi1, Espl1, Zfp68, Spock2, Mcm9, Upf1, Ube2e2, Phf21a, Zfp85, Smim4, Abcel, Phtf1, Rrp1b, Txndc9, Syt17, Map2k5, Znrf2, Dhcr7, Gm15590, Rp15, Tmed9, Gpc6, Cela2a, Tmod3, Rp130, Zfp110, Eif4b, Tubb2a, Ccser2, Memo1, 0610030E20Rik, Egln2, Prr19, Rnf169, Col13a1, Zfp516, Deaf1, Col25a1, Rsl1, Clqtnf3, Gm10053, Fam57b, Hdhd5, Vwa8, Hnrnpa3, Sh2d3c, Nap114, Ntrk3, Pdela, Skap2, Mtfmt, Tafa1, Hnrnpm, Foxb1, Septin9, Usp47, Zc3 h4, Cdyl, Eda, Zfp3613, Slc14a1, Wdr83os, Fxn, Ppp2r5d, Hadhb, Ptk2b, Zfp426, 2610301B20Rik, Kbtbd2, Nhs, Tcea2, Trp53, Calcrl, Taf1b, Zfp637, Akap17b, Rcan3, Slx1b, Rps26-ps1, Nrm, Rgs3, At12, Zfp874b, Ntng1, Zfp422, Ptp4a3, Zfp930, Tmem40, 4930453N24Rik, Grb2, Ntm, Zfp74, Taok2, Nptx2, Chpt1, Kif13b, H2aj, Dhps, Tmem26, Rp2, Prmt7, Gemin2, Tpt1, Gm10076, Serpinb6a, Pop5, Zdhhc8, Gm11353, Zfp462, Cend1, Gtf2i, Ap2a1, Ppih, 2610008E11Rik, Bckdha, Zfp128, Chst8, Pantr1, Zfp868, Sntb1, Irak2, Zfp266, Tmem219, Tnfrsf19, H2-Q7, Mfap2, Nr1 h2, H4c9, Plaat3, H4c3, Mrps9, Slc9a6, Cd302, 1700019A02Rik, Plekhh1, Ipmk, Nsa2, Gmfg, Intu, Gstp1, Ube2s, Zbtb40, Acyp2, Trmt61a, Dmkn, Irx1, H4c8, Copz1, Rrs1, Clasrp, Rrp1, Haghl, Cdh3, Zfp143, Sap18b, Mcrip1, Dnajc30, Prcp, Ppm1b, Prpf40a, Mam13, Fnip2, Sfmbt2, H2-K1, Exosc5, Slc38a10, Cxcl12, Fiz1, Zcchc14, Tmem151a, Stx17, Nol10, Mrps36, Rps7, Snrpa, Sox21, Zic2, Wdr61, Dpp6, Dot11, Akap6, Mdc1, U2af1, H2ac4, Med9, Cd2ap, Dlgap4, Tnnt3, Ext1, Mospd2, Rps17, Rufy4, Suclg2, Ssbp3, Zscan20, Rbak, Myt11, Ppp4r1, Rcc11, Rpl34, Hsh2d, Gmfb, Pgghg, Pgk1, Lmnb2, Gm10110, Rai1, Zfp954, Cttnbp2n1, Cnih4, Tgif2, Hs6st2, 2700097009Rik, Btbd9, Gspt1, Erbb4, Rapgef2, Gak, Nectin2, Rpp25, Erbb2, Rpl17, Serpinb9e, Itgb1bp1, Tmem65, Tubb3, Ftl1-ps1, Zfp706, Ap3b2, Gm8623, Ns11, Zfp534, Zfp398, Timm17a, Armc9, Tubb4a, Srpk2, Rps3a2, 2310039H08Rik, Ncs1, Atp5g2, H2bc12, Ppox, Zfp512, Ei24, Zfp599, Hikeshi, Actg1, Zfp28, Klh124, Hdac10, Acadm, Cfl2, Mtap, Stat4, 9130023H24Rik, Rp135, Ccni, Ing2, Lrrc40, Sox7, Ctnna2, Mapk3, Kif1b, Zfp26, Bbs5, Clip2, Numbl, Rps41, 1110038F14Rik, Grm4, Scoc, Parp10, Naa15, Usp31, Tmed5, Cyp26b1, Wscd2, B9d2, Nmrall, Eno1, Nup98, Col23a1, Jazf1, Klhdc3, Sox11, Slc22a21, Zbtb18, Brwd3, Cycs, Sp3os, Rnf217, Chchd1, Ap3s2, Lin28b, Gpatch1, Alms1, Ppcdc, Chd4, Ptcd3, Rpl711, Crem, Zkscan8, Dpp3, Srrm4, Pepd, Brpf3, Amd2, Nr6a1, Klk8, Gpn1, Trerf1, Mtx1, Fbln2, Cadm2, Mocs1, Med14, Cenpj, Fam172a, Arih2, Rab3ip, Ano6, Plcxd1, Platr26, Zfp428, Hvcn1, Tank, Clasp1, Hhip, Sival, mt-Rnr2, mt-Nd1, mt-Nd2, mt-Co2, mt-Co3, mt-Nd4, mt-Nd5, mt-Cytb, Selenop, Cpped1, Aurkaip1, Zfp979, Zfp987, Dhrs3, Hnrnpr, Med18, Phactr4, Prpf4, Cdc26, Slc31a1, Fkbp15, Slc31a2, Anks6, Ipo7, Trim12a, Vps37b, Numa1, Impad1, Wdr6, Gm10263, Ms12, Zfyve26, Mthfs, Gm16551, Rpl17-ps9, 4931406P16Rik, Insyn1, Zfp932, Tecpr1, Ttc32, Fbx118, Wdr35, Lefty2, Ifi208, Zfp575, Gm10175, Vkorc111, Zbtb6, Zfp810, Zfp772, Oas1g, Nck2, Gm10184, Zfp617, Fbx112, Vsig10, Suds3, Bub3, Capza1-ps1, Hes6, Polrlc, Xpo5, Eiflax, Rplp0, Capn6, Bmpr2, Trmt2b, B3galt4, Zfp563, Dmrtc1c2, Syngap1, Tuba3a, Xlr5c, Nnat, Blcap, My19, Romo1, Arfgef1, Zic3, Htatsf1, Vxn, Sptbn2, Zfp991, Rex2, Rtl8b, Rtl8a, Gtf2f2, Gm14648, Lrch1, Cenpm, Gm10232, Gm11808, Phf11d, Slc4a5, Aup1, Htra2, Dok1, Chrac1, Rp134-ps1, Uty, Gng5, Zfp467, Actc1, Gjd2, Efcab1, Clec16a, Tmem167b, Trp53i11, Sars, Celsr2, Cry2, Psrc1, Sort1, Il3ra, Gstm6, Rapla, Csde1, Sf3b4, Dap3, Zbtb34, Gm5641, Slc25a31, Ddx3y, Eif2s3y, Ubaly, Cdk7, Pde7a, Rps18-ps6, Adgrv1, Nr2f1, Zfp72, Zfp874a, Zfp825, Dusp22, H2ac8, H3c6, Epc2, Dazap1, Ank3, Gm10273, Marcks, Zfp975, Arid1b, Zfp125, Sco1, Irgm2, Atxn11, Spd11, Ell, Ssbp4, Sp110, Slc35g2, Rnf213, Prss36, Capza1, Hs3st3b1, Zscan25, Rnf121, Il18 bp, Serpinh1, Tlnrd1, Cldn3, Fam217b, Chchd2, Rgma, Nsmce3, Ccdc190, Zfp992, Sars2, Gtsf11, Gm10282, Tmem200b, Rbm44, Tmem35b, Dgkd, Pnmal2, Cited4, Bicra, Zscan18, Gm1673, Ccnyl1, Rraga, Tgfbrap1, Rabepk, Dnajc25, Ndufb6, Safb, Zfp827, Yipf3, Jund, Nr2c2ap, Srsf3, Smim15, Wfikkn1, Cecr2, Zfp65, Zfp58, 2610044015Rik8, Rps8-ps1, Tial, Gm10330, Hpcall, Dtnb, 1110002L01Rik, Zfp777, Gm10340, 2510002D24Rik, Cebpd, Eef1g, Tut1, Mta2, Em13, B3gat3, Ganab, Ints5, 1810009A15Rik, Uqcc3, Bscl2, Gng3, Hnrnpul2, Zbtb3, Sms, Gspt2, Rhox1, 6820431F20Rik, Apcdd1, Ppia, 6720489N17Rik, Conf, Tubala, Trim6, Xlr5b, Phf2011, Lratd2, Ptrh2, Zfp9, Adamla, Tmem254a, Tmem254c, 1500011B03Rik, Smim1011, Grcc10, Ahnak2, Cep170b, G530011006Rik, A530017D24Rik, Rybp, Gpr27, 4933439C10Rik, Gm12258, Trpc5os, Acot1, Tmsb151, Bhlhb9, Armcx5, Vamp5, Srp54a, Lrrc61, Xlr3b, Gm1141, Vma21, Tmem185a, 5031425E22Rik, 1810058I24Rik, 9030624G23Rik, 2410018L13Rik, Zfp449, 5730507C01Rik, Ccdc160, Gm9, H2-Q6, Gm10501, H2-Ke6, Zfp414, Arhgdig, Wdr90, Ifi204, Cep76, D2hgdh, Fbxo36, Nbeall, Pgap1, Faap20, Cenps, 5730409E04Rik, Gm10575, Tufm, Rbmx12, Olfr713, Mob3b, Trim68, Rhog, Ggh, Exoc8, Mlycd, Snrnp40, Nlrc5, Zfp976, Zfp788, Plekhf1, Gm6658, Sdhaf1, Misp3, Spint2, Aplar, Dda1, Rec114, D930028M14Rik, Gm12522, Zfp94, Peak1, Ccdc61, Ehd2, Zfp865, Zfp628, Spc24, Gm14296, Kcng1, Mocs3, Zfas1, Arfgef2, Tox2, Mafb, Tmem267, 3110070M22Rik, Sox2, Cpne1, BC029722, Eif2s2, Ube2n, 2410141K09Rik, Platr25, Zfp934, Ifit3, Grem1, Fibin, Qser1, Nrbf2, Fjx1, Mir670 hg, Prdm11, Zfp408, Yaeld1, Znhit2, Amd1, Cenpw, Ttc30a1, Ttc30a2, Ttc30b, Wipf1, Aldh3b2, Sp5, Rprm, Rc3 h2, 2810410L24Rik, Hoxc4, Smim41, Fnbp1, Dolk, Alg10b, Commd6, Hoxb2, Nrbp2, Nynrin, Zc3 h3, Ly6a, Selenot, Selenos, Selenoi, Txnrd2, Sox4, Selenoh, Ass1, Rab11b, Srsf3-ps, Gm12184, Chml, Fam43b, Hmgalb, Gm2606, Foxd1, AI593442, F8a, Sf3b5, Ctdsp2, Ppil6, Zfp984, Gm13212, Zfp990, Ddi2, Emc1, Aunip, Nkain1, Bnip3, 1110065P20Rik, Ndufaf8, Ube2d3, AU022252, Hs3st4, Psme3, Fam174b, Chd2, Casc3, Tomm5, Haus5, U2af114, Zfp566, Rad54b, Rbis, BC024978, Dact3, H2aw, Igtp, Zfp931, Gm14418, Gm14305, Gm2026, Gm14288, 2210418010Rik, Zfp965, Gm4724, Gm14440, Gm14443, Gm14391, Ifi47, Tgtp2, Tgtp1, Ube2v1, Pdf, Ak6, Lncenc1, Gapdh-ps16, Wdr92, Sec61g, Zfp429, Samd1, Slc45a4, Col22a1, Ccdc82, Pms2, Kdelr2, Mphosph8, Zfp664, Trim71, Hoxd3, Rab9, Gm2897, Atg4a, Arpc4, Rpl36a, St6galnac4, B3gnt7, Cldn34c1, Gm7609, Gm14966, Pigb, Utp14b, Rab7, Znrd2, Gm9112, Med12, Apoo, Zfx, Gm42688, Tmsb10, Kifc1, Marchf2, Hoxa3, Maea, Gm364, Seh1l, Rhox7b, Rhox4a, Eloc, Fdx2, 3110001I22Rik, Gm4984, Brms1, AU015836, BC022960, Rpl18-ps1, Gm13680, Slc48a1, Fzd10, Rpl17-ps10, Dnajb3, Rpl17-ps8, Gm14276, Gm12943, Vamp7-ps, Gm14336, Gm15387, Rps13-ps5, Gm14150, Fam220a, Gm13233, Gm13340, Rnf138rt1, Gm13436, Rps15a-ps4, Ube2d-ps, Znf41-ps, Ub15, C030047K22Rik, 4933421010Rik, Abhd11os, 1700086P04Rik, Snhg15, E130006D01Rik, A430018G15Rik, Gm12940, Snhg17, Zfp335os, Trmt61b, Gm11772, 4930526A20Rik, Hoxaas3, 8430419K02Rik, Gm4285, Gm15832, Rtl1, Wipf3, Ubap11, Nbdy, Gt (ROSA) 26Sor, Gm14204, Susd5, Gm13205, Isoc2a, Snhg20, Gm15708, 4933431E20Rik, Gm13889, A830082K12Rik, BC064078, Gm15133, E130307A14Rik, C78197, Gm16062, D330023K18Rik, Gm12925, Tmem170b, Kantr, Cers1, Gm15501, 2810001G20Rik, Platr27, Zfp111, E130018N17Rik, Mia, Galnt2, Mir17 hg, Gm20431, Ier51, Tmsb15b1, Slc5a3, Rbm12, Shkbp1, Timm10b, Gm44503, Rps6kc1, Mfsd14a, Uckl1, Tmsb15b2, Ier3ip1, Gm15446, Cdk5r2, Rnf8, Srpx, Gm14399, Cmc4, Usp49, Kcnell, Pou3f1, Uba52, Acad11, Tmem189, Churc1, Mett123, Tarbp1, Gm3512, Gm17455, Gm4675, Gm6576, Tex9, Zfp712, My16, Rps13, Ccdc711, Lrrc32, Gm5141, Vgl13, Smim13, Gm3248, Eid1, Gent4, Gm3739, Cerox1, Gm17066, Lamtor3, Gm6548, Gm8206, Gm3752, Lsm5, Siah3, Gm5093, Gm3636, Zfp964, Sco2, Cox16, Inafm1, Ube2d2a, Gm9844, C2cd4b, Hspala, Gm6793, 1110038B12Rik, Gm20522, Zfp963, Malat1, Smim40, Gpank1, Med20, BC051226, Gm20427, Khdc3, D130058E05Rik, Lrch4, Smim17, Rpl41, Gm20667, Gm20716, Gm4425, Tomm20, Gm5784, Hmgcs1, Gm16867, Gm2237, Fam177a2, Gm38394, Purb, Rpl29-ps2, Zfp131, D430019H16Rik, Rbm4, Gm3604, Gm12537, Ccdc85b, Rhox5, Gm20939, Zfp799, Plac9a, Zfp870, Rps2-ps6, Zfp748, Fignl2, Entpd4, Gm21983, Fam177a, Rps7-ps3, Gm8935, Gm8281, Nimlk, Gm10845, D830030K20Rik, H1f0, Gm2916, Cdkn2d, Tpbgl, Mfsd4b4, Psmb9, Lbhd1, Gm47283, Zfp955b, Zfp850, 2500002B13Rik, Snhg18, Gm17435, Mir9-3 hg, Pvt1, 1600020E01Rik,, 1110002J07Rik, Gm10524, Gm3839, 2310010J17Rik, 2700038G22Rik, Gm2694, 2900076A07Rik, Zfp87, Mirg, AU020206, Rian, 2610037D02Rik, Mir124a-1 hg, Gm26639, G730003C15Rik, C920006011Rik, 4930461G14Rik, A330094K24Rik, Gm16973, Miat, Gm807, Zfp82, 2700099C18Rik, Bvht, Rassf10, Ccdc166, Sowahc, Snhg6, Pigbos1, Gm28043, Kctd12, Commd1b, Zfp953, Tmem185b, Gm28036, 2810039B14Rik, Platr10, 1700097N02Rik, Xndc1, Gm29253, 1700030C10Rik, Gm5619, 2410022M11Rik, Gm28439, 2610306M01Rik, 2010320M18Rik, Gm28437, Gm19412, 2310040G24Rik, 1700096K18Rik, 9130401M01Rik, 1810026B05Rik, H3c11, Iqschfp, Zc3h11a, Gm38110, Tstd1, Gm37607, Tigd5, Gm37567, Gm32585, Rpl21-ps10, Gm43247, Gm43566, Gm35986, Gm42517, Gm6204, AI506816, Gm4332, Gm43720, Gm43549, Peg13, Gtf3c2, Mrp133, 0610012G03Rik, Gm42742, Mpv17, Zfp862-ps, 4833403J16Rik, Gm44183, Gm44597, Snhg1, Particl, Gm44645, Gm7972, Samd4b, Gm49396, Nup62, Gdf1, Gm35315, Hspa14, Chmp1b, Exosc6, Gm19196, Zscan4-ps2, Pde2a, Gm45871, Gm38947, Gm42031, Gm10033, Gm10358, Gm31659, Gm31138, Gm48271, Gpx4-ps2, Nudt8, Gm26564, Gm19531, Gm38431, Gm49373, Gm47163, Gm47939, Srp54b, Gm48764, Gm47164, A730063M14Rik, D430020J02Rik, Gm3333, Gm48754, Gm36839, Gm46430, Gm19428, Gm49371, Eprn, Gm49328, Gm49359, Gm48309, Gm49327, Gm48035, Gm7240, Adat3, Gm49329, Gm49686, Gm47486, Gm38699, Gm49602, Gm48701, Gm48799, Scamp4, Gm46339, Gm20336, Gm36298, Gm48244, Gm49336, Gm7968, Gm46522, Eeflakmt4, Gm49747, Gm49378, Pnp, Rps12-ps2, Gm49527, Septin2, C030006K11Rik, Gm10362, Gm49492, Gm32885, Gm44502, Gm30124, Gm49769, Rps2-ps7, Gm49909, Gm49804, Gm30938

List 2. ATP13A2, CHMP2B, EPHA4, LGALSL, TARDBP, ATXN2, CHRNA3, ERBB4, LRP12, TBK1, BSCL2, CYLD, FIG. 4, PIKFYVE, TIA1, C9ORF72, DCTN1, FUS, RNF13, TUBA4A, CACNA1H, DDHD1, GLT8D1, SCFD1, UBQLN2, CCNF, DNAJC7, HNRNPA1, SOD1, VAPB, CHCHD10, ELP3, hnRNPA2B1, SS18L1

In an embodiment, the target MN comprises a MN of a subject comprising histone hypermethylation. In an embodiment, the target MN comprises a MN of a subject comprising a H3K9me3 hypermethylation. In an embodiment, the target MN comprises H3K9me3 hypermethylation.

In an embodiment, the administration of a therapeutically effective amount of any embodiment of the degenerative motor neuron treatment composition of the present invention to a target MN of a subject suppresses the H3K9me3 level in the target MN of the subject to about 50%, about 55%, about 60%, about 65%, about 70%, about 75%, about 80%, about 85%, about 90%, about 95%, about 100%, about 125%, or about 150% of the H3K9me3 level in a MN of the subject when healthy. In an embodiment, the administration of a therapeutically effective amount of any embodiment of the degenerative motor neuron treatment composition of the present invention to a target MN of a subject suppresses the H3K9me3 level in the target MN of the subject to about 50%, about 55%, about 60%, about 65%, about 70%, about 75%, about 80%, about 85%, about 90%, about 95%, about 100%, about 125%, or about 150% of the H3K9me3 level in a MN of the subject when not suffering from a MND. In an embodiment, the administration of a therapeutically effective amount of any embodiment of the degenerative motor neuron treatment composition of the present invention to a target MN of a subject suppresses the H3K9me3 level in the target MN of the subject to about 50%, about 55%, about 60%, about 65%, about 70%, about 75%, about 80%, about 85%, about 90%, about 95%, about 100%, about 125%, or about 150% of the H3K9me3 level of an average subject. In an embodiment, the administration of a therapeutically effective amount of any embodiment of the degenerative motor neuron treatment composition of the present invention to a target MN of a subject suppresses the H3K9me3 level in the target MN of a subject to about 50%, about 55%, about 60%, about 65%, about 70%, about 75%, about 80%, about 85%, about 90%, about 95%, about 100%, about 125%, or about 150% of the H3K9me3 level of an average healthy subject having the same biometrics of the subject such as age, sex, height, weight, etc. or a combination thereof.

In an embodiment, the administration of a therapeutically effective amount of any embodiment of the degenerative motor neuron treatment composition of the present invention to a target MN of a subject suppresses the H3K9me3 level in the target MN of the subject by at least about 5%, about 10%, about 15%, about 20%, about 25%, about 30%, about 40%, about 50%, about 60%, about 70%, about 80%, about 90%, about 100%, about 150%, about 200%, about 250%, about 300%, about 350%, about 400%, about 450%, or about 500%.

In an embodiment, the administration of a therapeutically effective amount of any embodiment of the degenerative motor neuron treatment composition of the present invention to a target MN of a subject restores the expressions of genes involved in synaptic function, RNA metabolism, chromatin modification, histone modification, or a combination thereof of the subject to about 50%, about 55%, about 60%, about 65%, about 70%, about 75%, about 80%, about 85%, about 90%, about 95%, about 100%, about 125%, or about 150% of the respective gene expression level in a MN of the subject when healthy. In an embodiment, the administration of a therapeutically effective amount of any embodiment of the degenerative motor neuron treatment composition of the present invention to a target MN of a subject restores the expressions of genes involved in synaptic function, RNA metabolism, chromatin modification, histone modification, or a combination thereof of the subject to about 50%, about 55%, about 60%, about 65%, about 70%, about 75%, about 80%, about 85%, about 90%, about 95%, about 100%, about 125%, or about 150% of the respective gene expression level in a MN of the subject when not suffering from a MND. In an embodiment, the administration of a therapeutically effective amount of any embodiment of the degenerative motor neuron treatment composition of the present invention to a target MN of a subject restores the expressions of genes involved in synaptic function, RNA metabolism, chromatin modification, histone modification, or a combination thereof of the subject to about 50%, about 55%, about 60%, about 65%, about 70%, about 75%, about 80%, about 85%, about 90%, about 95%, about 100%, about 125%, or about 150% of the respective gene expression level in a MN of an average subject. In an embodiment, the administration of a therapeutically effective amount of any embodiment of the degenerative motor neuron treatment composition of the present invention to a target MN of a subject restores the expressions of genes involved in synaptic function, RNA metabolism, chromatin modification, histone modification, or a combination thereof of the subject to about 50%, about 55%, about 60%, about 65%, about 70%, about 75%, about 80%, about 85%, about 90%, about 95%, about 100%, about 125%, or about 150% of the respective gene expression level in a MN of an average healthy subject having the same biometrics of the subject such as age, sex, height, weight, etc. or a combination thereof.

The present invention further provides a method of treatment of MND of a subject comprising the step of administering a therapeutically effective amount of any embodiment of the degenerative motor neuron treatment composition of the present invention to the subject. In an embodiment, the MND comprises amyotrophic lateral sclerosis (ALS) comprising familial ALS, sporadic ALS, or a combination thereof, progressive bulbar palsy, primary lateral sclerosis, progressive muscular atrophy, spinal muscular atrophy, Kennedy's disease, or post-polio syndrome. In an embodiment, ALS comprises C9orf72-associated ALS, SOD1-associated ALS, TDP43-associated ALS, FUS-associated ALS, ALS associated with abnormalities derived from one or more genes comprising CCNF, NEK1, VCP, SQSTM1, OPTN, UBQLN2, PFN1, TUBA4A, MATR3, CHCHD10, TBK1, KIF5A, NEFH, NEFH, SETX, DCTN1, VAPB, CHMP2B, SPG11, HNRNPA2B1, HNRNPA1, CFAP410, ANXA11, ERLIN1, GLT8D1, DNAJC7, HTT, or SPTLC1, or a combination thereof.

In an embodiment, dosage of betaine chloride in any embodiment of the degenerative motor neural treatment composition of the present invention comprising betaine chloride is based on concentration range from about 50 nM to 1000 nm such as about 50 nM, 75 nM, 100 nM, 120 nM, 140 nM, 160 nM, 180 nM, 200 nM, 250 nM, 300 nM, 400 nM, 500 nM, 600 nM, 700 nM, 800 nM, 900 nM or 1000 nM including any concentrations or concentration values falling within these concentration values. In an embodiment, dosage of folic acid (B9) in any embodiment of the degenerative motor neural treatment composition of the present invention comprising folic acid (B9) is based on concentrations of folic acid (B9) ranging from about 10 nM to 500 nm such as about 10 nM, 20 nM, 30 nM, 40 nM, 50 nM, 60 nM, 70 nM, 80 nM, 90 nM, 100 nM, 200 nM, 300 nM, 400 nM, or 500 nM including any concentrations or concentration values falling within these concentration values.

In an embodiment, dosage of chaetocin in any embodiment of the degenerative motor neural treatment composition of the present invention comprising chaetocin is based on concentration of chaetocin ranging from about 0.1 nM to 10 nM such as about 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, or 10 nM, including any concentrations or concentration values falling within these concentration values. In an embodiment, dosage of F5446 in any embodiment of the degenerative motor neural treatment composition of the present invention comprising F5446 is based on concentration of F5446 ranging from about 0.1 nM to 10 nM such as about 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, or 10 nM, including any concentrations or concentration values falling within these concentration values.

In an embodiment, dosage of B12 in any embodiment of the degenerative motor neural treatment composition of the present invention comprising B12 is based on concentration of B12 ranging from about 10 nM to 500 nm such as about 10 nM, 20 nM, 30 nM, 40 nM, 50 nM, 60 nM, 70 nM, 80 nM, 90 nM, 100 nM, 200 nM, 300 nM, 400 nM, or 500 nM including any concentrations or concentration values falling within these concentration values.

In an embodiment, dosage of FB23-2 in any embodiment of the degenerative motor neural treatment composition of the present invention comprising FB23-2 is based on concentration of FB23-2 ranging from about 0.1 nM to 10 nM such as about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM including any concentrations or concentration values falling within these concentration values. In an embodiment, dosage of IOX1 in any embodiment of the degenerative motor neural treatment composition of the present invention comprising IOX1 is based on concentration of IOX1 ranging from about 0.1 nM to 10 nM such as about 0.1 μM, 0.2 μM, 0.3 μM, 0.4u M, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM including any concentrations or concentration values falling within these concentration values. In an embodiment, dosage of SAM in any embodiment of the degenerative motor neural treatment composition of the present invention comprising SAM is based on concentration of SAM ranging from about 10 nM to 500 nm such as about 10 nM, 20 nM, 30 nM, 40 nM, 50 nM, 60 nM, 70 nM, 80 nM, 90 nM, 100 nM, 200 nM, 300 nM, 400 nM, or 500 nM including any concentrations or concentration values falling within these concentration values.

The present invention also provides a MN degeneration model comprising a model subject wherein the model subject is deficient in METTL3 expression, METTL14 expression, or a combination thereof. In an embodiment, the model subject comprises a subject capable of serving as the MN degeneration model. In an embodiment, the model subject comprises a cell or an animal such as but not limited to a human or a mouse. In an embodiment, the model subject comprises a cell derived from an animal such as an iPSC or a differentiated cell thereof such as but not limited to an iPSC-derived MN. In an embodiment, the model subject comprises a cholinergic neuron.

In an embodiment, model subject of the the MN degeneration model of the present invention comprises a cholinergic neuron deficient in METTL3 expression, METTL14 expression, or a combination thereof. In an embodiment, the cholinergic neuron comprises a MN.

In an embodiment, the expression level of METTL3 in the model subject of the MN degeneration model of the present invention is at least about 5% to about 100% such as about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or any percentages or percentage ranges falling within these percentage values less than the expression level of METTL3 in a subject not deficient in METTL3 expression, METTL14 expression, or a combination thereof.

In an embodiment, the expression level of METTL14 in the model subject of the MN degeneration model of the present invention is at least about 5% to about 100% such as about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or any percentages or percentage ranges falling within these percentage values less than the expression level of METTL14 in a subject not deficient in METTL3 expression, METTL14 expression, or a combination thereof.

In an embodiment, the m⁶A level in the model subject of the MN degeneration model of the present invention is at least about 5% to about 100% such as about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or any percentages or percentage ranges falling within these percentage values less than the m⁶A level in a subject not deficient in METTL3 expression, METTL14 expression, or a combination thereof.

The present invention further provides a method of preparation of MN degeneration model of the present invention comprising the step of reducing the expression of METTL3, METTL14, or a combination thereof in a model subject. In an embodiment, the model subject comprises a cell or an animal such as but not limited to a human or a mouse. In an embodiment, the model subject comprises a cell derived from an animal such as an iPSC or a differentiated cell thereof such as but not limited to an iPSC-derived MN. In an embodiment, the model subject comprises a cholinergic neuron.

In an embodiment, the step of reducing the expression of METTL3, METTL14, or a combination thereof in a model subject comprises treating the model subject with a METTL3 inhibitor, a METTL14 inhibitor, or a combination thereof. In an embodiment, the METTL3 inhibitor comprises a small molecule compound, an oligonucleotide, or a combination thereof. In an embodiment, the METTL3 inhibitor comprises STM2457, STC15, or a combination thereof. In an embodiment, the nucleotide sequence of the oligonucleotide capable of targeting METTL3 is at least about 80%, about 85%, about 90%, about 95%, or about 100% identical to SEQ ID NO. 274, SEQ ID NO. 275, SEQ ID NO. 276, or SEQ ID NO. 277.

SEQ ID NO. 274:
CGTCAGTATCTTGGGCAAGTT
SEQ ID NO. 275:
GCCAAGGAACAATCCATTGTT
SEQ ID NO. 276:
CCGGCGTCAGTATCTTGGGCAAGTTCTCGAGAACTTGCCCAAGATACTG
ACGTTTTTG
SEQ ID NO. 277:
CCGGGCCAAGGAACAATCCATTGTTCTCGAGAACAATGGATTGTTCCTT
GGCTTT

In an embodiment, the METTL14 inhibitor comprises a small molecule compound, an oligonucleotide, or a combination thereof. In an embodiment, the nucleotide sequence of the oligonucleotide capable of targeting METTL14 is at least about 80%, about 85%, about 90%, about 95%, or about 100% identical to SEQ ID NO. 278, SEQ ID NO. 279, SEQ ID NO. 280, or SEQ ID NO. 281.

SEQ ID NO. 278:
GCCGTGGACGAGAAAGAAATA
SEQ ID NO. 279:
GCTAATGTTGACATTGACTTA
SEQ ID NO. 280:
CCGGGCCGTGGACGAGAAAGAAATACTCGAGTATTTCTTTCTCGTCCAC
GGCTTTTT
SEQ ID NO. 281:
CCGGGCTAATGTTGACATTGACTTACTCGAGTAAGTCAATGTCAACATT
AGCTTTTT

In another embodiment, the step of reducing the expression of METTL3, METTL14, or a combination thereof in model a subject comprises generating an animal line comprises a Cre-loxP system for a conditional knockout of the METTL3 gene, the METTL14 gene, or a combination thereof. In an embodiment, the expression of Cre recombinase in the animal line comprising the Cre-loxP system of the present invention is driven by choline O-acetyltransferase (ChAT) so that the METTL3 gene, the METTL4 gene, or a combination thereof is knockout in at least a cholinergic neuron of the animal line comprising a Cre-loxP system of the present invention. In another embodiment, the expression of Cre recombinase in the animal line comprising the Cre-loxP system of the present invention is driven by oligodendrocyte transcription factor 2 (Olig2) so that the METTL3 gene, the METTL4 gene, or a combination thereof is knockout in at least a MN or an oligodendrocyte of the animal line comprising a Cre-loxP system of the present invention. In an embodiment, the METTL3 gene, METTL14 gene, or a combination thereof is knockout in a MN of the animal line comprising a Cre-loxP system of the present invention.

The present invention also provides a method of using the MN degeneration model of the present invention for studying the pathology of a MND, developing therapeutic strategy for a MND, or a combination thereof. In an embodiment, the MND comprises amyotrophic lateral sclerosis (ALS) comprising familial ALS, sporadic ALS, or a combination thereof, progressive bulbar palsy, primary lateral sclerosis, progressive muscular atrophy, spinal muscular atrophy, Kennedy's disease, or post-polio syndrome. In an embodiment, ALS comprises C9orf72-associated ALS, SOD1-associated ALS, TDP43-associated ALS, FUS-associated ALS, ALS associated with abnormalities derived from one or more genes comprising CCNF, NEK1, VCP, SQSTM1, OPTN, UBQLN2, PFN1, TUBA4A, MATR3, CHCHD10, TBK1, KIF5A, NEFH, NEFH, SETX, DCTN1, VAPB, CHMP2B, SPG11, HNRNPA2B1, HNRNPA1, CFAP410, ANXA11, ERLIN1, GLT8D1, DNAJC7, HTT, or SPTLC1, or a combination thereof.

It is to be understood that both the foregoing general description and detailed description are exemplary and explanatory only and are not restrictive of the invention, as claimed. In general, the terms used in the disclosure should not be construed to limit the technology to the specific embodiments disclosed in the specification, unless the above detailed description explicitly defines such terms. Accordingly, the actual scope of the technology encompasses the disclosed embodiments and all equivalent ways of practicing or implementing the technology.

### Examples

### Materials and Methods

Human iPSC Culture

The SOD1^{+/L144F} ALS iPSC mutant line (female) and a healthy control line were acquired from the Harvard Stem Cell Institute iPS Core Facility. TDP43^{G298S} (male), C90RF72^{exp} (male), an isogenic control line of C9ORF72^{exp}, and sporadic ALS iPSC lines (male) were acquired from the Answer ALS project of Cedars-Sinai. Cells were maintained in feeder-free Essential 8 (Life Science) conditions and subcultured by 0.5 mM EDTA treatment. Cells were cultured in a 5% CO₂ humidified atmosphere at 37° C. The influence of sex was not assessed in this study.

### Generation of an Inducible METTL14-Knockdown SH-SY5Y Cell Line

SH-SY5Y cells were maintained in DMEM/F12 containing Glutamax (Thermo) supplemented with 10% FBS (Thermo). To generate an inducible METTL14-knockdown SH-SY5Y cell line, we generated a lentivirus containing a doxycycline-inducible shRNA cassette for METTL14 from HEK293 cells. We transferred the lentivirus-containing medium into SH-SY5Y cells for 2 days. Transduced cells were selected with puromycin (2 ug/mL) for one week. To assess METTL14-knockdown efficiency, inducible METTL14-knockdown SH-SY5Y cells were cultured in a medium with 2 μg/mL doxycycline hydrochloride (Sigma D3447) for four days, and the cell lysate was harvested for quantitative PCR, m⁶A dot blot, and m⁶A ELISA assay.

### Inducible METTL14-Knockdown SH-SY5Y Cell Line Differentiation

We adopted a protocol for SH-SY5Y cell differentiation described previously 37. In brief, 5×10⁵ SH-SY5Y cells were plated on a 35-mm dish on day 0. On day 1, the cells were cultured in DMEM with 2.5% FBS and 10 μM Retinoic Acid (RA) (Sigma-Aldrich) for 7 days. On day 7, the cells were re-plated on a new 35-mm dish with 2.5% FBS DMEM plus 10 μM RA. On days 8 and 9, the SH-SY5Y cells were cultured in DMEM with 1% FBS and 10 μM RA. On day 10, they were re-plated on dishes coated with poly-L-ornithine and laminin in 1% FBS DMEM with 10 μM RA. On day 11, the SH-SY5Y cells were cultured in a Neural basal medium supplemented with 1× B27 (Life Technology), 20 mM KCL (Sigma-Aldrich), 2 mM db-cAMP (Medchemexpress), 50 ng/ml BDNF (Peprotech), and 10 UM RA until day 18. To induce METTL14 knockdown, 2 μg/mL doxycycline was added into the differentiation medium from day 14 to day 18. The SH-SY5Y differentiation medium was usually refreshed every two days.

### Mouse Crosses

Mice carrying the mutant human SOD1^{G93A} transgene (B6SJL-Tg (SOD1*G93A) 1Gur/J) were purchased from the Jackson Laboratory (JAX: 002726). Mettl14^{floxed} mice were obtained from Chuan He in the University of Chicago ¹⁸, and Olig2-Cre was a gift from Tom Jessell in Columbia University. ChAT-IRES-Cre (Aneo) (Jackson Laboratory stock: 031661), in which the neomycin cassette was removed to avoid the ectopic expression sometimes observed in the ChAT-IRES-Cre line, were bred with the Mettl14^{floxed} allele to generate ChAT-Cre; Mettl14^{f/+} and ChAT-Cre; Mettl14^{floxed} mice. WT and conditional knockout mice were generated by crossing ChAT-Cre; Mettl14^{f/+} males and Mettl14^{floxed} females. CAG-Sun1/sfGFP mice were purchased from JAX (stock no. 021039; B6;129-Gt(ROSA)26Sor^{tm5(CAG-Sun1/sfGFP)Nat}/J) and crossed with ChAT-Cre; Mettl14^{floxed} mice. F1 heterozygous reporter mice were maintained to P100~P120 and then sacrificed for subsequent single-nucleus multiome experiments. All live animals were kept in an SPF animal facility, housed at ~55% humidity, 25° C., on a 12:12-hour light/dark cycle, and approved and overseen by IACUC, Academia Sinica.

Differentiation and Survival Assay of Human iPSC-Derived MNs

Human iPSCs were differentiated into MNs using an improved protocol ³⁸. Specifically, iPSCs were dissociated into single cells using accutase (eBioscience) at day 0. Next, 1.5~2×10⁵ cells were resuspended in 10 mL N2/B27 medium [1:1 of DMEM-F12 and Neurobasal medium containing N2 (Life Technologies), B27 (Life Technologies), 1% penicillin-streptomycin, 200 mM Glutamax, 0.2 mM 2-mercaptoethanol, and 0.5 mM ascorbic acid (Sigma-Aldrich)], supplemented with 10 mM Y-27632 (STemGent), 20 mM SB431542 (Merck), 0.1 mM LDN 193189 (Sigma-Aldrich), three mM CHIR-99021 (Merck), and 10 ng/mL bFGF (Peprotech). Small embryoid bodies (EBs) should become visible after two days of differentiation. SB431542 and LDN 193189 were kept for four days and supplemented with 100 nM retinoic acid and 0.5 mM smoothened agonist from day 2 to day 16. Then, 10 ng/mL BDNF (Peprotech) was added from day 7, and 10 mM DAPT (Calbiochem) was included from day 9 to day 16. After HB9^{on} nascent MNs had been generated (day 11), EBs were dissociated utilizing accutase (Gibco), and the dissociated MNs were plated onto poly-L-Ornithine/laminin-coated four-well plates at a density of 5×10⁴ cells per well and 7.5×10⁵ cells per 6 cm plate. Dissociated MNs were maintained in MN culture medium [CultureOne Supplement medium (Thermo Fisher Scientific Inc.) containing 10 mM Y-27632, 10 ng/mL BDNF, 10 ng/ml GDNF (Peprotech) and 10 mM 5-fluoro-20-deoxyuridine/Uridine (to inhibit proliferating cells) (Merck)]. Medium was replenished every three to five days.

To accelerate MN degeneration, the CultureOne Supplement medium of day 31 MN culture was replaced with N2/B27 medium only (no BDNF and GDNF), for which CPA was supplemented to accelerate degeneration for another seven days. FB23-2 was added with the CPA for 15~30 days to extend disease onset or rescue the degenerative process. MNs were revealed by SMI32 immunostaining and captured by an ImageXpress® Micro XLS High-Content Imaging System (Molecular Devices). The degeneration index was calculated at the indicated time point after CPA treatment.

### Quantification of Neurite Degeneration

To ensure accurate measurement of neurites, images were captured from blindly selected regions with well-separated axon tracts. Neurite fragmentation was then quantified using an automated image analysis method. The extent of neurite degeneration was expressed as a degeneration index (DI), defined as the ratio of the fragmented neurite area to the total neurite area. To process images for DI calculation, gray intensity in images was first normalized using the auto-level function of GNU Image Manipulation Program (GIMP) software, ensuring consistent background intensity across all images. Subsequently, ImageJ and Ilastik software were employed to binarize the images and remove cell bodies, resulting in a black-and-white rendering of neurites. While intact neurites exhibit continuity, degenerating neurites display disrupted, particulate structures due to blebbing and fragmentation. To quantify the fragmented areas of these degenerating neurites, the Particle Analyzer algorithm in ImageJ was used, with detection parameters set for size (20-10,000 pixels) and circularity (0.2-1.0). The total area of identified neurite fragments was then divided by the overall black neurite area to calculate the DI. Consistent with previous studies, the DI ranged from 0, representing completely intact neurites, to 1.0, indicating complete degeneration into fragmented particles ^{39,40}

### m⁶A Methylation and Quantification

The total RNA or mRNA was extracted from samples using Trizol (Life Technologies) and m⁶A methylation was quantified using the m⁶A RNA Methylation Assay Kit (ab185912, Abcam), with absorbance read at 450 nm in biological triplicates using an EnSpire Multimode reader and EnSpire software. To quantify relative m⁶A RNA methylation, the percentage m⁶A content in total RNA was calculated as: m⁶A %=(Sample OD-negative control OD)/the amount of input sample RNA×100% (positive control OD-negative control OD)/the amount of input positive control

### m⁶A Dot Blot Assay

Total RNA was extracted with Trizol (Life Technologies) and purified into mRNA using Invitrogen PolyA⁺ RNA selection (Dynabeads mRNA Purification Kit). After mRNA purification, 100~200 ng mRNA was dropped onto the Hybond-N+ membrane (Amersham Hybond™-N+ Membranes) for UV crosslinking (1200 μJ X100; UV Stratalinker 2400). Hybridized mRNA was blocked in 5% milk in 0.5% Triton X-100/PBS for one hour and then incubated with anti-m⁶A antibody (1:1,000, Synaptic) in a blocking solution at 4° C. overnight. After three TBST washes, the mRNA was incubated with an anti-HRP secondary antibody (1:10,000; Santa Cruz) in a blocking solution for 30 minutes at room temperature. The signal was developed with enhanced chemiluminescence (ImageQuant LAS 4000). The hybridized mRNA was stained with 0.2% methylene blue in 0.3 M sodium acetate (pH 5.2) as the loading control of mRNA.

### Immunostaining

Immunostaining was performed on cryostat sections as described previously ⁴¹. Spinal cord sections were permeabilized in 0.5% Triton X-100/PBS for one hour and then blocked in 3% bovine serum albumin (BSA) in 0.5% Triton X-100/PBS for one hour. The sections were incubated with indicated primary antibodies in blocking solutions at 4° C. for two days. After five PBS washes, the sections were incubated with secondary antibodies and DAPI in a blocking solution for 1.5~2 hours at room temperature. After five PBS washes, the sections were mounted with Aqua-Poly/Mount (18606-5; Polysciences Inc.). Dissociated MNs from iPSC differentiation were fixed and permeabilized in 0.1% Triton X-100/PBS for 5~10 minutes and then blocked in 10% FBS in 0.1% Triton X-100/PBS for 30 minutes. The sections were incubated with indicated primary antibodies in a blocking solution at 4° C. overnight. After three PBS washes, the sections were incubated with secondary antibodies and DAPI in a blocking solution for 45 minutes at room temperature.

### Spinal Motor Neuron Quantification

At each indicated stage, ChAT^{on} MNs in the lumbar ventral horns with DAPI nuclear signal were counted on one side of a 20 μm-thick sectioned spinal cord. MNs that did not show regular nuclear shapes were excluded. The quantification bar charts represent average MN counts from at least three sections per mouse (n≥3 mice) of the same age and genotype.

### Survival Analyses

Mice were assessed weekly for baseline weight from P60. The onset of weight decline is defined as the age at which the animal lost 5% of peak body weight. We used the Kaplan-Meier method with a log-rank test to compare onset-free survival for each group. The disease end-point was defined as the day when mice could not right themselves within 15 seconds ⁴², when they were sacrificed. Survival analysis was performed using Kaplan-Meier analysis.

### NMJ Analysis

To reveal neuromuscular junctions (NMJs) in whole-mount muscles, tissues were permeabilized and blocked in 3% bovine serum albumin (BSA) in 2% Triton X-100/PBS at room temperature for two hours, followed by incubation with anti-neurofilament antibody (DSHB, 1:250) together with anti-SV2 (DSHB, 1:500) to label axonal endfeet in blocking buffer at 4° C. for three days. After five 2% Triton X-100/PBS washes, the muscle tissues were incubated overnight at 4° C. with secondary antibodies, together with Alexa Fluor 555 labeled a-bungarotoxin [α-BTX, Thermo Fisher Scientific, B13422] to detect nicotinic acetylcholine receptors (AChR). Muscles were teased apart and flattened before mounting on slides. Z-stack images were acquired using a Zeiss LSM780 confocal microscope. All figures containing confocal images are projections of Z stacks. Innervated NMJs were counted when the AChR^{on} endplates overlapped with axon terminals (as revealed by neurofilament staining), whereas the denervation ratio was calculated according to the colocalization between neurofilament, SV2 and the a-BTX signals of each picture, with values normalized against total endplate area. All NMJ areas were analyzed in ImageJ using threshold adjustment. Herein, we only examined the total denervation ratio, as represented by marker non-colocalization in NMJs.

### Behavioral Assays

Locomotor activity was measured according to open field, rotarod, and treadmill tests. Age-matched wild-type (WT) mice from the littermate control line were used for experimental comparisons. Both sexes of indicated ages (P40, P70, P100, P130, and P160 for ChAT-Cre; Mettl14^{floxed} cKO mice) were used in this study. One week before intrathecal injection, the SOD1^{G93A} mice were tested every two weeks for the rotarod test and every three weeks for the CMAP examination from P60 onward. The experimenters conducting all behavioral assays were blind to mouse genotypes.

### Rotarod

A commercially available rotarod apparatus (47600 Rota-Rod, Ugo Basile, Italy) with a rotating rod of 5 cm diameter was used. Mice were transferred to the testing room and habituated in the home cage at least 15 minutes before testing. In the training phase, three trials with a constant speed of 4 rpm and a 60-second cut-off time were used to ensure that all test mice could stay on the rod for a training trial before moving to the test phase. After a 30-minute rest interval, the mice were evaluated during the test phase, with the rod accelerating from 4 to 40 rpm with a 300-second cut-off time in a series of three trials. The longest falling latency, as well as the rotating speed when the mouse fell off the apparatus, were used to represent the motor coordination of each mouse 43.

### Grip Strength

A grip strength meter (MK-380 CM/R; Muromachi) was used to measure forelimb grip strength. As a mouse grasped the bar, the peak pull force in grams was recorded on a digital force transducer. During the test, a mouse was allowed to grasp the bar mounted on the force gauge. We performed four consecutive measurements per test at one-minute intervals.

### Open-Field Test

A square arena with opaque walls (area 48×48 cm and height 35 cm) was used. Mice were transferred to the testing room and habituated in the home cage one hour before testing and then allowed to explore the test chamber for another hour during which all behaviors were videotaped and tracked using a video-tracking system mounted on top of the arena (Clever System, Reston, VA). Total distance and average velocity were analyzed for each mouse ⁴³.

### Treadmill Locomotion Analysis

A TreadScan apparatus (CleverSys, Reston, VA) was used to analyze gait. Mice were placed on a stationary treadmill for acclimation and trained at a speed of 10 cm/seconds for five minutes before testing. Four test speeds were analyzed (10, 15, 20, and 25 cm/seconds) for each trial, which were recorded at 79 frames/s for 10 seconds using TreadScan software. For data analyses, the successful trials in which a mouse was able to maintain treadmill speed with continuous locomotion for each 10-second recording was selected and further analyzed using TreadScan software. Only gait analyses from the trials conducted at 15 cm/s are shown in the present study. The gait parameters of stride for each limb were automatically and unbiasedly calculated, and average values were used for statistical analysis.

### Mouse Motor Neuron Differentiation

Mouse embryonic stem cells were differentiated into motor neurons following published protocols ^{44,45}. Specifically, in this condition, we cultured the 3D embryoid bodies for five days, then attached the culture of the embryoid bodies to poly-ornithine and laminin-coated plates with an enrichment of neurotrophic factors (30 ng/mL GDNF, Peprotech) and cultured them for an extra 5 days (Day12). Change the medium every two days. Mouse ESC~MNs were used for imaging-based experiments, qPCR, and nanopore direct RNA sequencing.

### Quantification of Neurite Thickness, Neurite Complexity, and Synaptic Puncta Numbers

Using an automated image analysis method, the synaptic puncta labeled with Syn1 and the neurite labeled with Smi32 were measured. For synapse quantification, images were processed with ImageJ and ilastik software to binarize the images and eliminate the background and embryonic body, resulting in an image featuring black synapses and neurites on a white background. The Particle Analyzer algorithm in ImageJ was utilized to quantify the number of synaptic puncta within the neurites, setting parameters for size (0-100 pixels) and circularity (0.2-1.0). The neurite length was measured using the Analyze Skeleton algorithm in ImageJ. The total number of the identified synaptic puncta was then divided by the overall neurite length to calculate the average number of synapses. For the neurite complexity and thickness measurement, the neurite outside of the embryonic body was imaged. Neurite diameter was measured manually at the segment of each neurite within the fields with ImageJ's line tool. To quantify the branch point, the image was processed with ImageJ and ilastik software and analysed with the Analyze Skeleton algorithm in ImageJ. The total number of branch points was then divided by the overall neurite length to calculate the complexity.

### Nanopore Direct RNA Sequencing and Data Preprocessing

Nanopore direct RNA sequencing was conducted following instructions provided by Oxford Nanopore Technologies (Oxford, UK) using a Direct RNA Sequencing Kit (SQK-RNA002) and MinION flowcells (FLO-MIN106 R9 version). After live base-calling using Guppy (22.10.7) in MinKNOW, reads that passed the quality threshold were subjected to post-run base-calling with the latest version of Guppy (6.3.9) under default parameters. A reference transcriptome file was generated from the mouse genome reference and corresponding annotation. The Nanopore data provided in FASTQ format were initially aligned to the mouse genome reference from GenCode M20 using the minimap2 aligner. The resulting SAM format output was then converted to a sorted BAM format using samtools. To predict m⁶A modification sites and stoichiometry, we employed both EpiNano ⁴⁶ and m⁶Anet ⁴⁷. For EpiNano prediction, the output was first filtered with the DRACH motif. Then, the probability of modification was taken from the ‘ProbM’ column and the results from the ‘prediction’ column. For m⁶Anet prediction, the probability was yielded from the ‘probability_modified’ column and then filtered for values >0.9 for the results. Each tool predicted m⁶A sites independently, which were subsequently converted into BED format for further analysis. To refine our predictions, we cross-referenced predicted sites from EpiNano and m⁶Anet for each sample. We then identified the shared sites across EpiNano and m⁶Anet, finalizing our list of predicted m⁶A sites. Each m⁶A modified site was annotated using Homer v4.11 to determine its genomic location, categorizing them as Transcription Start Site (TSS), Transcription Termination Site (TTS), Exon (Coding), 5′ UTR Exon, 3′ UTR Exon, Intronic, or Intergenic.

### m⁶A Immunoprecipitation and Quantitative Real-Time PCR

To verify m⁶A sites, total RNA was isolated from mouse embryonic stem cell (mESC)-differentiated MNs using Trizol (Life Technologies). Total RNA samples were pooled to a total RNA amount of ~75 μg/sample and processed using Invitrogen PolyA+ RNA selection (Dynabeads mRNA Purification Kit, Cat. #61006) according to the manufacturer's protocol. Input mRNA (1%) was reserved for reverse transcription. Magnetic A beads (#88802, Thermofisher Scientific) were prepared and washed, adding 2 μl of N6-Methyladenosine Antibody/per sample. m⁶A-tagged transcripts of interest were pulled down using a rabbit polyclonal anti-m⁶A antibody (Synaptic Systems). The m⁶A-tagged mRNAs were competitively eluted from beads using N6-Methyladenosine 5′-monophosphate sodium salt. Input m⁶A pull-down mRNA was reverse transcribed by using the SuperScript III First-Strand Synthesis System for RT-PCR (Thermo Fisher). cDNA was then used for SYBR-green-based quantitative real-time PCR. Enrichment of m⁶A-tagged genes in m⁶A pull-down over input was calculated by comparing relative concentrations using Ct values (2-Ct) and dividing each concentration by the relative concentration of the input. The concentrations of the immunoprecipitated RNA were then divided by the concentration in the input RNA and multiplied by 100, to obtain the percentage of transcripts in the m⁶A immunoprecipitate relative to the input. This value was then normalized to low enrichment in m⁶A-tagged genes, which was also calculated using relative concentrations to determine a percentage of the input. Primers used are listed in Table 3.

TABLE 3
Primers used in the study
qPCR primers

| Gene | F sequence | R sequence |
|---|---|---|
| GAPDH | AATCCCATCACCATCTTCCA | TGGCTCCACGACGTACTCA |
| | (SEQ ID NO. 282) | (SEQ ID NO. 283) |
| METTL3 | GTGTCGGAGGTGATTCCAG | CTGCGCATCTCATCATCTGT |
| | T (SEQ ID NO. 284) | (SEQ ID NO. 285) |
| METTL14 | TGGACCTTGGAAGAGTGTG | TGCCAATTTCAGGTTCTTCT |
| | TT (SEQ ID NO. 286) | GTG (SEQ ID NO. 287) |
| FTO | TCCTGAGTGGCAGAAGAGG | CAGGTACTGTGGGAGGCAT |
| | T (SEQ ID NO. 288) | T (SEQ ID NO. 289) |
| ALKBH5 | TTCAAGCCTATTCGGGTGTC | CGGGGTGCATCTAATCTTGT |
| | (SEQ ID NO. 374) | (SEQ ID NO. 375) |
| Slc5a7 | TCGATCGCTTCACCCCCT | CTACTTCATCAAATCTTCTT |
| | (SEQ ID NO. 290) | TGCGT (SEQ ID NO. 291) |
| Syn1 | ATGCTCAGCAGCACAACAT | TCCAGTTACCCGACACTGAT |
| | A (SEQ ID NO. 292) | (SEQ ID NO. 293) |
| Fos | TTTCAACGCCGACTACGAG | CTGCGCAAAAGTCCTGTGT |
| | G (SEQ ID NO. 294) | G (SEQ ID NO. 295) |
| Col5a3 | AGCTACCTCCCTGGTTGTGA | GTGGAGGGAGGTCTGGTTC |
| | (SEQ ID NO. 296) | T (SEQ ID NO. 297) |
| Bag3 | GGACCCTAACCCAGCATGA | GGCTGAAGATGCAGTGTCC |
| | G (SEQ ID NO. 298) | TTA (SEQ ID NO. 299) |
| Aox1 | TCTGGCCACAGATGAGGTA | CAACATTCCCACACTCCAGC |
| | CA (SEQ ID NO. 300) | (SEQ ID NO. 301) |
| Spp1 | CTGGCTGAATTCTGAGGGA | TTCTGTGGCGCAAGGAGAT |
| | CT (SEQ ID NO. 302) | T (SEQ ID NO. 303) |
| Kcnj14 | AGATGGCCAAGCCCAAGAA | GGTCACACGGGGCTGC (SEQ |
| | A (SEQ ID NO. 304) | ID NO. 305) |
| Tardbp high m6A- | TTTCCACCACCAAGTCTCTG | CCGAGGCATGAAAGGCTAG |
| modified site 1 | C (SEQ ID NO. 306) | TA (SEQ ID NO. 307) |
| Tardbp high m6A- | TGCAAAGCACACCACAAGC | GTGTGGGAACGTGAACTGA |
| modified site 2 | (SEQ ID NO. 308) | AG (SEQ ID NO. 309) |
| Tardbp low m6A- | GCTCTTCAGCAGTCATGTCC | CCCAGACGAGCCTTTGAGA |
| modified site | T (SEQ ID NO. 310) | A (SEQ ID NO. 311) |
| Atp13a2 high | AGACAGTACCAGTCACCTC | TCAGCACTTCCCCAGGAGA |
| m6A-modified site | CC (SEQ ID NO. 312) | G (SEQ ID NO. 313) |
| Atp13a2 low m6A- | CCAAGGCCCATCAGCTTCA | GCAGCCACAAACTTCATGC |
| modified site | (SEQ ID NO. 314) | T (SEQ ID NO. 315) |
| Dctn1 high m6A- | CGACACCCCTCCACTAAAG | CAAGGGGTGAAGTCCTCAG |
| modified site | AT (SEQ ID NO. 316) | TC (SEQ ID NO. 317) |
| Dctn1 m6A low | TCAGCTTATGGAACAAGTG | TTCTCAATGGTGTCACTCAG |
| confidence | GCT (SEQ ID NO. 318) | GG (SEQ ID NO. 319) |
| C9orf72 high | CACACACTCTGTGAAGTGG | GGAATGGAGATCGGAGCAC |
| m6A-modified site | GA (SEQ ID NO. 320) | TT (SEQ ID NO. 321) |
| C9orf72 low m6A- | TGTCTTCAGGAACACTGTGG | CCCATGCTTACTGGGGAAG |
| modified site | G (SEQ ID NO. 322) | T (SEQ ID NO. 323) |
| Epha4 m6A high | CAATGGCACACCCTGGGTA | CAGAACAAAGCCAGTTCCG |
| mºA-modified site | A (SEQ ID NO. 324) | C (SEQ ID NO. 325) |
| Epha4 low mºA- | TTGGGTGGTCAAACTGTCCC | TCTGTGTGGCCATCAAGACT |
| modified site | (SEQ ID NO. 326) | C (SEQ ID NO. 327) |
| Cacnalh high | AGGTACAGTCCTTGCTCCTT | TTTGGGGGCTATAGTGCCTC |
| m6A-modified site | C (SEQ ID NO. 328) | T (SEQ ID NO. 329) |
| Cacnalh low m6A- | CTGAGGCAGAGCTTGAACC | TACTGAAGATGGCCACAGG |
| modified site | A (SEQ ID NO. 330) | C (SEQ ID NO. 331) |
| Glt8d1 high m6A- | TCCAGATGACTACCACGGC | ATGCTGGAGTTCTCTGTCCT |
| modified site | T (SEQ ID NO. 332) | TC (SEQ ID NO. 333) |
| Glt8d1 low m6A- | ATACACCACTGAAGCCAGG | AGTGGAGGCTGAGTCACAA |
| modified site | AC (SEQ ID NO. 334) | T (SEQ ID NO. 335) |
| Chrna3 high m6A- | ACATGTCCCATCTTGGCAGG | GGTGCTGAGCTCTCAAACCT |
| modified site | (SEQ ID NO. 336) | (SEQ ID NO. 337) |
| Chrna3 low m6A- | AAAGCACGATGTCTGGTTTC | GTGGAGTTCATGCGAGTCC |
| modified site | C (SEQ ID NO. 338) | C (SEQ ID NO. 339) |
| Fig4 high m6A- | AGGTGCTGGGGAGTCCTTT | GTGTCCTCTGTGAGAAGGC |
| modified site | A (SEQ ID NO. 340) | G (SEQ ID NO. 341) |
| Fig4 low m6A- | CATCTGAGCAACGGGAACA | AGCTGGGGCAAAGTCAACA |
| modified site | C (SEQ ID NO. 342) | TA (SEQ ID NO. 343) |
| Hnrnpa2b1 high | AGCTTCTTAACTCTACACAC | AGCCAGGATCATGGTGTAA |
| m6A-modified site | GCA (SEQ ID NO. 344) | TAAGA (SEQ ID NO. 345) |
| Hnrnpa2b1 low | TCCTAGAACTTTGGACTTCC | CACAATGCAGAAGTTAGAA |
| m6A-modified site | TGC (SEQ ID NO. 346) | AGGCA (SEQ ID NO. 347) |
| Ubqln2 high m6A- | TCCAGCTCTGCACCTACTGA | GGTCCAGATTCCGAGGTTG |
| modified site | (SEQ ID NO. 348) | G (SEQ ID NO. 349) |
| Ubqln2 low m6A- | AAACCCAGAAATCAGCCAC | TCCAGCGTCTGCCTCATTAT |
| modified site | CT (SEQ ID NO. 350) | (SEQ ID NO. 351) |
| Hnrnpal high | GGTAAACTTCCCCAACAGT | TGGCACCACCCTGAAGGAA |
| m6A-modified site | GTG (SEQ ID NO. 352) | T (SEQ ID NO. 353) |
| Hnrnpal low m6A- | GAGGCTATGGAAGTGGTGG | TGTCATAGCTGCCACTCCCG |
| modified site | AC (SEQ ID NO. 354) | (SEQ ID NO. 355) |
| Tuba4a high m6A- | ACTCTGTCAGGTCCACATTG | ATCACAGCTTCCTTGCGCTT |
| modified site | AG (SEQ ID NO. 356) | T (SEQ ID NO. 357) |
| Tuba4a low mºA- | GTCCAGCACTGGGTCGATG | CTATGCCCGTGGTCATTACA |
| modified site | AT (SEQ ID NO. 358) | CC (SEQ ID NO. 359) |
| Sod1 high m6A- | GCTTGGCCTGTGGAGTGATT | CTCAGACCACACAGGGAAT |
| modified site | (SEQ ID NO. 360) | GT (SEQ ID NO. 361) |
| Sod1 low m6A- | GCAATGTGACTGCTGGAAA | CGATCTTCAATGGACACATT |
| modified site | GG (SEQ ID NO. 362) | GGC (SEQ ID NO. 363) |
| Chmp2b high | GCAGGTGCCAAGCACATTT | ACCCCCTTTGGGAGAACATT |
| m6A-modified site | T (SEQ ID NO. 364) | (SEQ ID NO. 365) |
| Chmp2b low m6A- | GCGAACGTTCTTGTCTTCTG | GAAGCGTGCAGGGTTTTAG |
| modified site | T (SEQ ID NO. 366) | C (SEQ ID NO. 367) |
| PIKfyve high | CCACTGAACACAAAGACCT | GCAGTCATGGACAGTCTAA |
| m6A-modified site | TTCA (SEQ ID NO. 368) | AGC (SEQ ID NO. 369) |
| PIKfyve low m6A- | TTTTCTCAATTAGAGGCCCA | CTAACTTCTTCTCAGTGTTG |
| modified site | CC (SEQ ID NO. 370) | TTCTG (SEQ ID NO. 371) |

Cloning primers

| Gene | F sequence | R sequence |
|---|---|---|
| shFto oligomer | GATCCCCCCAGGGAGACTG | GGCCGCAAAAAGGTCCCTC |
| | CTATTTCATTTCAAGAGATA | TGACGATAAAGTATCTCTTG |
| | CTTTATCGTCAGAGGGACCT | AAATGAAATAGCAGTCTCC |
| | TTTTGC (SEQ ID NO. 372) | CTGGGGG (SEQ ID NO. 373) |

### Single-Nucleus Multiome

### Nuclei Collection

Mice were euthanized with isoflurane according to IACUC Academica Sinica guidelines. Mice were decapitated and spinal cords were extracted using a hydraulic extrusion approach. To do so, a blunt 25 G ¼ inch needle filled with ice-cold 1× PBS was placed into the caudal end of the vertebral column and a rapid hydraulic pressure was introduced to extract the entire spinal cord. Lumbar segments were dissected based on morphology and dissociated two at a time in 2 mL pre-chilled lysis buffer with a 7 mL Dounce Homogenizer (pestle A: eight strokes; pestle B: 5 strokes). An additional 3 mL lysis buffer was added, and the entire homogenate was filtered through a 100 μm strainer. Then, 5 mL of 50% iodixanol was added to create a 10 mL suspension of 25% iodixanol. The suspension was inverted to mix well before equally splitting into two 15 mL tubes. Iodixanol (40%, 2 mL) was layered gently at the bottom of the homogenate and centrifuged at 1,000 g for 12 minutes at 4° C., with a swing bucket centrifuge (brand). The compositions of the lysis buffer and iodixanol solution were listed in Table 4. The interphase nuclei were transferred to a new 15 mL tube and resuspended with 1:1 volume of 10 × wash buffer (10 mM Tris-HCl pH7.4, 10 mM NaCl, 3 mM MgCl₂, 1% BSA, 0.1% Tween 20, 1 mM DTT, and 1 U/μL RNase inhibitor), followed by centrifuging at 500 g for 10 minutes at 4° C. The supernatant was discarded and the pellet was resuspended in 800 μL diluted nuclei buffer (20× Nuclei Buffer from Chromium Next GEM Single Cell Multiome ATAC+Gene Expression Kit supplemented with 1 mM DTT, and 1 U/μL RNase inhibitor), before conducting FANS (fluorescence-activated nuclei sorting) using a BD FACS Aria III cell sorter (BD BioSciences, USA). GFP^{on} nuclei were collected in diluted nuclei buffer and centrifuged at 500 g for 7 minutes at 4° C. Nuclei were adjusted to an appropriate concentration following the manufacturer's protocol (10× Genomics, 1000285) with diluted nuclei buffer. The quality of nuclei was examined using an AxioImager Z1 upright microscope (Zeiss, Germany) at 40-fold magnification.

TABLE 4
Compositions of the lysis buffer and iodixanol solution

| | | Final | | |
|---|---|---|---|---|
| | | conc. | | (μl) |
| HB lysis buffer | | | | |
| 2.5M | Sucrose | 0.25M | | 600 |
| 2M | KCl | 25 | mM | 75 |
| 1M | MgCl2 | 5 | mM | 30 |
| 1M | Tris-HCl pH 7.4 | 20 | mM | 120 |
| 0.1M | DTT | 1 | mM | — |
| 40 U/μl | Rnase i | 1 | U/μl | — |
| 10% | IGEPAL-CA630 | 0.30% | | 180 |
| | Nuclease-free water | | | 5000 |
| | Total volume | | | 6 ml |
| Diluent buf (for | | | | |
| diluting iodixanol) | | | | |
| 2M | KCl | 150 | mM | 750 |
| 1M % | MgCl2 | 3 | mM | 30 |
| 1M % | Tris-HCl pH 7.4 | 120 | mM | 1200 |
| | Nuclease-free water | | | 8020 |
| | Total volume | | | 10 ml |

| | | Final | |
|---|---|---|---|
| | | conc. | (ml) |
| 50% iodixanol | | | |
| (freshly prep) | | | |
| 60% | Opti-prep | 50% | 5 |
| | Diluent buf | | 1 |
| | Total volume | | 6 |
| 40% iodixanol | | | |
| (freshly prep) | | | |
| 50% | Opti-prep | 40% | 3.2 |
| | HB buf | | 0.8 |
| | Total volume | | 4 |

### Single-Nucleus RNA and ATAC Sequencing

Transposition, nuclei isolation, and single-nucleus multiome libraries were prepared following the Chromium Next GEM Single Cell Multiome ATAC+Gene Expression protocol CG000338 Rev F (10× Genomics, Pleasanton, CA). The generated snRNA and ATAC libraries were sequenced to a minimum depth of 25,000 and 35,000 mean paired-end reads per nuclei, respectively, using a Nextseq 500 sequencer (RNA: 28-10-10-90 bps (base pairs), ATAC: 50-8-8 using an Illumina Nextseq 500 sequencer (Illumina, Inc., USA) and 16-49 bps). Raw sequencing data were processed using the standard Cell Ranger ARC pipeline (version 7.0.0, 10× Genomics) for demultiplexing, mapping to the mm 10 reference genome, barcode, and UMI (unique molecular identifier) counting, and generating the gene count matrix.

Joint Processing of snRNA and snATAC-Seq Data

Analysis was performed in RStudio and R (version 3.6.1) using multiple packages: Seurat, Signac, DoubletFinder, ClusterProfiler, ggplot2. Sample quality was examined to ensure the nuclei conformed to the following criteria: based on gene expression data-nFeature_RNA <10,000, nFeature_RNA >200, and nCount_RNA >500; and based on chromatin accessibility data-pct_reads_in_peaks >15, blacklist_fraction <5, nucleosome_signal <4, and TSS.enrichment >1. Doublets were estimated using DoubletFinder and removed from the analysis. The ATAC counts were normalized with TFIDF (term frequency-inverse document frequency), followed by dimensionality reduction with SVD (singular value decomposition). Clustering was performed using the second to tenth LSI (Latent Semantic Indexing) components. The gene expression counts were normalized whereby the expression value of each gene was divided by the total expression in each cell, multiplied by 10,000, and log-transformed. Highly variable genes across nuclei were selected and scaled. PCA (principal component analysis) was performed, and the top 30 principal components were selected for downstream clustering, as determined using an elbow plot. ATAC and RNA libraries for each sample were integrated using reciprocal LSI and MNN-CCA (Mutual Nearest Neighbor-Canonical Correlation Analysis). A WNN (weighted nearest neighbor) graph represented a weighted combination of RNA and ATAC assays with the first 60 and the second to tenth dimensions, respectively. Unbiased clustering of the RNA-ATAC integrated data at a resolution of 0.3 was used for cluster annotation. Major clusters were annotated using Seurat label transfer prediction scores with published single-cell RNA-seq clusters as reference ^{48,49}.

### Differential Expression and Chromatin Accessibility

Based on gene expression data, differential expression analysis was performed using the Seurat function FindMarkers and applying a likelihood-ratio test. Genes with Bonferroni-adjusted p-values of less than 0.05 were considered to be differentially expressed between Sun1^{sfGFP}; ChAT-Cre; Mettl14^{floxed} and littermate control. Differential chromatin accessibility analysis, based on chromatin accessibility data, was conducted using the Presto function wilcoxauc. In Presto, the p-value of the Wilcoxon rank sum test is computed based on Gaussian approximation and further adjusted by the Benjamini-Hochberg method. A peak is considered to be open in one condition and closed in the other if the adjusted p-value is less than 0.01, the logarithmic (base 2) fold-change is greater than 0.1, and the percentage of cells with a non-zero value in the first condition is greater than 5. Otherwise, the peak was denoted as neutral.

### Peak Annotation

Peak annotation, i.e., to determine if a peak is in the transcription start site (TSS), transcription termination site (TTS), Exon, 5′ UTR Exon, 3′ UTR Exon, intronic, or intergenic region was performed by HOMER using the annotatePeaks.p1 command on the mm10 genome reference.

### Identification of Gene-to-Peak Linkage

Identification of gene-to-peak linkage was performed for each cell type using ArchR's framework. First, to reduce the sparsity of chromatin data, aggregation of similar cells was performed using the k-nearest neighbor graph based on LSI reduction, and the cell aggregate-by-peak and cell aggregate-by-gene matrices were created and log-normalized. Aggregates with greater than 80% overlap with any other aggregates were filtered out. Then, all possible peak-to-gene pairs were selected if the peak was located within ±250 kilobase pairs flanking the gene start. Finally, the Pearson correlation was calculated across all cell aggregates for each possible peak-gene pair. Peak-to-gene linkages were then defined as those with Pearson correlation coefficients no less than 0.45 and adjusted p-values (by Benjamini-Hochberg method) no greater than 0.0001.

### H3K9Me3 and γH2AX Quantification

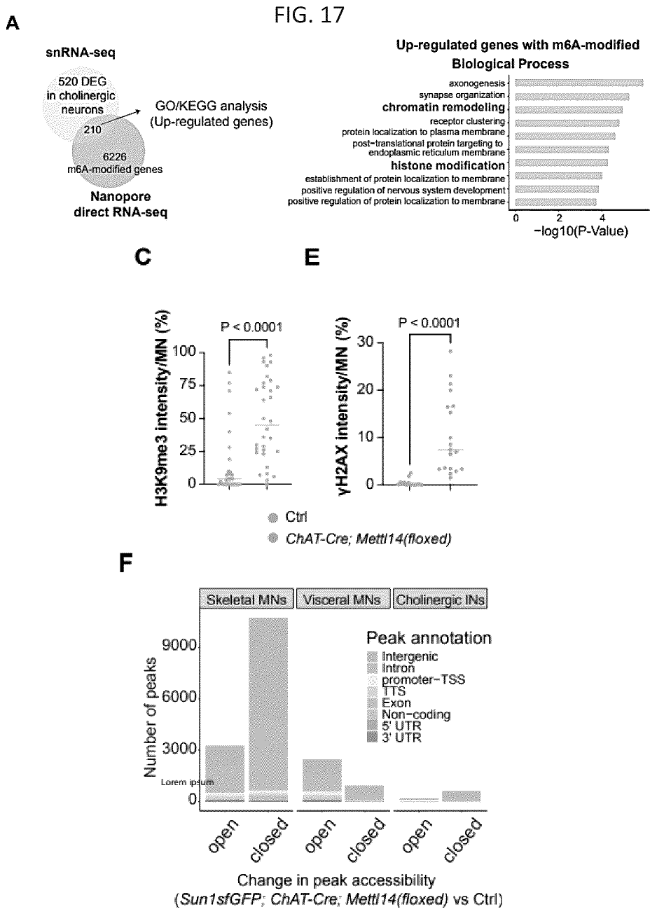

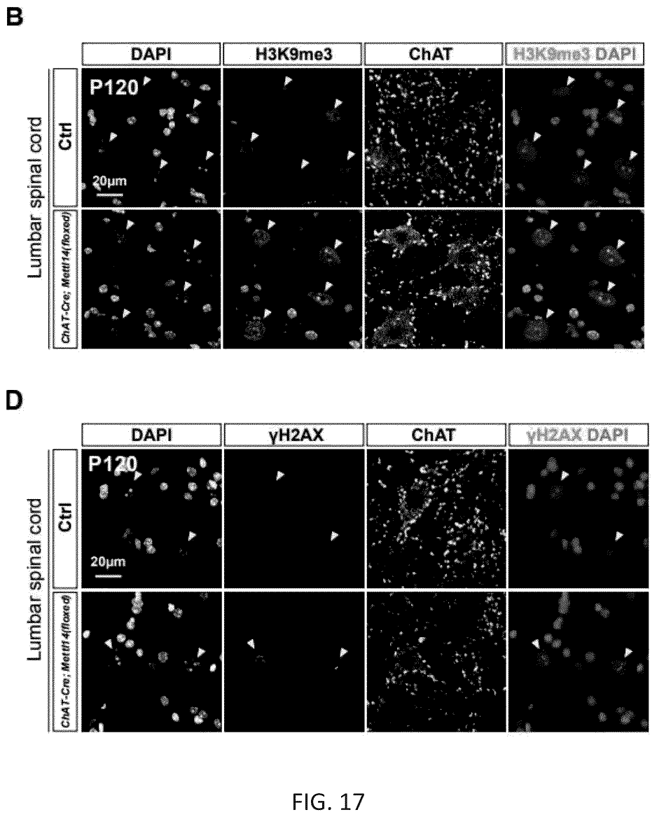

At the P120 stage, the signal of H3K9me3 or γH2AX was quantified (ImageJ) after training by machine learning (ilastik), and divided into the area of DAPI in each ChATon MN. Both the strength of the laser and the exposure time for H3K9me3 or γH2AX were fixed to facilitate quantification. In FIGS. 17C, 17D, 23A, 18A, 18B, and 24A, each point represents the quantified result for each ChATon MN, and those quantifications were counted from at least three mice (n≥3 mice) of the same age and genotype.

### Bulk RNA-Seq

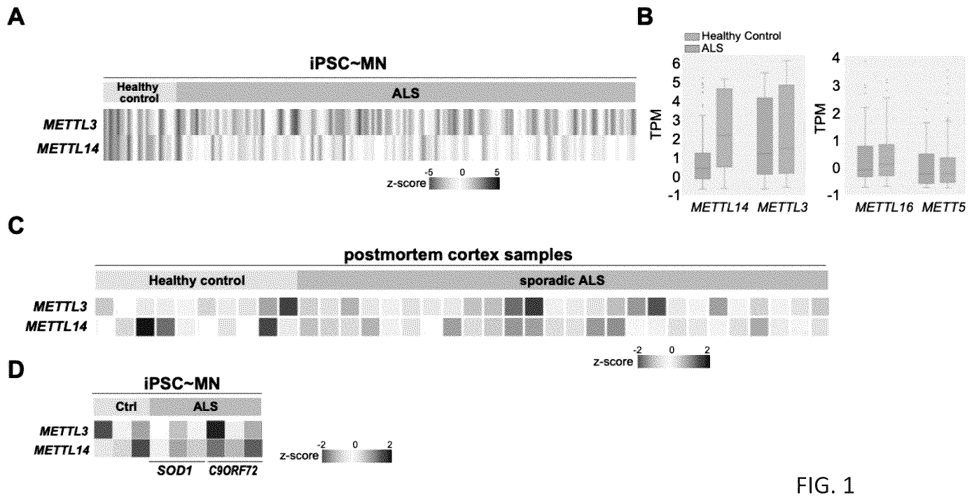

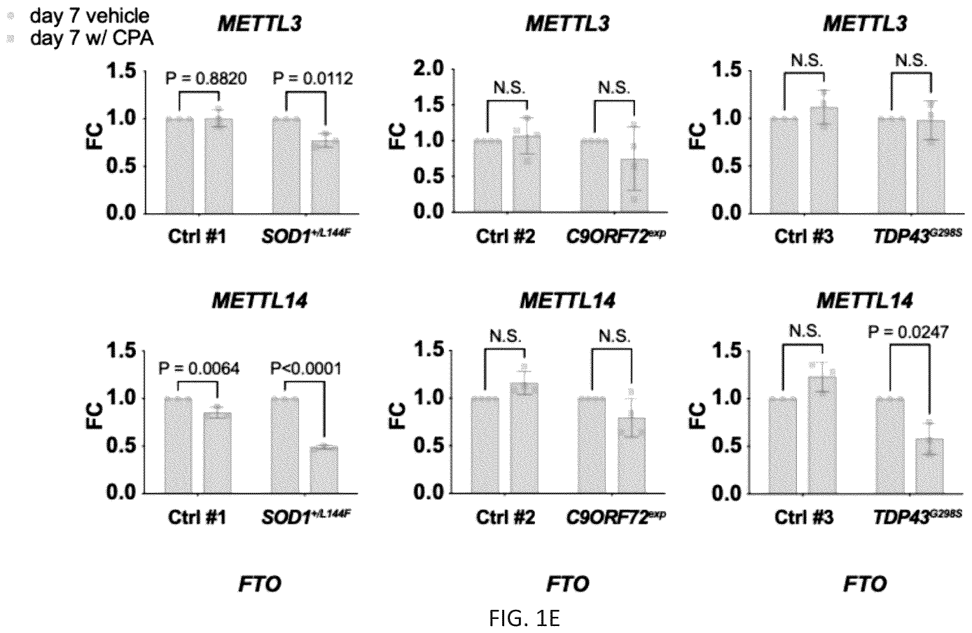

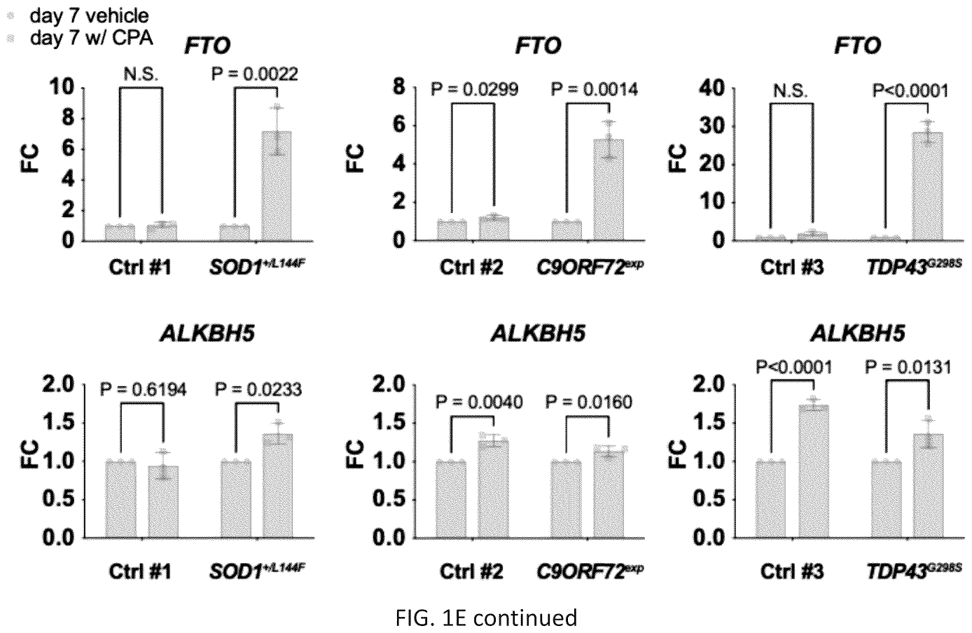

For bulk RNA-seq, RNA was extracted from ALS iPSC~MNs, and its quality was evaluated using a Bioanalyzer 2100 RNA pico kit. The cDNA libraries were prepared from the human iPSC~MNs according to the manufacturer's instructions for the TruSeq Stranded mRNA library prep kit (Illumina). The concentration and size distribution of the completed libraries were determined using an Agilent Bioanalyzer 2100 DNA high-sensitivity kit and Qubit fluorometry (Invitrogen). Libraries were sequenced by following Illumina's standard protocol using the Illumina NextSeq500 HighOutput kit V2.5. For the gene expression z score analysis (including published data revealed in FIG. 1) by aligning raw RNA-seq data to the GRCh38 genome using the STAR aligner. Subsequently, gene expression was estimated using the GenCode v41 gene annotation file (gtf), focusing on gene types categorized as protein_coding and lncRNA. Genes originating from the mitochondrial genome (chrM) were also excluded from further analysis. The expression levels of each gene were then quantified using the salmon tool. Next, differential expression analysis was conducted using the edgeR package. Raw read count information was processed to identify genes showing significant changes in expression across different conditions. The filterByExpr function was applied to remove genes with low expression levels to ensure robust results. The remaining genes were subjected to TMM normalization and dispersion estimation methods to identify differentially expressed genes accurately. Subsequently, genes were categorized as upregulated or downregulated based on fold change values. The expression trends of the same genes across different samples were analyzed by calculating z-scores, allowing for the observation of consistent or divergent expression patterns among compared samples. This analysis pipeline enabled the identification of differentially expressed genes and provided insights into their expression trends across various conditions.

scAAV9 Plasmid Construction

To construct scAAV9-shFto plasmid, a 65 basepair short hairpin sequence targeting the fat mass and obesity-associated (Fto) gene (RNAi #TRCN0000183897) was subcloned into scAAV9-H1-CB-EGFP plasmid. A scAAV9-EGFP plasmid with the same backbone was provided by the AAV Core Facility in Academia Sinica. To verify the expression of the scAAV9-shFto construct, we used pLKO.1-shFto and pLKO.1-sh-Scramble (as a control group) and packaged them into lentivirus from the RNAi Core Facility of Academia Sinica. We transduced lentivirus (at a multiplicity of infection of 100) with 8 mg/mL protamine sulfate (Sigma-Aldrich) in a growth medium (DMEM) containing 20% fetal bovine serum. After 48 hours of transduction, the medium was changed with the selection drug (8 μg/mL puromycin) for one week. We collected RNA/protein of puromycin-selected C2C12 cells, and levels of Fto were determined by qPCR/Western blotting, with m⁶A methylation levels being determined by m⁶A ELISA.

scAAV9 Virus Preparation and Injection

scAAV9-shFto and scAAV9-EGFP were packaged by the AAV Core Facility in Academia Sinica. For intrathecal injection, mice at P60 were anesthetized by isoflurane. The lumbar spine was exposed through a 1.5 cm window surgically cut on the back of the mice. We injected 20 μL of either scAAV9-EGFP or scAAV9-shFto (5×10⁹ vg/μL) at a rate of 4 μL/minute into the groove between the L6 and S1 segments using a 27G needle. A flick of the mouse's tail indicated a successful injection.

### Compound Muscle Action Potential (CMAP)

Evoked CMAP of gastrocnemius muscle was measured on isoflurane-anesthetized mice at the indicated age (P60, P140, P160). Before recording, hairs on the right hind limb and the lower back were shaved and completely removed using a depilatory cream. A reference recording electrode was then placed on the ankle of the right hind limb, and an active recording electrode was placed on the belly of the right gastrocnemius. To stimulate the sciatic nerve, a pair of stimulating anode and cathode needles were inserted near the sciatic nerve at the ipsilateral paraspinal site and the region of the proximal hind limb, respectively. A ground electrode was placed on the tail to minimize artifacts. To obtain maximal CMAP responses, we gradually increased the stimulus intensity from 3 mA and determined the supramaximal stimulation as ~120% of the stimulus intensity that no longer increased the response amplitude. The baseline-to-peak and peak-to-peak CMAP amplitudes evoked by supramaximal stimulation (mostly ~7 to 8 mA) were summed. We recorded at least 30 maximal CMAP responses for each mouse at each stage. The top five successful and maximal CMAP responses in each mouse were averaged and used for comparison between groups.

### Results

m⁶A Levels are Reduced in Human ALS iPSC-Derived Motor Neurons and Hypo-m⁶A Leads to Motor Neuron Degeneration

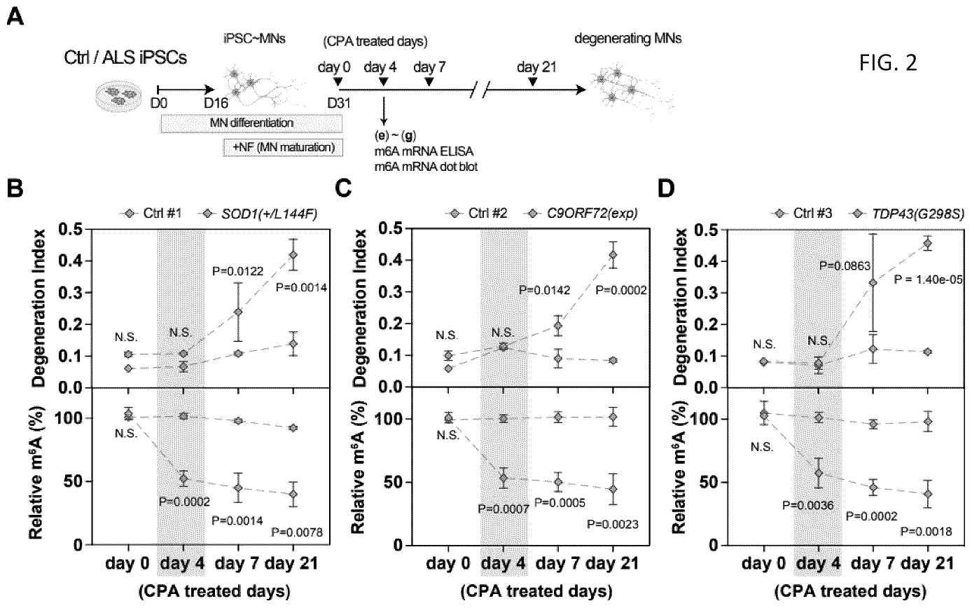

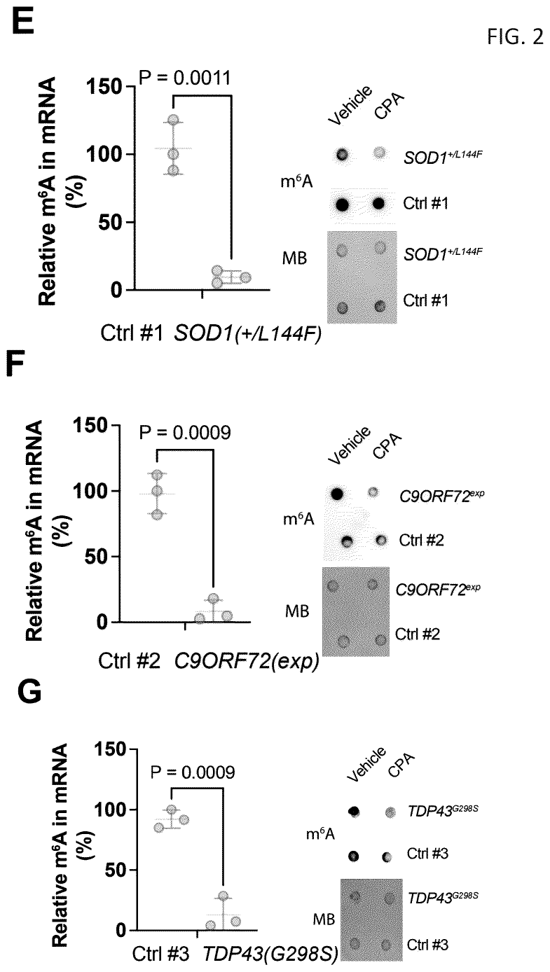

Given existing contradictory results ^{35,36}, we examined the extensive transcriptomic dataset derived from Answer ALS (https://www.answerals.org/) to establish if m⁶A hypermethylation (hyper-m⁶A) or hypo-m⁶A is associated with ALS. Our analysis focused on assessing expression levels of the methyltransferases METTL3/5/14/16, in which METTL3/14 are the ‘writer’ complex responsible for m⁶A for mRNAs, while METTL16 is largely for ncRNA and METTL5 is known for rRNA m⁶A modifications 3 (FIG. 1). Interestingly, only METTL3/14 exhibited a trend of down-regulation in numerous sporadic or familial human ALS iPSC-derived spinal motor neurons (iPSC~MNs). (FIGS. 1A and 1B). In agreement with this outcome, we observed reduced METTL3 and METTL14 expression in the majority of postmortem cortex samples from ALS patients (FIG. 1C), as well as reduced protein levels as examined by Li et al. 36. To confirm this result, we assessed another independent study that conducted transcriptome analysis on a different set of human ALS iPSC~MNs and observed that most of those iPSC~MNs also exhibited consistent down-regulation of METTL3 and METTL14 (FIG. 1D). Together, these findings indicate that compromised m⁶A pathways might be an overlooked aspect of ALS. To scrutinize if compromised m⁶A writer expression leads to hypo-m⁶A in a human context, we differentiated three familial ALS iPSC lines (SOD1^{+/L144F}, C9ORF72^{exp-800 G4C2}, and TDP43^{G298S}), together with their corresponding isogenic rescue controls (Ctrl), into spinal MNs, and then profiled their m⁶A dynamics along the differentiation process under stress-induced conditions (illustrated in FIG. 2A, results in FIGS. 2B~2G) ^{45,50}. To accelerate ALS disease progression, we applied cyclopiazonic acid (CPA), an endoplasmic reticulum stressor, as CPA has been shown previously to act as a selective stressor to accelerate the degeneration of human SOD1^{G93A} iPSC~MNs but not wild-type controls ^{45,50}. Consistent with this scenario, we found that all of our Ctrl-MNs were relatively resistant to CPA stress, unlike the ALS iPSC~MNs that exhibited drastic loss after seven days of CPA treatment (FIGS. 2B~2D). No obvious degeneration was displayed by either Ctrl or ALS iPSC~MNs on day 4 (FIGS. 2B~2D). Thus, we could capture the progressive MN degeneration displayed by the familial ALS iPSC lines. Notably, even before the drastic MN loss following stress treatment on day 4, we consistently detected reduced m⁶A levels in the ALS iPSC~MNs (FIGS. 2B~2D). This trend was sustained at day 7 with a concomitant decrease in METTL3/14 expression, together with significantly increased expression of the demethylases FTO and ALKBH5 in most of the ALS iPSC~MNs (FIG. 1E). In agreement with these data, we found that all three ALS iPSC lines displayed hypo-m⁶A before the MN loss caused by stress treatment (FIGS. 2E~2G) revealed by m⁶A ELISA and dot blot (see methods for details).

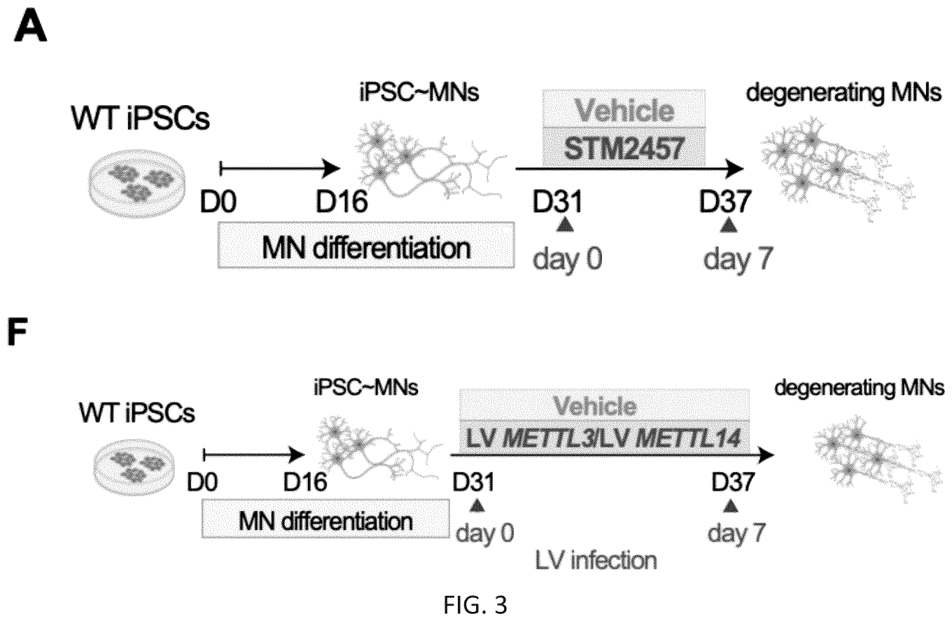

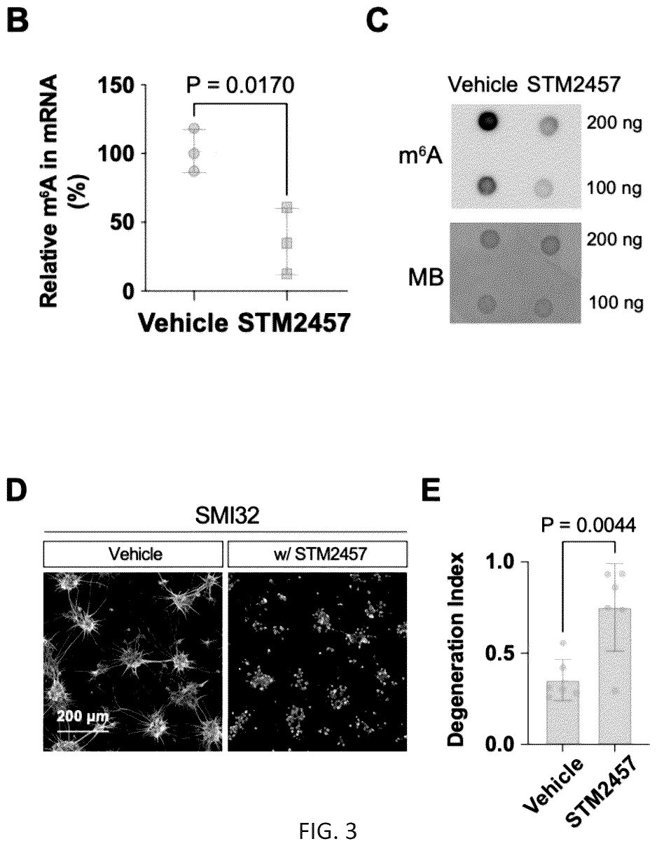

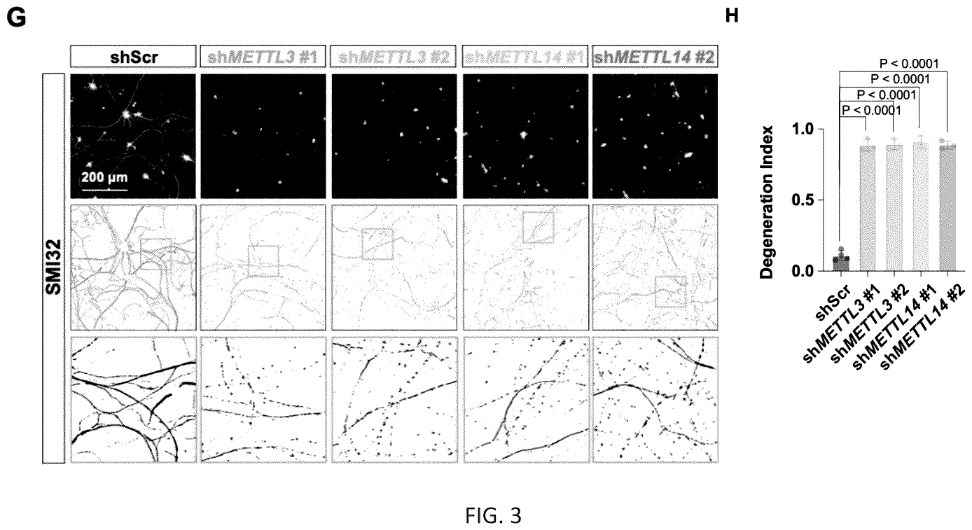

To confirm if hypo-m⁶A leads to MN degeneration, we adopted two approaches. First, we used a specific METTL3 inhibitor (METTL3i), STM2457 ²², to impair m⁶A production during human MN differentiation (FIG. 3A), which revealed that METTL3i reduces the m⁶A-mRNA repertoire assayed by m⁶A ELISA and dot blot (FIGS. 4B and 4C), with concomitantly drastic neurite degeneration and a reduced MN population (FIGS. 3D and 3E). Secondly, we infected the human iPSC~MNs by METTL3 or METTL14-shRNA (FIG. 3F), and revealed a reduction in m⁶A-mRNA levels (FIG. 4) with a concomitant neurite degeneration (FIGS. 3G and 3H). Thus, our results together with the large set of available ALS patient data support the notion that METTL3/METTL14 might be a critical regulatory complex linked to both familial and sporadic ALS disease, and that MNs appear to be more sensitive to METTL3/METTL14 down-regulation. Most notably, global down-regulation of the m⁶A methylation repertoire elicited obvious MN degeneration.

### Impairment of the m⁶A Production Enzyme Mettl14 in Spinal MNs Elicits MN Degeneration

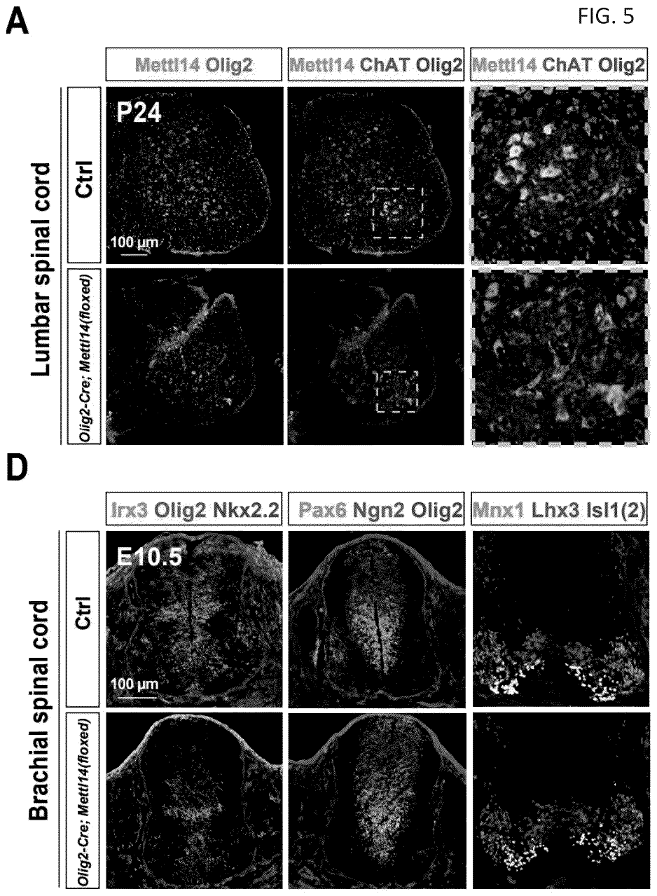

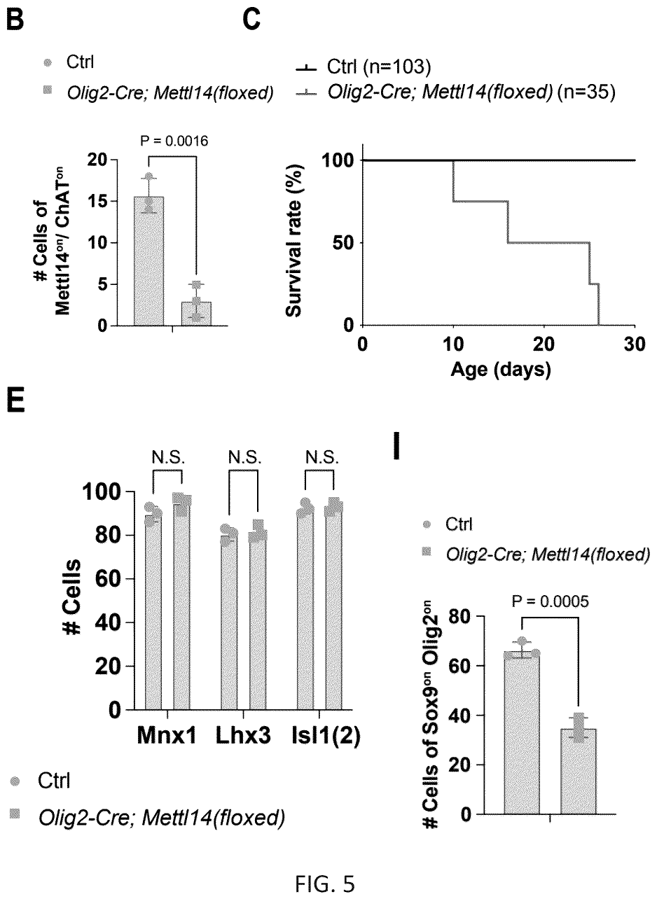

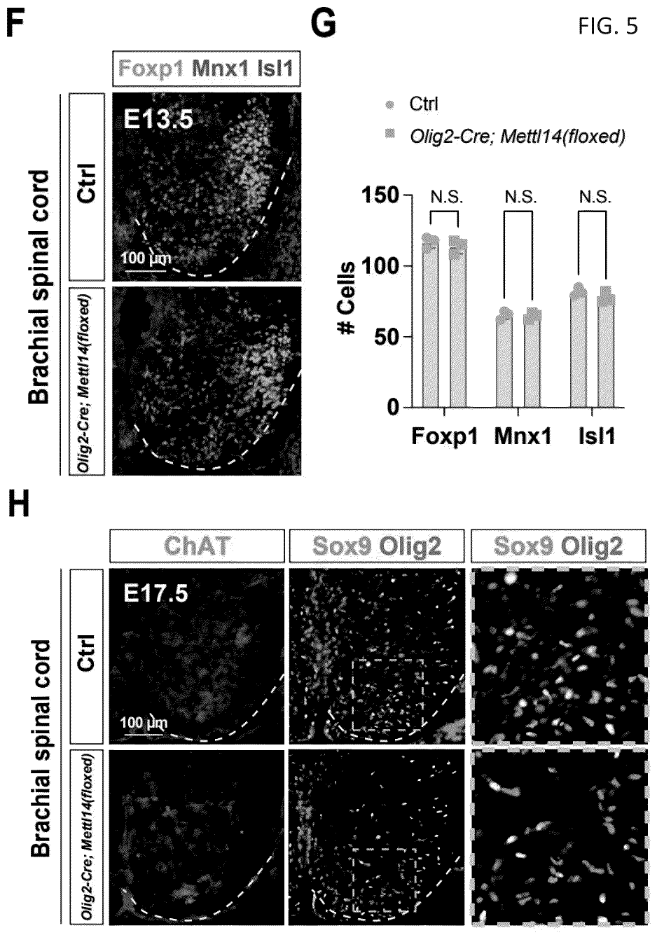

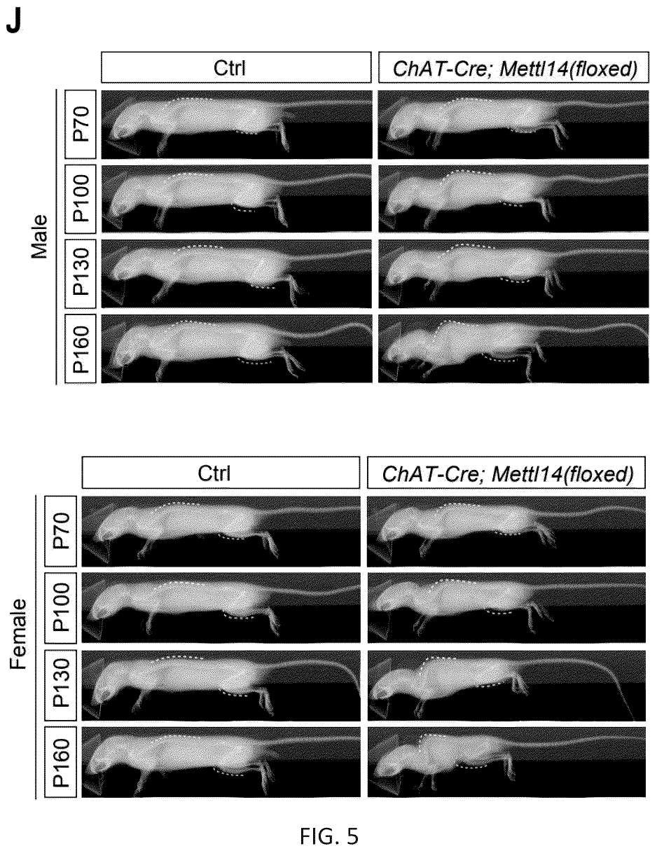

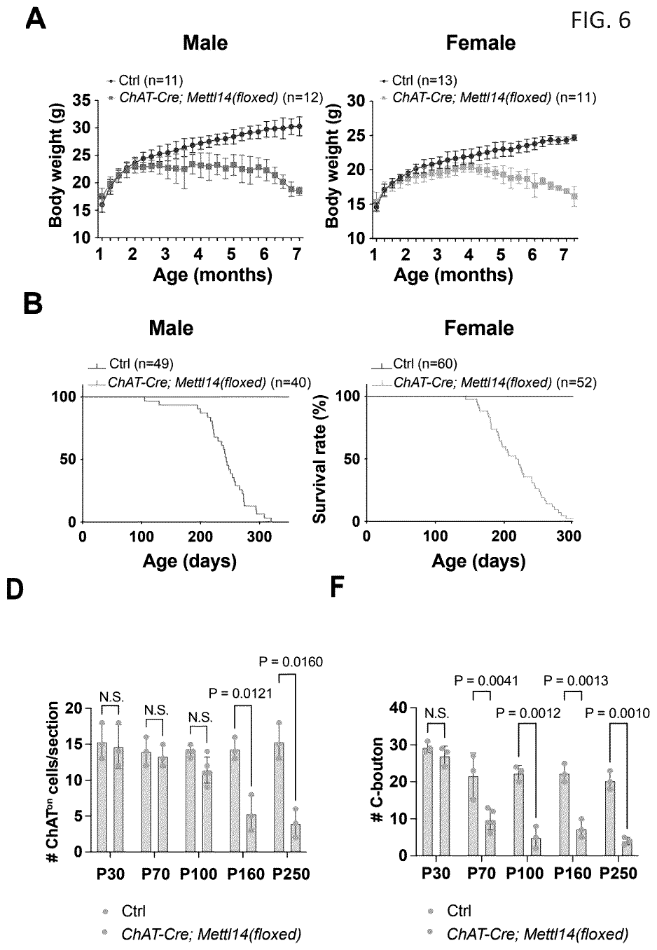

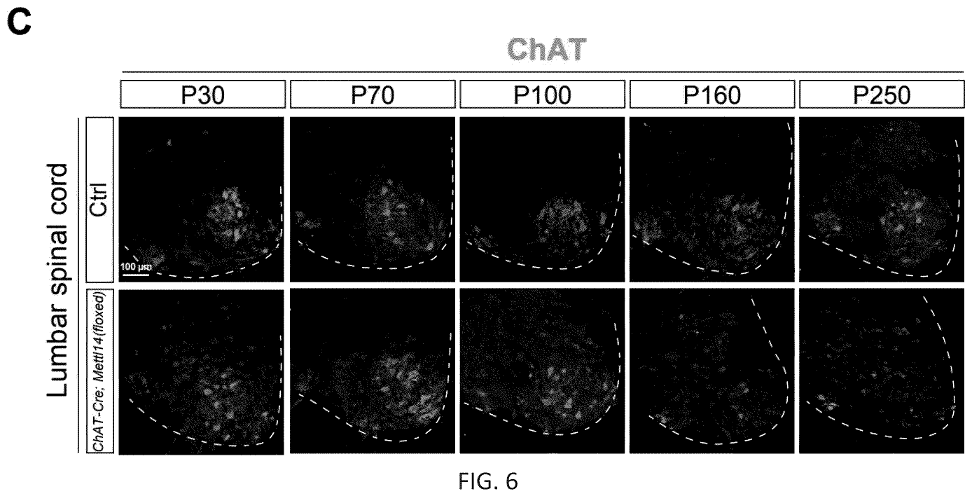

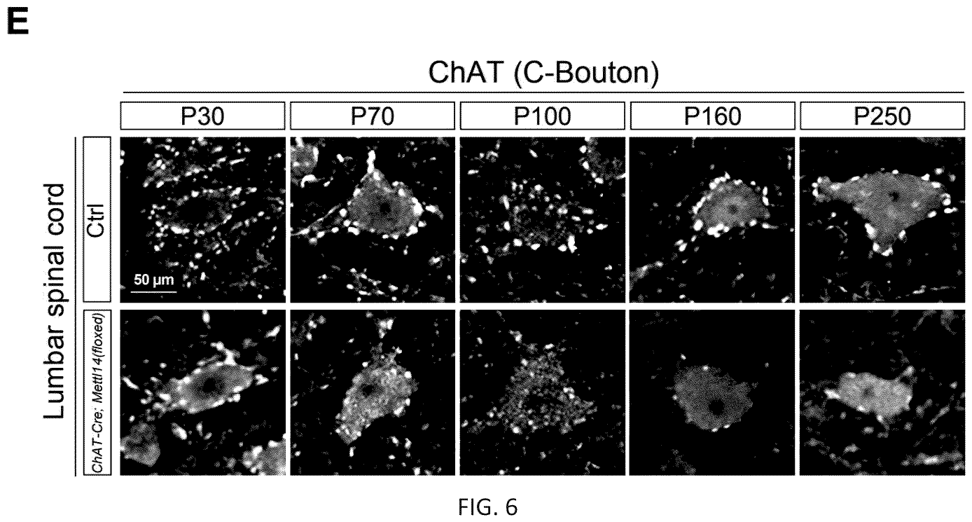

To determine if hypo-m⁶A promotes MN degeneration in vivo, we conditionally deleted Mettl14 specifically in MNs either at the developmental stage by using Olig2-Cre ⁵¹ or at the postmitotic and postnatal stage by adopting ChAT-Cre (see Methods for details) 18. First, we verified that both conditional mouse lines displayed a significant reduction in the population of Mettl14^{on} cells in the ventral horn of the spinal cord (FIGS. 5A and 5B). Although Olig2-Cre; Mettl14^{floxed} mice mostly exhibited early postnatal lethality (~P24 to P28, FIG. 5C) with a shivering phenotype (Movie S1), all MN subtypes appeared normal based on immunostaining (FIGS. 5D~5G). As Olig2 is known to be expressed at a later stage in oligodendrocytes, we consider that the shivering phenotype might be a reflection of compromised oligodendrocyte precursors (Olig2^{on} and Sox9^{on} double-positive cells) (FIGS. 5H and 5I), consistent with a previous study ⁵². Conversely, the ChAT-Cre; Mettl14^{floxed} mice displayed normal MN development and an ordinary appearance at the postnatal and juvenile stages (FIG. 6A). Nevertheless, two months later, we observed a gradual decline in body weight from P70 (FIG. 6A) and a kyphosis phenotype from P100 for all of the ChAT-Cre; Mettl14^{floxed} mice (FIG. 5J). Strikingly, all of the ChAT-Cre; Mettl14^{floxed} mice (both male and female, n>90) exhibited premature death at P160~P300 (FIG. 6B).

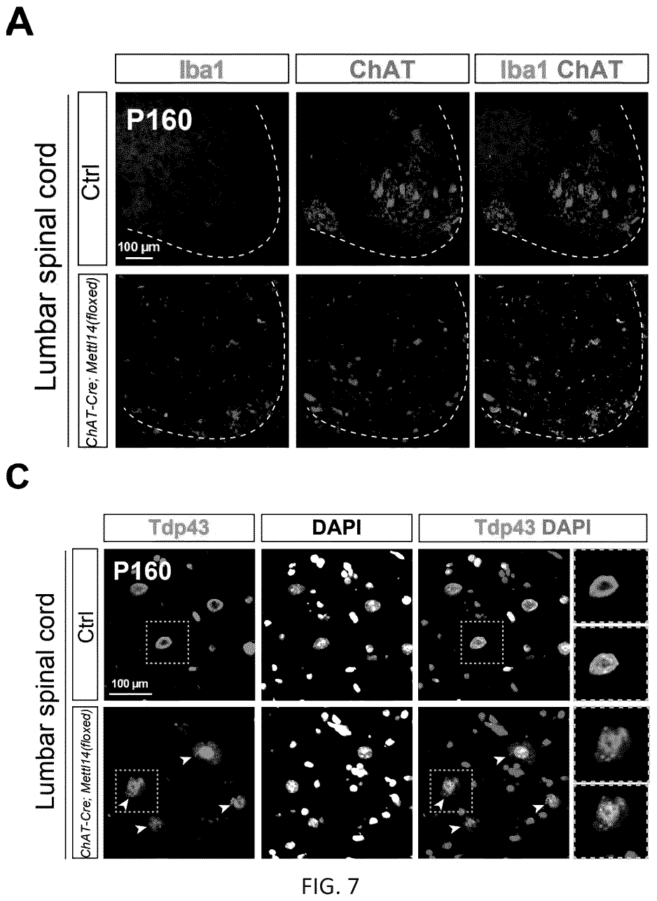

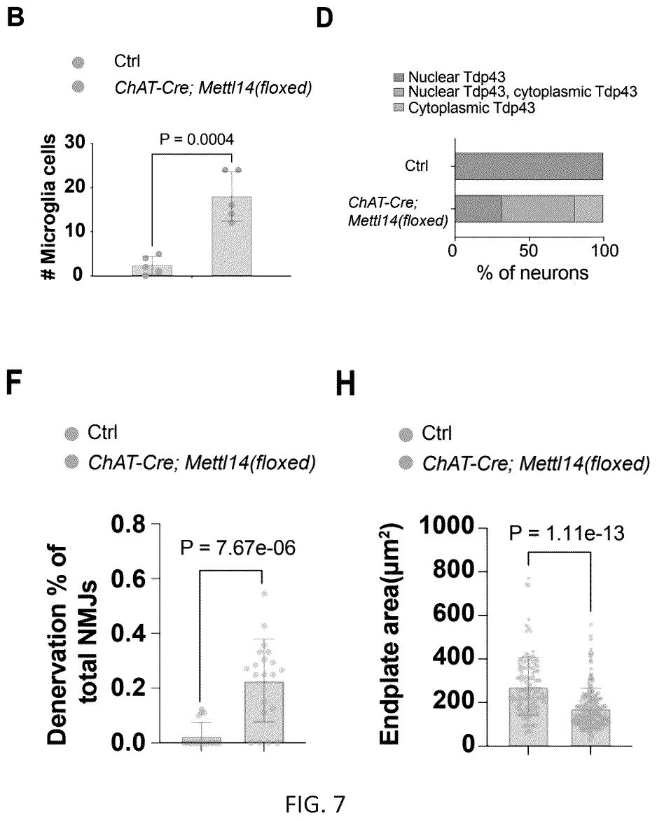

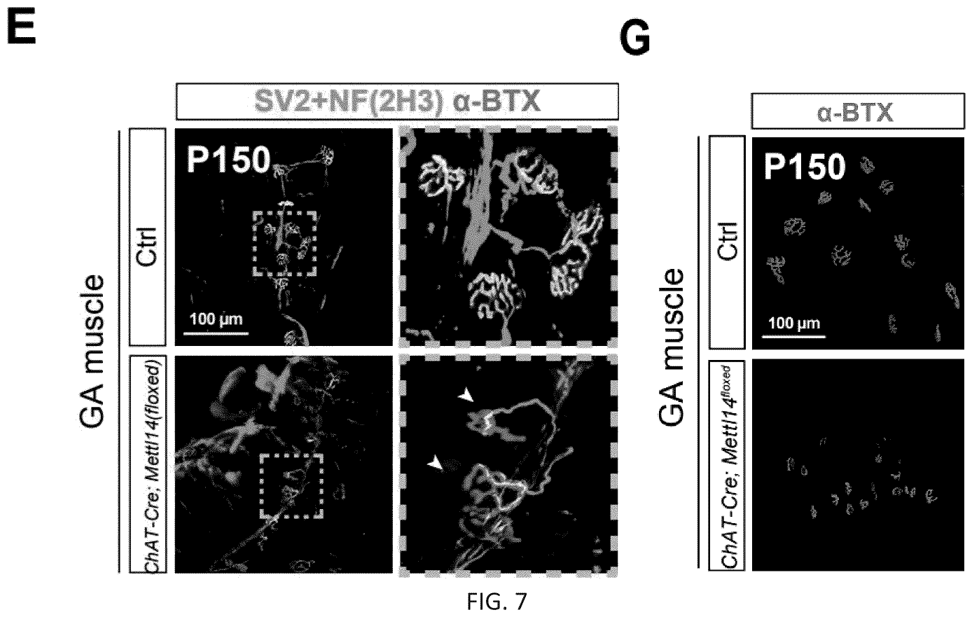

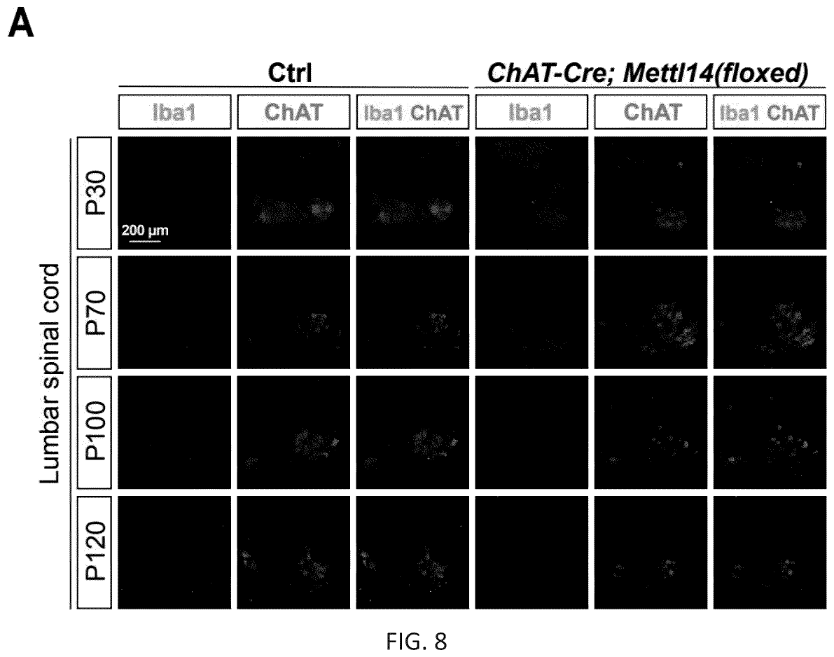

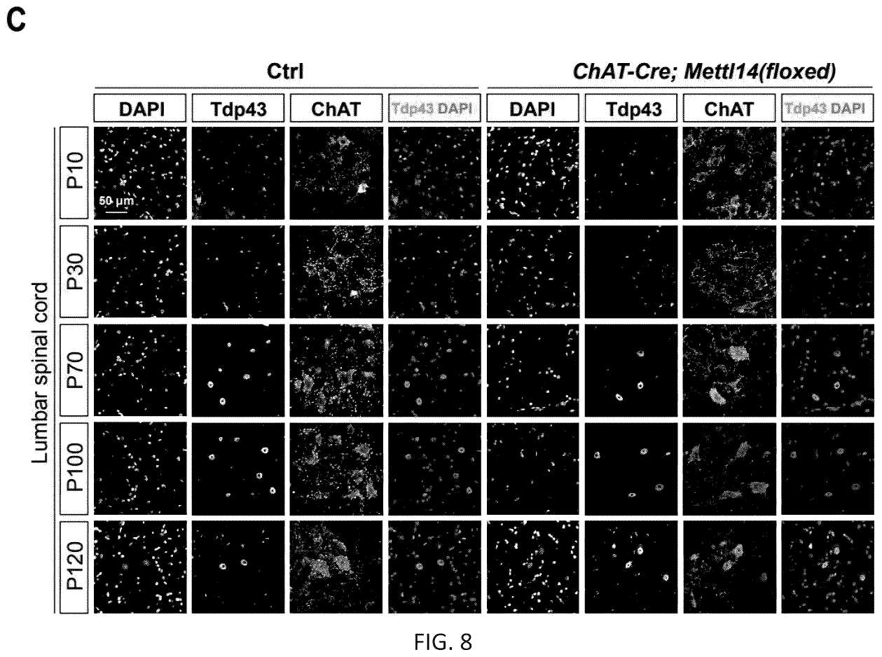

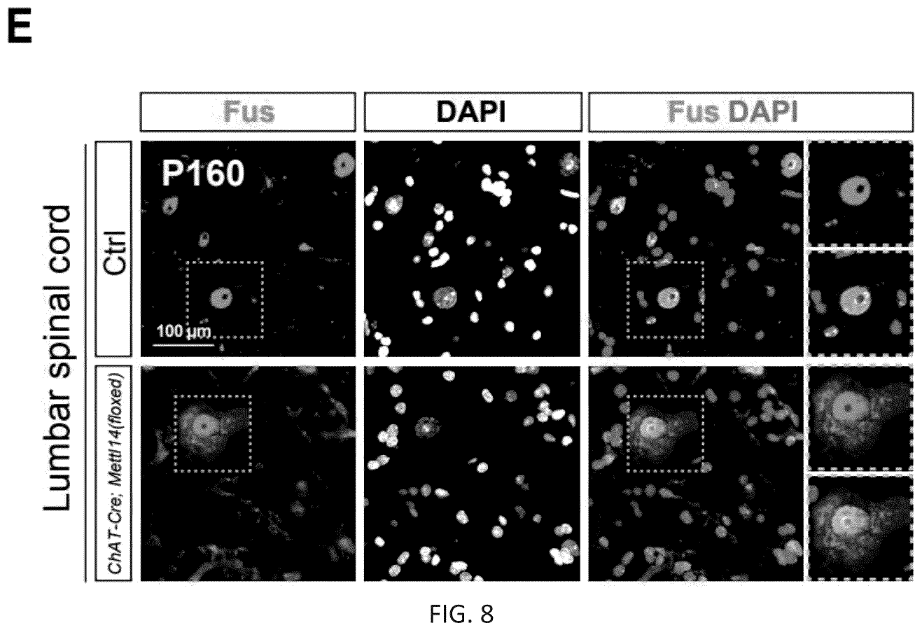

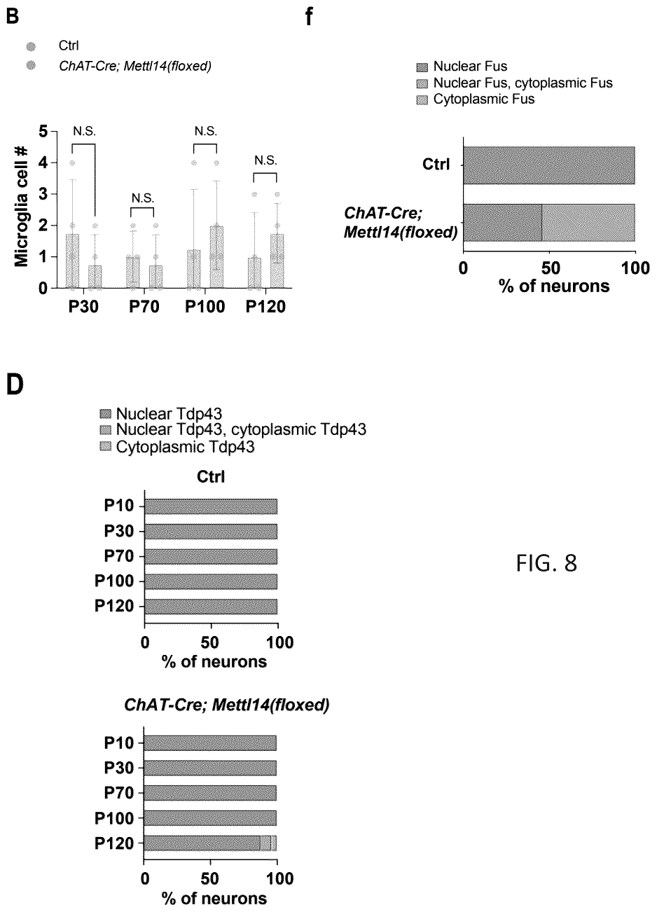

Apart from their kyphotic appearance and movement defects, we further investigated a series of molecular and cellular ALS disease features in the ChAT-Cre; Mettl14^{floxed} mice. At the molecular level, we observed: (1) that the numbers of ChAT^{on} MNs in the lumbar region of spinal cords were comparable before P70, but gradually declined after P100 (FIGS. 6C and 6D). However, C boutons, a source of cholinergic input to MNs, already showed a prominent decrease from P70 (FIGS. 6E and 6F); (2) prominent neuroinflammation upon microglia (Iba1^{on}) activation in the spinal cords of the ChAT-Cre; Mettl14^{floxed} mice relative to controls at P160 (FIGS. 7A and 7B), but not before P120 (FIGS. 8A and 8B); and (3) significant cytoplasmic aggregation of Tdp43 in the ChAT-Cre; Mettl14^{floxed} mice, whereas control littermates mainly presented nuclear localizations for that protein after P120 (FIGS. 7C, 7D, 8C, and 8D). Notably, another RNA-binding protein, Fus, which is often shown as mislocalization in ALS patients, also exhibited cytoplasmic in the KO mice (FIGS. 8E and 8F). Furthermore, the ChAT-Cre; Mettl14^{floxed} mice exhibited drastically reduced endplate area and muscle denervation (FIGS. 7E~7H). Overall, our findings indicate that the ChAT-Cre; Mettl14^{floxed} mouse model demonstrates progressive MN degeneration, mirroring several key molecular pathological features observed in human ALS patients.

Mice with m⁶A Hypomethylation Recapitulate ALS-Associated Behavioral Phenotypes

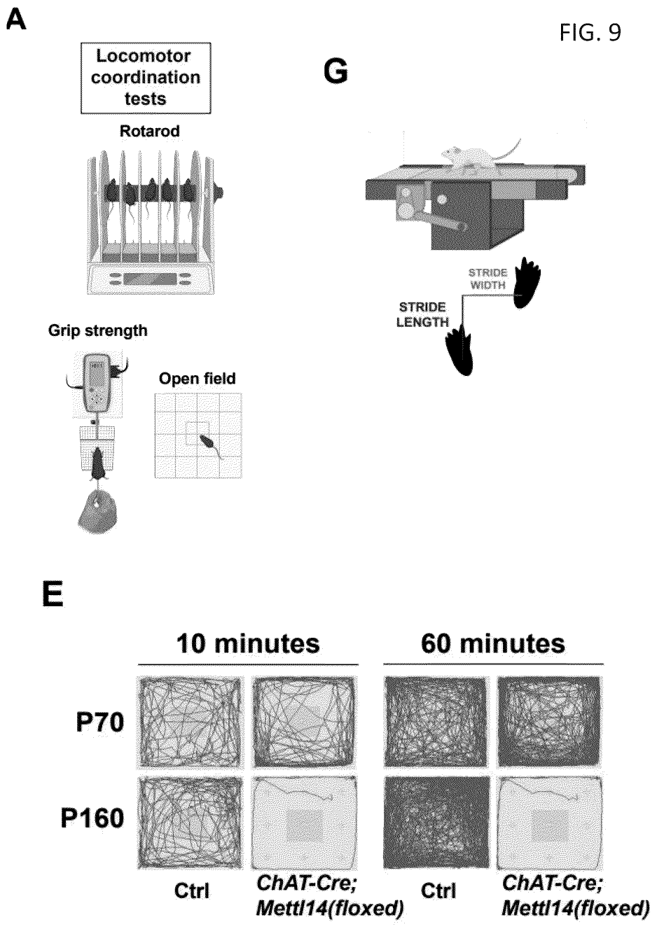

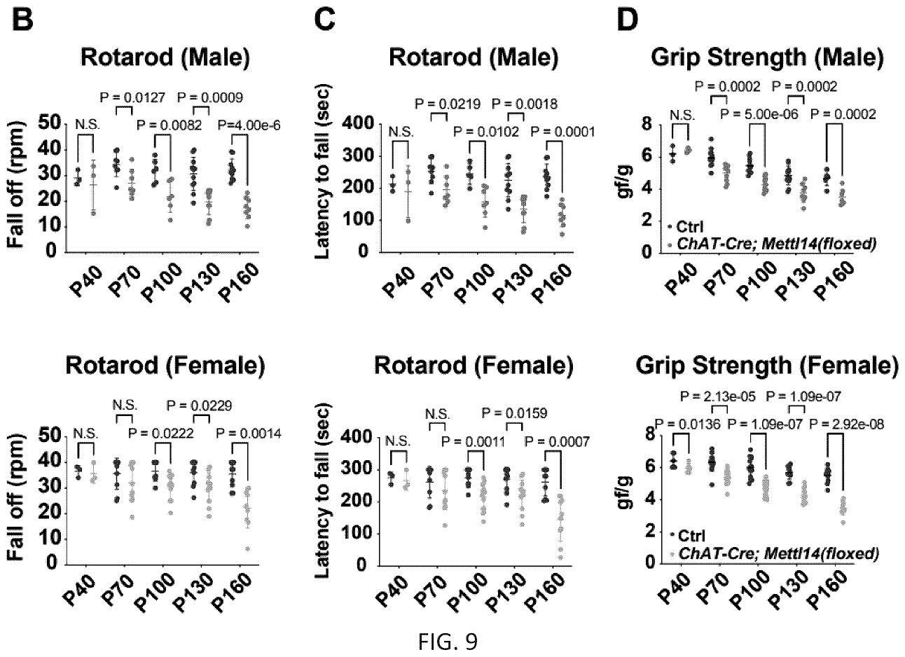

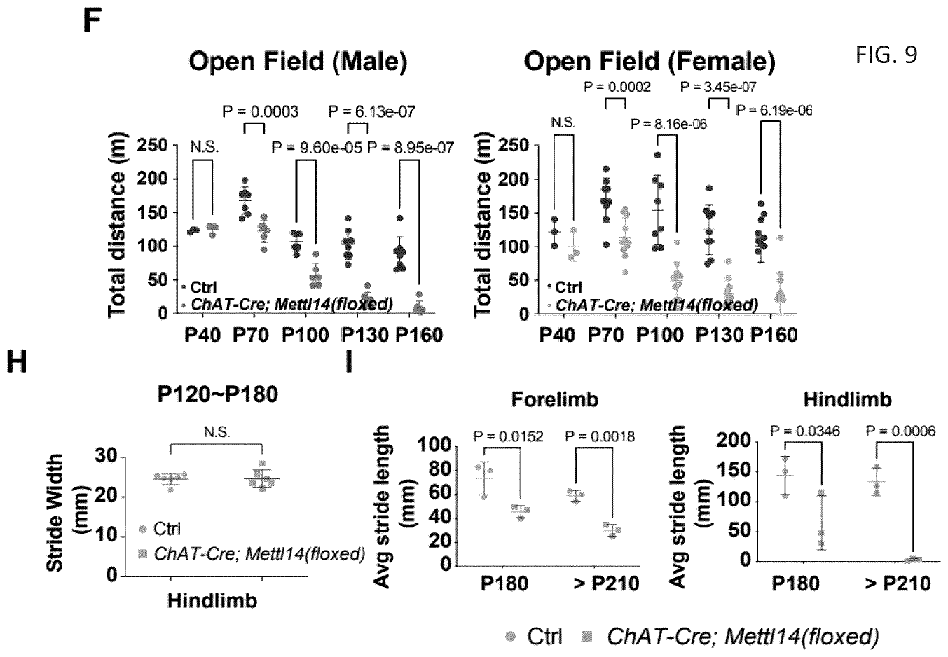

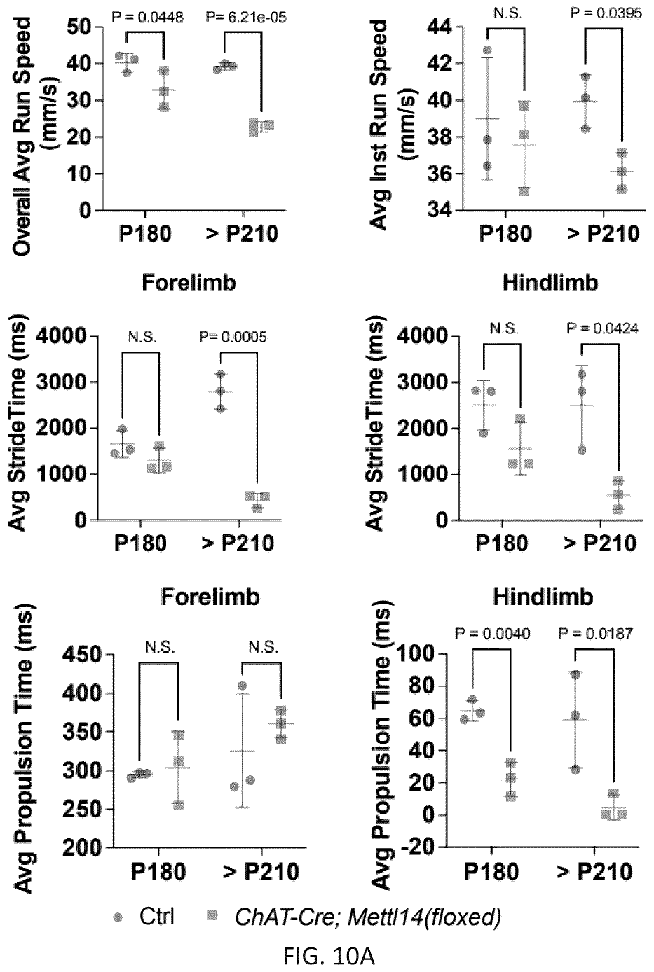

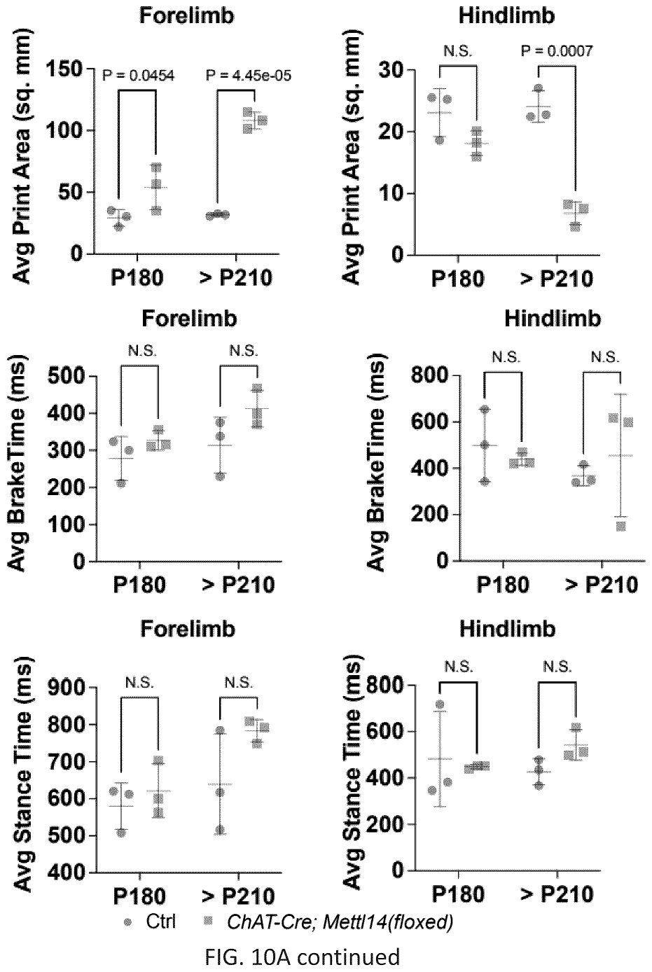

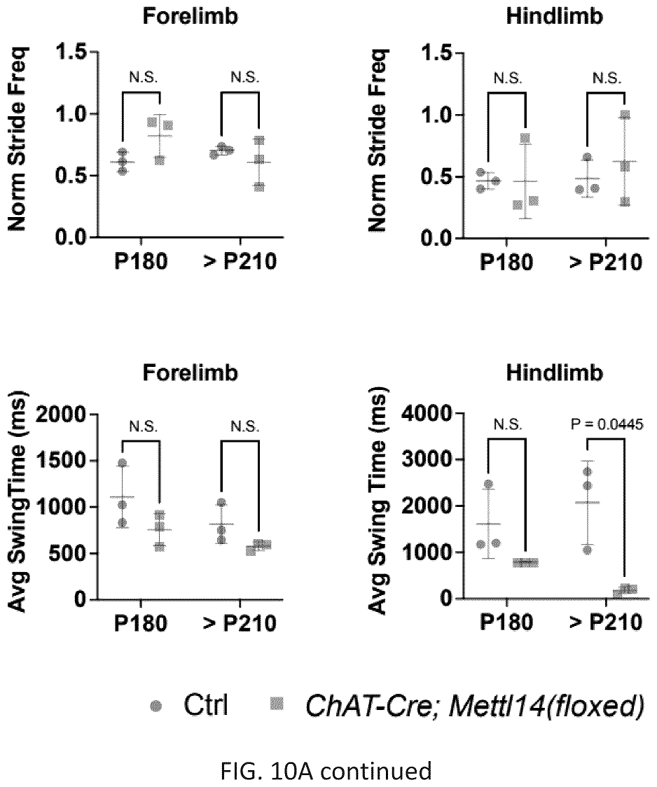

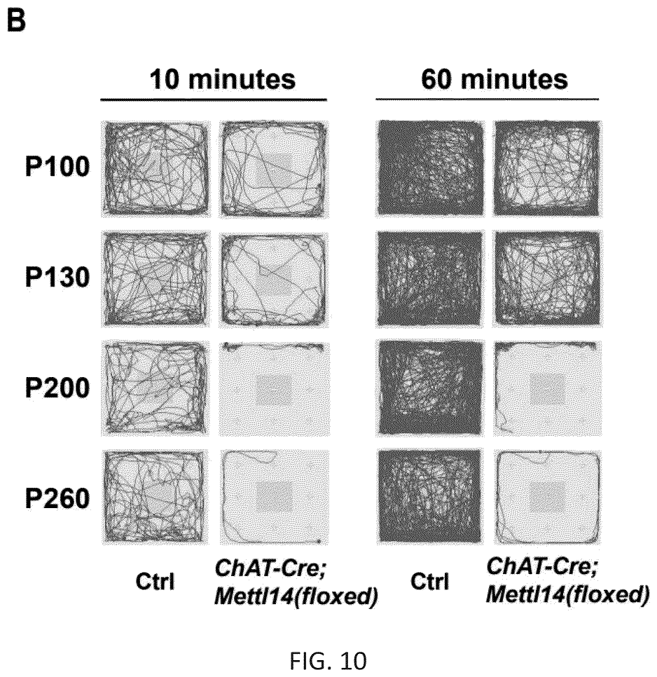

Although some previous fALS mouse models exhibit molecular hallmarks of ALS pathology, their MN physiology or gross behaviors appeared relatively normal 30,34. To examine if the ChAT-Cre; Mettl14^{floxed} mice represent an improved potential ALS mouse model, we conducted a series of behavioral assays on the ChAT-Cre; Mettl14^{floxed} mice to further characterize the phenotypes observed from P40 to P210. First, the results of a rotarod test and forelimb grip strength assay corroborated that motor ability gradually declined, together with concomitant muscle weakness, recapitulating two major pathological manifestations observed for ALS patients (FIGS. 9A~9D). Next, through an open field test, we noted that general activity levels of the ChAT-Cre; Mettl14^{floxed} mice gradually became compromised and their exploratory behavior in a novel environment was reduced, reflecting a motor deficit and a frontotemporal dementia (FTD)-like phenotype (FIGS. 9E, 9F and 10). Finally, we performed a kinematic analysis, which revealed that whereas the spinal interneuron circuit remained largely intact, motor outputs were interrupted in the ChAT-Cre; Mettl14^{floxed} mice (FIGS. 9G~9I, 10A). This detailed scrutiny of the ChAT-Cre; Mettl14^{floxed} mice at the molecular, cellular, physiological, and behavioral levels indicates that the m⁶A reservoir is a critical factor in maintaining adult MN function, and that compromising m⁶A levels prompts a MN degeneration process that recapitulates ALS disease progression.

Uncovering the Dysregulated m⁶A-Modified Genes Leading to MN Degeneration

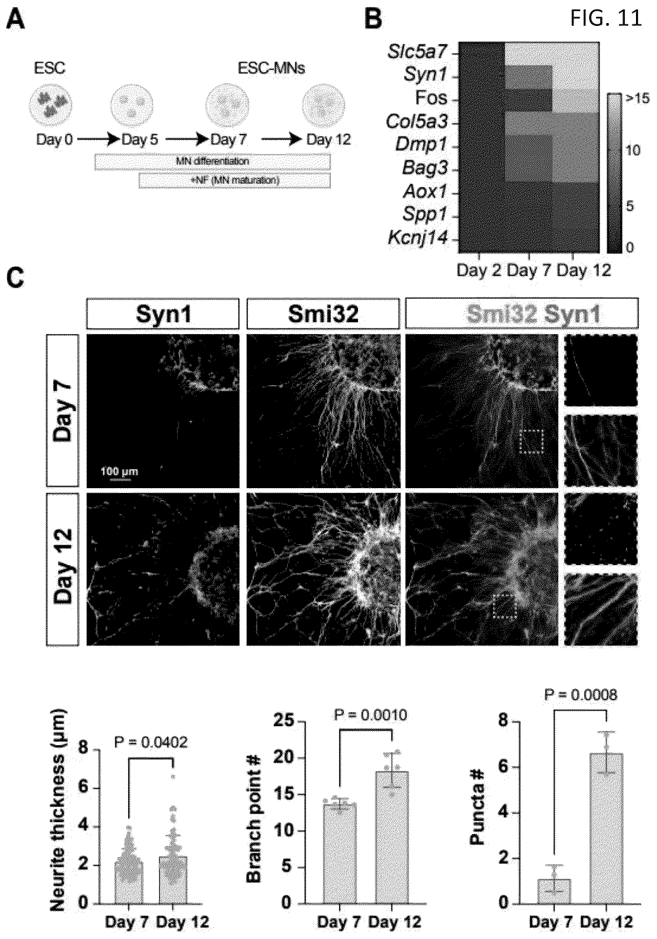

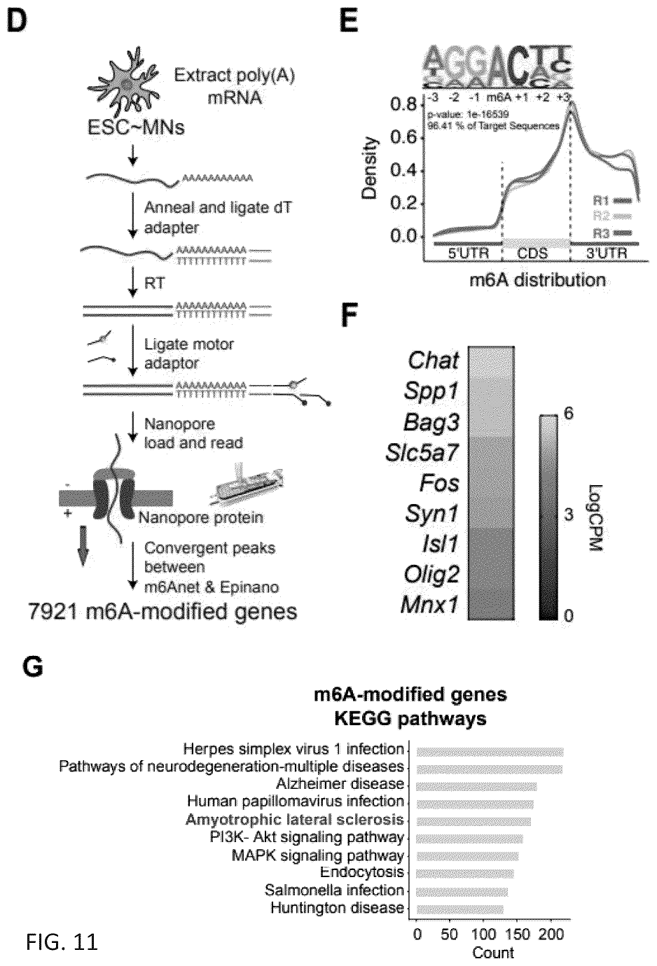

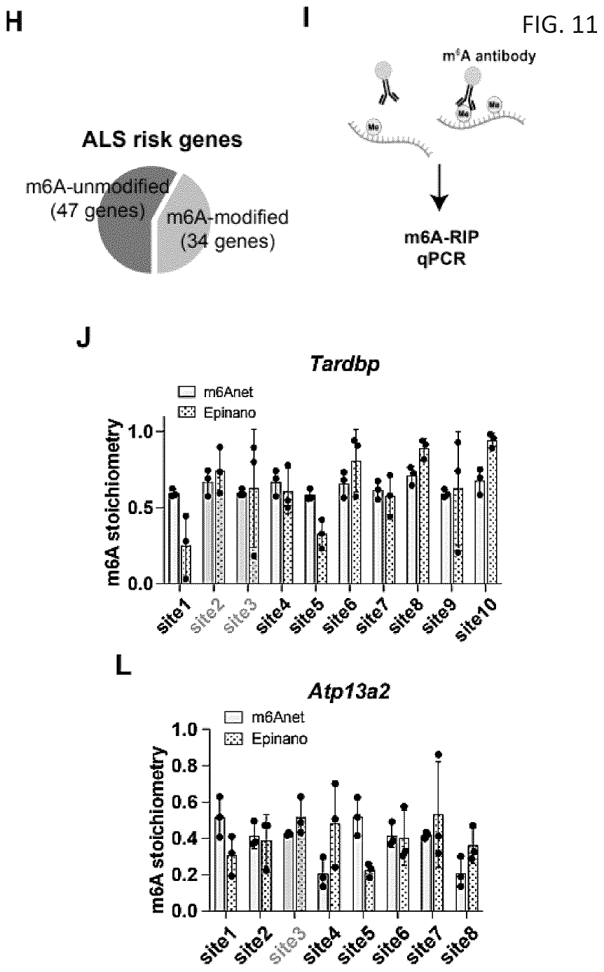

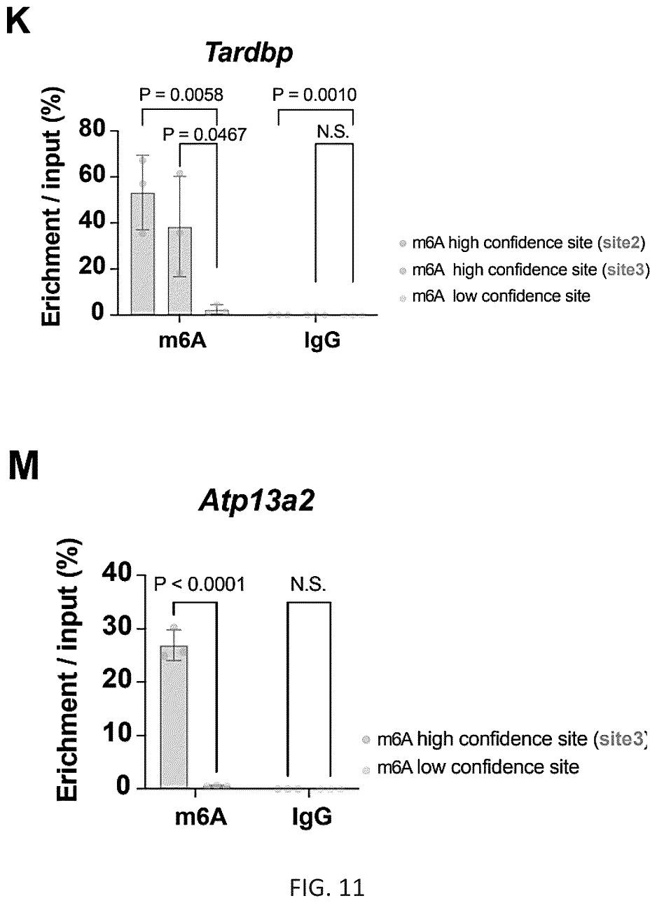

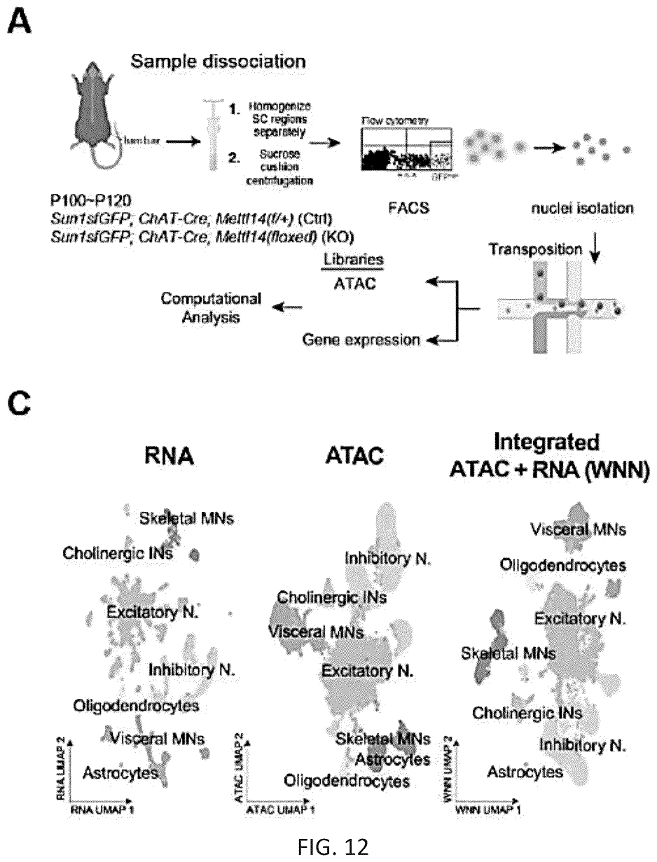

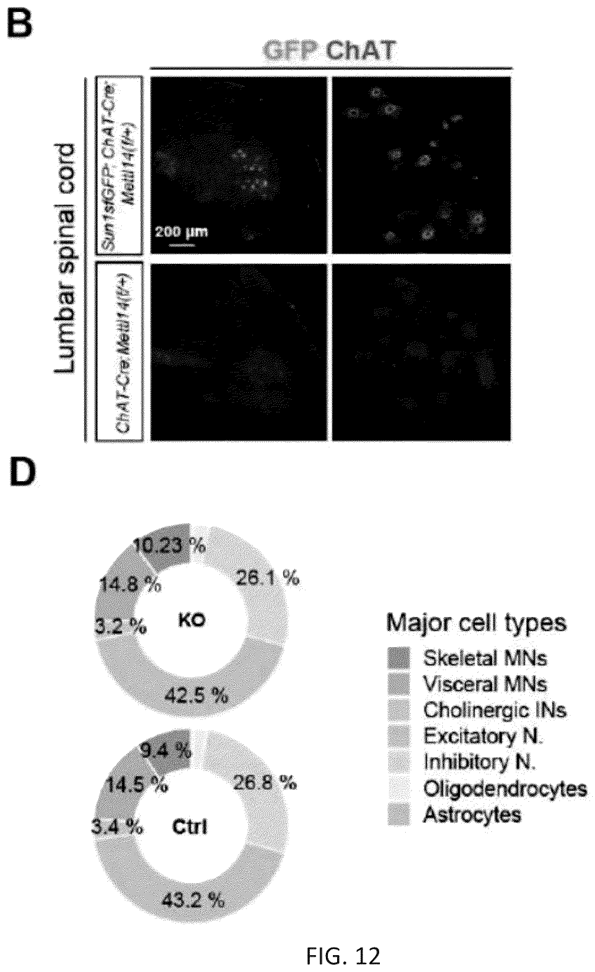

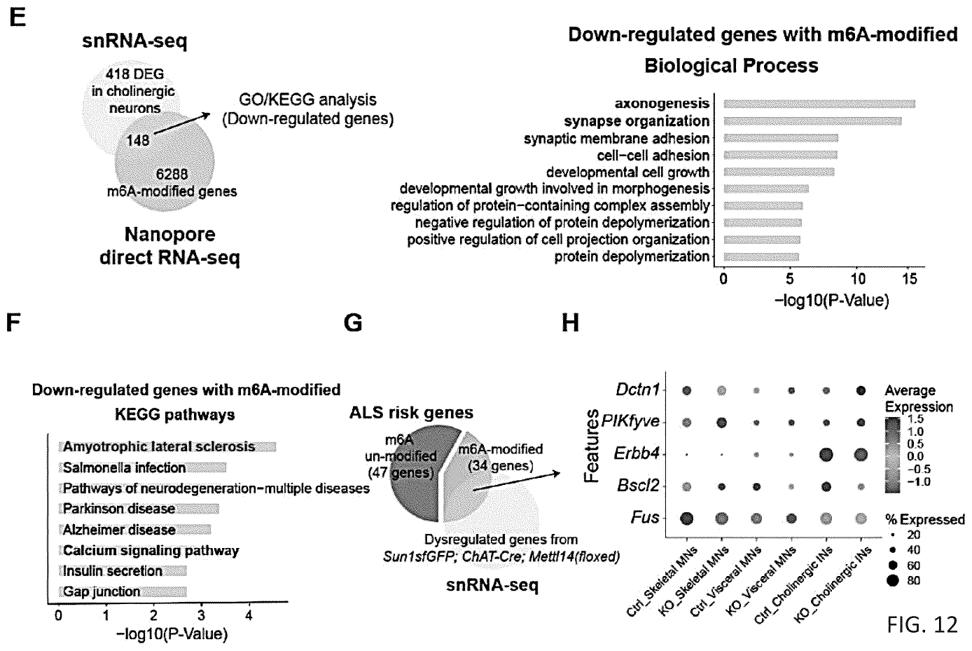

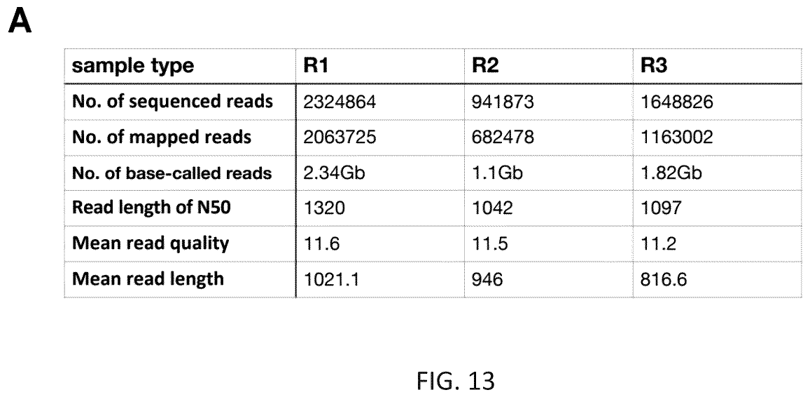

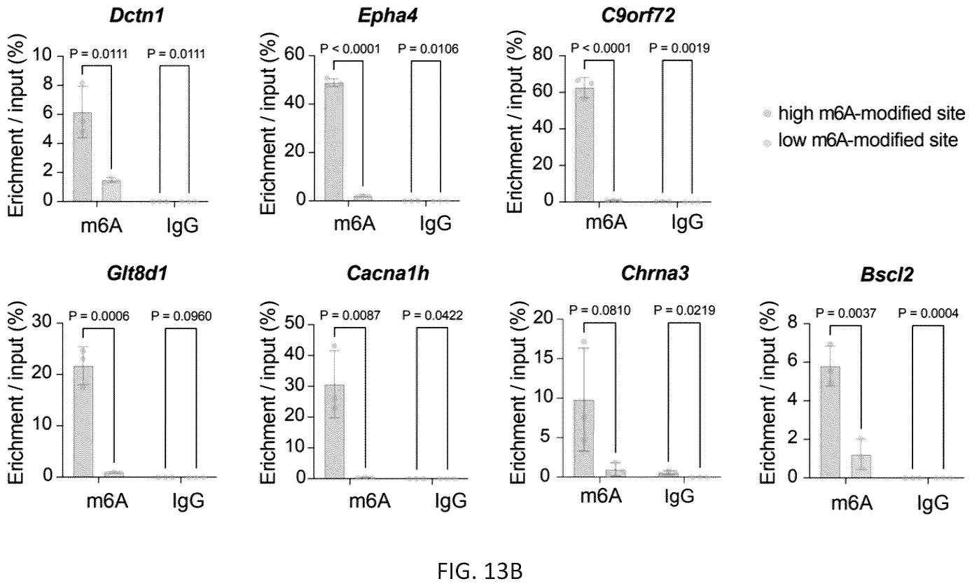

To gain insights into the potential mechanisms underlying how hypo-m⁶A promotes MN degeneration, we aimed to systematically identify the dysregulated genes possessing m⁶A modifications in our ChAT-Cre; Mettl14^{floxed} mice (FIGS. 11 and 12). To identify m⁶A-modified transcripts, we adopted a direct RNA sequencing platform, which enables the identification of the MN m⁶A epitranscriptome at single-nucleotide resolution (FIG. 11). Since it is technically challenging to obtain sufficient adult MNs from the spinal cord for direct RNA sequencing, we employed an enhanced method using mouse ESC-derived MNs, matured with a conditioned medium (FIG. 11) ^{44,45}. First, we confirmed that this approach successfully generated MNs expressing mature neuronal markers, with longer and more complex neurite structures (FIGS. 11B and 11C). We then subjected these mature MNs to the ONT Nanopore platform, which provides a powerful framework for detecting RNA modifications through advanced machine-learning algorithms applied to sequencing metrics (FIG. 11D). We employed two supervised machine learning tools, namely EpiNano and m⁶Anet ^{46,47}, setting a stringent criterion that the predicted m⁶A sites need to occur in at least two samples for either one of the algorithms (see Methods for details). We identified 30,340 high-confidence m⁶A modification sites corresponding to 7,921 genes (FIG. 11D, genes of interest are listed in List 1; refer to the Methods section for details). Consistent with previous findings ²⁹, our analysis revealed enriched distributions of m⁶A sites in coding sequences (CDSs) and 3′ UTRs, especially near the stop codons (FIG. 11E). By analyzing the enrichment of Gene Ontology (GO) and KEGG pathways for the m⁶A-modified transcripts, we noticed a striking enrichment for ALS-related genes (FIG. 11G). In addition, among the 81 identified ALS risk genes to date, our direct RNA sequencing demonstrated that 34 of them are m⁶A-modified (p=2.06*10⁻⁶, one-tailed hypergeometric test; FIG. 11H and List 2). To validate our computationally predicted m⁶A sites (FIG. 11I), we used Tardbp (Tdp43) as a benchmark to validate our methodology. Consistent with a previous report ³⁵, we confirmed the existence of a previously identified m⁶A site in Tardbp via m⁶A antibody pull-down (FIG. 11K). Additionally, we uncovered and validated an additional high-confidence m⁶A site within the Tardbp transcript from Nanopore sequencing (FIG. 11K). Moreover, we substantiated the existence of predicted m⁶A sites in Atp13a2 (Park9) (FIG. 11M). ⁵³, which is an ALS risk gene that has not been shown previously to have m⁶A modifications in MNs. Finally, we further verified several newly identified m⁶A-modified sites in ALS risk genes, including Dctn1, Epha4, C9orf72, Glt8d1, Cacna1h, and Bscl2 (FIG. 13B). Thus, these observations confirm the sensitivity, accuracy, and reliability of Nanopore technology to identify m⁶A-modified sites in MNs.

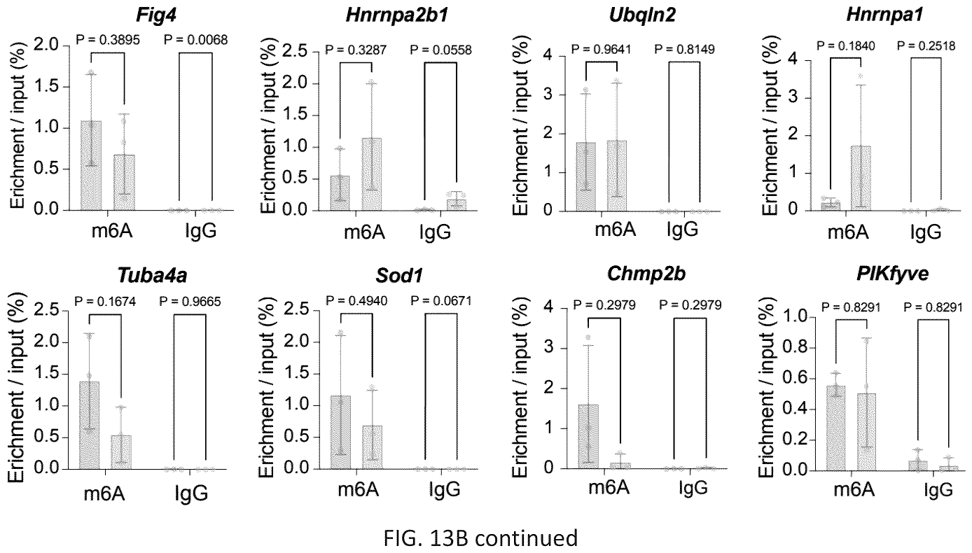

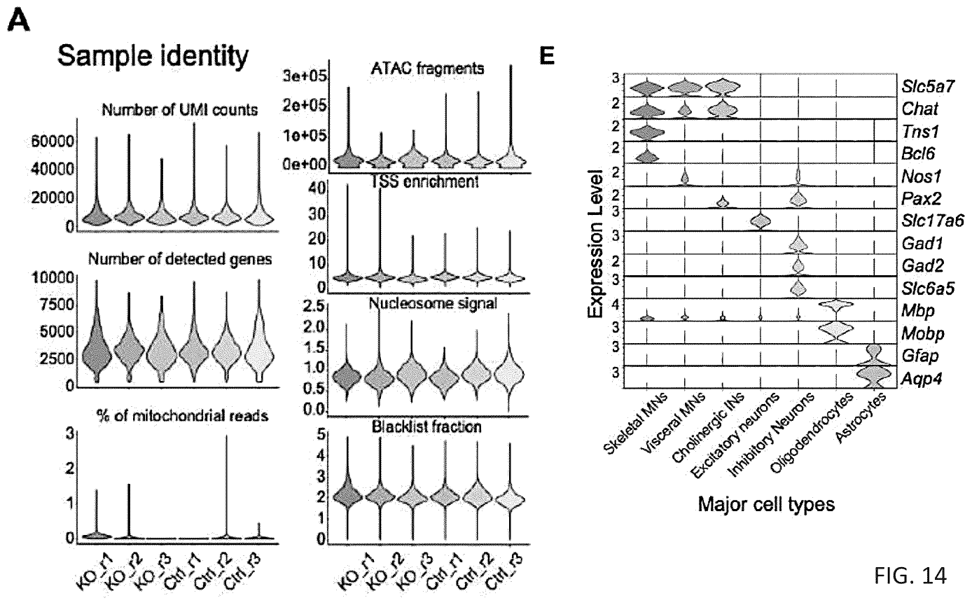

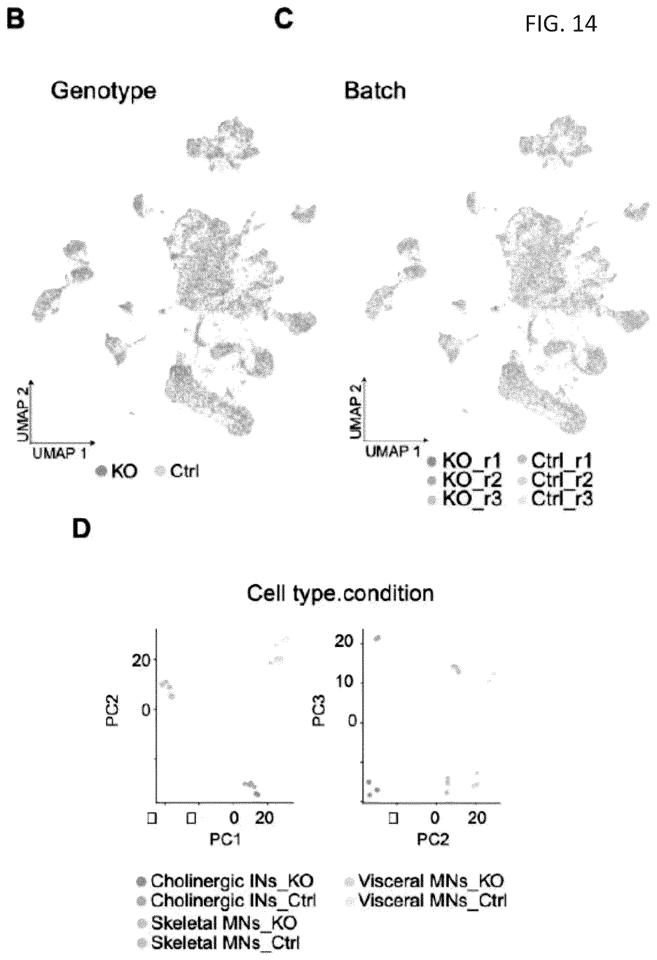

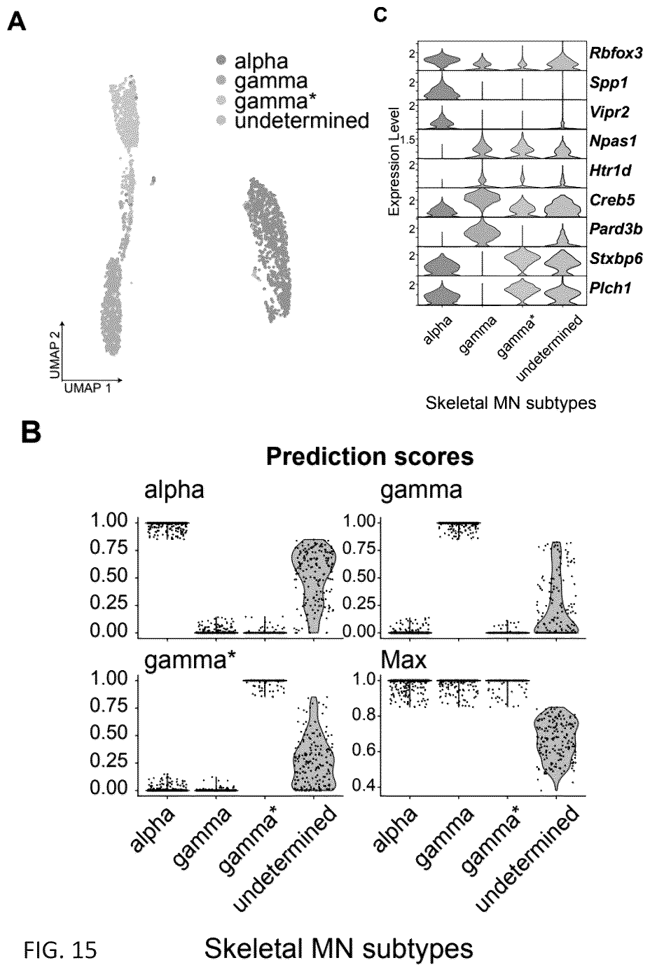

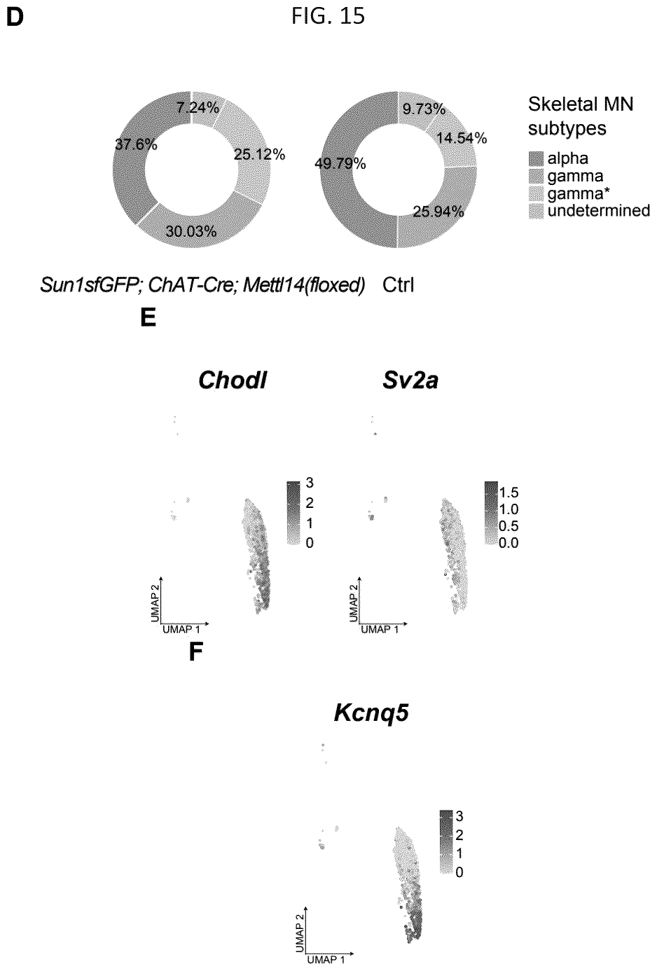

Next, we reasoned that most m⁶A-modified ALS risk genes could potentially contribute to the observed ALS-like pathologies upon m⁶A impairment in the ChAT-Cre; Mettl14^{floxed} mice, so we probed the consequence of hypo-m⁶A for MNs by performing 10× Genomics single-nuclei multimodal profiling of ATAC/RNA (snATAC/RNAseq) on Ctrl (ChAT-Cre; Mettl14^{f/+}) and ChAT-Cre; Mettl14^{floxed} mice, allowing us to assess chromatin accessibility and gene dysregulation in a range of MN subtypes simultaneously (FIGS. 12A and 12B). To selectively enrich for nuclei from spinal cholinergic neurons, we bred ChAT-Cre; Mettl14^{floxed} mice expressing the nuclear envelope reporter CAG-Sun1/sfGFP ⁴⁸ (FIG. 12B). We harvested lumbar spinal cords at P100~120, a stage when the MN population is not greatly diminished, and collected GFP^{on} cells by fluorescence-activated cell sorting (FACS) for single-nuclei multimodal profiling (FIGS. 12C and 10A). Each sample underwent rigorous quality control (QC) measures and was subsequently filtered to retain only cells that only met our QC criteria (FIG. 14A) (see Methods for details). We then integrated three replicates from the Ctrl and Sun1^{sfGFP}; ChAT-Cre; Mettl14^{floxed} (KO) samples (FIGS. 14B and 14C). Principal component analysis (PCA) revealed negligible sequencing and batch confounding variables among our sample preparations (FIG. 14D). PC1 and PC2 largely separated the major cell types in the population, and PC3 segregated the total population based on whether the cells were from the Ctrl or Sun1^{sfGFP}; ChAT-Cre; Mettl14^{floxed} cohorts. Using recognized markers for spinal cord cell type annotation 48,49, we detected the three major cholinergic cell populations, i.e., skeletal MNs (Tns1^{on}/Bcl6^{on}), visceral MNs (Nos1^{on}), and cholinergic INs (Pax2^{on}) (FIG. 12E). We did not observe any changes in the proportions of these major cell types, consistent with our in vivo characterization of the pre-onset stage of ChAT-Cre; Mettl14^{floxed} mice (FIGS. 12C and 12D). When we further analyzed subtypes within the skeletal MNs, we identified α (Htr1d ^{low}, Rbfox3 ^{high}, Vipr2^{on}), γ (Htr1d ^{high}, Rbfox3 ^{low}, Spp1^{low}, Creb5^{on}, Pard3b^{on}) and γ* (Htr1d ^{high}, Rbfox3 ^{low}, Spp1^{low}, Stxbp6^{on}, Plch1^{on}) MNs (FIGS. 13A~13D). Among α MNs, we could further distinguish fast-fatigue-resistant (Chodl^{on}, Kcnq5^{on}), slow-firing (Sv2a^{on}), and fast-fatigable (Chodl^{on}, Kcnq5^{off}) cell types (FIG. 15). Therefore, our snATAC/RNAseq dataset encompasses all major adult MN subtypes identified from other studies ^{48,49}.

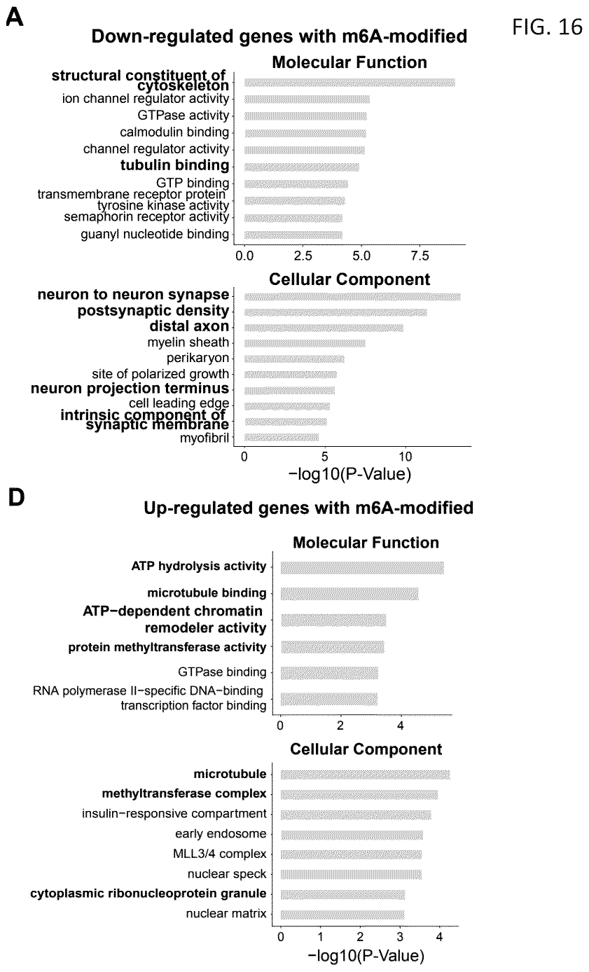

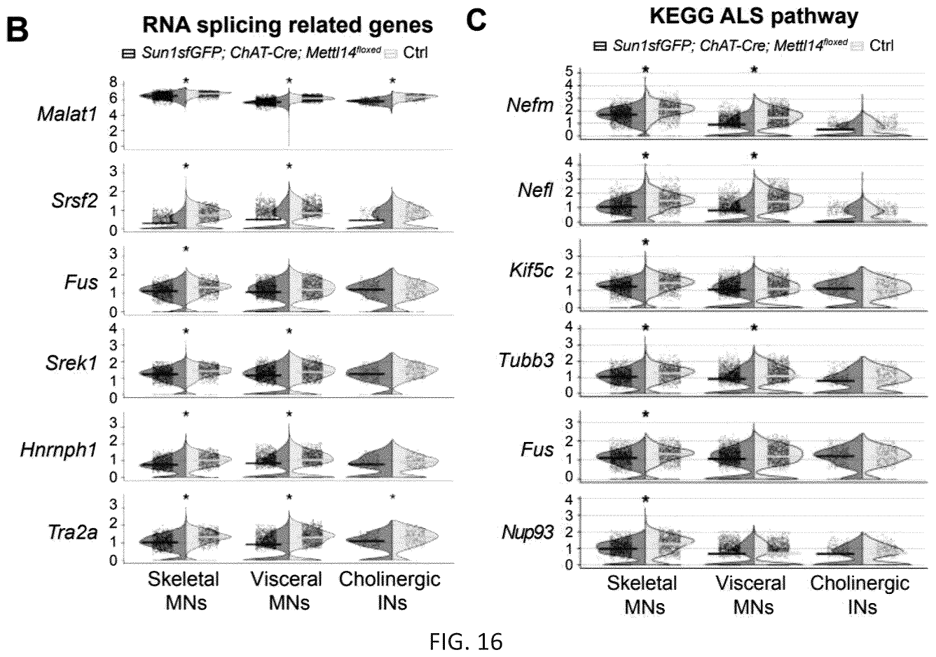

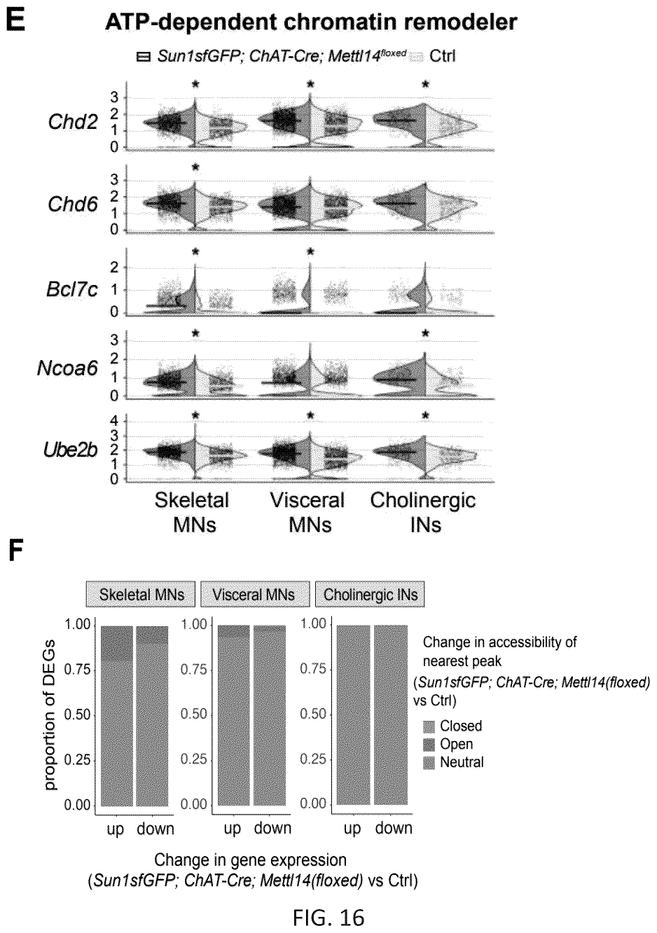

To discern the molecular alterations underlying the MN degeneration observed in Sun1^{sfGFP}; ChAT-Cre; Mettl14^{floxed} mice, we concentrated on differentially expressed genes (DEGs) within distinct types of cholinergic neurons. By performing a differential expression analysis between ChAT-Cre; Mettl14^{floxed}, and Ctrl samples in individual cell types, we identified 652,500, and 604 DEGs (with p-adjusted <0.05) for skeletal MNs (down-regulated=291 and up-regulated=361), visceral MNs (down-regulated=190 and up-regulated=310), and cholinergic INs (down-regulated=165 and up-regulated=439), respectively. To identify specific candidates presenting direct dysregulation due to m⁶A modifications and thus potentially contributing to neuronal degeneration, we conducted GO and KEGG pathway analyses focusing on DEGs exclusively recognized as m⁶A-modified based on Nanopore sequencing across all cholinergic neurons from ChAT-Cre; Mettl14^{floxed} mice (FIG. 12E). In alignment with our findings of MN denervation and compromised neuromuscular junction (NMJ) size in the ChAT-Cre; Mettl14^{floxed} mice (FIGS. 7E~7H), together with neurite degeneration in METTL3i-treated and knockdown iPSC-derived MNs (FIG. 3), GO analysis of m⁶A-modified and down-regulated genes revealed enrichment for pathways related to axonogenesis, synapse organization, cytoskeleton, and tubulin-related gene terms (FIGS. 12E and 16A). Notably, we observed down-regulation of RNA splicing-related genes (e.g., Malat1, Srsf2, Fus, Srek1, Hnrnph1, Tra2a) in the ChAT-Cre; Mettl14^{floxed} samples (FIG. 16B), corroborating previous studies showing that m⁶A-mediated splicing induces changes in gene expression 3. Among this group of analyzed genes, we noted that ALS-associated genes were prominent in our KEGG pathway analysis (FIG. 12F). Specifically, in MNs, these down-regulated genes are linked to neurofilament (Nefm, Nefl), tubulin (Kif5c, Tubb3), gene encoding DNA/RNA binding protein (Fus), and nucleoporin (Nup93) (FIG. 16C). Mutation or aberrant expression of these genes has been shown to contribute to ALS pathologies, such as cytoskeletal defects and nucleocytoplasmic transport ³¹. Moreover, the down-regulation of calcium signaling, which is often linked to neurodegenerative diseases ⁵⁴, appeared as the top enriched pathway for m⁶A-modified and down-regulated genes in cholinergic neurons (FIG. 12F). Among the m⁶A-modified ALS risk genes (34 out of 81 genes in FIG. 8), expression of two genes—Fus and Bscl2—was significantly reduced, whereas that of Pikfyve was increased in the skeletal MNs of ChAT-Cre; Mettl14^{floxed} mice (FIGS. 12G and 12H), in accordance with dysregulated ALS genes identified from patients ⁵⁵. These alterations are likely major contributors to MN degeneration and the subsequent motor behavior deficits observed in our ChAT-Cre; Mettl14^{floxed} mice. Taken together, these findings strongly imply a direct association between decreased m⁶A levels, neurodegeneration, and ALS pathology.

### Hypo-m⁶A MNs Exhibit an Increase of Closed Chromatin Regions

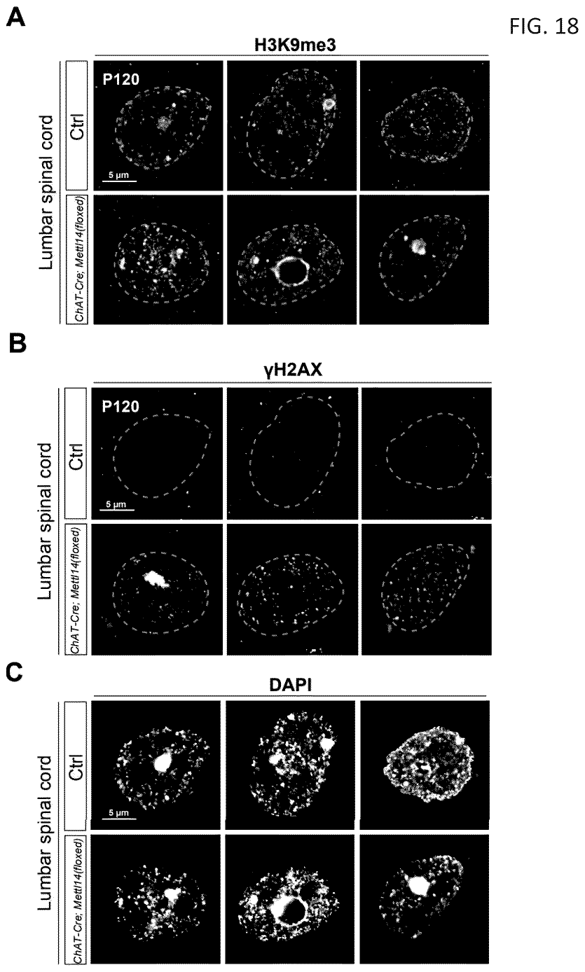

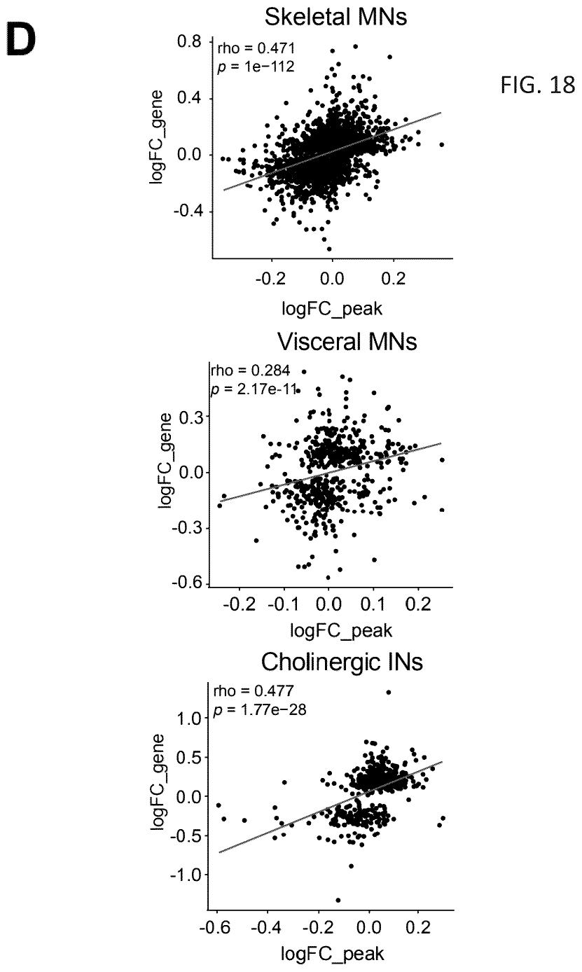

In contrast to the down-regulated DEGs, the up-regulated DEGs with m⁶A modifications exhibited conspicuous enrichment in genes responsible for regulating chromatin and histone modification (FIGS. 17A and 16D). Notably, several of these genes up-regulated in response to hypo-m⁶A—including members of the ATP-dependent Chd family, Bcl7c, Ncoa6, and Ube2b—have been implicated in the DNA damage response and apoptosis. These pathways are commonly implicated in diverse neurodegenerative diseases 56 (FIG. 16E). Accordingly, we noticed that the MNs of ChAT-Cre; Mettl14^{floxed} mice displayed a drastic increase of γH2AX signals, together with a striking increase in repressive histone modification marks (i.e., H3K9me3) (FIGS. 17B~17E, 18A, and 18B). Our discovery of up-regulated expression of genes linked to chromatin/histone modification prompted us to examine if changes in chromatin are associated with the aforementioned identified DEGs. To do so, we probed changes in chromatin accessibility in our snATAC/RNAseq dataset. Among the cholinergic cell types, ChAT-Cre; Mettl14^{floxed} skeletal MNs displayed the most drastic peak changes (open peaks=3,278, closed peaks=10,744), followed by visceral MNs (open peaks=2,495, closed peaks=920), with cholinergic INs exhibiting the fewest peak changes (open peaks=171, closed peaks=602), most of these peaks are in intergenic or intronic regions (FIG. 17F). Subsequently, we integrated and scrutinized the snRNAseq and snATACseq data, revealing that only a modest subset of DEGs align with alterations in chromatin accessibility (FIG. 16F). To further investigate the relationship between changes in chromatin accessibility and dysregulated gene expression, we first identified linked peak-to-gene associations and performed a correlation analysis between gene expression and changes in chromatin accessibility following Mettl14 ablation (FIG. 18D). Our analysis revealed a low correlation between these two factors. Notably, only 17%, 11%, and 5% of the genes associated with regions of differential chromatin accessibility for skeletal MNs, visceral MNs, and cholinergic neurons, respectively, were both m⁶A-modified and differentially expressed upon Mettl14 ablation. Together, these discoveries underscore the critical importance of preserving a nuanced equilibrium in the m⁶A transcriptome within adult MNs to maintain neuronal homeostasis. Diminished m⁶A levels may lead to compromised expression of pivotal neuronal and disease-associated genes governed by versatile regulatory mechanisms, i.e., either through direct modification of m⁶A-affected transcripts or by reshaping the chromatin landscape within MNS.

### Restoring m⁶A Homeostasis Rescues MN Degeneration in Both Sporadic and Familial ALS Models

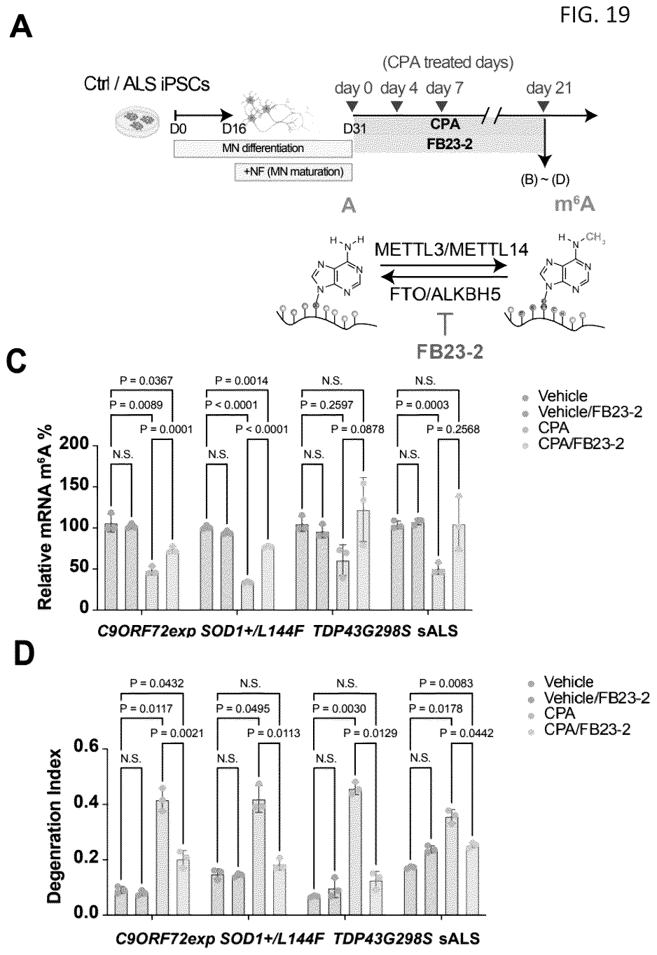

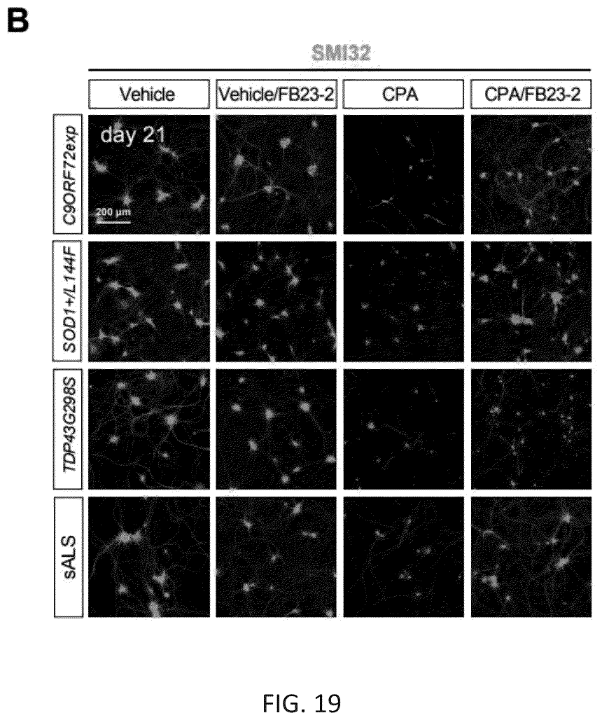

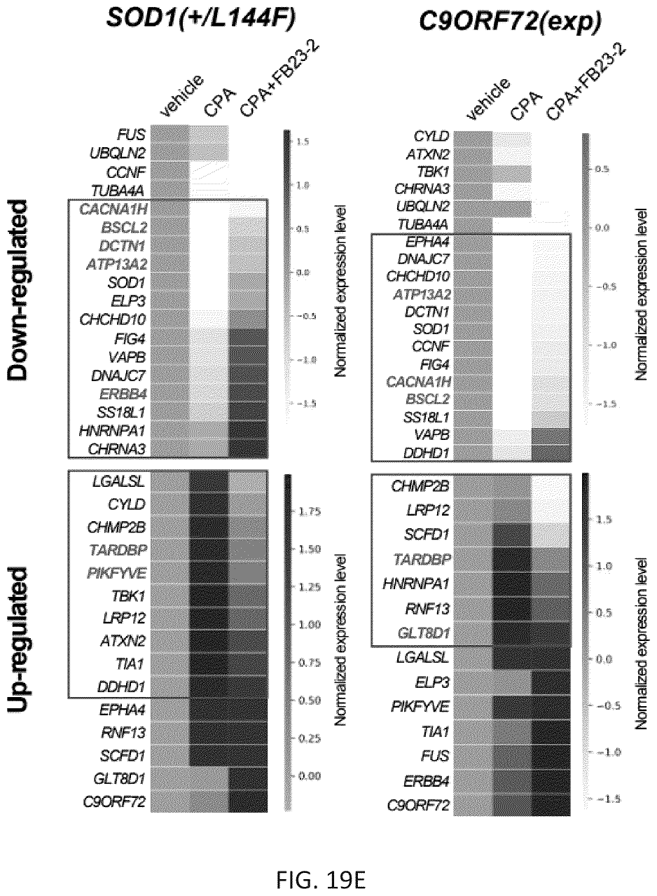

The consistent manifestation of hypo-m⁶A in human ALS iPSC~MNs, together with our ChAT-Cre; Mettl14foxed mice recapitulating ALS pathology, prompted us to explore if bolstering the m⁶A reservoir could represent a therapeutic strategy. Thus, we deployed several fALS patient (C9ORF72^{exp-800 G4C2}, SOD1^{+/L144F}, TDP43^{G298S}) iPSC~MN lines and one sALS iPSC~MN line to reflect MN degeneration (FIG. 19A~19D). Then, we treated these lines with FB23-2, an inhibitor of FTO (an m⁶A eraser) ⁵⁷, to see if this approach could be applied to rescue MN degeneration (FIG. 19A). First, we differentiated the ALS iPSCs under defined conditions to cause MN degeneration through a selective ER stressor, CPA (FIGS. 19A and 19B). Subsequently, we applied FB23-2 to determine if doing so could elevate m⁶A levels and thereby restore the m⁶A repertoire to rescue MN degeneration in different contexts of ALS. As expected, we observed a consistently significant increase in m⁶A levels upon applying FB23-2, albeit to varying degrees (FIG. 19C). By using SMI32 to assess MN degeneration and neurite complexity, we observed that FB23-2 promotes MN survival upon CPA stressor treatment for both familial and sporadic ALS MNs (FIG. 11D). Thus, our results indicate that fortifying basal m⁶A levels by adding a m⁶A eraser inhibitor can rescue human ALS iPSC-derived MNs from degeneration.

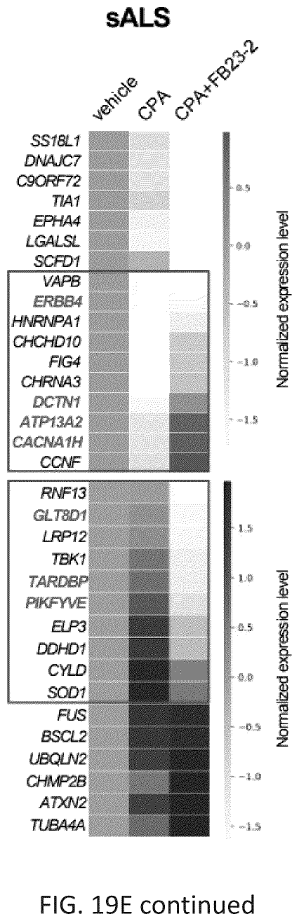

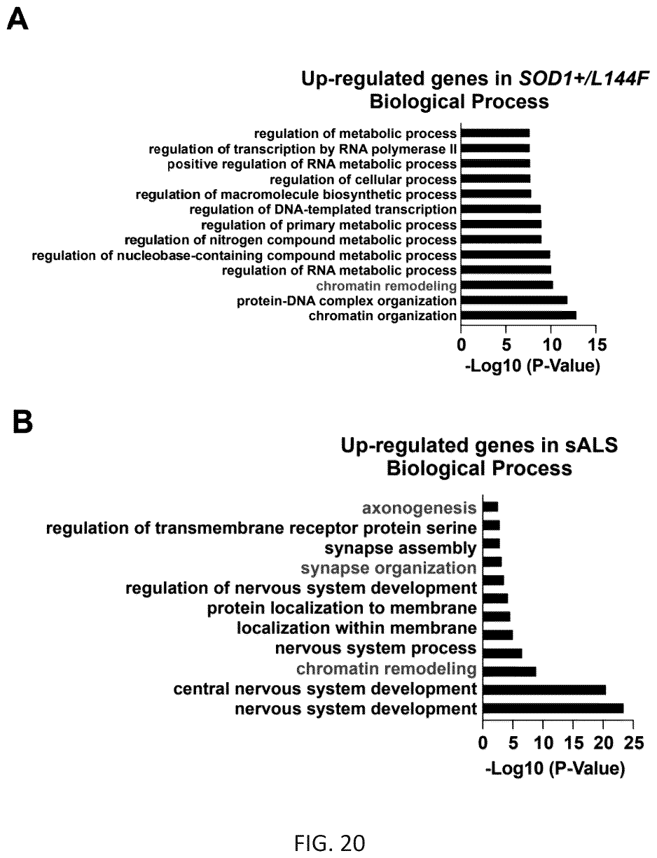

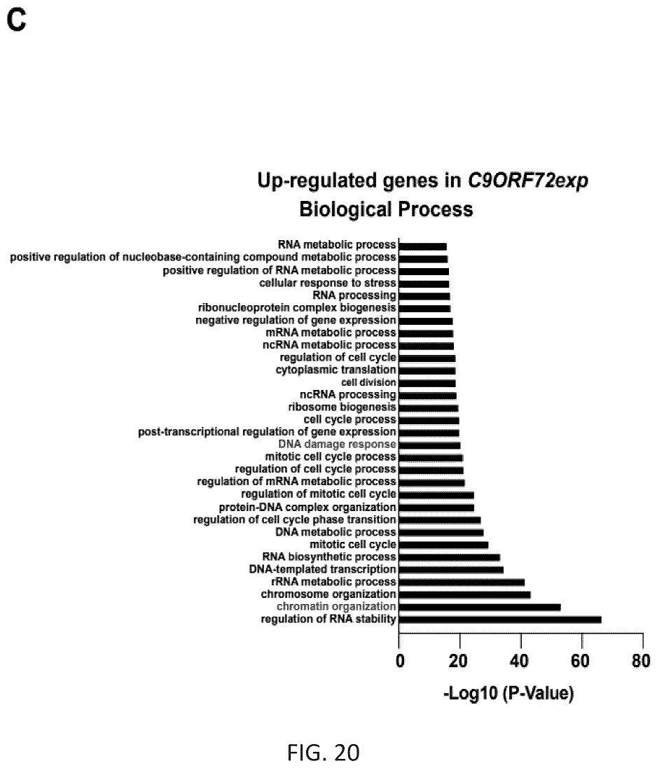

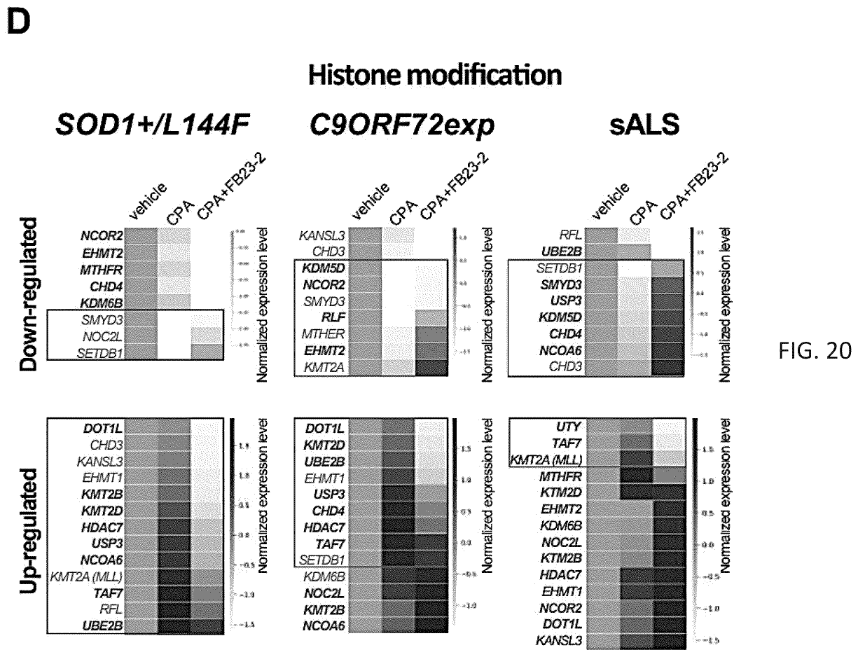

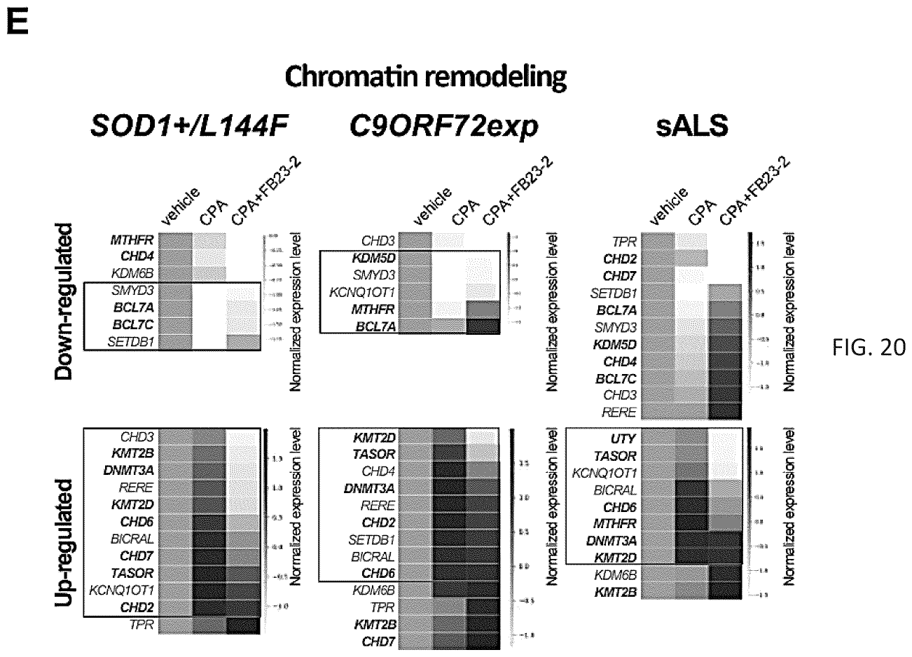

To determine if the neuroprotective effects of the FTO inhibitor on MN degeneration in ALS are mediated through its regulation of m⁶A-modified ALS risk genes (FIGS. 12G and 12H), we performed RNA-seq analysis on ALS iPSC-derived MNs treated with FB23-2. Differential expression analysis revealed that following FB23-2 treatment, several m⁶A-modified genes involved in synaptic function, RNA metabolism, and chromatin and histone modifications were restored to levels similar to controls (FIG. 20). Notably, the expression of multiple m⁶A-modified ALS risk genes was returned to control-like levels (vehicle-treated), suggesting that FTO inhibition may mitigate MN degeneration in ALS by modulating m⁶A-modified gene expression (FIG. 19E). These findings indicate that enhancing m⁶A levels in ALS iPSCs using small molecules could help restore the balance of the m⁶A epitranscriptome and preserve MN integrity.

FTO Gene Therapy Extends the Lifespan of SOD1G93A Mice and Ameliorates their MN Degeneration

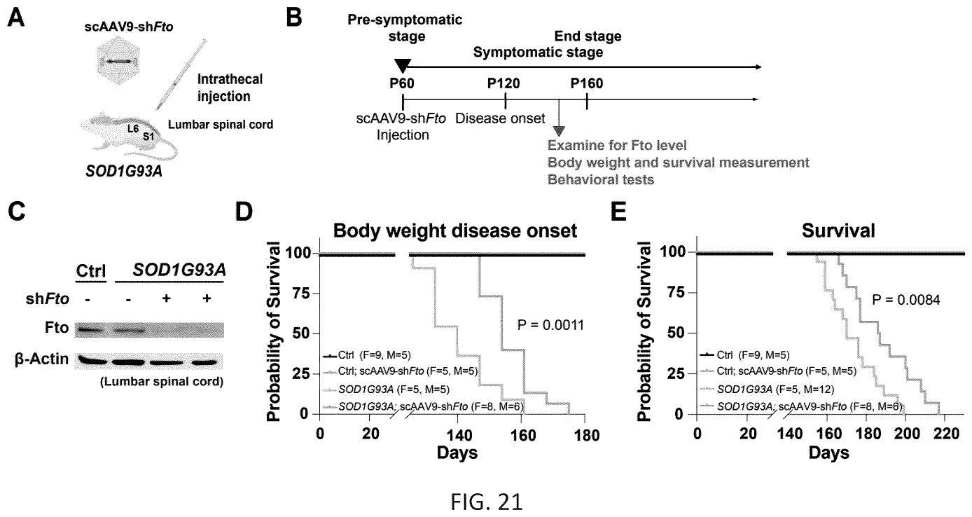

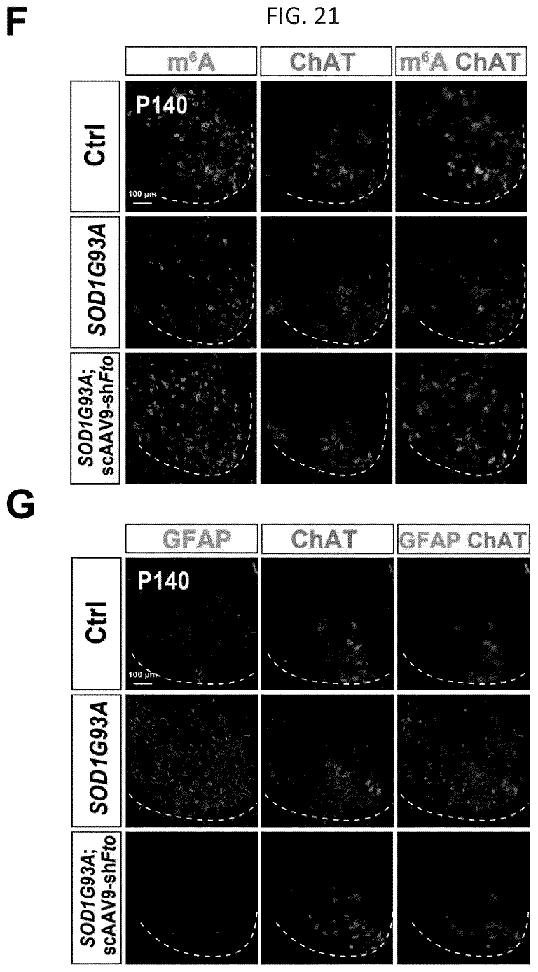

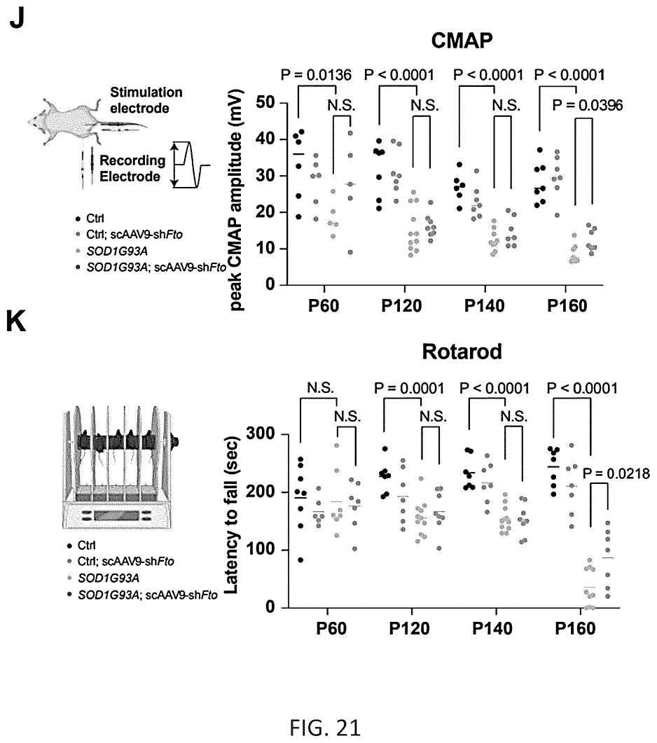

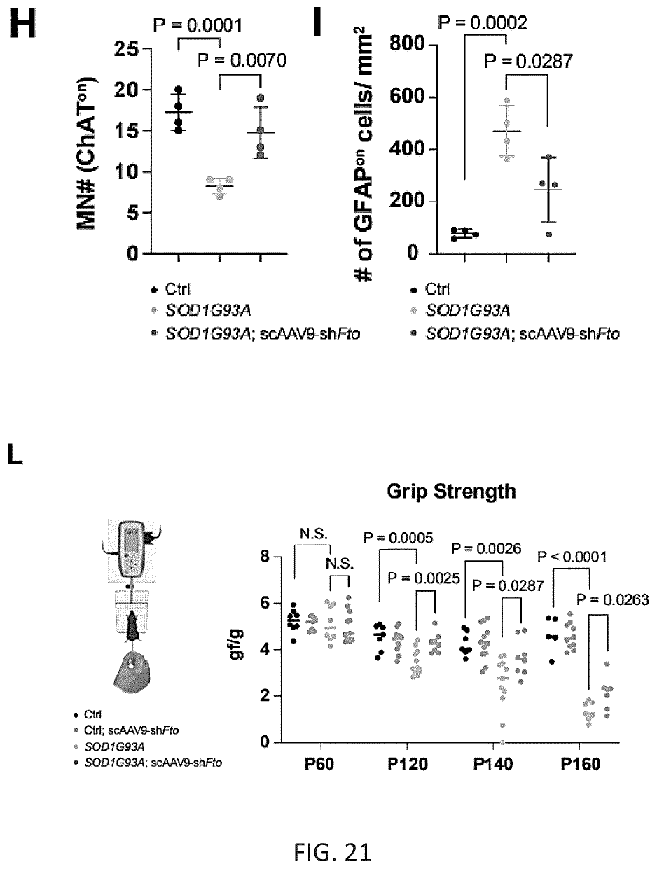

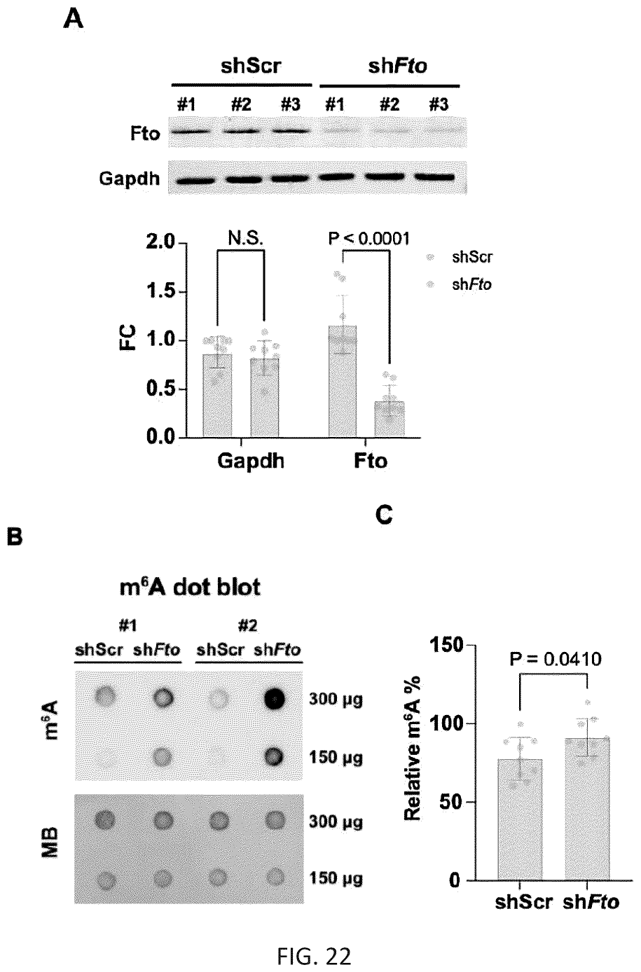

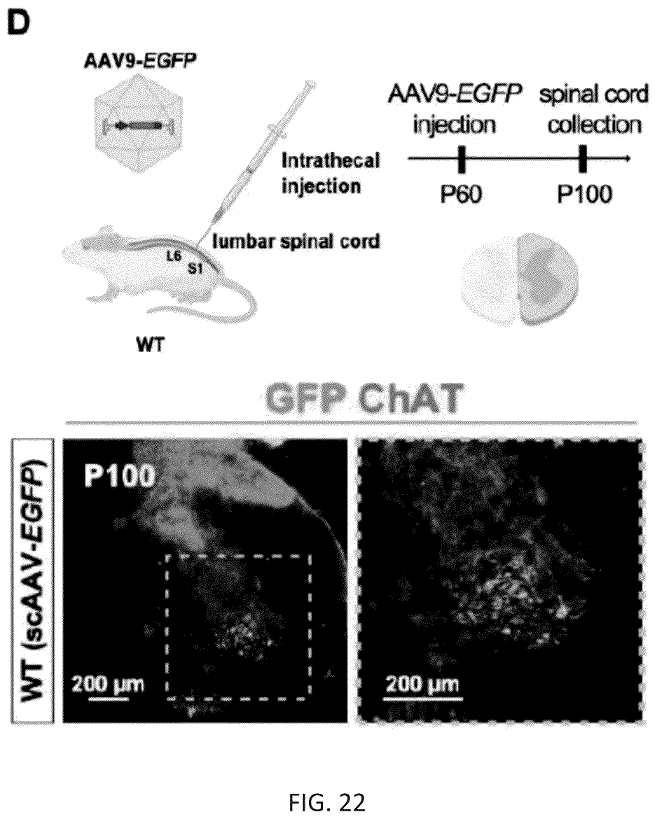

Although human iPSCs may be used as an effective platform for drug screening, they preclude further investigation of treatment efficacy at the tissue/organ/behavioral levels. Moreover, the penetrance of FTO inhibitors through the blood-brain barrier is poor and so systematic appliance of such inhibitors might cause adverse effects ⁵⁸. To circumvent this issue, we adopted gene therapy as an alternative approach and delivered self-complementary adeno-associated vector serotype 9 (scAAV9) of Fto-shRNA to knock down Fto expression in the spinal cord (FIGS. 21A, 21B, and 22). We first verified the knockdown efficiency of three Fto-shRNAs and their restoration of m⁶A level in C2C12 cells (FIGS. 22A~22C). Then we subcloned the Fto-shRNAs into scAAV9 driven by the H1 promoter (scAAV9-shFto) ⁴⁵. To test the efficiency of viral infection, we injected scAAV9-EGFP into control mice and observed sustained GFP expression in their spinal MNs and in some dorsal cells at 40 days post-injection (FIG. 22D). Subsequently, scAAV9-shFto was injected into SOD1^{G93A} mice and their wild-type littermates. Then we verified a dramatic reduction in Fto expression in the spinal cords (FIG. 21C). Prominently, scAAV9-shFto gene therapy delayed the disease onset (FIG. 21D), robustly prolonged the median survival of SOD1^{G93A} mice to ~14 days (FIG. 21E), and enhanced MN survival with a significant reduction of gliosis (FIGS. 21F~21I).

To evaluate the mice in a more clinically relevant setting, we assayed MN and gastrocnemius (GA) muscle connectivity by measuring the compound motor action potential (CMAP). We performed the CMAP assay from P60 (directly before AAV treatment) to P160 (FIG. 21J). Consistent with a previous study, CMAP amplitude is already reduced in SOD1^{G93A} mice at P60 and gradually declines further over time, whereas scAAV9-shFto treatment mildly ameliorates neuromuscular function at P160. (FIG. 21J). This moderate improvement was also reflected by the enhanced behavioral performance of SOD1^{G93A} mice from P120 following scAAV9-shFto treatment (FIG. 21K), with a substantial increase of muscle strength (FIG. 21L), a major clinical measurement for ALS motor score.

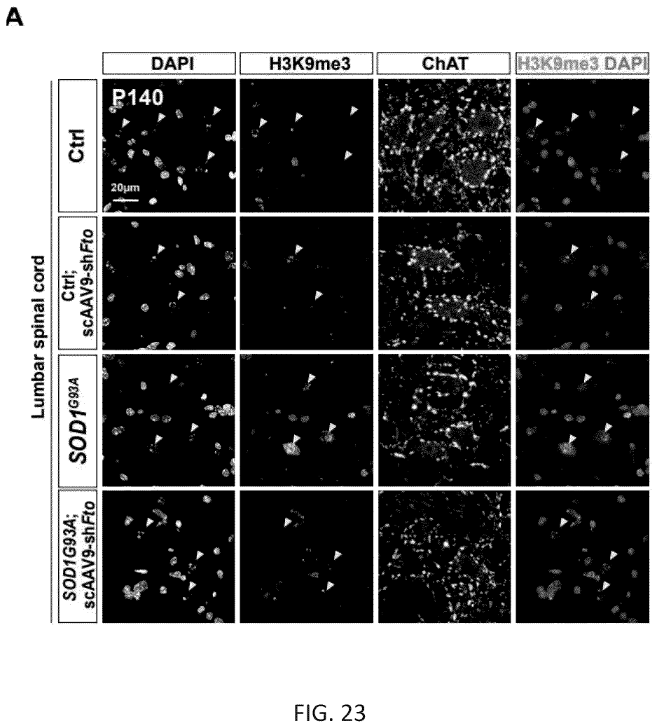

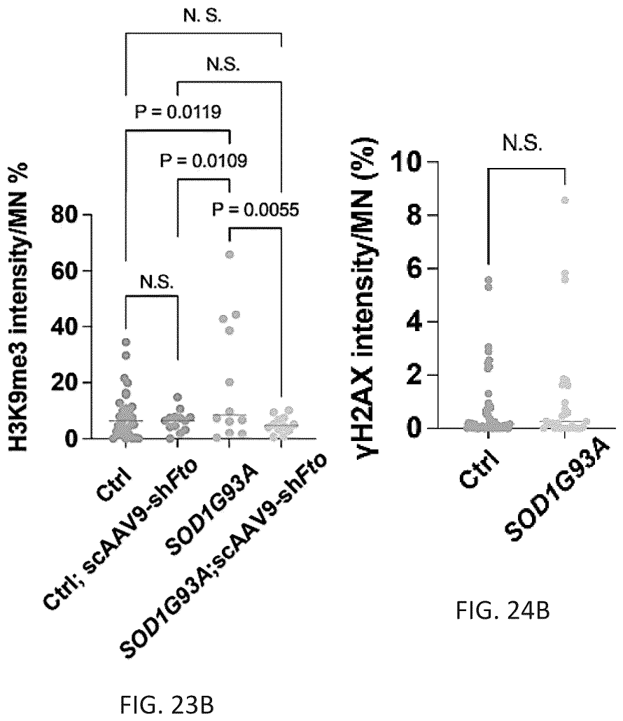

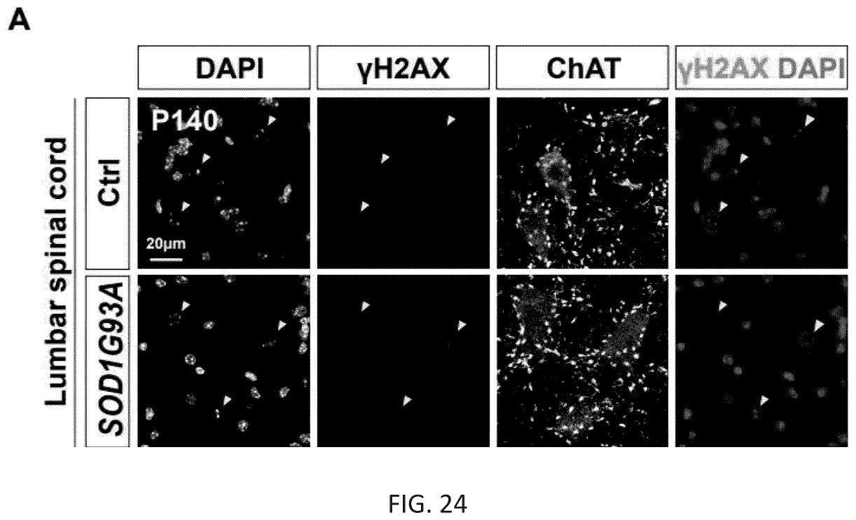

To determine if scAAV9-shFto treatment rescues SOD1^{G93A} mice through m⁶A-mediated molecular changes, such as histone modifications in H3K9me3 and γH2AX, we assessed these markers in four groups-wild-type control (Ctrl), Ctrl treated with scAAV9-shFto (Ctrl; scAAV9-shFto), SOD1^{G93A}, and SOD1^{G93A} treated with scAAV9-shFto (SOD1^{G93A}; scAAV9-shFto)—at postnatal day 140 (P140), i.e., when SOD1^{G93A} mice are at an early symptomatic stage (FIG. 23). Remarkably, H3K9me3 levels were significantly elevated in the ventral horn of the SOD1^{G93A} mice and were restored to control levels following scAAV9-shFto treatment (FIG. 23), suggesting that epigenetic dysregulation may be a novel hallmark of ALS pathogenesis mediated by hypo-m⁶A. In contrast, no significant changes in γH2AX were observed in the spinal cords of the SOD1^{G93A} mice (FIG. 24).

Overall, our results support that m⁶A hypomethylation promotes ALS and, significantly, augmenting the m⁶A reservoir can mitigate the disease phenotypes of sporadic and familial human ALS iPSC~MNs. In a SOD1-linked ALS context, enhancing m⁶A levels in adult MNs either via small molecule treatment or scAAV9 delivery delays the onset of MN degeneration and enhances motor function. The discoveries we have presented herein reveal m⁶A as a potential prognostic indicator for MN degeneration and a promising therapeutic candidate in individuals afflicted with ALS. We discuss the significance and implications of our study in detail below.

### DISCUSSION

In this study, we generated two conditional spinal MN-Mettl14 knockout mouse lines, using the Olig2-Cre; Mettl14^{floxed} mice to impair m⁶A homeostasis from the embryonic stage and the ChAT-Cre; Mettl14^{floxed} mice to disrupt m⁶A levels from the late embryonic/early postnatal stage. Although Olig2-Cre; Mettl14^{floxed} mice exhibit early postnatal lethality (~P24 to P28), their MNs are relatively normal. The ChAT-Cre; Mettl14^{floxed} mice also display normal MN development and ordinary gross appearance at the postnatal and juvenile stages. These results indicate that m⁶A homeostasis appears to be less critical for embryonic spinal MN development. This scenario is different to conditional Mettl14 knockout in mouse neural progenitor cells (NPCs) using Nestin-Cre that impairs NPC differentiation, prolongs cell cycle progression of radial glia, and extends cortical neurogenesis into postnatal stages 18,19. Thus, our results, together with these previously published results, emphasize the neuron-type context-dependent role of m⁶A homeostasis, prompting the notion that the m⁶A epitranscriptome is both dynamic and diverse, and it operates in a cell and developmental context-dependent manner. Our Olig2-Cre; Mettl14 ^{floxed} mice display a shivering phenotype, likely a reflection of compromised oligodendrocytes, consistent with a previous study using a different Olig2-Cre line ⁵². We have shown previously that the Olig2-Cre line we deployed in this study displays more efficient floxed allele removal activity than the other line ⁵⁹ used in that previous study, so it is understandable that we observed a more lethal shivering postnatal phenotype than the other study that only showed oligodendrocyte defects at the adult stage ⁵².

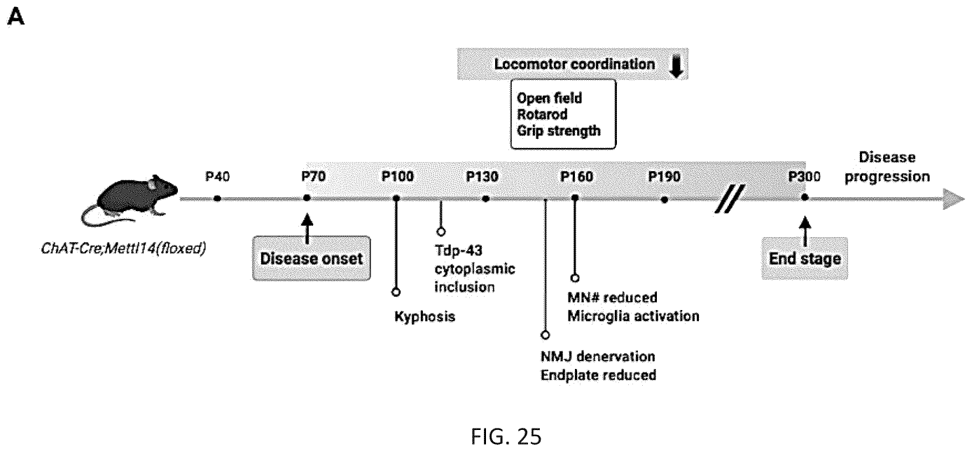

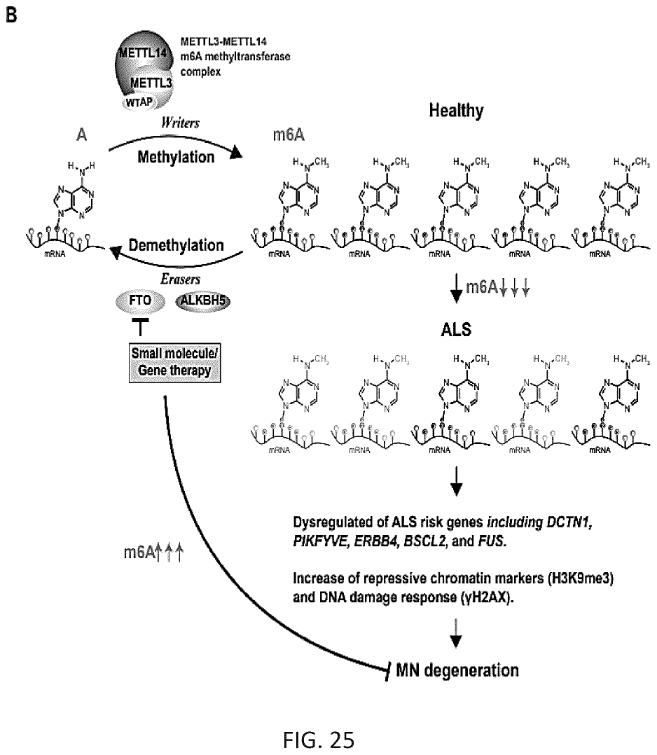

Despite sporadic ALS cases predominating in the real world, current animal models often rely on familial genetic mutations. The expanding genetic spectrum of ALS has prompted continuous efforts to establish rodent models that emulate human physiological deficits and pathological manifestations ^{33,55,60}. Nevertheless, translation of effective treatment outcomes in rodent models to clinical success in humans remains limited, emphasizing the crucial need for an authentic rodent ALS model that mirrors human symptoms. Thus, even though rodent models of familial ALS are prevalent, creating sporadic ALS animal models is pivotal for pathomechanistic insights and establishing novel therapeutics. Various sporadic ALS pathologies in MNs have been simulated in mice, such as reduced ADAR2 protein levels or filament alterations ^{61,62}. Environmental toxin exposure, including metals and β-N-methylamino-L-alanine, has also been explored alongside patient-derived fluid injections ⁶³. These models partially replicate ALS features, but their status as true sporadic ALS models is arguable. Here, our ChAT-Cre; Mettl14^{floxed} mice exhibit several salient features of human ALS pathologies and symptoms (FIG. 25A). At the phenotypic level, our ChAT-Cre; Mettl14^{floxed} mice exhibit late-stage paralysis with early adult lethality, diminished body weight, kyphosis with muscle atrophy, as well as coordination and motor deficits. At the pathological level, our ChAT-Cre; Mettl14^{floxed} mice display gradual MN loss, neuroinflammation, axonopathy, neuromuscular junction abnormality, and muscle denervation. Of paramount significance, our novel mouse model distinctly manifests early-onset cytoplasmic aggregations of Tardbp (Tdp43) and Fus, representing a principal hallmark observed in the post-mortem tissues of both familial and sporadic ALS cases, yet this phenotype has been conspicuously rare in existing rodent models. Moreover, we observed consistent m⁶A hypomethylation in several familial and sporadic ALS lines we tested, congruent with a recent study indicating that the expression of m⁶A methyltransferases and levels of m⁶A RNA modification are down-regulated in C9ORF72-ALS/FTD patients ³⁶. Thus, taken together, we assert that m⁶A hypomethylation leads to ALS and that impairing m⁶A modification in adult MNs has provided us with one of the most reliable rodent models of ALS. Although ChAT-Cre; Mettl14^{floxed} mice exhibited several ALS-like phenotypes, we were unable to determine whether these effects were specifically attributable to C-boutons synapsing on the soma and proximal dendrites of MNs, the MNs themselves, or both. This is an intriguing question, as recent reports have suggested that spinal inhibitory neurons, including C-boutons, might degenerate before MNs in a mouse model of ALS 64,65. Currently, the lack of a Cre driver that specifically targets adult MNs limits examining this question. However, our single-nuclei ATAC-seq data offer a promising avenue for identifying enhancers specific to adult cholinergic interneurons (INs) and MNs. Generating Cre lines driven by these enhancers could allow for a more precise dissection of the respective contributions of INs and MNs to the observed phenotypes.

As a hallmark of ALS, why does Tardbp (Tdp43) and Fus move out of the nucleus to form cytoplasmic aggregations in the ChAT-Cre; Mettl14^{floxed} mice? A previous study has revealed that a lack of specific RNA modifications may affect global and/or local translation rates, consequently increasing protein aggregation ⁶⁶. Thus, it has been proposed that RNA modifications serve as conduits of information linking a cell's metabolic condition with its translational productivity ⁶⁷. Consequently, any disruption in regulating RNA modifications could potentially perturb the equilibrium between metabolic processes and protein synthesis. Further work is needed to disentangle the causal relationship between dysregulation of RNA modifications and Tdp43 translocation. Additionally, the nuclear m⁶A reader YTHDC1 has been shown to exert an important role in modulating many biological processes and to contribute to disease, especially cancers. YTHDC1 might be the main mediator of a series of m⁶A readers, thereby controlling their activity in neuronal functions ⁶⁸. Moreover, YTHDC1 RNAs have been discovered as binding to TDP43 protein in human SH-SY5Y neuroblastoma cells ⁶⁹, and peripheral blood sample transcriptional profiling of a huge heterogeneous ALS cohort (not only sporadic cases) revealed YTHDC1 to be differentially expressed ⁷⁰. These results point to YTHDC1-mediated m⁶A nuclear events and nucleocytoplasmic trafficking as contributing to ALS. Further experiments are warranted to dissect this potential disease mechanism in detail.

Based on four lines of evidence, we believe m⁶A hypomethylation is one of the key factors leading to ALS. First, the reduced presence of the m⁶A writer complex (METTL3/METTL14) is manifested in a random yet comprehensive (n>800) selection of familial and sporadic ALS iPSC~MNs and postmortem tissues, consistent with a recently published study 36. Second, a global reduction in the m⁶A epitranscriptome of several familial ALS iPSC~MNs precedes MN degeneration. Third, ChAT-Cre; Mettl14^{floxed} mice phenocopy ALS symptoms at molecular, cellular, and phenotypic levels. Fourth, restoring the m⁶A repertoire significantly mitigates ALS pathology in human iPSC and mouse models. However, the discrepancy between the current study's findings (i.e., the impact of hypo-m⁶A) and another recent study reporting m⁶A hypermethylation in the spinal cord of sporadic ALS patients warrants critical examination ³⁵. Several plausible explanations may account for this disparity. For instance, experimental model limitations, with previous studies predominantly utilizing cellular models and postmortem tissues, thereby potentially limiting direct correlations to in vivo pathology. The timing of sample collection, particularly at the end stage of the disease, might not adequately capture the initial contributors to ALS pathology. We suggest that studies involving animal models are necessary to establish a more direct link between manipulating m⁶A levels and ALS progression. Alternatively, the discrepancy could be attributable to the multifaceted and complex nature of sALS, encompassing diverse pathological mechanisms. To detect m⁶A levels, different assays have been used by our study and those of others, including antibody-based pull down, dot blot, ELISA, and microarray method ⁵⁸. We have further applied Nanopore ONT direct RNAseq to uncover m⁶A stoichiometry, which appears to provide better sensitivity. Each of these methods has advantages and disadvantages in terms of the sensitivity and feasibility of large-scale studies ⁵⁸. We advocate screening m⁶A levels in a larger cohort of sALS cases using advanced techniques such as direct Mass Spectrometry in future. Our study acknowledges the pivotal role of an optimal m⁶A reservoir in maintaining adult MN function, highlighting that both m⁶A hypomethylation and hypermethylation could be detrimental ³. This nuanced perspective underscores the need for a balanced understanding of m⁶A modifications in ALS pathophysiology and it calls for further investigation using diverse methodologies and models to comprehensively understand the role of m⁶A dysregulation in ALS progression.

In our proposed model (depicted in FIG. 25B), we postulate that the aberrant down-regulation of the two m⁶A methyltransferases, namely METTL3 and METTL14, in ALS reflects an anomaly likely associated with the natural aging process compounded by unidentified exacerbating factors ²⁹. This reduced expression of m⁶A methyltransferases has multifaceted implications. Firstly, it instigates widespread dysregulation across the mRNA transcriptome, notably enriching pathways associated with ALS risk genes, synaptic activity, and neuronal functional pathways. Simultaneously, m⁶A modification of chromatin regulators, crucial for modulating heterochromatin repression, is diminished. This global dysregulation of m⁶A-mediated epitranscriptomic processes disrupts RNA stability and perturbs the repressive chromatin landscape, potentially exacerbating the degeneration of MNs in the context of ALS. Strikingly, we observed a significant increase in closed chromatin regions in the ChAT-Cre; Mettl14^{floxed} mice, with only a small fraction of these chromatin changes correlating with dysregulated gene expression. Given that previous studies have shown FTO-mediated LINE1 RNA m⁶A demethylation plays a role in regulating chromatin state and gene expression during mouse oocyte and embryonic development 71, and that retrotransposon reactivation has been observed in some ALS postmortem tissues ⁶⁹, it would be intriguing to explore in future studies whether Mettl14-mediated m⁶A impairment leads to retrotransposon reactivation through its interaction with chromatin state.

In this study, we adopted two approaches to test possible treatments of ALS by bolstering the m⁶A reservoir. First, by applying a small-molecule FTO inhibitor (FB23-2) in our familial and sporadic ALS iPSC~MNs, we prevented the MNs from degenerating. FTO inhibition notably restored to control levels the expression patterns of several genes associated with ALS risk and chromatin-regulated pathways. This outcome strongly suggests that the FTO inhibitor's efficacy in preserving MNs from degeneration primarily stems from its ability to restore m⁶A-modified genes relevant to ALS pathology. Second, we observed a significant enhancement of motor and neuromuscular functions, together with a significant delay in disease onset following scAAV9-shFto intrathecal injection in our SOD1^{G93A} mice. Building on this exciting outcome, we are currently testing some new synthesized FTO inhibitors with stronger activity and better blood-brain barrier penetrance. Second, we observed a significant enhancement of motor and neuromuscular functions, together with a significant delay in disease onset following scAAV9-shFto intrathecal injection in our SOD1^{G93A} mice. Building on this exciting outcome, we are currently testing some new synthesized FTO inhibitors with stronger activity and better blood-brain barrier penetrance. We knocked down FTO using a ubiquitous promoter as an initial attempt. Future optimization to knock down FTO in a cell type-specific manner might minimize possible adverse effects, although targeting FTO might also represent a relatively safe treatment strategy as Fto knockout mice are morphologically normal and only display mild learning defects ^{72,73}. Future experiments to develop a new drug cocktail by combining small molecules together with gene therapy to fortify the m⁶A reservoir of both familial and sporadic ALS patients offer tantalizing treatment prospects. While we have validated several ALS risk genes with m⁶A-modified sites that are dysregulated in the ChAT-Cre; Mettl14^{floxed} mice, the direct contribution of these genes as a cohort to MN degeneration in ALS remains to be systematically tested. Furthermore, FTO is not only an m⁶A demethylase, but also functions as a demethylase for the m²-isoform (N⁶,2′-O-dimethyladenosine, m⁶Am) in small nuclear RNAs (snRNAs) ¹⁴ and mRNAs. Although we have demonstrated that FTO inhibition rescues the expression of many ALS pathway- and chromatin regulator-related genes, it remains to be investigated if this therapeutic effect involves m⁶Am-modified snRNAs.

Additionally, though we identified an increase in H3K9me3 and closed chromatin regions as potential new features of ALS, the precise mechanism of how hypo-m⁶A induces these chromatin changes remains unclear. It is also uncertain if the increase in H3K9me3 is directly correlated with closed chromatin regions. Future large-scale investigations using human ALS iPSC~MNs will be crucial to further explore these potential pathological features. Moreover, it would be valuable to assess if combinatorial treatments targeting both H3K9me3 and m⁶A modifications could offer enhanced benefits in preventing MN degeneration. Extending beyond the challenge of ALS, perturbations of the m⁶A reservoir is a recurring theme of other neurodegenerative conditions, including Alzheimer's disease, Parkinson's disease, and multiple sclerosis ³. As these pioneering paradigms relating to m⁶A modification ripple through the scientific landscape, we envisage their transformative potential will extend to diverse ailments in the future.

Combinational Treatments to Rescue the CPA-Induced Degeneration of ALS iPSC-Derived Motor Neurons

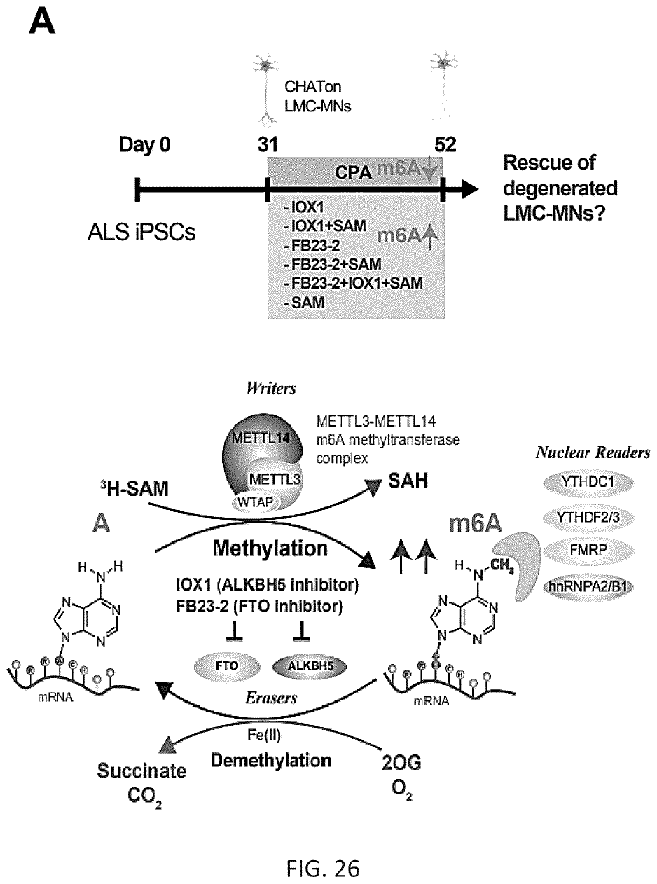

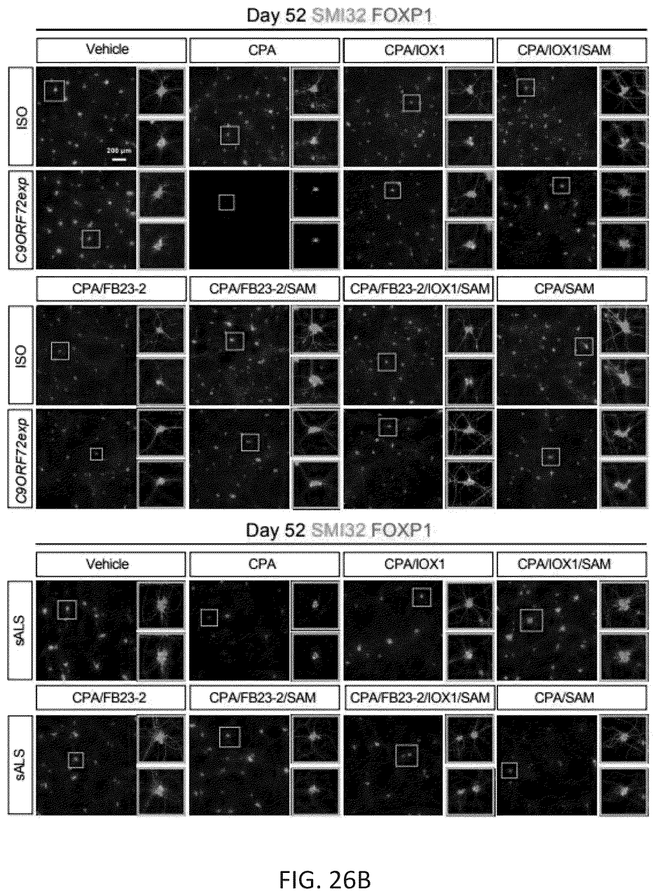

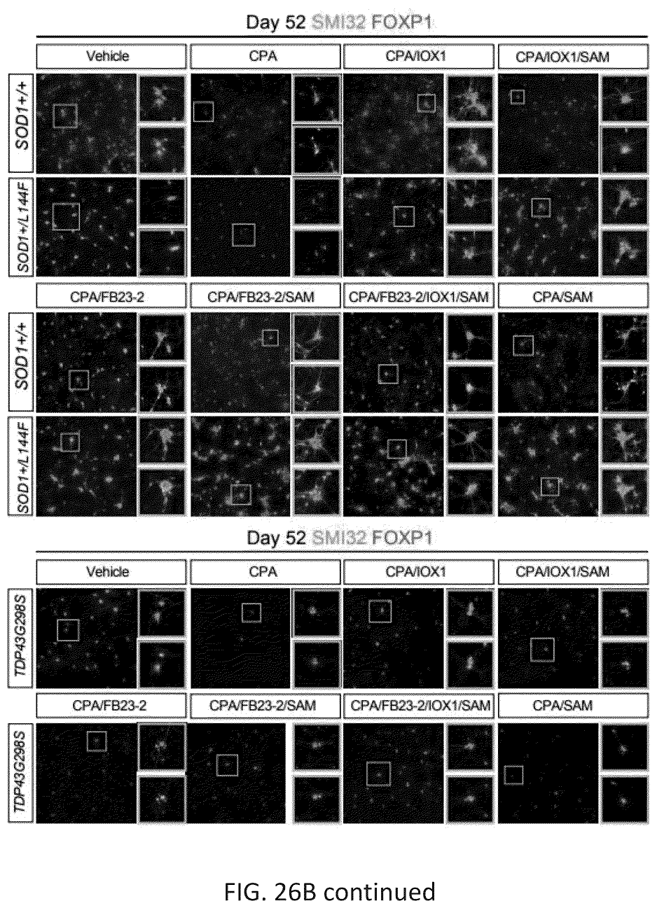

To analyze the effect of m⁶A eraser inhibitor and/or methyl donor on rescuing MN degeneration, we applied single treatment of IOX1 (20 μM), SAM (1 nM), or FB23-2 (1 μM), or a combination of these treatments in different ALS contexts (FIG. 26A). While single treatment of IOX1, SAM, or FB23-2 effectively rescue CPA-induced MN degeneration as indicated by positive staining of SMI32 and FOXP1, combinational treatments such as IOX1+SAM, FB23-2+SAM, and FB23-2+IOX1+SAM show comparable or even better activity (FIGS. 26B and 26C), indicating the potential of using these combinations for ALS treatments.

Verification of the shRNA-FTO Knockdown Efficiency

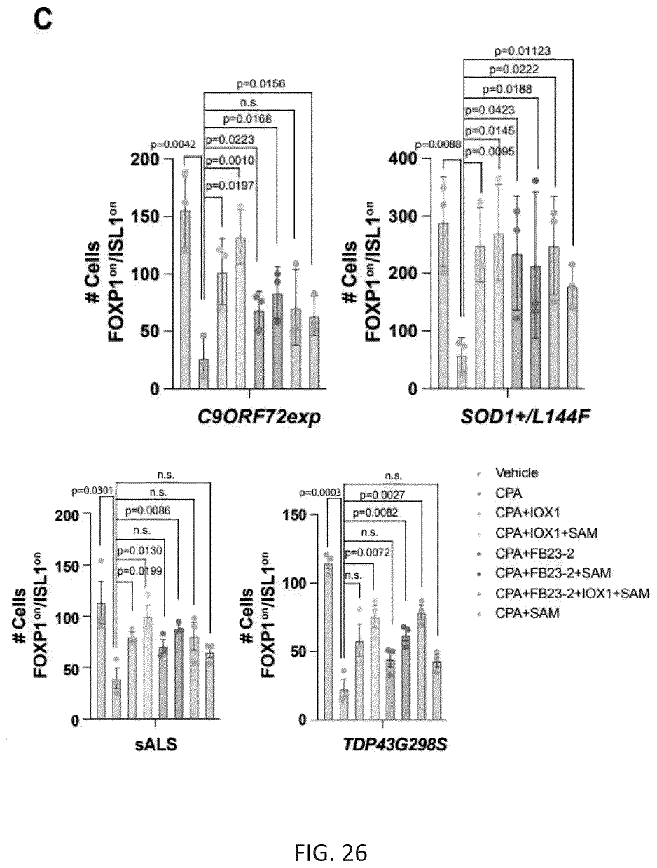

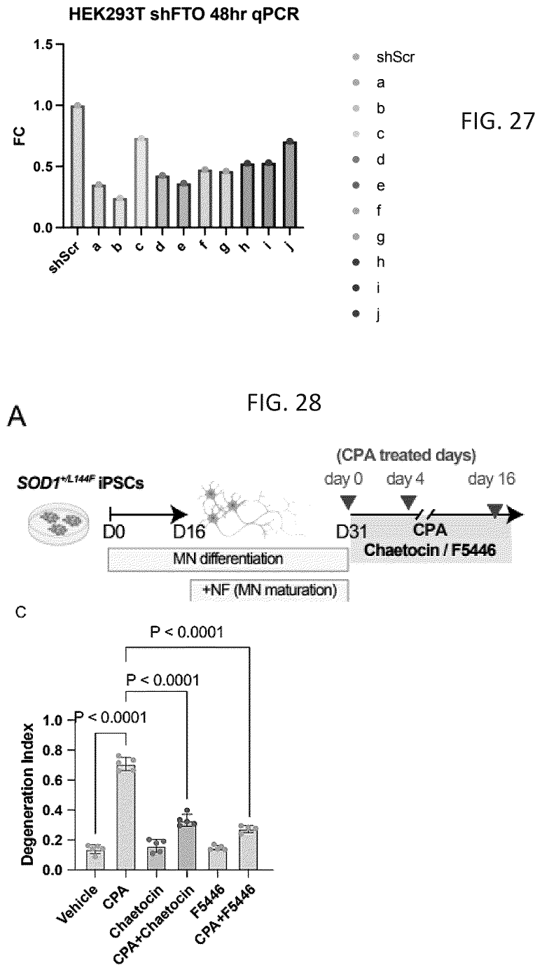

The shRNA-FTO oligonucleotide sequences a-j (Table 1 and 2) was generated from website prediction (VectorBuilder). To test the FTO knockdown efficiency of selected shRNA-FTO oligonucleotides, the oligonucleotides were cloned into the pLKO_TRC005 vector. These constructs were further transfected into HEK293T cells in about 70% confluent. HEK293T cells were maintained in DMEM containing L-Glutamine (Thermo) supplemented with 10% FBS (Thermo). Cells were cultured in a 5% CO₂ humidified atmosphere at 37° C. RNA was harvested by Trizol. The gene expression was measured by qPCR analysis using GAPDH as an internal control and normalized with the scramble group (shScr). Inhibition efficiency of the shFTOs is from about 20% to about 80% compared to the shScr control group (FIG. 27).

Single or Combinational Treatments to Rescue the CPA-Induced Degeneration of ALS iPSC-Derived Motor Neurons

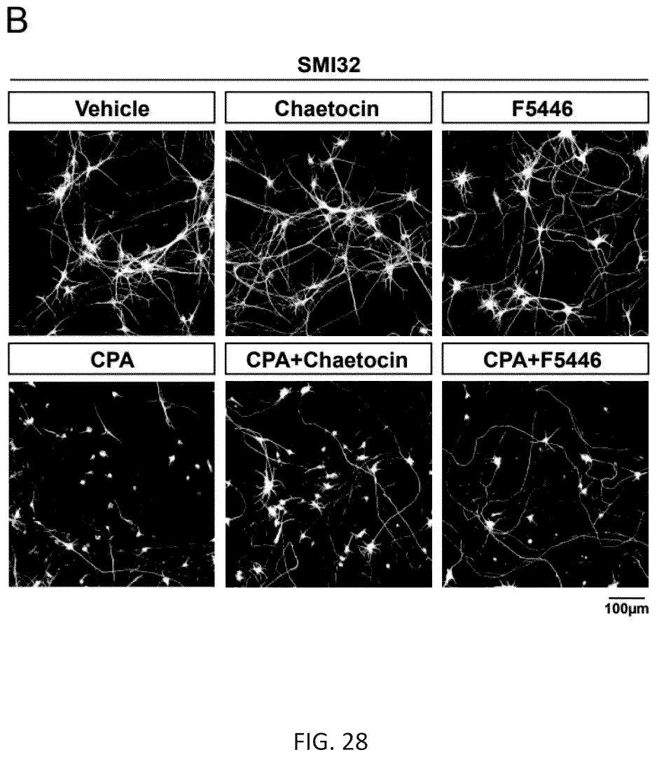

To analyze the effect of histone methylation regulator, we applied single treatment of chaetocin (0.5 nM), or F5446 (1 nM) in SOD1^{+/L144F} ALS iPSC~MNs (FIG. 28). Treatment of chaetocin, or F5446 effectively rescue CPA-induced MN degeneration as indicated by positive staining of SMI32 (FIGS. 28B and 28C), indicating the potential of using these combinations for ALS treatments.

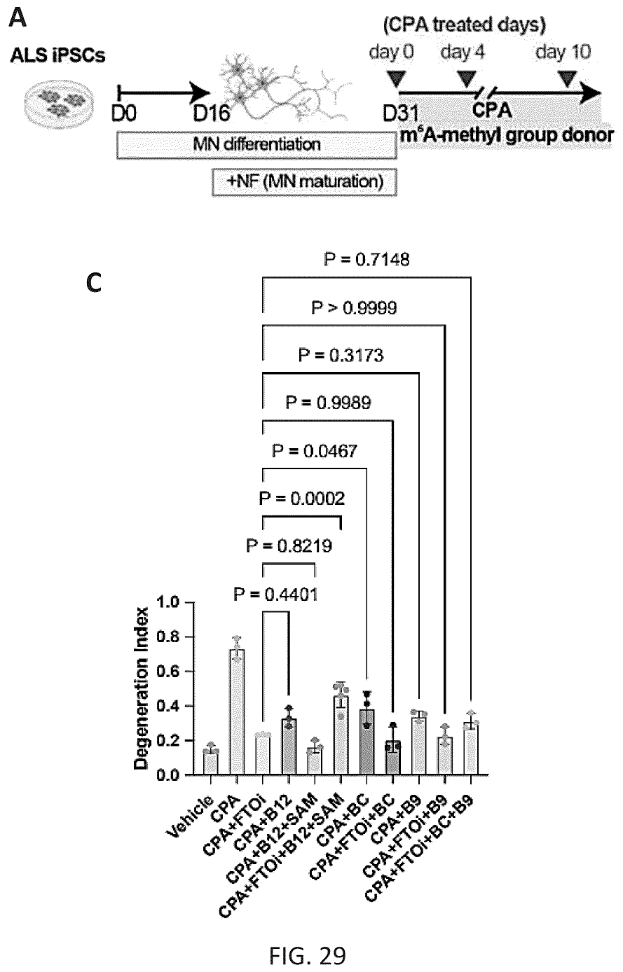

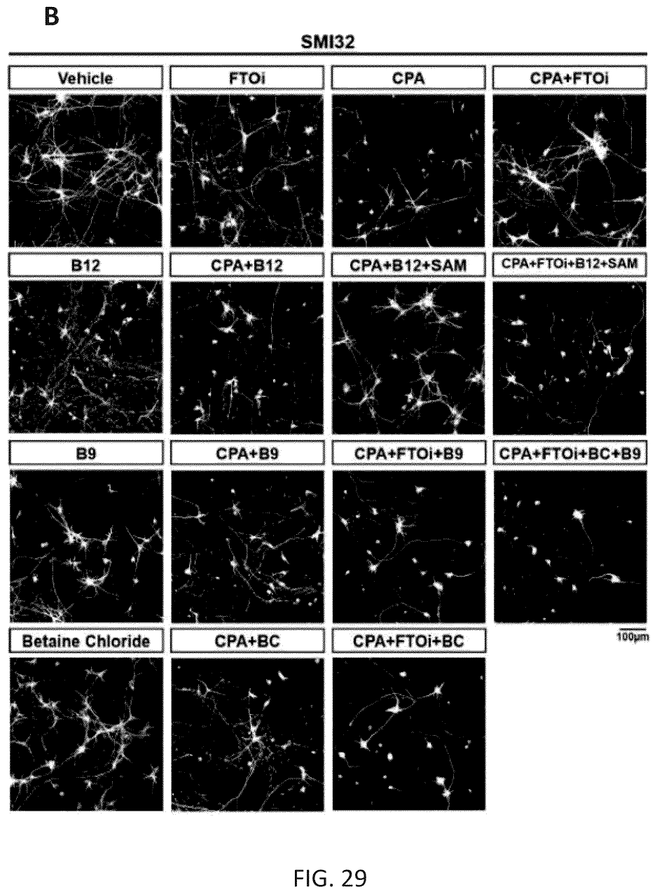

To analyze the effect of m⁶A eraser inhibitor and/or methyl donor on rescuing MN degeneration, we applied single treatment of FB23-2 (1 μM), vitamin B12 (300 nM), SAM (100 nM), vitamin B9 (100 nM), betaine chloride (300 nM), or a combination of these treatments in ALS iPSC-derived MNs (FIG. 29). All the tested treatments effectively rescue CPA-induced MN degeneration as indicated by positive staining of SMI32 (FIGS. 29B and 29C), indicating the potential of using these combinations for ALS treatments.

It can be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

### REFERENCES WHICH ARE HEREBY EACH INCORPORATED IN THEIR ENTIRETY

1 He, C. Grand Challenge Commentary: RNA epigenetics? Nature Chemical Biology 6, 863 (2010). https://doi.org/10.1038/nchembio.482
2 Li, X., Xiong, X. & Yi, C. Epitranscriptome sequencing technologies: decoding RNA modifications. Nature Methods 14, 23 (2016). https://doi.org/10.1038/nmeth.4110 https://www.nature.com/articles/nmeth.4110 #supplementary-information
3 Zhang, F., Ignatova, V. V., Ming, G. L. & Song, H. Advances in brain epitranscriptomics research and translational opportunities. Mol Psychiatry (2023). https://doi.org/10.1038/s41380-023-02339-x
4 He, P. C. & He, C. m (6) A RNA methylation: from mechanisms to therapeutic potential. EMBO J 40, e105977 (2021). https://doi.org/10.15252/embj.2020105977
5 Liu, J. et al. A METTL3-METTL14 complex mediates mammalian nuclear RNA N6-adenosine methylation. Nat Chem Biol 10, 93-95 (2014). https://doi.org/10.1038/nchembio.1432
6 Ping, X. L. et al. Mammalian WTAP is a regulatory subunit of the RNA N6-methyladenosine methyltransferase. Cell Res 24, 177-189 (2014). https://doi.org/10.1038/cr.2014.3
7 Wang, X. et al. N6-methyladenosine-dependent regulation of messenger RNA stability. Nature 505, 117-120 (2014). https://doi.org/10.1038/nature12730
8 Wang, X. et al. Structural basis of N (6)-adenosine methylation by the METTL3-METTL14 complex. Nature 534, 575-578 (2016). https://doi.org/10.1038/nature18298
9 Ruzicka, K. et al. Identification of factors required for m (6) A mRNA methylation in Arabidopsis reveals a role for the conserved E3 ubiquitin ligase HAKAI. New Phytol 215, 157-172 (2017). https://doi.org/10.1111/nph.14586
10 Wen, J. et al. Zc3h13 Regulates Nuclear RNA m (6) A Methylation and Mouse Embryonic Stem Cell Self-Renewal. Mol Cell 69, 1028-1038 e1026 (2018). https://doi.org/10.1016/j.molcel.2018.02.015
11 Yue, Y. et al. VIRMA mediates preferential m (6) A mRNA methylation in 3′UTR and near stop codon and associates with alternative polyadenylation. Cell Discov 4, 10 (2018). https://doi.org/10.1038/s41421-018-0019-0
12 Zheng, G. et al. ALKBH5 is a mammalian RNA demethylase that impacts RNA metabolism and mouse fertility. Mol Cell 49, 18-29 (2013). https://doi.org/10.1016/j.molcel.2012.10.015
13 Jia, G. et al. N6-methyladenosine in nuclear RNA is a major substrate of the obesity-associated FTO. Nat Chem Biol 7, 885-887 (2011). https://doi.org/10.1038/nchembio.687
14 Mauer, J. et al. FTO controls reversible m (6) Am RNA methylation during snRNA biogenesis. Nat Chem Biol 15, 340-347 (2019). https://doi.org/10.1038/s41589-019-0231-8
15 Wei, J. et al. Differential m (6) A, m (6) A (m), and m (1) A Demethylation Mediated by FTO in the Cell Nucleus and Cytoplasm. Mol Cell 71, 973-985 e975 (2018). https://doi.org/10.1016/j.molcel.2018.08.011
16 Deng, X. et al. RNA N6-methyladenosine modification in cancers: current status and perspectives. Cell Research 28, 507-517 (2018). https://doi.org/10.1038/s41422-018-0034-6
17 Yen, Y. P. & Chen, J. A. The m (6) A epitranscriptome on neural development and degeneration. J Biomed Sci 28, 40 (2021). https://doi.org/10.1186/s12929-021-00734-6
18 Yoon, K.-J. et al. Temporal Control of Mammalian Cortical Neurogenesis by m (6) A Methylation. Cell 171, 877-889.e817 (2017). https://doi.org/10.1016/j.cell.2017.09.003
19 Weng, Y.-L. et al. Epitranscriptomic m6A Regulation of Axon Regeneration in the Adult Mammalian Nervous System. Neuron 97, 313-325.e316 (2018). https://doi.org/https://doi.org/10.1016/j.neuron.2017.12.036
20 Meyer, Kate D. et al. Comprehensive Analysis of mRNA Methylation Reveals Enrichment in 3-UTRs and near Stop Codons. Cell 149, 1635-1646 (2012). https://doi.org/https://doi.org/10.1016/j.cell.2012.05.003
21 Wu, Z. et al. m (6) A epitranscriptomic regulation of tissue homeostasis during primate aging. Nat Aging 3, 705-721 (2023). https://doi.org/10.1038/s43587-023-00393-2
22 Yankova, E. et al. Small-molecule inhibition of METTL3 as a strategy against myeloid leukaemia. Nature 593, 597-601 (2021). https://doi.org/10.1038/s41586-021-03536-w
23 Barbieri, I. et al. Promoter-bound METTL3 maintains myeloid leukaemia by m (6) A-dependent translation control. Nature 552, 126-131 (2017). https://doi.org/10.1038/nature24678
24 Vu, L. P. et al. The N (6)-methyladenosine (m (6) A)-forming enzyme METTL3 controls myeloid differentiation of normal hematopoietic and leukemia cells. Nat Med 23, 1369-1376 (2017). https://doi.org/10.1038/nm.4416
25 Batista, P. J. et al. m (6) A RNA modification controls cell fate transition in mammalian embryonic stem cells. Cell Stem Cell 15, 707-719 (2014). https://doi.org/10.1016/j.stem.2014.09.019
26 Hsu, P. J., Shi, H. & He, C. Epitranscriptomic influences on development and disease. Genome Biol 18, 197 (2017). https://doi.org/10.1186/s13059-017-1336-6
27 Jonkhout, N. et al. The RNA modification landscape in human disease. RNA 23, 1754-1769 (2017). https://doi.org/10.1261/rna.063503.117
28 Chen, Y. G. et al. N6-Methyladenosine Modification Controls Circular RNA Immunity. Mol Cell 76, 96-109 e109 (2019). https://doi.org/10.1016/j.molcel.2019.07.016
29 Castro-Hernandez, R. et al. Conserved reduction of m (6) A RNA modifications during aging and neurodegeneration is linked to changes in synaptic transcripts. Proc Natl Acad Sci USA 120, e2204933120 (2023). https://doi.org/10.1073/pnas.2204933120
30 Todd, T. W. & Petrucelli, L. Modelling amyotrophic lateral sclerosis in rodents. Nat Rev Neurosci 23, 231-251 (2022). https://doi.org/10.1038/s41583-022-00564-x
31 Kim, G., Gautier, O., Tassoni-Tsuchida, E., Ma, X. R. & Gitler, A. D. ALS Genetics: Gains, Losses, and Implications for Future Therapies. Neuron 108, 822-842 (2020). https://doi.org/10.1016/j.neuron.2020.08.022
32 Mandrioli, J., Mediani, L., Alberti, S. & Carra, S. ALS and FTD: Where RNA metabolism meets protein quality control. Semin Cell Dev Biol 99, 183-192 (2020). https://doi.org/10.1016/j.semcdb.2019.06.003
33 Droppelmann, C. A., Campos-Melo, D., Ishtiaq, M., Volkening, K. & Strong, M. J. RNA metabolism in ALS: when normal processes become pathological. Amyotroph Lateral Scler Frontotemporal Degener 15, 321-336 (2014). https://doi.org/10.3109/21678421.2014.881377
34 Chen, T. H. & Chen, J. A. Multifaceted roles of microRNAs: From motor neuron generation in embryos to degeneration in spinal muscular atrophy. Elife 8 (2019). https://doi.org/10.7554/eLife.50848
35 McMillan, M. et al. RNA methylation influences TDP43 binding and disease pathogenesis in models of amyotrophic lateral sclerosis and frontotemporal dementia. Mol Cell 83, 219-236 e217 (2023). https://doi.org/10.1016/j.molcel.2022.12.019
36 Li, Y. et al. Globally reduced N (6)-methyladenosine (m (6) A) in C9ORF72-ALS/FTD dysregulates RNA metabolism and contributes to neurodegeneration. Nat Neurosci 26, 1328-1338 (2023). https://doi.org/10.1038/s41593-023-01374-9
37 Thams, S. et al. A Stem Cell-Based Screening Platform Identifies Compounds that Desensitize Motor Neurons to Endoplasmic Reticulum Stress. Mol Ther 27, 87-101 (2019). https://doi.org/10.1016/j.ymthe.2018.10.010
38 Tung, Y. T. et al. Mir-17 approximately 92 Confers Motor Neuron Subtype Differential Resistance to ALS-Associated Degeneration. Cell Stem Cell 25, 193-209 e197 (2019). https://doi.org/10.1016/j.stem.2019.04.016
39 Tung, Y. T. et al. Mir-17 approximately 92 Governs Motor Neuron Subtype Survival by Mediating Nuclear PTEN. Cell Rep 11, 1305-1318 (2015). https://doi.org/10.1016/j.celrep.2015.04.050
40 Xu, H. et al. m (6) A mRNA Methylation Is Essential for Oligodendrocyte Maturation and CNS Myelination. Neuron 105, 293-309 e295 (2020). https://doi.org/10.1016/j.neuron.2019.12.013
41 Patel, T. et al. Transcriptional dynamics of murine motor neuron maturation in vivo and in vitro. Nat Commun 13, 5427 (2022). https://doi.org/10.1038/s41467-022-33022-4
42 Liu, H., Begik, O. & Novoa, E. M. EpiNano: Detection of m (6) A RNA Modifications Using Oxford Nanopore Direct RNA Sequencing. Methods Mol Biol 2298, 31-52 (2021). https://doi.org/10.1007/978-1-0716-1374-0_3
43 Hendra, C. et al. Detection of m6A from direct RNA sequencing using a multiple instance learning framework. Nat Methods 19, 1590-1598 (2022). https://doi.org/10.1038/s41592-022-01666-1
44 Spataro, R. et al. Mutations in ATP13A2 (PARK9) are associated with an amyotrophic lateral sclerosis-like phenotype, implicating this locus in further phenotypic expansion. Hum Genomics 13, 19 (2019). https://doi.org/10.1186/s40246-019-0203-9
45 Blum, J. A. et al. Single-cell transcriptomic analysis of the adult mouse spinal cord reveals molecular diversity of autonomic and skeletal motor neurons. Nat Neurosci 24, 572-583 (2021). https://doi.org/10.1038/s41593-020-00795-0
46 Russ, D. E. et al. A harmonized atlas of mouse spinal cord cell types and their spatial organization. Nature Communications 12 (2021). https://doi.org/ARTN 5722 10.1038/s41467-021-25125-1
47 Bezprozvanny, I. Calcium signaling and neurodegenerative diseases. Trends Mol Med 15, 89-100 (2009). https://doi.org/10.1016/j.molmed.2009.01.001
48 Hung, S. T. et al. PIKFYVE inhibition mitigates disease in models of diverse forms of ALS. Cell 186, 786-802 e728 (2023). https://doi.org/10.1016/j.cell.2023.01.005
49 Price, B. D. & D′Andrea, A. D. Chromatin remodeling at DNA double-strand breaks. Cell 152, 1344-1354 (2013). https://doi.org/10.1016/j.cell.2013.02.011
50 Huang, Y. et al. Small-Molecule Targeting of Oncogenic FTO Demethylase in Acute Myeloid Leukemia. Cancer Cell 35, 677-691 e610 (2019). https://doi.org/10.1016/j.ccell.2019.03.006
51 Cerneckis, J., Ming, G. L., Song, H., He, C. & Shi, Y. The rise of epitranscriptomics: recent developments and future directions. Trends Pharmacol Sci 45, 24-38 (2024). https://doi.org/10.1016/j.tips.2023.11.002
52 Chen, J. A. & Wichterle, H. Apoptosis of limb innervating motor neurons and erosion of motor pool identity upon lineage specific dicer inactivation. Front Neurosci 6, 69 (2012). https://doi.org/10.3389/fnins.2012.00069
53 Ma, X. R. et al. TDP-43 represses cryptic exon inclusion in the FTD-ALS gene UNC13A. Nature 603, 124-130 (2022). https://doi.org/10.1038/s41586-022-04424-7
54 Hideyama, T. et al. Induced loss of ADAR2 engenders slow death of motor neurons from Q/R site-unedited GluR2. J Neurosci 30, 11917-11925 (2010). https://doi.org/10.1523/JNEUROSCI.2021-10.2010
55 Beaulieu, J. M., Jacomy, H. & Julien, J. P. Formation of intermediate filament protein aggregates with disparate effects in two transgenic mouse models lacking the neurofilament light subunit. J Neurosci 20, 5321-5328 (2000). https://doi.org/10.1523/JNEUROSCI.20-14-05321.2000
56 Mishra, P. S. et al. Transmission of ALS pathogenesis by the cerebrospinal fluid. Acta Neuropathol Commun 8, 65 (2020). https://doi.org/10.1186/s40478-020-00943-4
57 Wells, T. L., Myles, J. R. & Akay, T. C-Boutons and Their Influence on Amyotrophic Lateral Sclerosis Disease Progression. J Neurosci 41, 8088-8101 (2021). https://doi.org/10.1523/JNEUROSCI.0660-21.2021
58 Montanana-Rosell, R. et al. Spinal inhibitory neurons degenerate before motor neurons and excitatory neurons in a mouse model of ALS. Sci Adv 10, eadk3229 (2024). https://doi.org/10.1126/sciadv.adk3229
59 Nedialkova, D. D. & Leidel, S. A. Optimization of Codon Translation Rates via tRNA Modifications Maintains Proteome Integrity. Cell 161, 1606-1618 (2015). https://doi.org/10.1016/j.cell.2015.05.022
60 Helm, M. & Alfonzo, J. D. Posttranscriptional RNA Modifications: playing metabolic games in a cell's chemical Legoland. Chem Biol 21, 174-185 (2014). https://doi.org/10.1016/j.chembiol.2013.10.015
61 Lence, T. et al. m (6) A modulates neuronal functions and sex determination in Drosophila. Nature 540, 242-247 (2016). https://doi.org/10.1038/nature20568
62 Tam, O. H. et al. Postmortem Cortex Samples Identify Distinct Molecular Subtypes of ALS: Retrotransposon Activation, Oxidative Stress, and Activated Glia. Cell Rep 29, 1164-1177 e1165 (2019). https://doi.org/10.1016/j.celrep.2019.09.066
63 La Cognata, V., Gentile, G., Aronica, E. & Cavallaro, S. Splicing Players Are Differently Expressed in Sporadic Amyotrophic Lateral Sclerosis Molecular Clusters and Brain Regions. Cells 9 (2020). https://doi.org/10.3390/cells9010159
64 Wei, J. et al. FTO mediates LINE1 m (6) A demethylation and chromatin regulation in mESCs and mouse development. Science 376, 968-973 (2022). https://doi.org/10.1126/science.abe9582
65 Ruud, J. et al. The Fat Mass and Obesity-Associated Protein (FTO) Regulates Locomotor Responses to Novelty via D2R Medium Spiny Neurons. Cell Rep 27, 3182-3198 e3189 (2019). https://doi.org/10.1016/j.celrep.2019.05.037
66 Hess, M. E. et al. The fat mass and obesity associated gene (Fto) regulates activity of the dopaminergic midbrain circuitry. Nat Neurosci 16, 1042-1048 (2013). https://doi.org/10.1038/nn.3449
67 Shipley, M. M., Mangold, C. A. & Szpara, M. L. Differentiation of the SH-SY5Y Human Neuroblastoma Cell Line. J Vis Exp, 53193 (2016). https://doi.org/10.3791/53193
68 Maury, Y. et al. Combinatorial analysis of developmental cues efficiently converts human pluripotent stem cells into multiple neuronal subtypes. Nat Biotechnol 33, 89-96 (2015). https://doi.org/10.1038/nbt.3049
69 Maor-Nof, M. et al. p53 is a central regulator driving neurodegeneration caused by C9orf72 poly (PR). Cell 184, 689-708 e620 (2021). https://doi.org/10.1016/j.cell.2020.12.025
70 Kraemer, B. R. et al. A role for the p75 neurotrophin receptor in axonal degeneration and apoptosis induced by oxidative stress. J Biol Chem 289, 21205-21216 (2014). https://doi.org/10.1074/jbc.M114.563403
71 Yen, Y. P. et al. Dlk1-D₁₀3 locus-derived lncRNAs perpetuate postmitotic motor neuron cell fate and subtype identity. Elife 7 (2018). https://doi.org/10.7554/eLife.38080
72 Scott, S. et al. Design, power, and interpretation of studies in the standard murine model of ALS. Amyotroph Lateral Scler 9, 4-15 (2008). https://doi.org/10.1080/17482960701856300
73 Chang, S. H., Su, Y. C., Chang, M. E. & Chen, J. A. MicroRNAs mediate precise control of spinal interneuron populations to exert delicate sensory-to-motor outputs. Elife 10 (2021). https://doi.org/ARTN e63768 10.7554/eLife.63768

What is claimed is:

1. A degenerative motor neuron treatment composition comprising one or more therapeutically effective amount of N⁶-methyladenosine (m⁶A) eraser inhibitor, or a histone methylation regulator, or a methyl donor or a combination thereof capable of inhibiting or reversing motor neuron degeneration in a subject.

2. The composition of claim 1, wherein the m⁶A eraser inhibitor comprises an oligonucleotide wherein the nucleotide sequence of the oligonucleotide is at least about 80% identical to SEQ ID NO. 265, SEQ ID NO. 266, SEQ ID NO. 267, SEQ ID NO. 268, SEQ ID NO. 269, SEQ ID NO. 270, SEQ ID NO. 271, SEQ ID NO. 272, SEQ ID NO. 264 or SEQ ID NO. 273.

3. The composition of claim 1, wherein the m⁶A eraser inhibitor comprises an oligonucleotide capable of targeting a sequence selected from SEQ ID NOs. 1-263.

4. The composition of claim 1, wherein the m⁶A eraser inhibitor comprises an oligonucleotide capable of targeting a sequence selected from SEQ ID NO. 22, SEQ ID NO. 25, SEQ ID NO. 26, SEQ ID NO. 29, SEQ ID NO. 34, SEQ ID NO. 35, SEQ ID NO. 40, SEQ ID NO. 43, SEQ ID NO. 46, or SEQ ID NO. 47.

5. The composition of claim 1, wherein the composition comprises a m⁶A eraser inhibitor and a methyl donor, wherein the m⁶A eraser inhibitor comprises FB23-2, IOX1 or a combination thereof and wherein the methyl donor comprises S-adenosyl-L-methionine, betaine chloride, vitamin B9, vitamin B12 or a combination thereof.

6. The composition of claim 5, wherein the composition comprises FB23-2 and S-adenosyl-L-methionine at a molar ratio from about 10000:1 to about 100:1.

7. The composition of claim 5, wherein the composition comprises FB23-2 and betaine chloride at a molar ratio from about 50:1 to about 1:5.

8. The composition of claim 5, wherein the composition comprises FB23-2 and folic acid at a molar ratio from about 100:1 to about 1:1.

9. The composition of claim 5, wherein the composition comprises FB23-2, vitamin B12, and S-adenosyl-L-methionine at a molar ratio of about 10:3:1.

10. The composition of claim 5, wherein the composition comprises FB23-2, betaine chloride, and folic acid at a molar ratio of about 10:3:1.

11. The composition of claim 5, wherein the composition comprises FB23-2, IOX1, and S-adenosyl-L-methionine at a molar ratio of about 1000:20000:1.

12. The composition of claim 5, wherein the composition comprises IOX-1 and S-adenosyl-L-methionine at a molar ratio from about 200000:1 to about 2000:1.

13. The composition of claim 1, wherein the composition comprises two or more methyl donors, wherein the methyl donor comprises S-adenosyl-L-methionine, betaine chloride, vitamin B9, or vitamin B12.

14. The composition of claim 13, wherein the composition comprises vitamin B12 and S-adenosyl-L-methionine at a molar ratio from about 50:1 to about 1:5.

15. The composition of claim 1, wherein the composition comprises a m⁶A eraser inhibitor and a histone methylation regulator, wherein the m⁶A eraser inhibitor comprises FB23-2, IOX1, or a combination thereof, and wherein the histone methylation regulator comprises chaetocin, F5446, or a combination thereof.

16. The composition of claim 15, wherein the composition comprises a m⁶A eraser inhibitor and chaetocin at a molar ratio from about 2000:1 to about 1:5000.

17. The composition of claim 15, wherein the composition comprises a m⁶A eraser inhibitor and F5446 at a molar ratio from about 1000:1 to about 5000:1.

18. The composition of claim 1, further comprising a nanoparticle capable of serving as a delivery vehicle encapsulating one or more therapeutically effective amount of the m⁶A eraser inhibitor, the histone methylation regulator, the methyl donor or a combination thereof.

19. The composition of claim 18, wherein the nanoparticle comprises a self-complementary adeno-associated virus (scAAV).

20. The composition of claim 19, wherein the scAAV comprises scAAV9.

21. A method of treatment of degenerative motor neuron (MN) of a subject comprising the step of administering a therapeutically effective amount of the composition of claim 1 to the subject.

22. The method of claim 21, wherein the subject suffers from a motor neuron disease (MND) and wherein the MND comprises amyotrophic lateral sclerosis (ALS) comprising familial ALS, sporadic ALS, or a combination thereof, progressive bulbar palsy, primary lateral sclerosis, progressive muscular atrophy, spinal muscular atrophy, Kennedy's disease, or post-polio syndrome.

23. The method of claim 22, wherein the ALS comprises C9orf72-associated ALS, SOD1-associated ALS, TDP43-associated ALS, FUS-associated ALS, ALS associated with abnormalities derived from one or more genes comprising CCNF, NEK1, VCP, SQSTM1, OPTN, UBQLN2, PFN1, TUBA4A, MATR3, CHCHD10, TBK1, KIF5A, NEFH, NEFH, SETX, DCTN1, VAPB, CHMP2B, SPG11, HNRNPA2B1, HNRNPA1, CFAP410, ANXA11, ERLIN1, GLT8D1, DNAJC7, HTT, or SPTLC1, or a combination thereof.

24. The method of claim 21, wherein the subject is deficient in a m⁶A writer, a m⁶A methyltransferase, or a combination thereof, wherein the subject overexpresses a m⁶A eraser comprising FTO, ALKBH5, or a combination thereof or wherein the subject is deficient in METTL3, METTL5, METTL 14, METTL16, VIRMA, HAKAI, ZC3H13, RBM15/15B, WTAP, or a combination thereof.

25. The method of claim 21, wherein the degenerative MN of the subject comprises histone hypermethylation or H3K9me3 hypermethylation.

26. The method of claim 21, wherein the step of administering a therapeutically effective amount of the composition of claim 1 to the subject results in raising the m⁶A level of a genetic sequence associated with one or more genes of List 1 or List 2.

27. A method of preparation of a MN degeneration model comprising the step of reducing the expression of METTL3, METTL14, or a combination thereof in a model subject.

28. The method of claim 27, wherein the step of reducing the expression of METTL3, METTL14, or a combination thereof in a model subject comprises administration of METLL3 inhibitor, a METTL14 inhibitor, or a combination thereof to the model subject.

29. The method of claim 28, wherein the METTL3 inhibitor comprises a small molecule compound comprising STM2457, STC15, an oligonucleotide capable of targeting METTL3, or a combination thereof.

30. The method of claim 29, wherein nucleotide sequence of the oligonucleotide capable of targeting expression of METTL3 is at least about 80% identical to a sequence selected from SEQ ID NO. 274, SEQ ID NO. 275, SEQ ID NO. 276, or SEQ ID NO. 277.

31. The method of claim 28, wherein the METTL14 inhibitor comprises a small molecule compound, an oligonucleotide capable of targeting METTL14, or a combination thereof.

32. The method of claim 31, wherein nucleotide sequence of the oligonucleotide capable of targeting expression of METTL14 is at least about 80% identical to a sequence selected from SEQ ID NO. 278, SEQ ID NO. 279, SEQ ID NO. 280, or SEQ ID NO. 281.

33. The method of claim 27, wherein the step of reducing the expression of METTL3, METTL14, or a combination thereof in a subject comprises the step of application to the model subject of a Cre-loxP system capable of effecting conditional knockout of the METTL3 gene, the METTL14 gene, or a combination thereof in the model subject.

34. The method of claim 33, wherein the expression of Cre recombinase is driven by choline O-acetyltransferase (ChAT) or oligodendrocyte transcription factor 2 (Olig2).

35. A MN degeneration model comprising a model subject prepared using the process of claim 27 to result in the model subject having deficiency in METTL3 expression, METTL14 expression, or a combination thereof.

36. The model of claim 35, wherein the m⁶A level in the model subject is at least about 5% to about 100% less than the m⁶A level in a subject not deficient in METTL3 expression, METTL14 expression, or a combination thereof.

37. The model of claim 35, wherein the model subject comprises a cell or an animal such as but not limited to a human or a mouse.

38. The model of claim 37, wherein the model subject comprises a cell derived from an animal such as an iPSC-derived MN.
